(19) United States
(12) Patent Application Publication
(10) Pub. No.: US 2024/0318160 A1
(43) Pub. Date: Sep. 26, 2024

(54) TWO COMBINED MUTATIONS THAT INTRODUCE THE SECOND ENTRY PATHWAY TO SYNTHESIZED LIGNIN FROM TYROSINE IN PLANTS

(71) Applicant: Wisconsin Alumni Research Foundation, Madison, WI (US)

(72) Inventors: Hiroshi Maeda, Madison, WI (US); Yuri Takeda-Kimura, Madison, WI (US); Bethany Moore, Madison, WI (US)

(21) Appl. No.: 18/611,181

(22) Filed: Mar. 20, 2024

Related U.S. Application Data
(60) Provisional application No. 63/491,152, filed on Mar. 20, 2023.

(51) Int. Cl. C12N 9/88; C12N 9/02; C12N 9/10; C12N 15/82

(52) U.S. Cl. CPC C12N 9/88; C12N 9/001; C12N 9/1085; C12N 15/8255; C12Y 103/01012; C12Y 103/01043; C12Y 205/01054; C12Y 403/01024

(57) ABSTRACT

The present invention provides engineered phenylalanine ammonia-lyase (PAL) enzymes comprising one or more mutations that increase the enzymes' tyrosine ammonia-lyase (TAL) activity. Also provided are plants comprising the engineered PAL enzymes and methods of using these plants to sequester CO₂ or produce phenylpropanoid-derived products.

### CROSS-REFERENCE TO RELATED APPLICATIONS

This application claims priority to U.S. Provisional Application No. 63/491,152, filed on Mar. 20, 2023, the contents of which are incorporated by reference in their entireties.

### STATEMENT REGARDING FEDERALLY SPONSORED RESEARCH

This invention was made with government support under grant number 1836824 awarded by the National Science Foundation. The government has certain rights in the invention.

### SEQUENCE LISTING

This application includes a sequence listing in XML format titled “960296.04479_ST26.xml”, which is 356,334 bytes in size and was created on Mar. 14, 2024. The sequence listing is electronically submitted with this application via Patent Center and is incorporated herein by reference in its entirety.

### BACKGROUND

Lignin is a complex organic polymer that is used as a structural material to support the tissues of land plants. It comprises up to 30% of plant dry mass and is the most abundant aromatic polymer on earth. Engineering the lignin biosynthesis pathway is a potential way to increase carbon sequestration in plants and to enhance the value of plant biomass for use in the production of bioenergy and biomaterials. Accordingly, there is a need in the art for methods of altering this pathway.

### SUMMARY

In a first aspect, the present invention provides engineered phenylalanine ammonia-lyase (PAL) enzymes that have increased tyrosine ammonia-lyase (TAL) activity. These engineered PAL enzymes comprise a first mutation at a position corresponding to residue 112 of SEQ ID NO: 28 and a second mutation at a position corresponding to residue 140 of SEQ ID NO: 28 in a wild-type PAL enzyme and have increased TAL activity relative to the wild-type PAL enzyme.

In a second aspect, the present invention provides polynucleotides encoding an engineered PAL enzyme described herein.

In a third aspect, the present invention provides constructs comprising a promoter operably linked to a polynucleotide described herein.

In a fourth aspect, the present invention provides vectors comprising a polynucleotide or construct described herein.

In a fifth aspect, the present invention provides cells comprising an engineered PAL enzyme, polynucleotide, construct, or vector described herein.

In a sixth aspect, the present invention provides seeds comprising an engineered PAL enzyme, polynucleotide, construct, vector, or cell described herein.

In a seventh aspect, the present invention provides plants grown from a seed described herein and plants comprising an engineered PAL enzyme, polynucleotide, construct, vector, or cell described herein.

In an eighth aspect, the present invention provides methods of making the plants described herein.

In a ninth aspect, the present invention provides methods for using the plants described herein to (1) produce a phenylpropanoid-derived product or (3) sequester carbon dioxide. The methods comprise growing the plants. The methods for producing phenylpropanoid-derived products further comprise purifying the phenylpropanoid-derived products produced by the plant.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1B show that grasses possess a tyrosine-derived lignin biosynthesis pathway. FIG. 1A shows a phylogenetic tree of Poales species. The tree was retrieved from Givnish et al. (2010) and Seetharam et al. (2021), with some modifications. FIG. 1B shows a schematic depiction of the lignin biosynthetic pathway in grasses. While most vascular plants mainly synthesize lignin from phenylalanine (L-Phe) using the enzyme phenylalanine ammonia-lyase (PAL), grasses can also synthesize lignin from tyrosine (L-Tyr) using the enzyme phenylalanine tyrosine ammonia-lyase (PTAL) via an additional shortcut pathway.

FIGS. 2A-2C show that PTAL enzymes emerged in the common ancestor of grasses and the non-grass graminids Joinvillea, just before the emergence of grasses. FIG. 2A shows a phylogenetic tree of PAL/PTAL genes in monocots, focusing on Poales species. The tree was built using RAxML-ng from the PAL/PTAL orthogroup from Orthofinder in plants. The PAL/PTAL homologs that are characterized in this study are highlighted. FIG. 2B is a graph showing the Km and kcat of the TAL activity of PTAL/PAL enzymes from the grasses Sorghum bicolor (SbPTAL and SbPAL) and Brachypodium distachion (BdPTAL and BdPAL) as well as PTAL homologs from Streptochaeta angustifolia (SaPTAL-a and SaPTAL-b), Joinvillea ascendens (JaPTAL and JaPAL), and Ecdeiocolea monostachya (EmoPTAL and EmoPAL). Michaelis-Menten curves for the TAL and PAL assays for JaPTAL and JaPAL are shown below. FIG. 2C is a graph showing the ratio of TAL and PAL activity (kcat/Km) of PAL and PTAL enzymes from grasses and non-grass graminids.

FIGS. 3A-3C demonstrate that multiple amino acid residues are critical for the transition from PAL to PTAL. FIG. 3A is a graph showing the Km and kcat of TAL activity for PTAL/PAL enzymes (i.e., SbPTAL, BdPTAL, SaPTAL-a, SaPTAL-b, EmoPTAL, JaPTAL, JaPAL, EmoPAL, SbPAL, and BdPAL) comprising a mutation at a position corresponding to residue 140 in JaPAL (SEQ ID NO: 28). FIG. 3B is a partial amino acid sequence alignment highlighting (1) residue His/Phe 140, which has been reported to be critical for recognition of the substrates phenylalanine and tyrosine (*), (2) residues that are highly conserved and distinct between PTAL or PAL enzymes (circle), and residues that are highly conserved among PTAL enzymes but not among PAL enzymes (triangle). A full-length alignment is provided in FIG. 8.

FIG. 3C is a set of graphs showing the Km and kcat of TAL and PAL activity for wild-type and mutant JaPTAL and JaPAL enzymes, including JaPAL mutants with mutations at residue 140 (JaPAL^{F140H}) as well as mutants with mutations at the 8 residues highlighted with circles in FIG. 3A (JaPAL^{F140H_MUT8}) and mutants with mutations at the 16 residues highlighted with circles and triangles in FIG. 3A (JaPAL^{F140H_MUT16}). Different letters indicate a significant difference (ANOVA with post hoc Tukey-Kramer method, p<0.05).

FIGS. 4A-4D demonstrate that the residue Ser 112 is critical for the acquisition of TAL activity. FIG. 4A is a graph showing the Km and kcat of TAL activity for JaPAL^{F140H_MUT8} variants in which one of the eight additional mutations has been reversed. FIG. 4B is a schematic depiction of a potential TAL reaction mechanism, showing hypothetical roles for the residues His 140 and Ile112 in PTAL enzyme catalysis. Ser/Ile 112 is located next to Tyr113, which is critical for catalysis, and these residues are in the ‘inner mobile loop’, which has been suggested to function in substrate binding and catalysis. FIG. 4C is a graph showing the Km and kcat of TAL activity for JaPAL enzymes with mutations at residue 140 (JaPAL^{F140H}), residue 112 (JaPAL^{S112I}), or both residue 140 and residue 112 (JaPAL^{F140H_S112I}). FIG. 4D is a graph showing the Km and kcat of TAL activity for Arabidopsis AtPAL1 enzymes with a mutation at a position corresponding to residue 140 of JaPAL (AtPAL1^{F144H}), a position corresponding to residue 112 of JaPAL (AtPAL1^{S116I}), or at positions corresponding to both residue 140 and residue 112 of JaPAL (AtPAL1^{F144H_S116I}). Different letters indicate a significant difference (ANOVA with post hoc Tukey-Kramer method, p<0.05).

FIG. 5 is a phylogenetic tree of PAL/PTAL genes in green plants. The tree was built using RAxML-ng from the PAL/PTAL orthogroup from Orthofinder in plants. Species used as input for the Orthofinder run are listed in Table 1.

FIG. 6 shows a phylogenetic tree of PAL/PTAL genes in monocots. The tree was built from the PAL/PTAL orthogroup from Orthofinder using monocot species and the basal species Amborella trichopoda. Genes from Amborella are the outgroup. The PTAL clade includes genes that are known to have PTAL function in grasses, whereas the PAL clade includes genes for which only PAL function is known in grasses. Species used as input for the Orthofinder run are listed in Table 2.

FIG. 7 shows high-performance liquid chromatography (HPLC) chromatograms for TAL and PAL reaction products produced by PTAL/PAL enzymes from B. distachyon and J. ascendans.

FIG. 8 is a full-length alignment of PTAL and PAL protein sequences from monocots (clade I). The sequences shown is the alignment are SEQ ID NO: 1-143, ordered from top to bottom. These sequences are detailed in Table 8. PTAL sequences (SEQ ID NO: 1-27) are shown at the top of each page. PAL sequences are divided into three categories below: basal grass PAL (SEQ ID NO: 28-30), grass PAL (SEQ ID NO: 31-88), and monocot PAL (SEQ ID NO: 89-143). Residues that are required for general aromatic ammonia-lyase activity are denoted with a square. The 16 residues identified by phylogeny-guided alignment analysis are denoted with triangles and circles. These residues include 8 residues that are highly conserved among both PTAL and PAL enzymes but different between them (circles) and 8 residues are highly conserved among PTALs but not among PALs (triangles).

FIGS. 9A-9B demonstrate that several different substitutions at residue 112 confer TAL activity. FIG. 9A is a phylogenetic tree of PAL/PTAL genes in green plants. The amino acids Ser and Ile are well conserved at positions corresponding to residue 112 in JaPAL (SEQ ID NO: 28) in angiosperm PAL enzymes, but basal non-flower PAL enzymes possess Ile, Thr, or Val at this position. FIG. 9B is a set of graphs showing the TAL and PAL activity of JaPAL and JaPTAL enzymes with mutations at residue 112. Substituting the Ile at this position in JaPAL^{F140H_S112I} with Thr or Val retains strong TAL activity but substituting it with Ser does not.

### DETAILED DESCRIPTION

The present invention provides engineered phenylalanine ammonia-lyase (PAL) enzymes comprising one or more mutations that increase the enzymes' tyrosine ammonia-lyase (TAL) activity. Also provided are plants comprising the engineered PAL enzymes and methods of using these plants to sequester CO₂ or produce phenylpropanoid-derived products.

Most vascular plants synthesize lignin from the amino acid phenylalanine using the enzyme phenylalanine ammonia-lyase (PAL). However, grass plants possess a bifunctional enzyme, phenylalanine tyrosine ammonia-lyase (PTAL), that allows them to synthesize lignin and other phenylpropanoids using either phenylalanine or tyrosine as a substrate. To better understand how PTAL enzymes evolved in grasses, the inventors identified orthologs of grass PTAL enzymes in other, closely related plants. Biochemical characterization of these orthologs revealed that PTAL enzymes are found, not only in grasses, but also in the non-grass graminid Joinvillea ascendans, which indicates that PTAL enzymes emerged before the evolution of grasses.

It was previously reported that a particular residue, referred to herein as His/Phe 140, determines whether PAL/PTAL enzymes have TAL activity in bacteria. However, the inventors discovered that both His 140 and an additional residue, Ile112, are required for TAL activity in plants. They demonstrate that introducing Ile 112 and His 140 into the monofunctional PAL enzymes of J. ascendans and Arabidopsis thaliana converts them into bifunctional PTAL enzymes. Thus, these residues represent novel gene editing targets that can be used to introduce the alternative TAL pathway into plants. Creating genetically engineered plants that can use both phenylalanine and tyrosine to synthesize lignin and phenylpropanoids should increase the carbon flow into these synthesis pathways and increase the amount of carbon sequestered by the plants. Further, it should increase the phenylpropanoid content of the plants, which may increase the value of their plant material, strengthen their disease resistance, and/or improve their nutritional quality.

While others have previously shown that overexpressing PAL enzymes (Phytochemistry, 64: 153-161, 2003) or expressing bacterial TAL enzymes in transgenic plants (Planta, 232: 209-218, 2010) have some effect on the production of phenylpropanoid-derived compounds, the inventors predict that engineering the native PAL enzymes of plants to introduce TAL activity will more effectively increase carbon flow into the phenylpropanoid synthesis pathway as compared to PAL overexpression (i.e., because TAL activity is more efficient than PAL activity, see below) while avoiding the need to introduce a transgene from another organism into the plant.

### Enzymes:

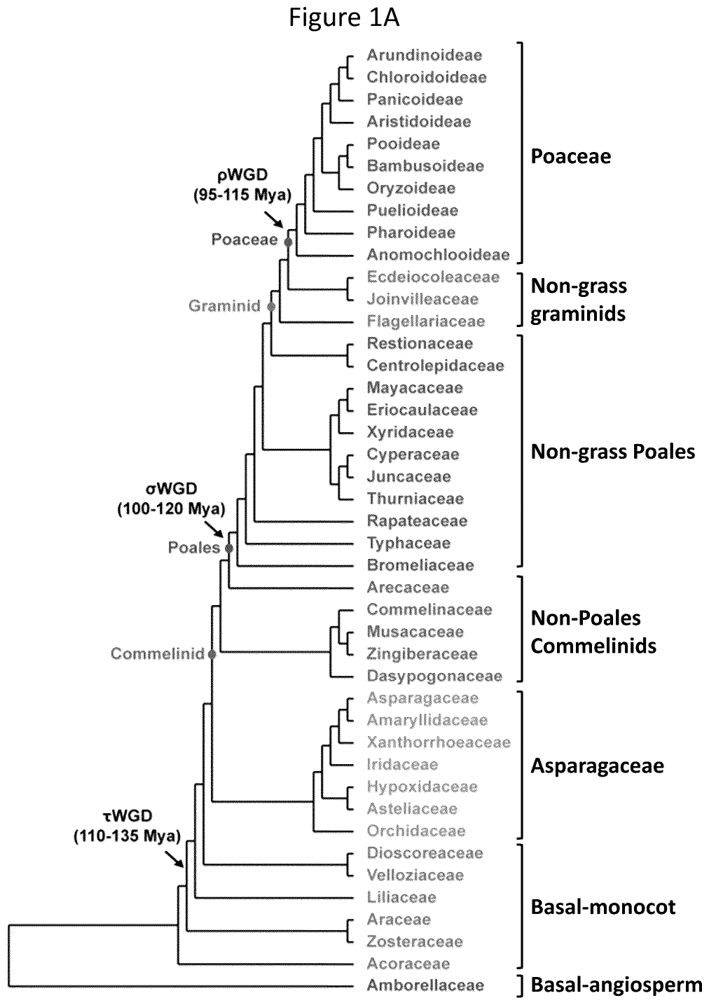

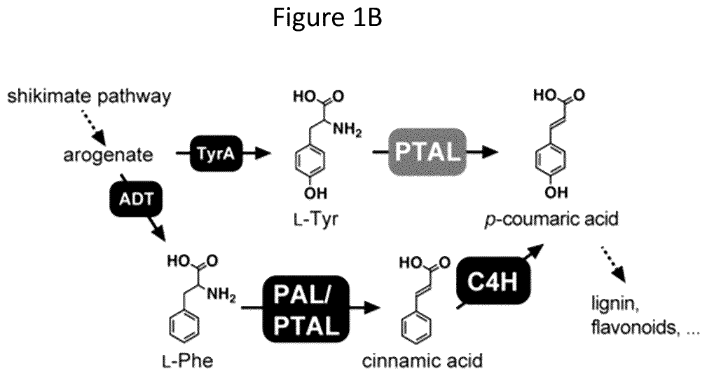

Land plants produce a diverse array of phenylpropanoid compounds, which include polymers, such as lignin, suberin, and condensed tannin, as well as soluble metabolites, such as flavonoids, coumarin, stilbenes, and phenylpropenes. In most plants, the first step in the phenylpropanoid biosynthetic pathway is the deamination of the amino acid phenylalanine into trans-cinnamic acid (FIG. 1B). This reaction is typically catalyzed by the monofunctional enzyme phenylalanine ammonia-lyase (PAL). The second step in this pathway is typically the hydroxylation of trans-cinnamic acid to p-coumaric acid, which is catalyzed by the enzyme cinnamate 4-hydroxylase (C4H). However, plants that express the bifunctional enzyme phenylalanine tyrosine ammonia-lyase (PTAL) can synthesize p-coumarate either (1) from phenylalanine using the same two-step, two-enzyme process, or (2) from tyrosine using a more efficient, one-step process that avoids the rate-limiting C4H step. Thus, in addition to having phenylalanine ammonia-lyase (PAL) activity, PTAL enzymes have tyrosine ammonia-lyase (TAL) activity. As a result, they can use either phenylalanine or tyrosine as a substrate.

The PAL and PTAL enzymes of the non-grass graminid Joinvillea ascendens are used as reference sequences herein. These enzymes are referred to as JaPAL (protein sequence: SEQ ID NO: 28, DNA sequence: SEQ ID NO: 147) and JaPTAL (protein sequence: SEQ ID NO: 27, DNA sequence: SEQ ID NO: 151).

“Tyrosine ammonia-lyase (TAL) activity” is enzyme activity that converts the amino acid tyrosine into p-coumaric acid via non-oxidative deamination. PAL enzymes naturally lack or have trace levels TAL activity, whereas PTAL enzymes naturally possess strong TAL activity. However, in the Examples, the inventors demonstrate that TAL activity can be introduced into or dramatically increased in PAL enzymes via the introduction of mutations at two specific residues. The TAL activity of an engineered PAL enzyme of the present invention may be increased by 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, or more as compared to the TAL activity of the corresponding wild-type PAL enzyme. The TAL activity of an enzyme can be assessed using TAL activity assays, in which the reaction products formed by the enzyme in the presence of the substrate tyrosine are measured. For example, TAL activity can be assessed by measuring the production of the product p-coumaric acid using high-performance liquid chromatography (HPLC) or by measuring absorbance at 309 nm (e.g., using a plate reader). TAL activity can also be assessed by measuring the release of ammonia from the reaction. See Example 1 for a description of such assays.

Thus, in a first aspect, the present invention provides engineered phenylalanine ammonia-lyase (PAL) enzymes that have increased tyrosine ammonia-lyase (TAL) activity. An “enzyme” is a protein or RNA molecule that acts as a catalyst in living organism. Enzymes decrease the activation energy required for a chemical reaction to occur by stabilizing the transition state.

The engineered PAL enzymes described herein may be full-length proteins or may be fragments of full-length proteins. As used herein, a “fragment” is a portion of a protein that is identical in sequence to, but shorter in length than, the full-length protein. For example, a fragment may comprise at least 5, 10, 15, 20, 25, 30, 40, 50, 60, 70, 80, 90, 100, 150, 250, or 500 contiguous amino acid residues of a full-length protein. Fragments may be preferentially selected from certain regions of a protein. A fragment may comprise an N-terminal truncation, a C-terminal truncation, or both an N-terminal and C-terminal truncation relative to the full-length protein. Preferably, the PAL enzyme fragments used with the present invention are functional fragments. As used herein, the term “functional fragment” refers to a fragment that retains at least 20%, 40%, 60%, 80%, or 100% of the PAL/TAL activity of the corresponding full-length protein.

The PAL enzymes described herein are “engineered,” meaning that they have been altered by the hand of man. Specifically, the PAL enzymes of the present invention have been engineered to comprise one or more mutations. As used herein, the term “mutation” refers to a difference in an amino acid sequence relative to a reference sequence (e.g., the sequence of a wild-type PAL enzyme). Mutations include insertions, deletions, and substitutions of an amino acid relative to a reference sequence. An “insertion” refers to a change in an amino acid sequence that results in the addition of one or more amino acid residues. An insertion may add 1, 2, 3, 4, 5, 10, 20, 30, 40, 50, 60, 70, 80, 90, 100, 150, 200, or more amino acid residues to a sequence. A “deletion” refers to a change in an amino acid sequence that results in the removal of one or more amino acid residues. A deletion may remove 1, 2, 3, 4, 5, 10, 20, 50, 100, 200, or more amino acids residues from a sequence. A “substitution” refers to a change in an amino acid sequence in which one amino acid is replaced with a different amino acid. An amino acid substitution may be a conversative replacement (i.e., a replacement with an amino acid that has similar properties) or a radical replacement (i.e., a replacement with an amino acid that has different properties).

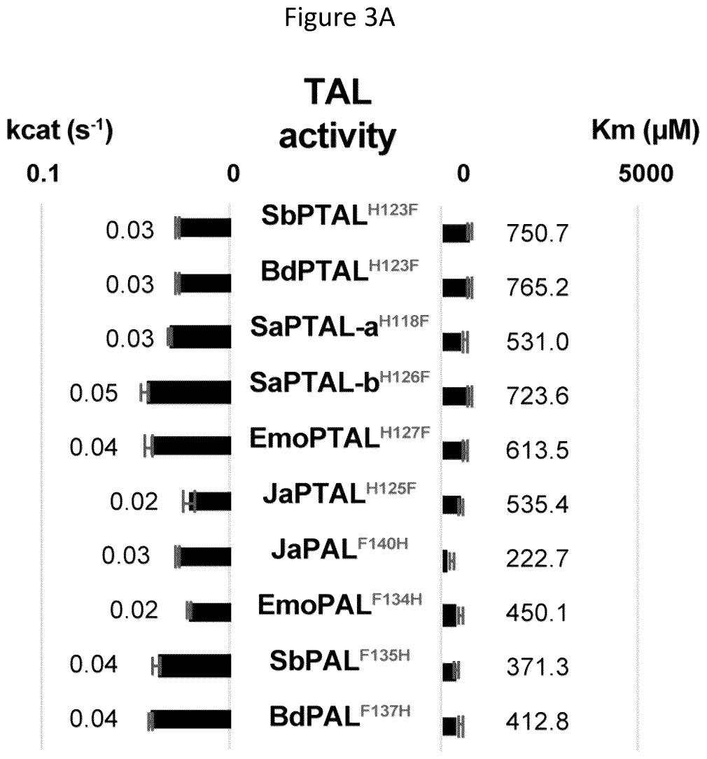

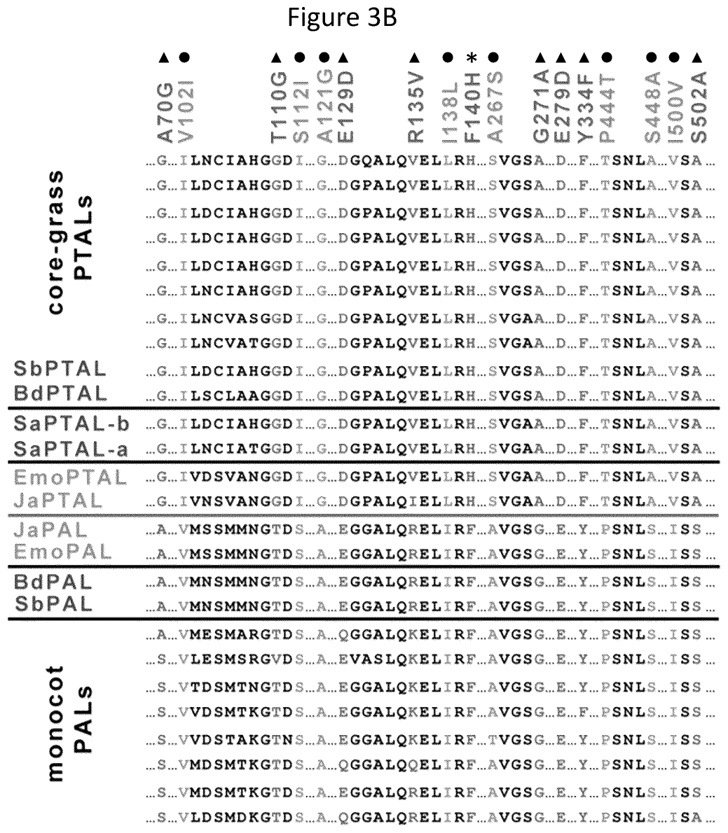

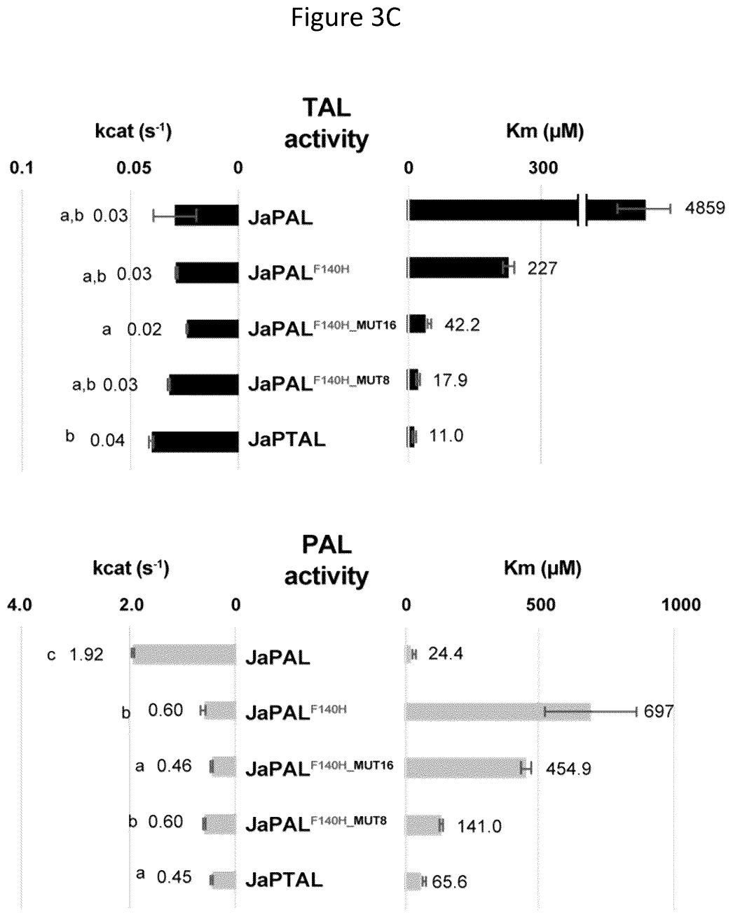

The engineered PAL enzymes of the present invention comprise one or more mutations relative to the corresponding wild-type PAL enzyme. The term “wild-type” is used herein to describe the non-mutated version of an enzyme that is most typically found in nature. Wild-type PAL enzymes comprise a serine at the position corresponding to residue 112 of SEQ ID NO: 28 (Ser112) and comprise a phenylalanine at the position corresponding to residue 140 of SEQ ID NO: 28 (Phe 140), whereas wild-type PTAL enzymes comprise an isoleucine at the position corresponding to residue 112 of SEQ ID NO: 28 (Ile112) and comprise a histidine at the position corresponding to residue 140 of SEQ ID NO: 28 (His140) (see, e.g., FIG. 3B). The engineered PAL enzymes of the present invention comprise a mutation at a position corresponding to residue 112 of SEQ ID NO: 28, and optionally further comprise a second mutation at a position corresponding to residue 140 of SEQ ID NO: 28.

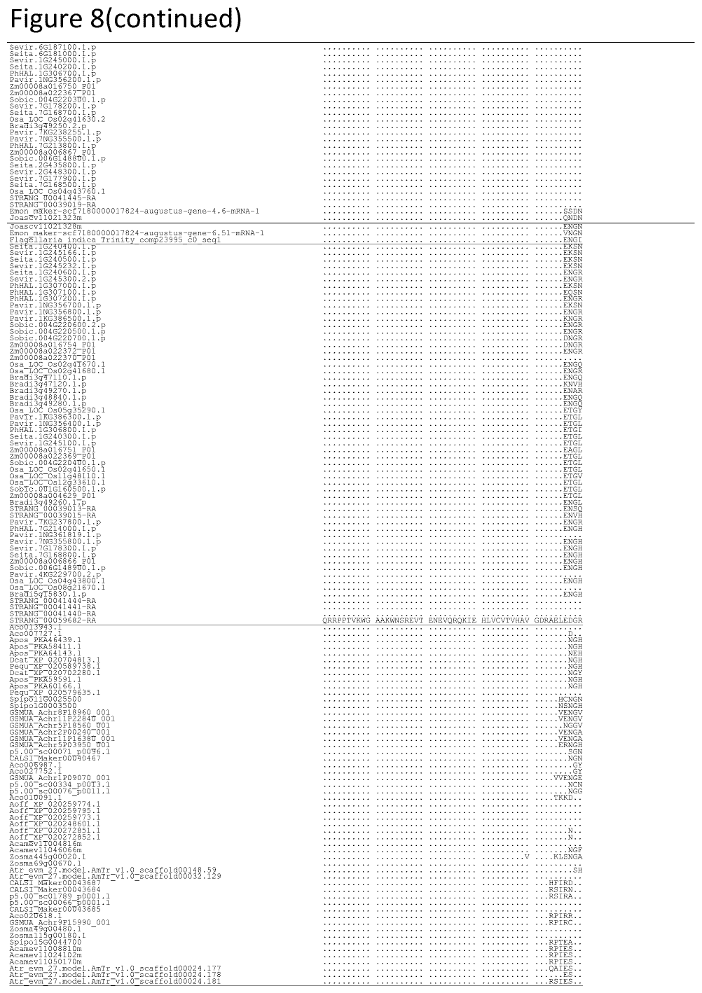

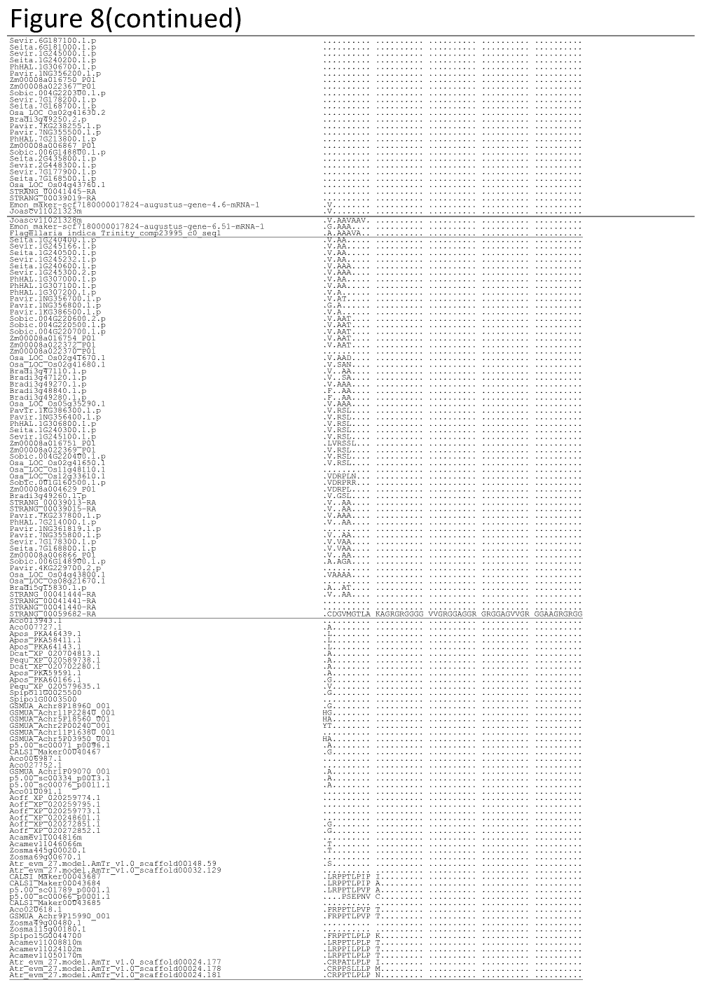

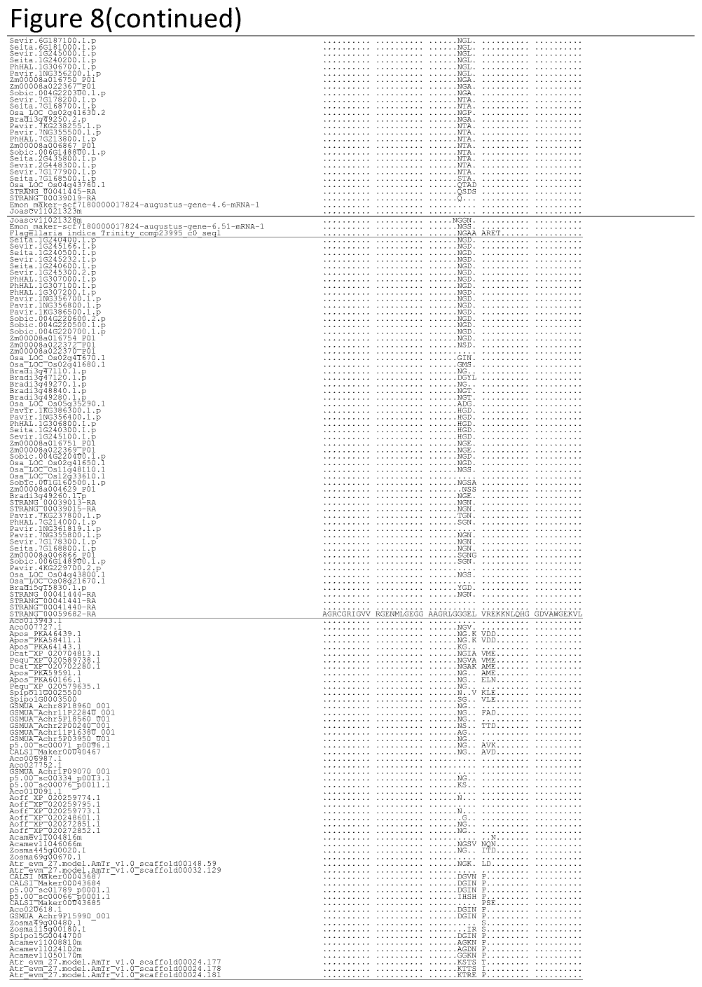

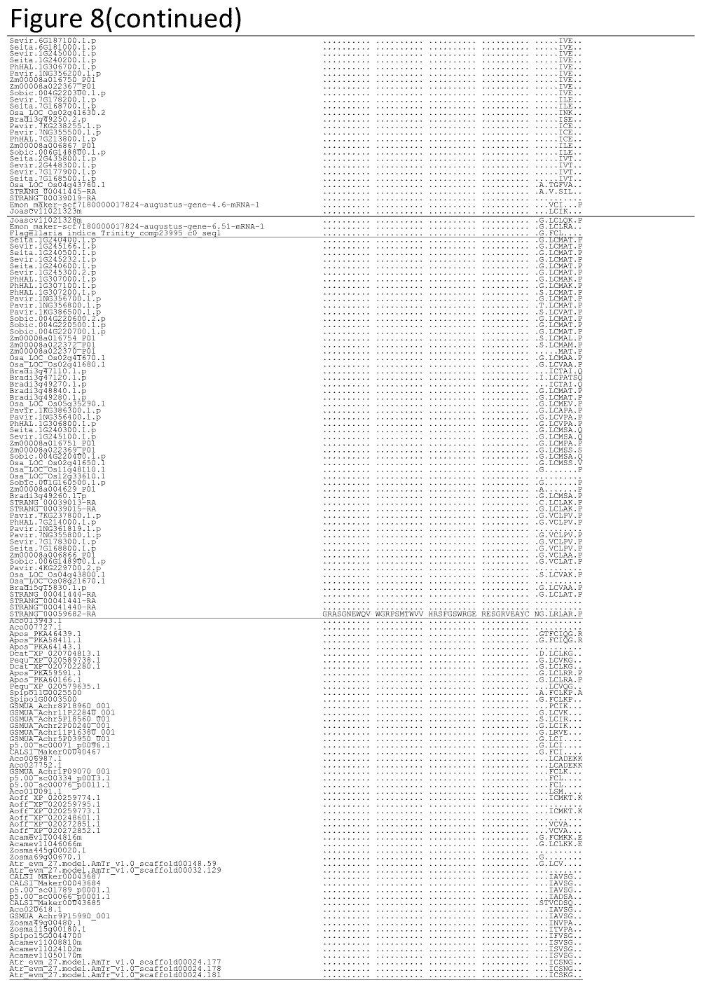

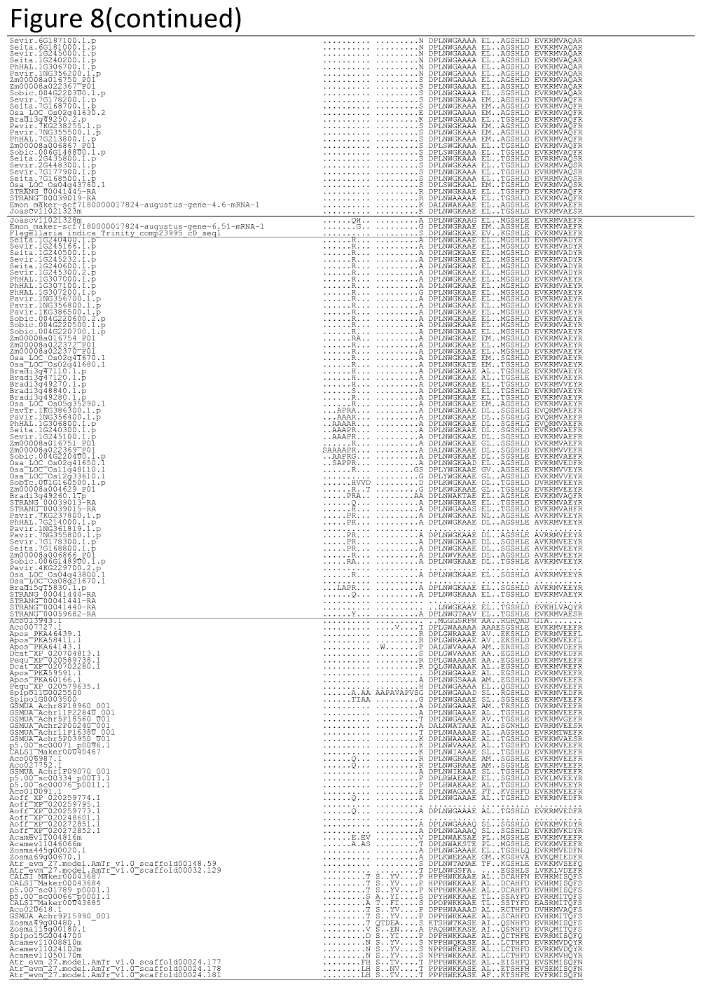

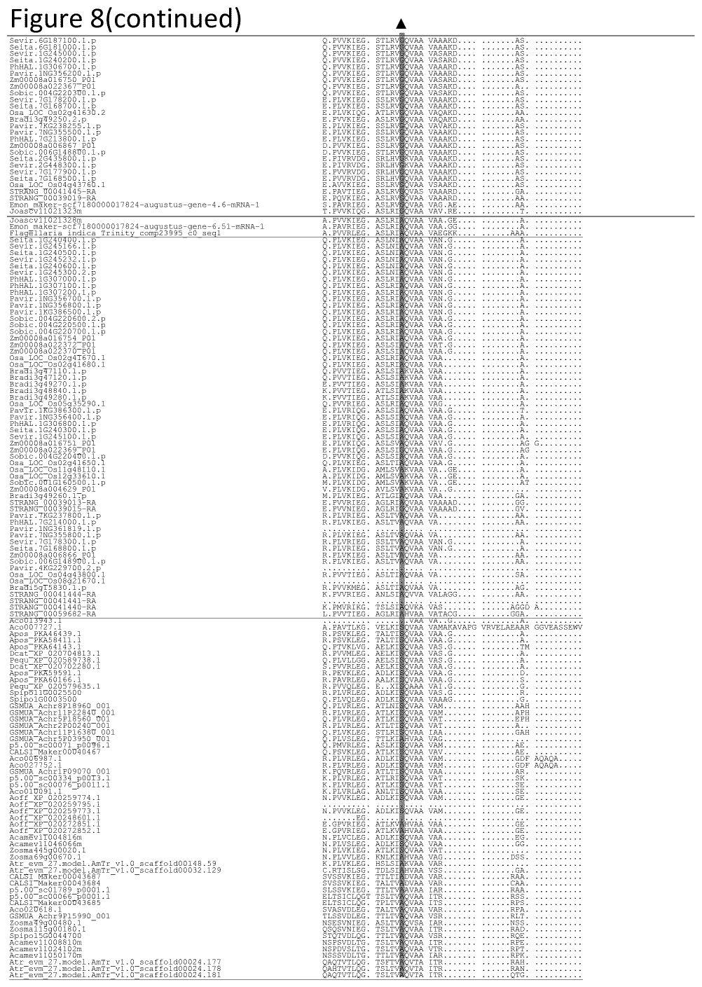

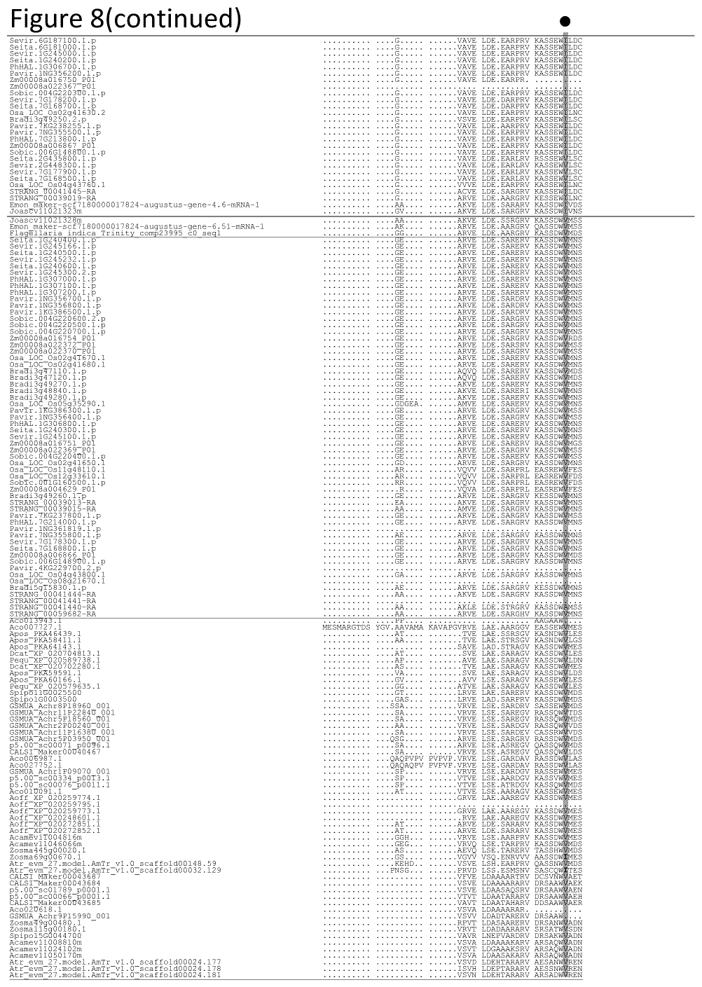

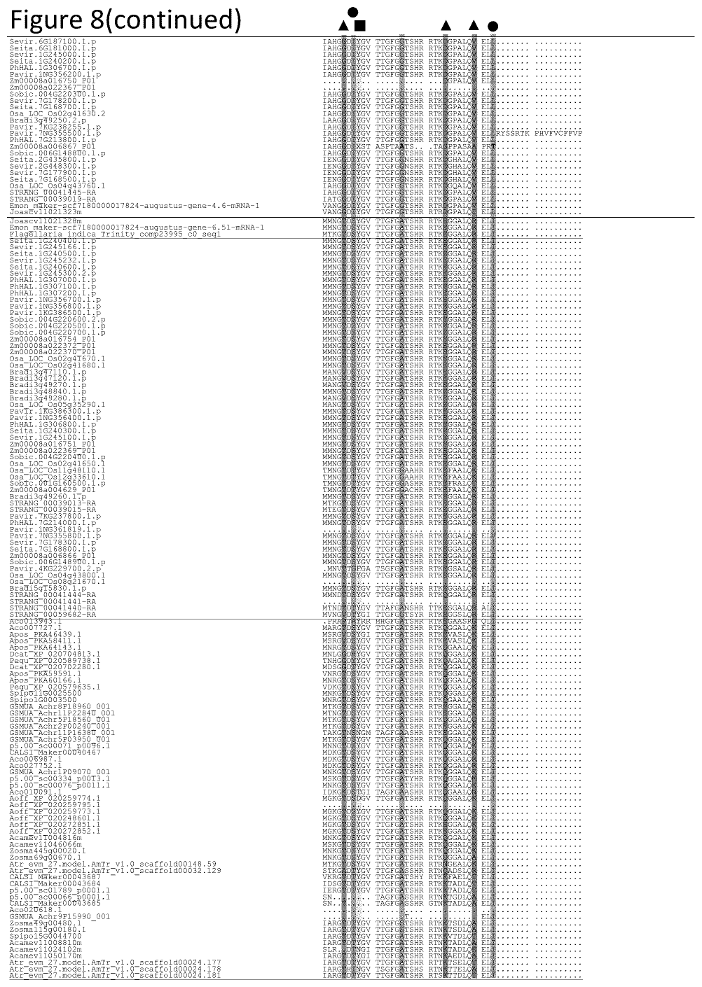

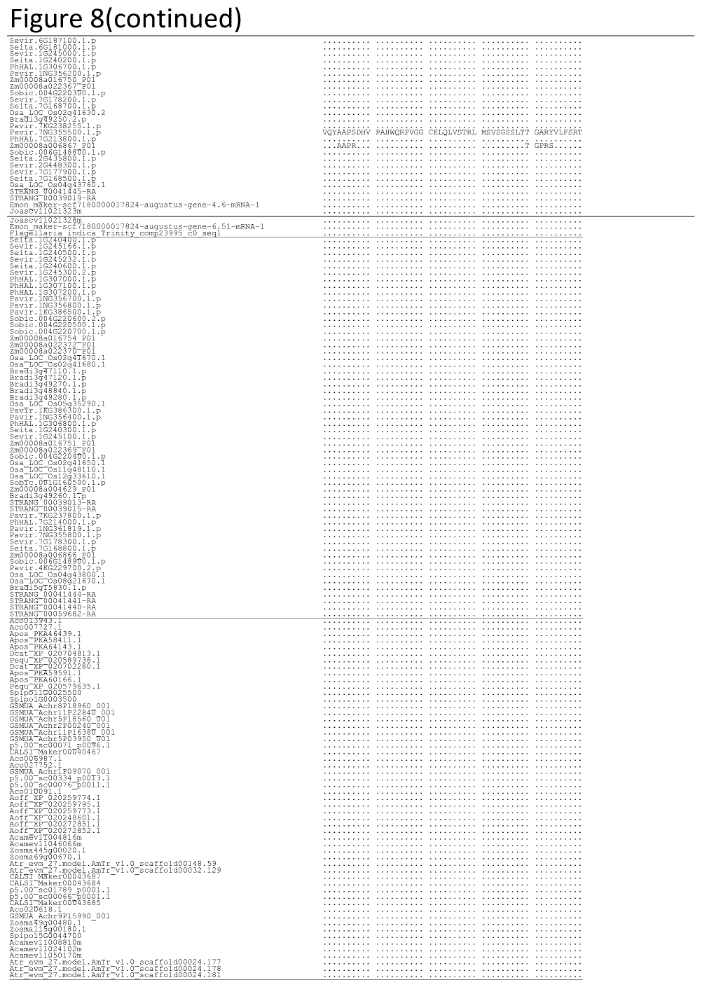

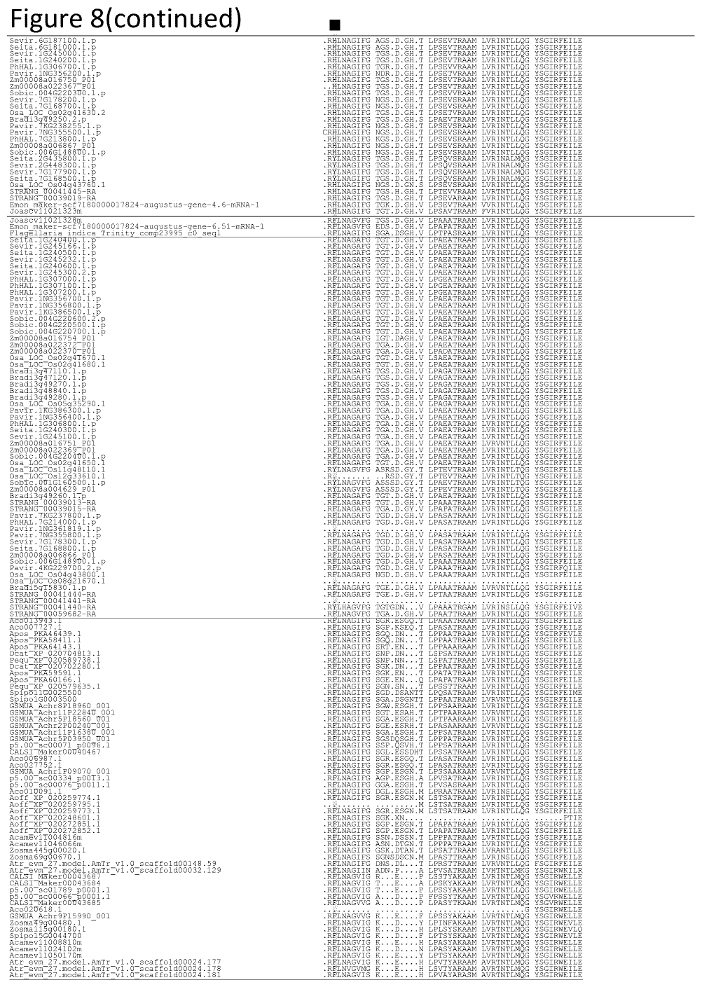

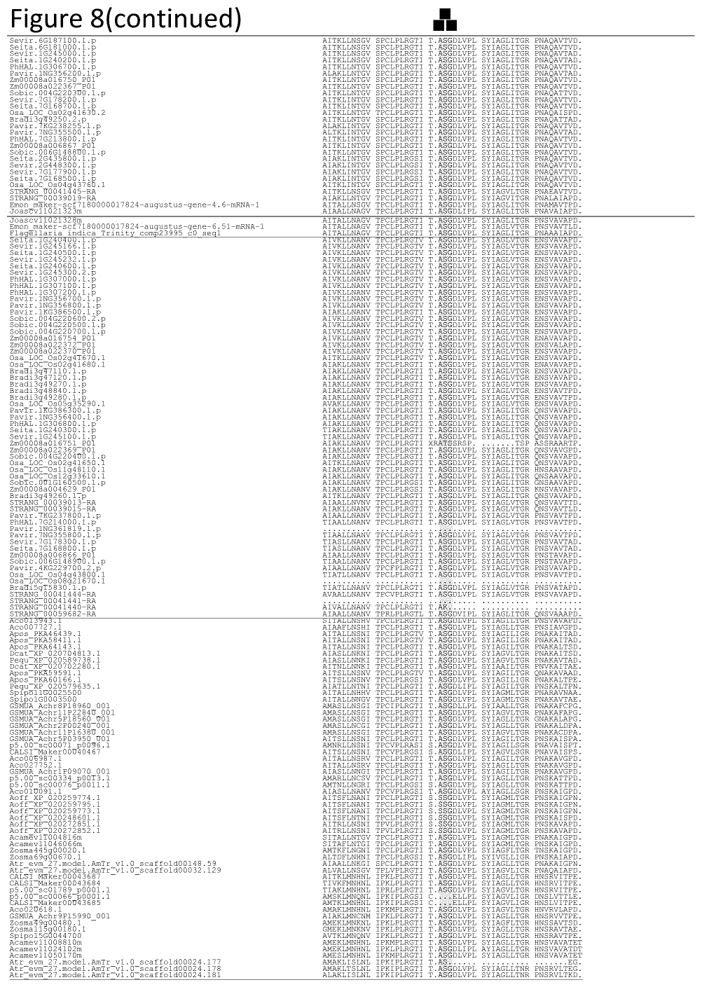

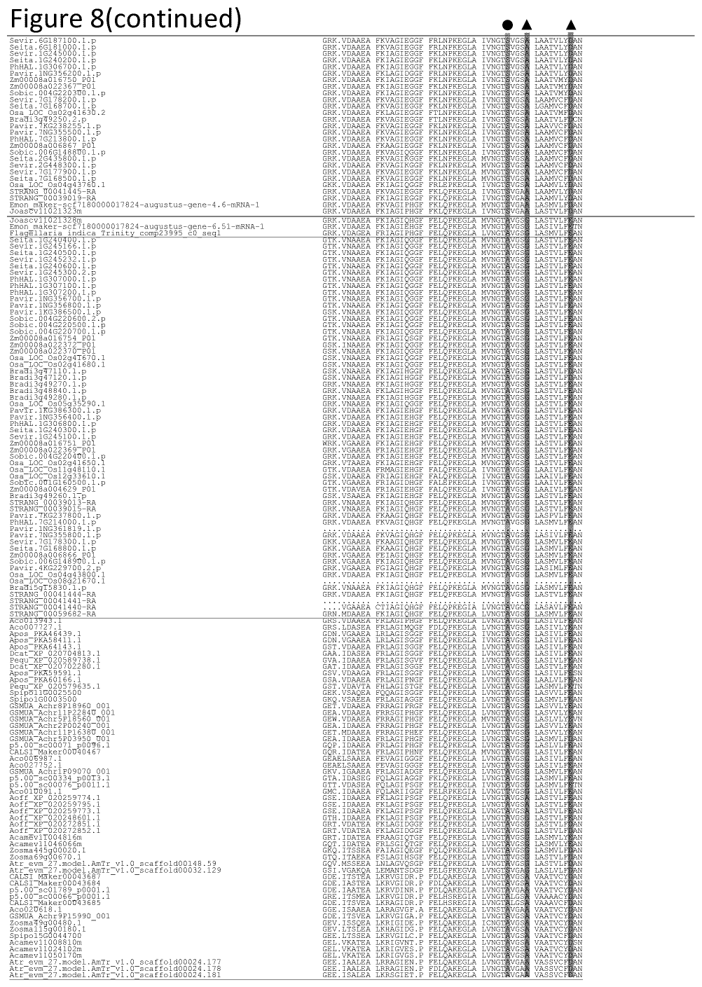

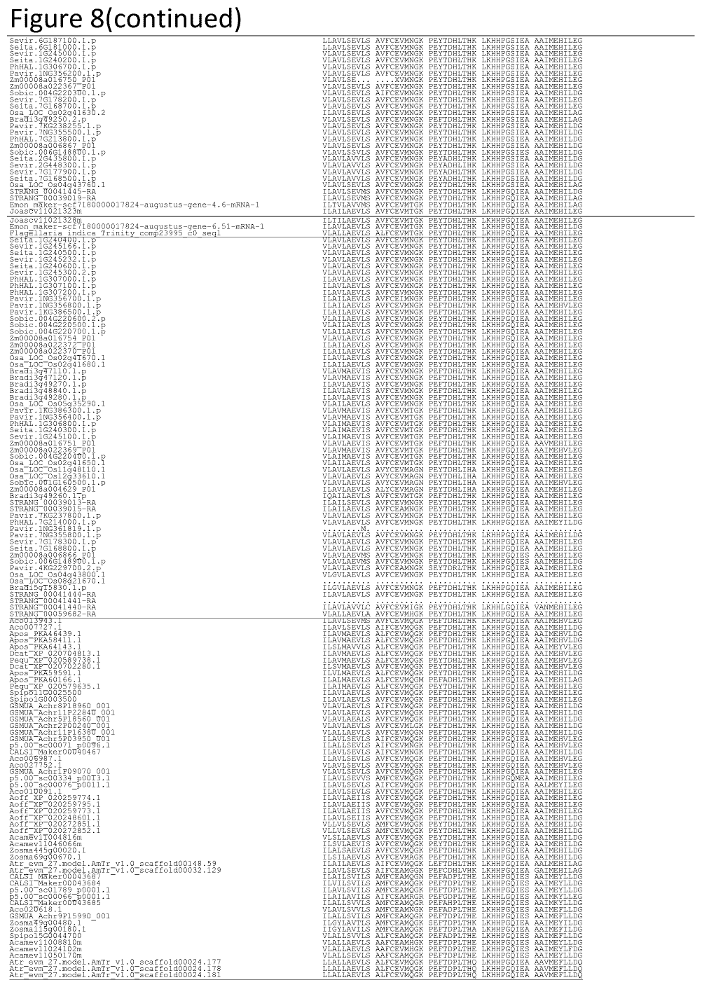

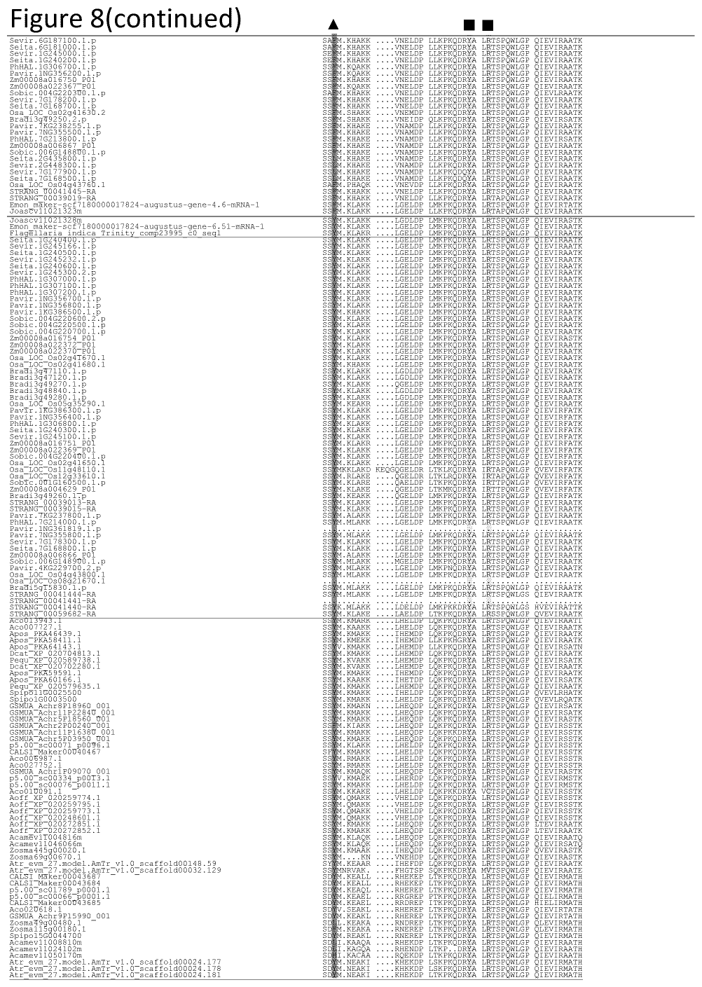

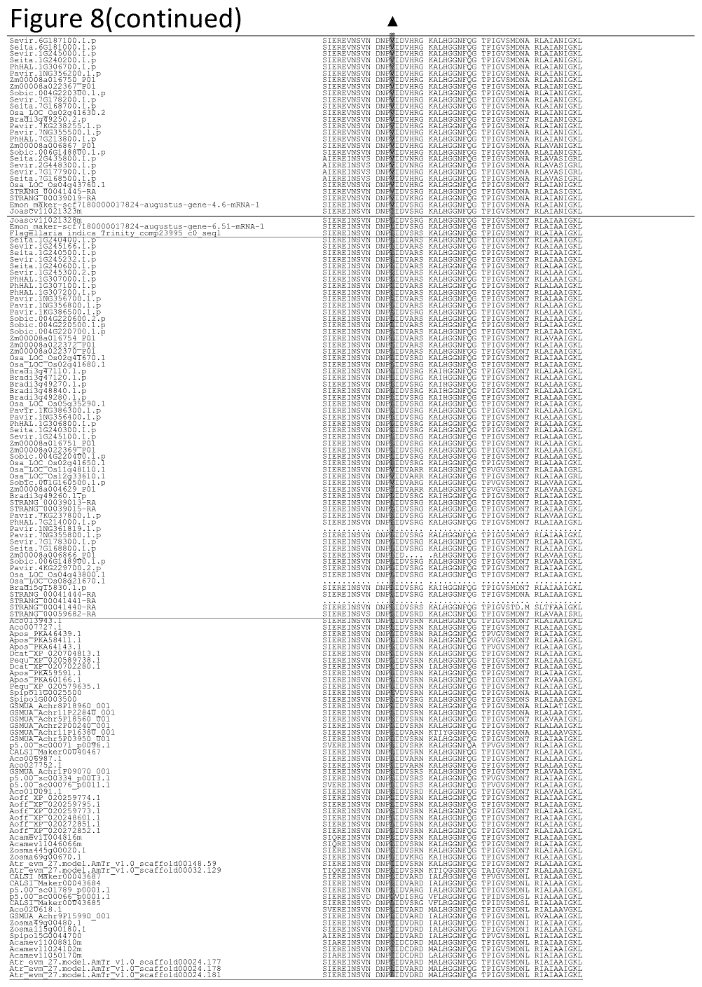

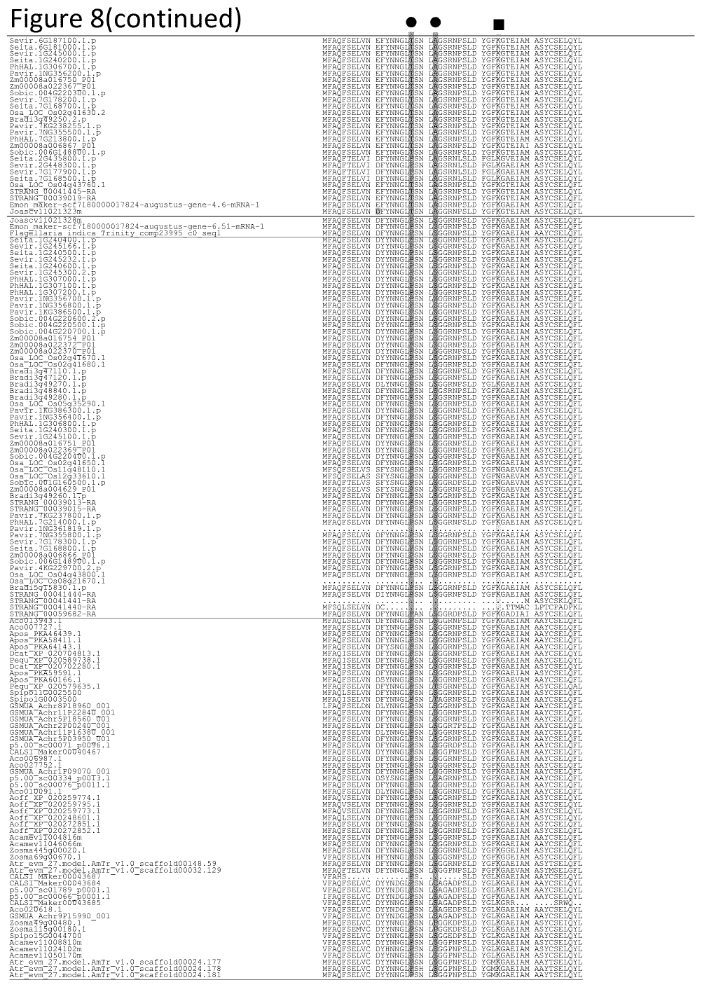

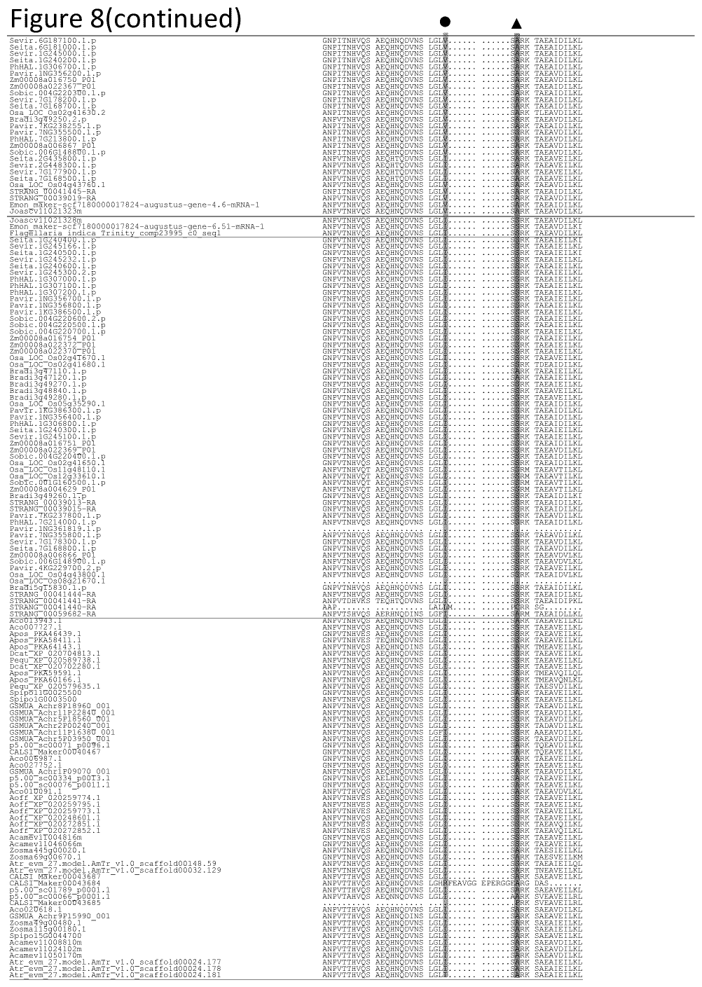

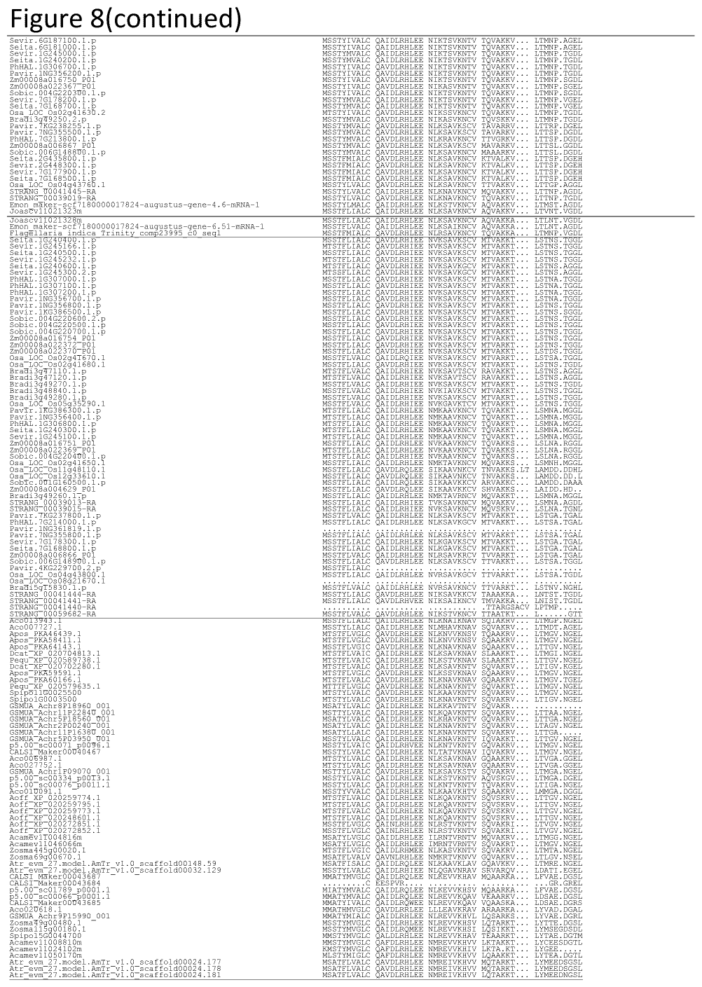

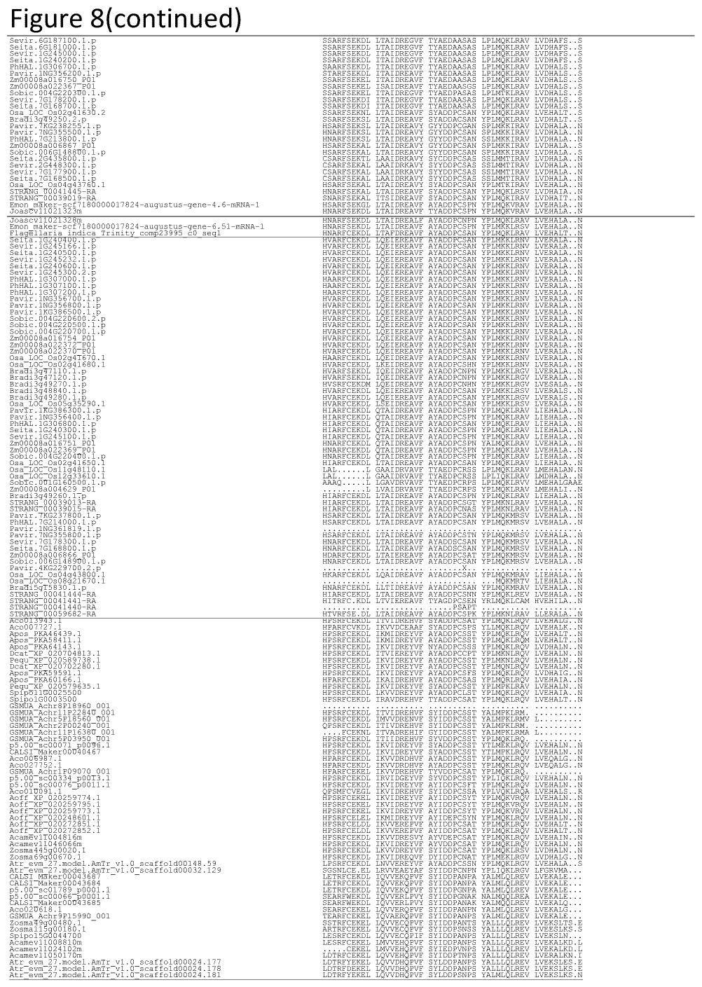

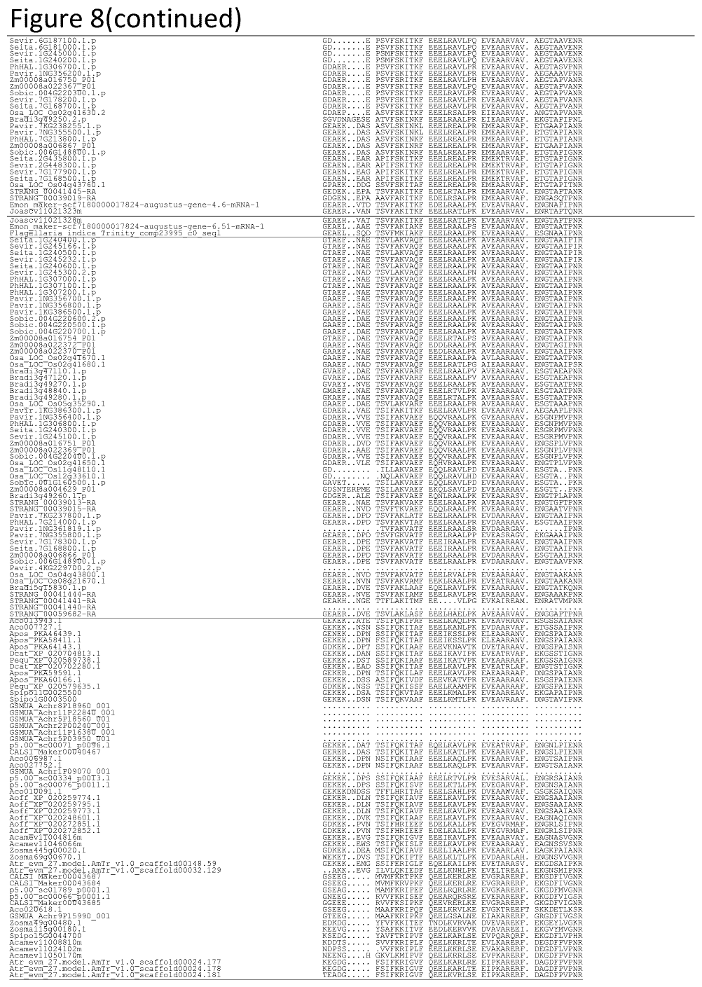

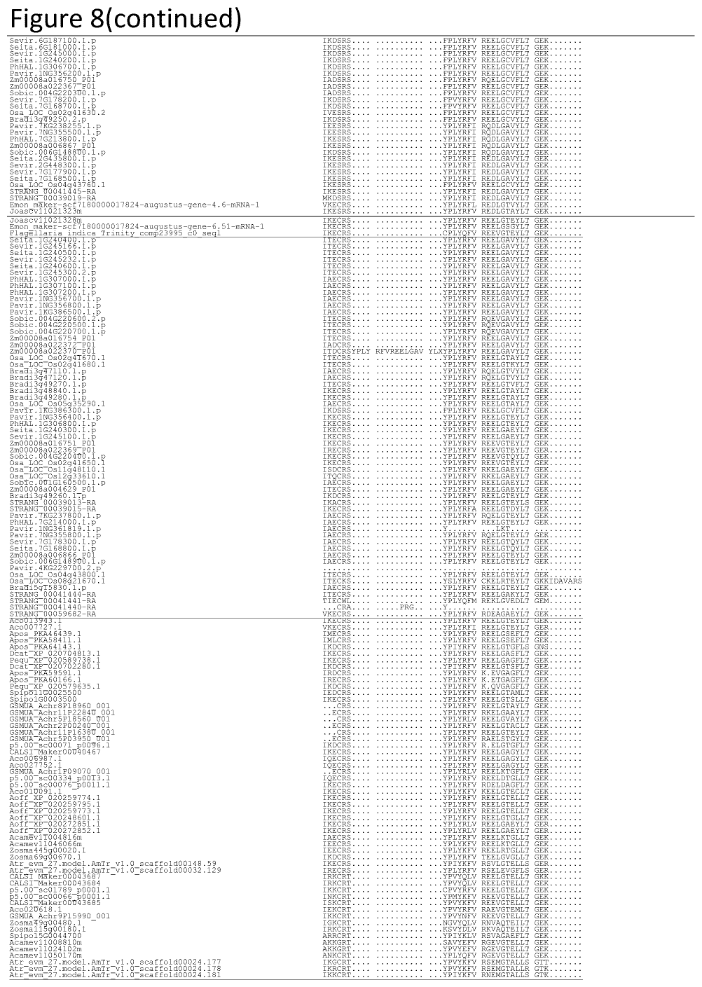

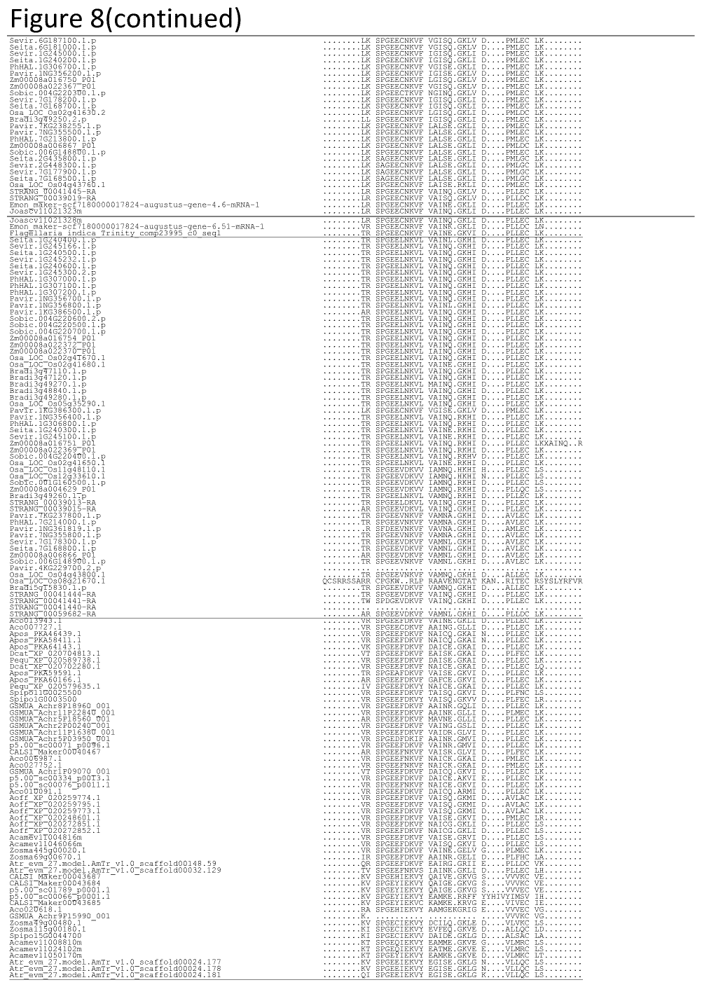

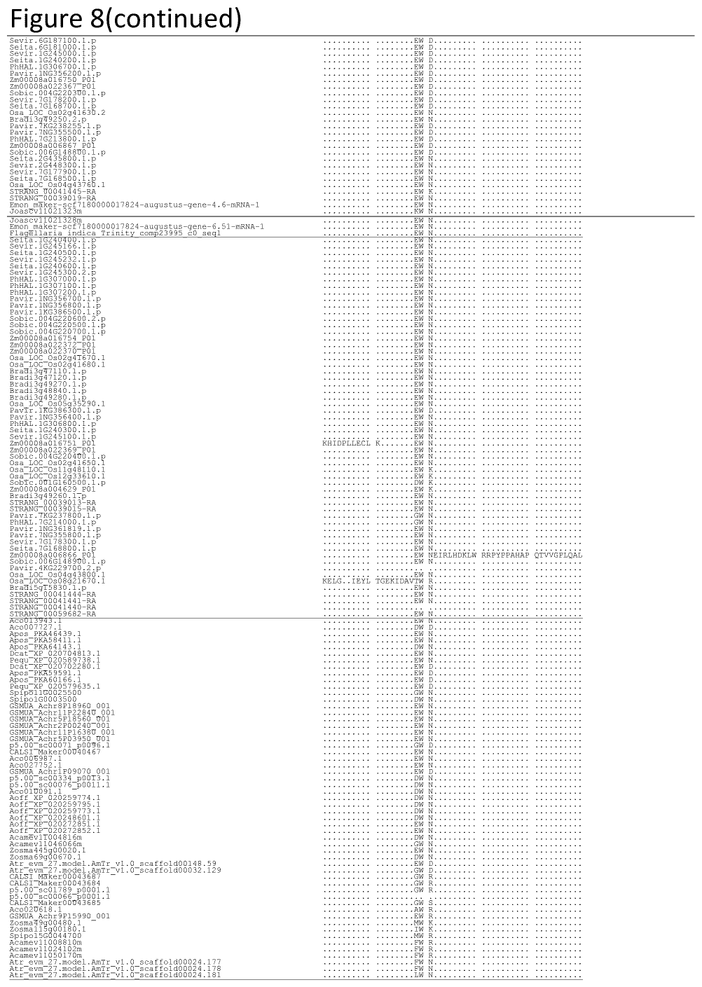

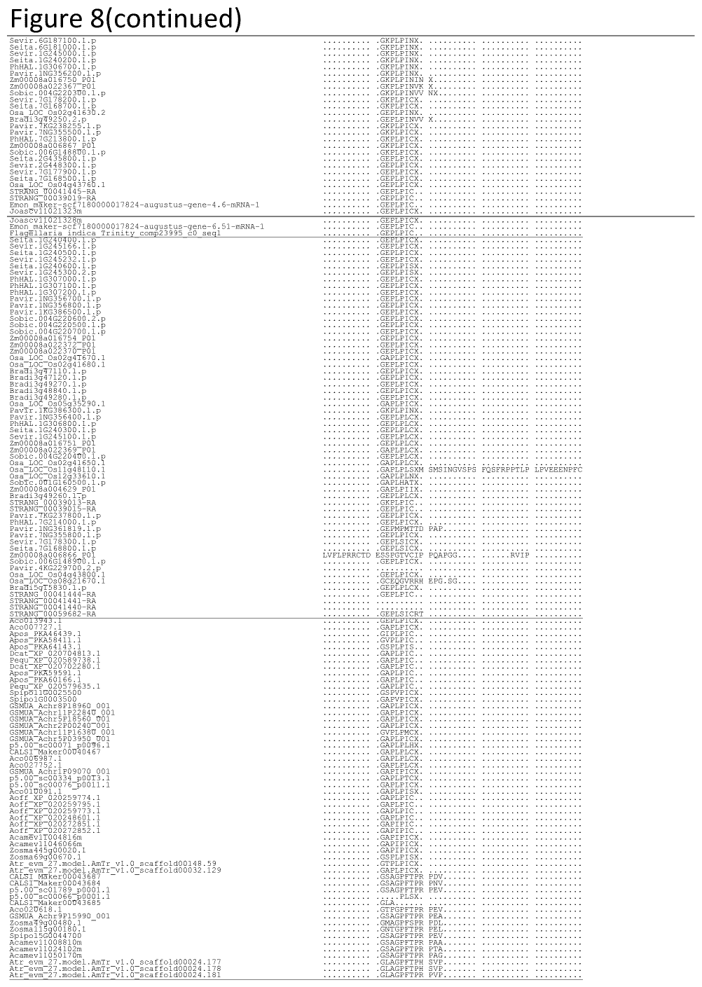

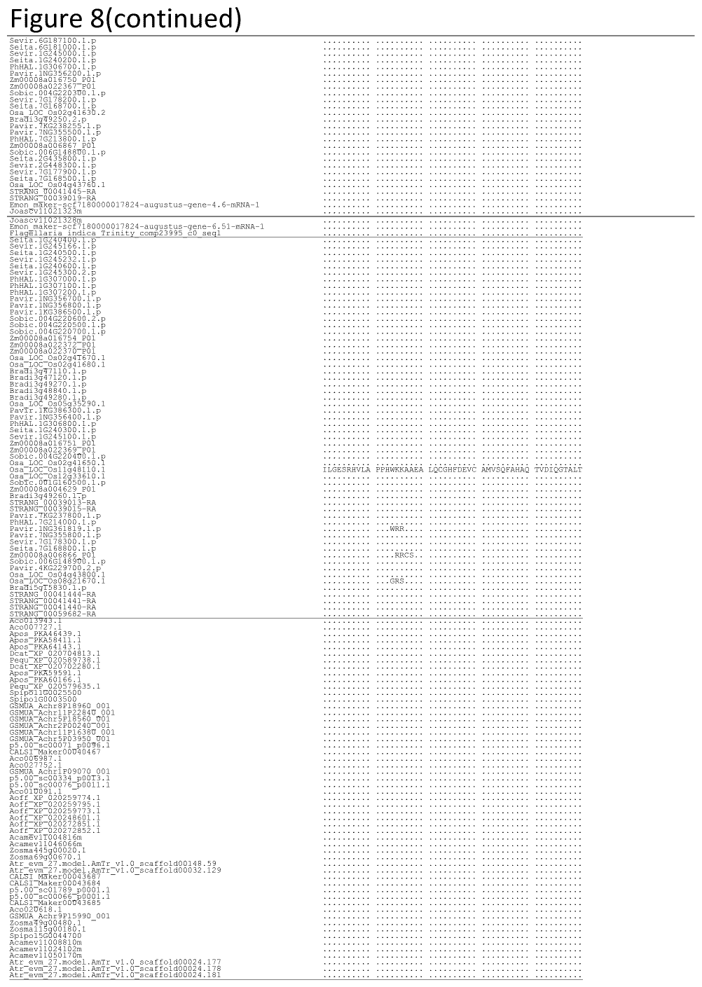

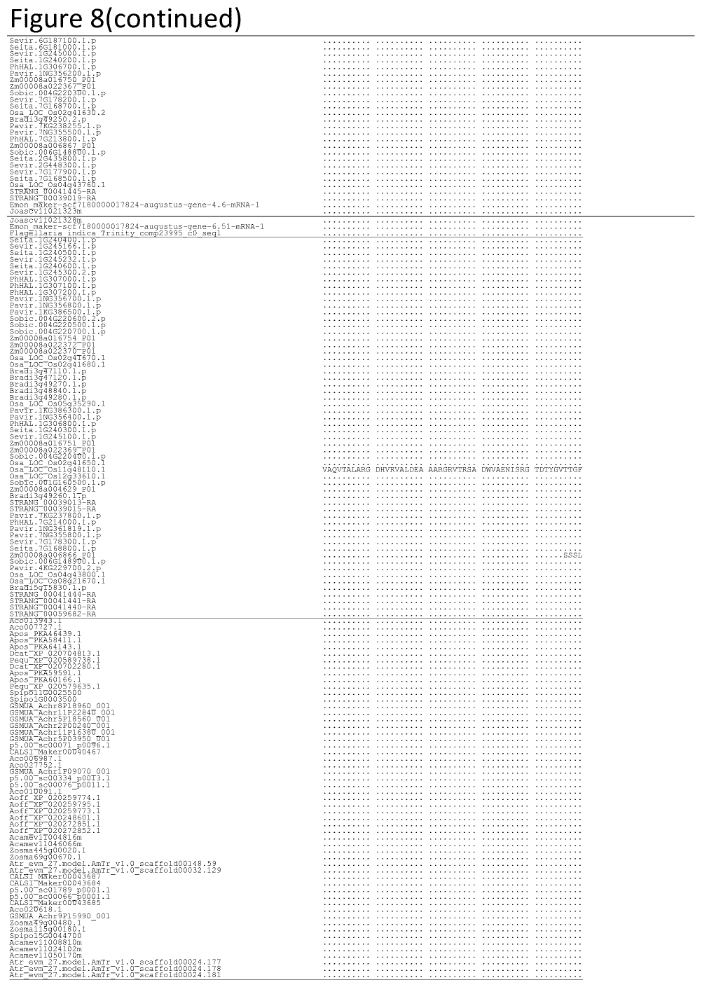

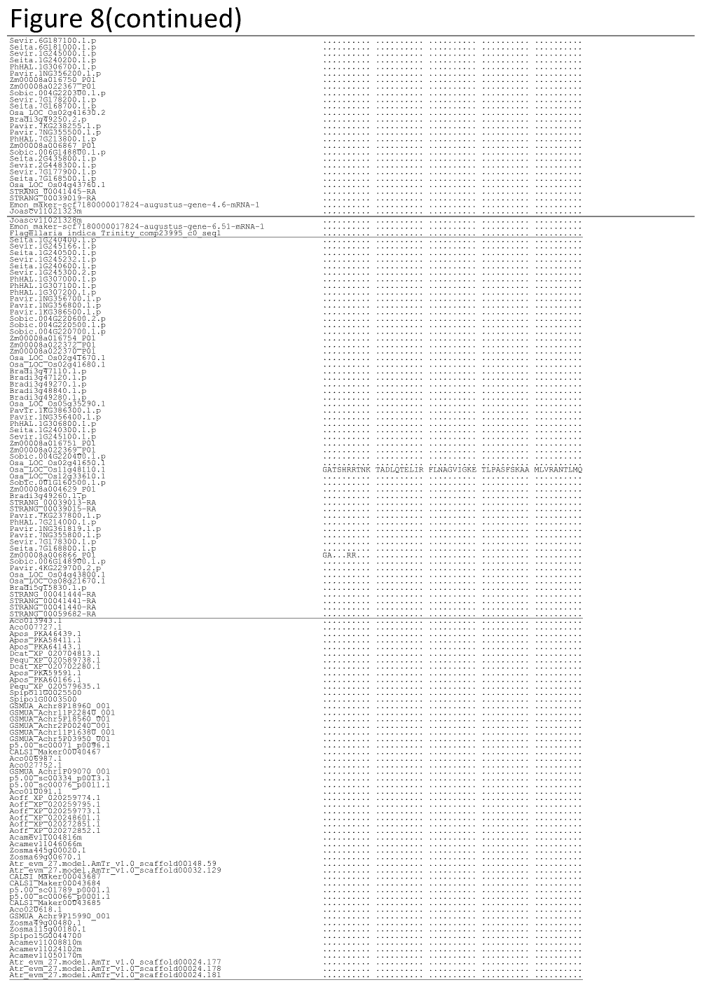

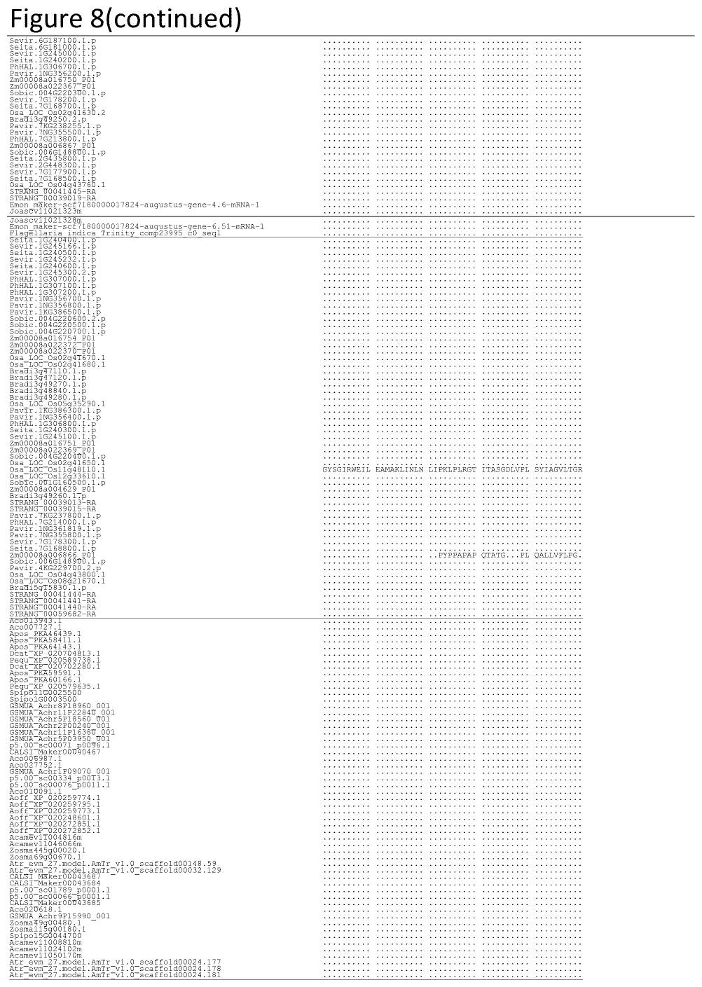

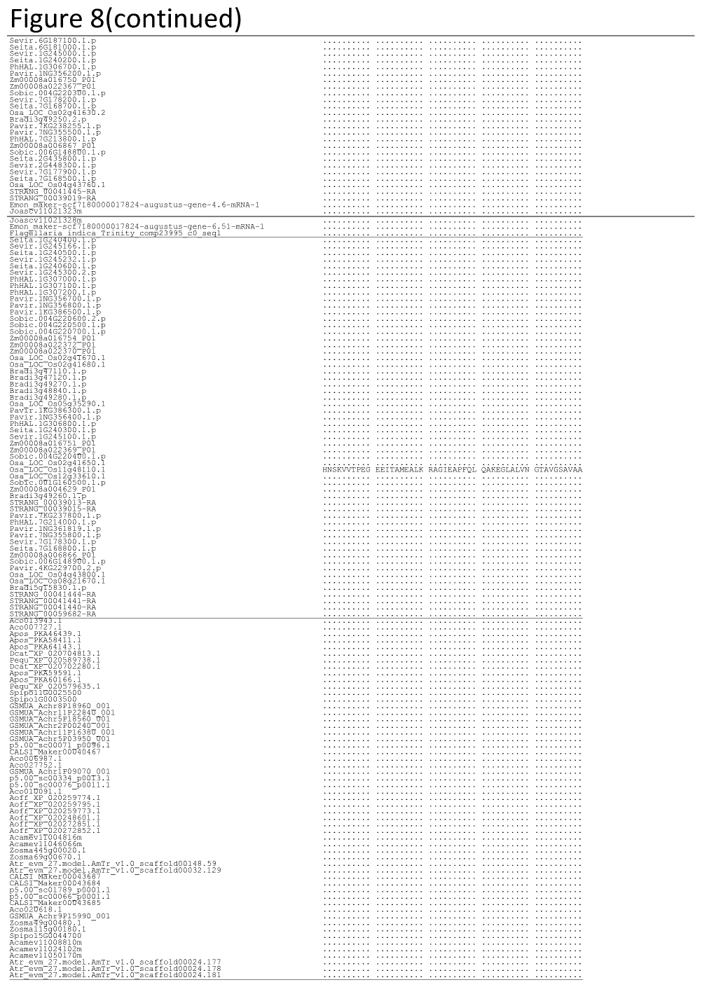

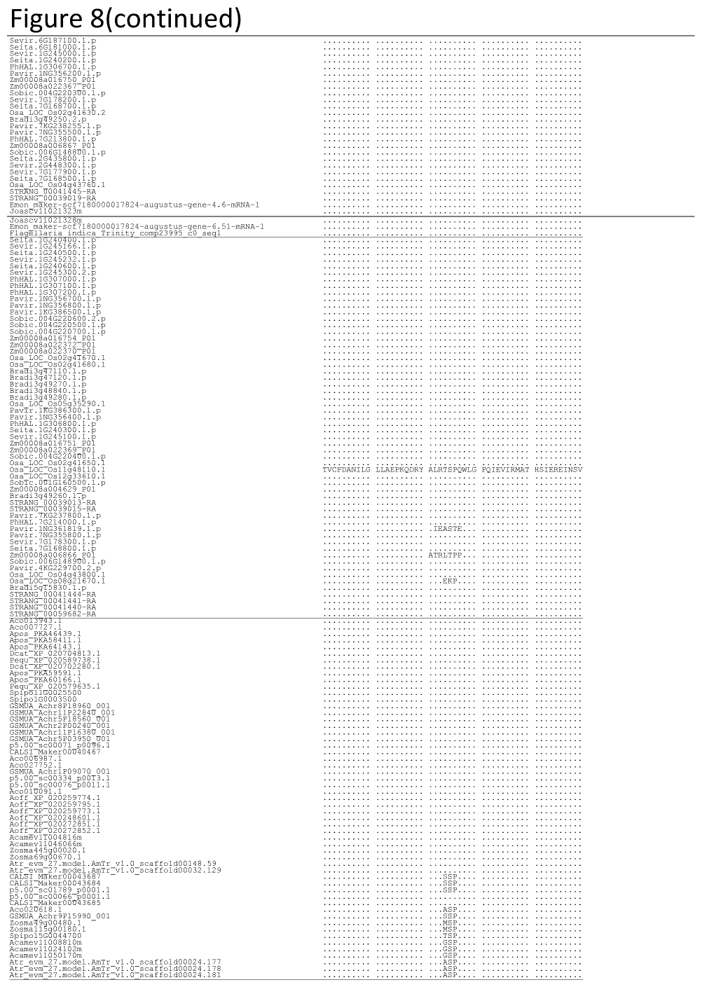

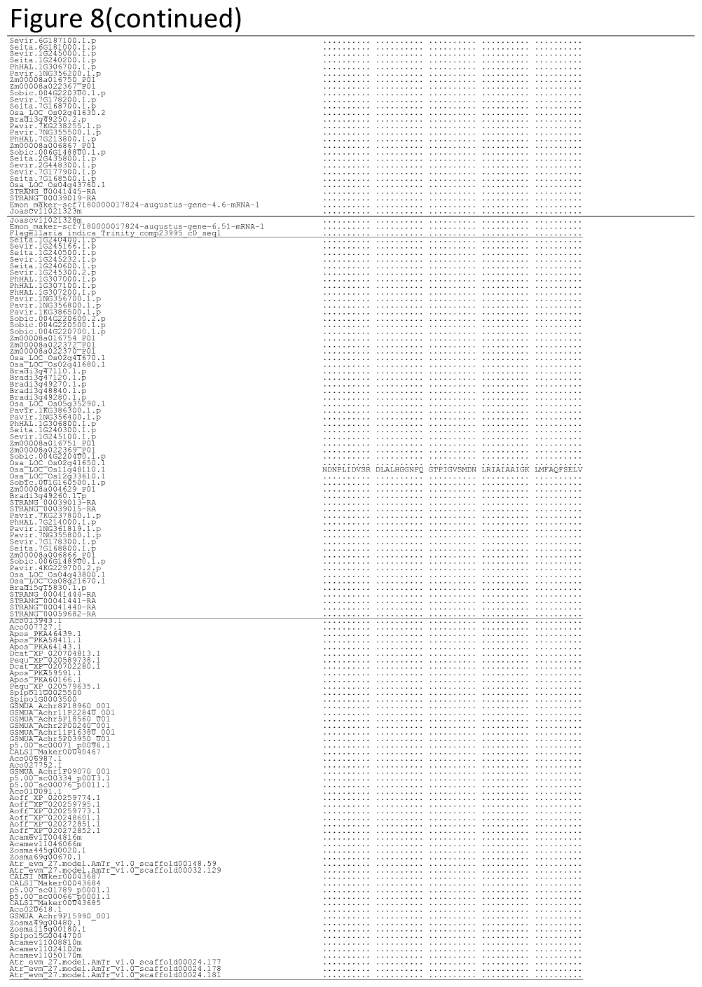

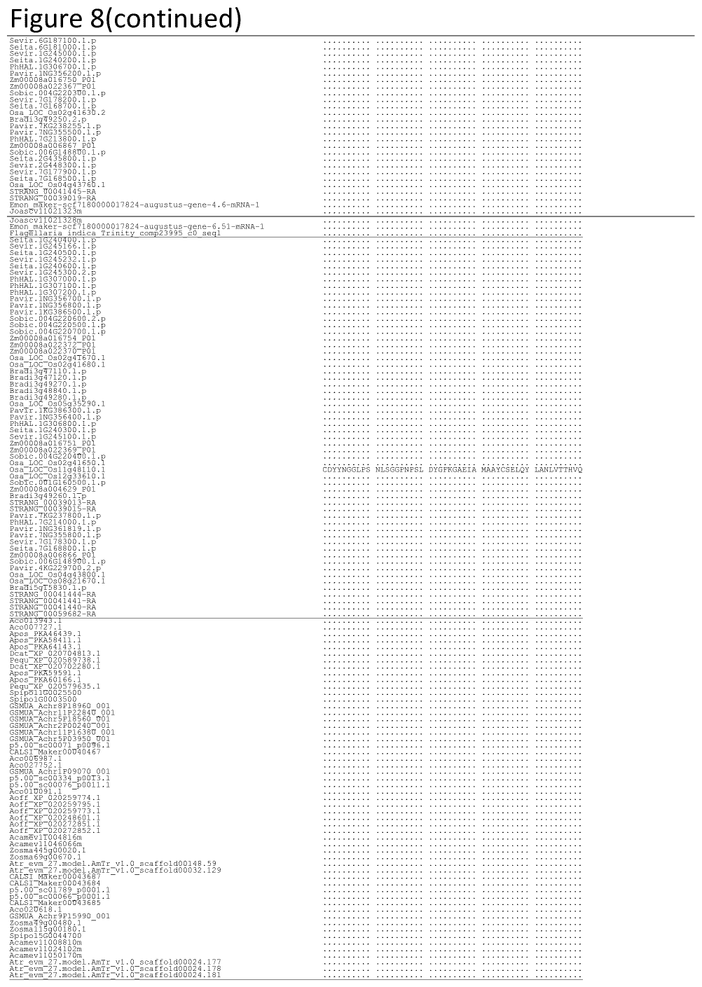

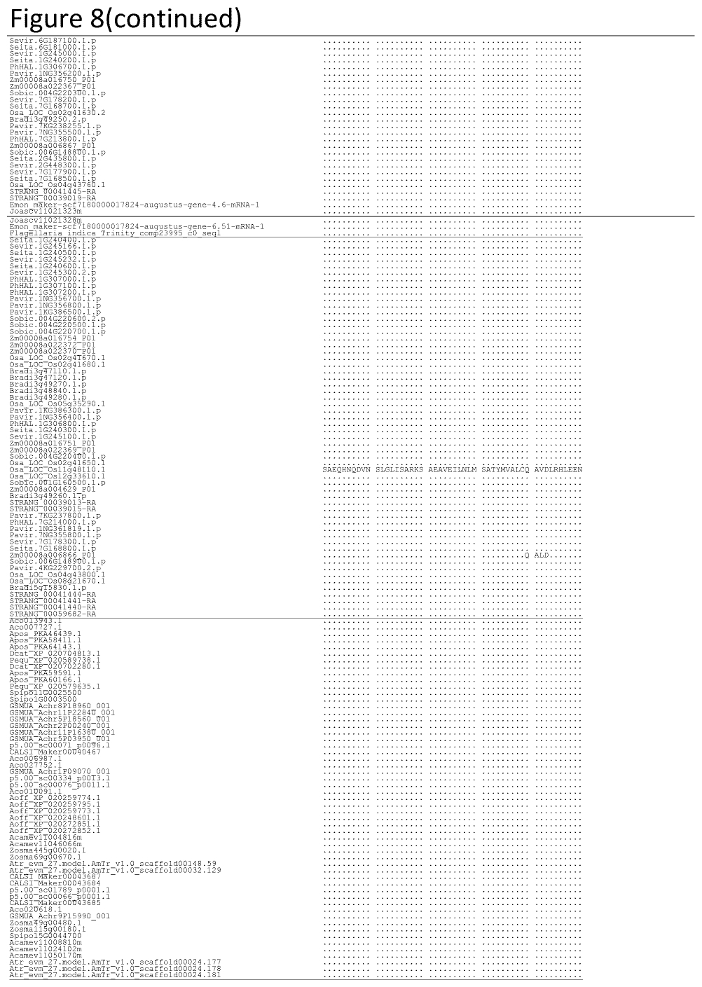

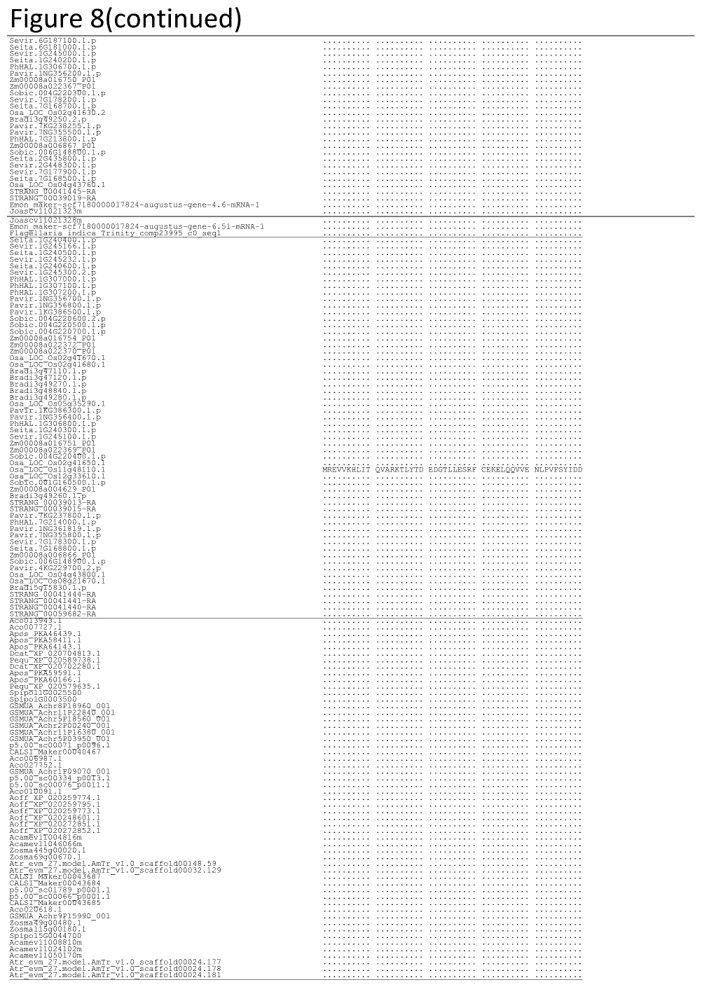

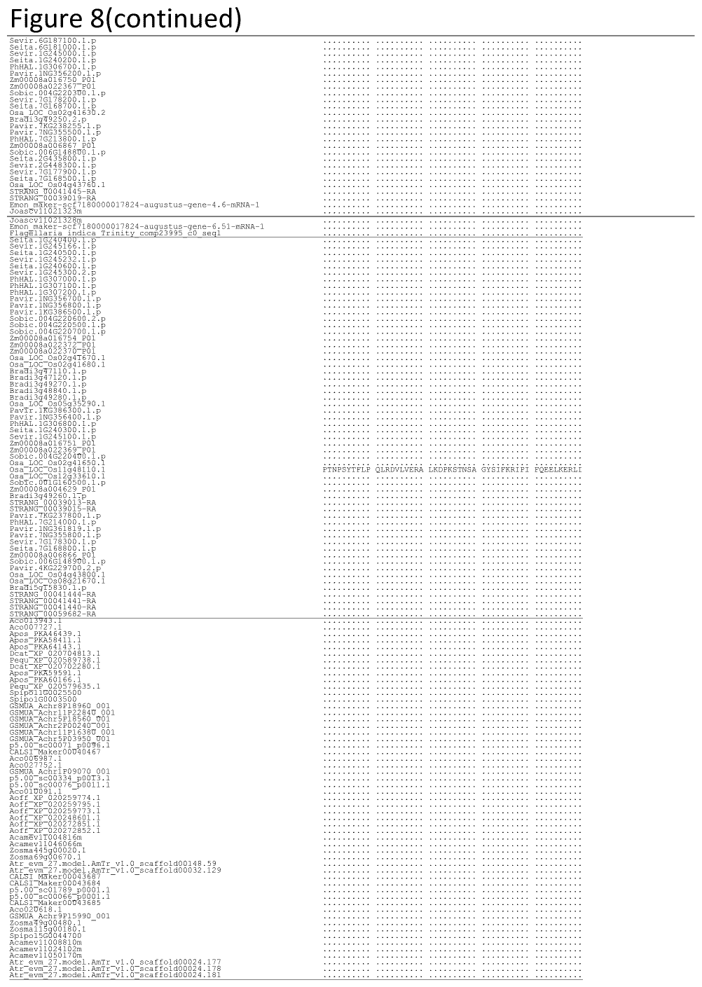

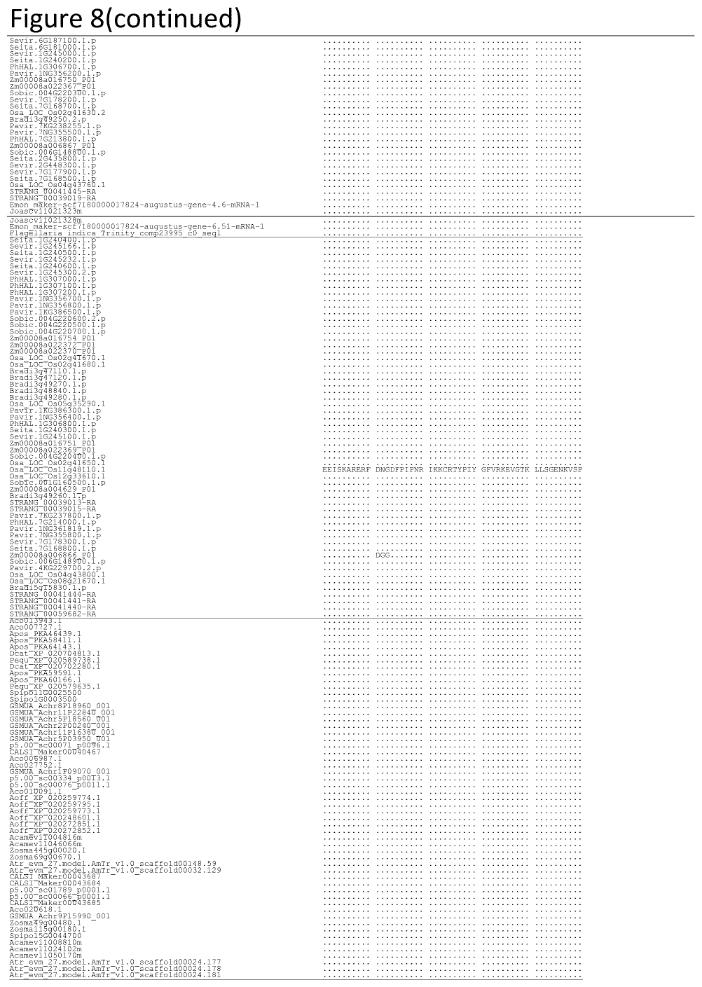

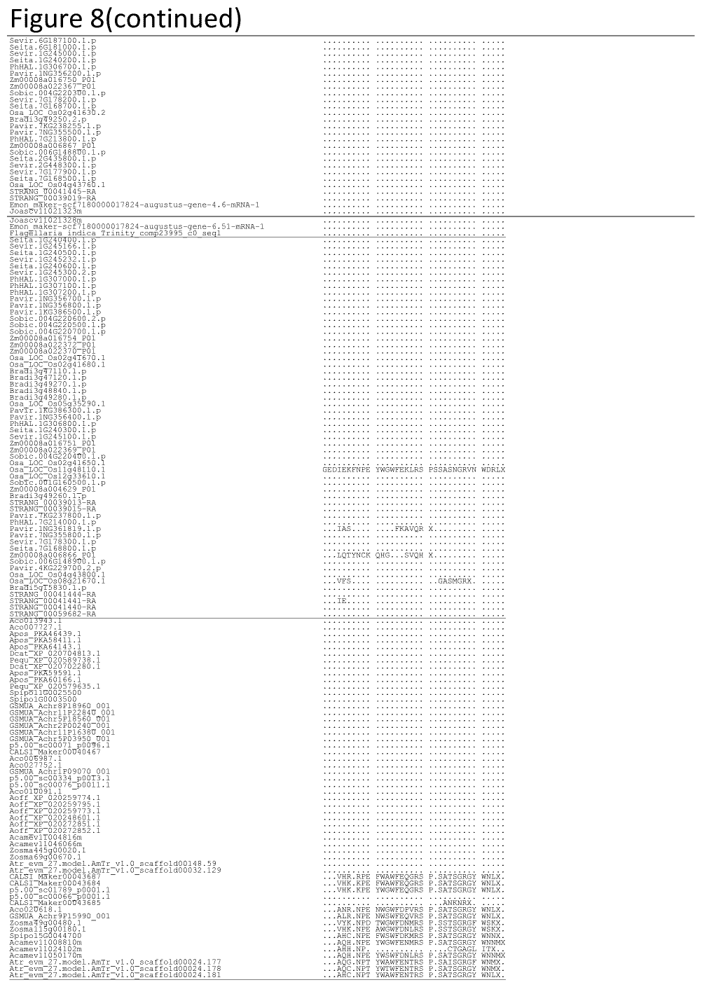

For simplicity, throughout this application, we have arbitrarily used the wild-type PAL enzyme of Joinvillea ascendens (JaPAL; SEQ ID NO: 28) as a reference sequence and have specified the positions of mutations in various PAL/PTAL enzymes using the residue numbering of this enzyme. Any mutation position can be converted to use the residue numbering of another PAL or PTAL enzyme using a sequence alignment, such as the alignment shown in FIG. 8. For example, residues 112 and 140 of JaPAL (SEQ ID NO: 28) correspond to residues 116 and 144 of AtPAL1 (SEQ ID NO: 144) and correspond to residues 97 and 125 of JaPTAL (SEQ ID NO: 27), as is demonstrated in FIG. 8. The use of a PAL enzyme as a reference sequence for a PTAL enzyme is warranted by the high degree of sequence conservation between these enzyme groups. For example, the sequence of JaPAL is 86.9% identical and 92.4% similar to the sequence of JaPTAL. Further, PAL and PTAL enzymes are classified as belonging to the same orthogroup (i.e., set of genes derived from a single gene in the last common ancestor).

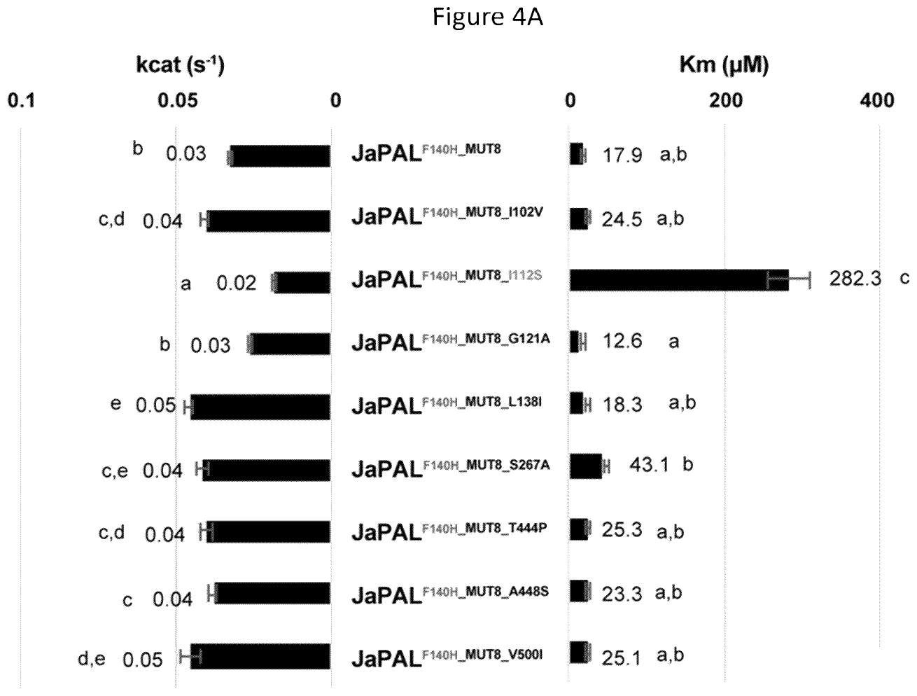

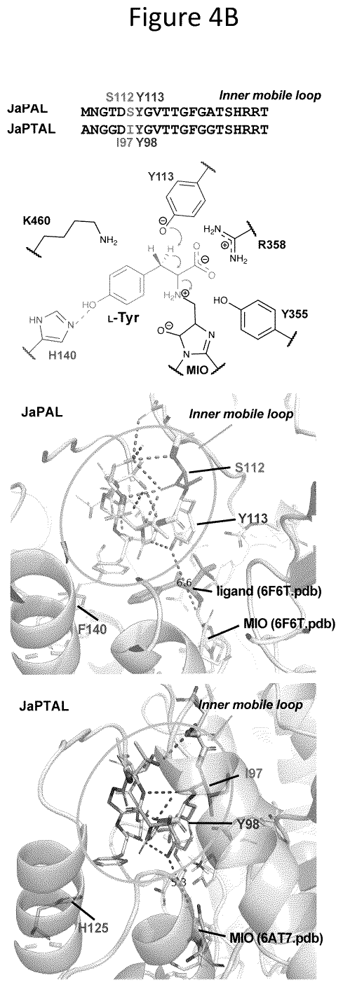

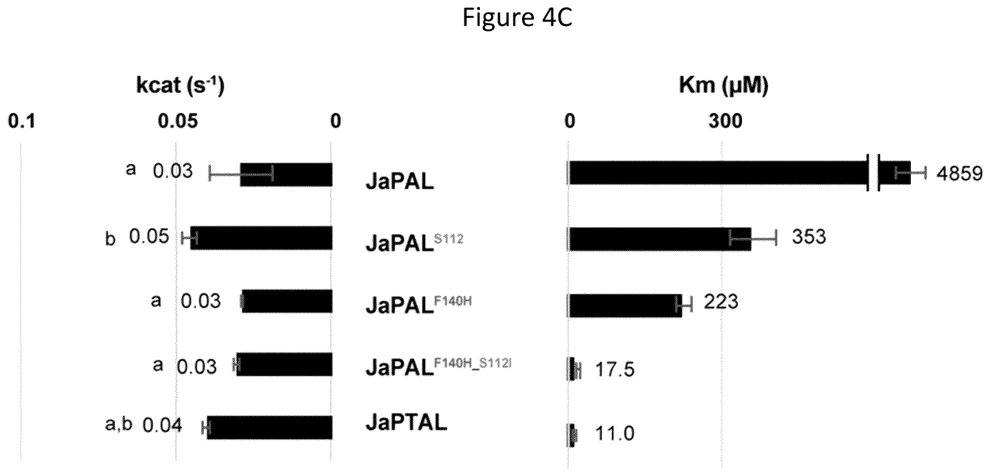

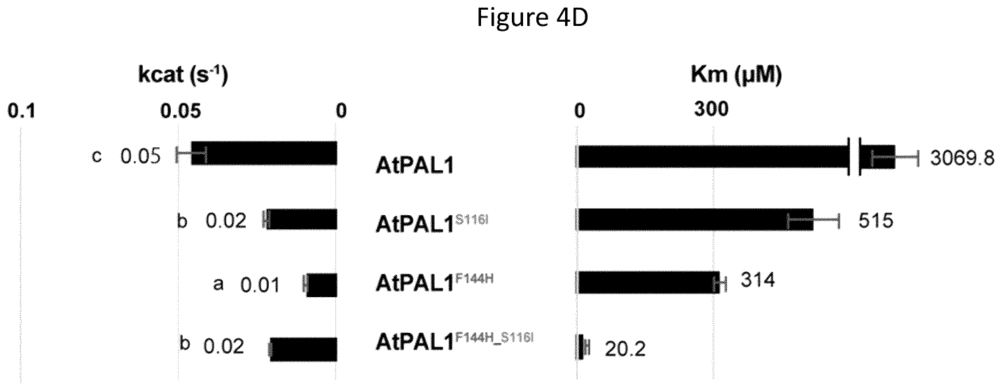

In Example 1, the inventors demonstrate that introducing the mutation S112I into the PAL enzyme of Joinvillea ascendens (JaPAL; SEQ ID NO: 28) or introducing the corresponding mutation (i.e., S116I) into the PAL enzyme of the distantly related plant Arabidopsis thaliana (AtPAL1; SEQ ID NO: 144) increases the TAL activity of these enzymes (FIGS. 4C-4D). Further, they show that introducing the two mutations S112I and F140H into JaPAL or introducing the corresponding mutations (i.e., S116I and F144H) into AtPAL1 converts these PAL enzymes into bifunctional PTAL enzymes, which are referred to herein as JaPAL^{F140H_S112}I (SEQ ID NO: 145) and AtPALIF^{144H_S116I} (SEQ ID NO: 146), respectively. Thus, in some embodiments, the wild-type PAL enzyme is a PAL enzyme is from Joinvillea ascendens or Arabidopsis thaliana. In specific embodiments, the wild-type PAL enzyme comprises SEQ ID NO: 28 or SEQ ID NO: 144. In some embodiments, the wild-type PAL enzyme comprises a sequence having at least 75%, 80%, 85%, 90%, 91%, 92%, 93%, 94%, 95%, 96%, 97%, 98%, 99%, or more sequence identity to SEQ ID NO: 28 or SEQ ID NO: 144.

As is noted above, the inventors have demonstrated that PAL enzymes from multiple, distantly related plants (i.e., Joinvillea ascendens (a monocot) and Arabidopsis thaliana (a dicot)) can be converted into bifunctional PTAL enzymes. PAL enzymes (which are found in bacteria, fungi, and plants) are highly conserved across a wide variety of land plants, as is demonstrated in FIG. 8. Thus, the engineered PAL enzymes of the present invention may be any wild-type PAL enzyme from a land plant into which the necessary mutation(s) (i.e., a mutation at a position corresponding to residue 112 of SEQ ID NO: 28 and, optionally, a second mutation at a position corresponding to residue 140 of SEQ ID NO: 28) have been introduced. For example, the wild-type PAL enzyme may be one of the PAL enzymes included in the sequence alignment of FIG. 8, i.e., SEQ ID NO: 28-143.

In some embodiments, the engineered PAL enzymes comprise a polypeptide or a functional fragment thereof having at least 75%, 80%, 85%, 90%, 91%, 92%, 93%, 94%, 95%, 96%, 97%, 98%, 99%, or more sequence identity to a polypeptide selected from SEQ ID NO: 28-143. “Percentage of sequence identity” is determined by comparing two optimally aligned sequences over a comparison window. The aligned sequences may comprise additions or deletions (i.e., gaps) relative to each other for optimal alignment. The percentage is calculated by determining the number of matched positions at which an identical nucleic acid base or amino acid residue occurs in both sequences, dividing the number of matched positions by the total number of positions in the window of comparison, and multiplying the result by 100. Protein and nucleic acid sequence identities can be evaluated using the Basic Local Alignment Search Tool (“BLAST”), which is well known in the art (Karlin and Altschul, Methods for assessing the statistical significance of molecular sequence features by using general scoring schemes. Proc. Natl. Acad. Sci. USA (1990) 87: 2267-2268; Altschul et al. Gapped BLAST and PSI-BLAST: a new generation of protein database search programs. Nucl. Acids Res. (1997) 25: 3389-3402). The BLAST programs identify homologous sequences by identifying similar segments between a query sequence and a test sequence, which is preferably obtained from a protein or nucleic acid sequence database. The BLAST programs can be used with the default parameters or with modified parameters provided by the user.

FIG. 3B and FIG. 8 show amino acid sequence alignments of PAL/PTAL enzymes from a variety of plant species (SEQ ID NO: 1-143). Based on these alignments, it is readily apparent that various amino acid residues may be mutated without substantially affecting the PAL/TAL activity of these enzymes. For example, a person of ordinary skill in the art would appreciate that substitutions in a PAL/PTAL enzyme could be selected based on the alternative amino acid residues that occur at the corresponding position in related PAL/PTAL enzyme from another plant species. For example, the Joinvillea ascendens PAL enzyme (SEQ ID NO: 28) has a methionine at position 103 while some of the other enzyme sequences shown in FIG. 3B have a leucine, threonine, or valine at this position. Thus, exemplary modifications that could be made in the Joinvillea ascendens PAL enzyme based on this sequence alignment include M103L, M103T, and M103V substitutions. Similar modifications could be made in any of SEQ ID NO: 1-143 at any position shown in the sequence alignment of FIG. 3B or FIG. 8. Additionally, a person of ordinary skill in the art could easily align other PAL/PTAL enzyme sequences with the sequences shown in FIG. 3B or FIG. 8 to identify additional mutations that could be included in the engineered PAL enzymes of the present invention.

Regardless of their origin, the engineered PAL enzymes of the present invention comprise a mutation at a position corresponding to residue 112 of JaPAL (SEQ ID NO: 28) and optionally further comprise a second mutation at a position corresponding to residue 140 of JaPAL. As used herein, the phrase “at a position corresponding to” refers to an amino acid position that aligns with an amino acid position in another protein in a protein sequence alignment or a protein structure alignment. For example, the phrase “a position corresponding to residue 112 of SEQ ID NO: 28” refers to an amino acid position in the sequence of protein X that aligns with the 112th amino acid residue of SEQ ID NO: 28 when the sequence of protein X is aligned with SEQ ID NO: 28. To determine whether a particular protein sequence has a mutation at a position “corresponding to” a position disclosed herein, one may align that particular protein sequence with SEQ ID NO: 28 using a conventional sequence alignment method (see, e.g., Bioinformatics (2007) 23(7): 802-8) and examine the alignment at the appropriate position.

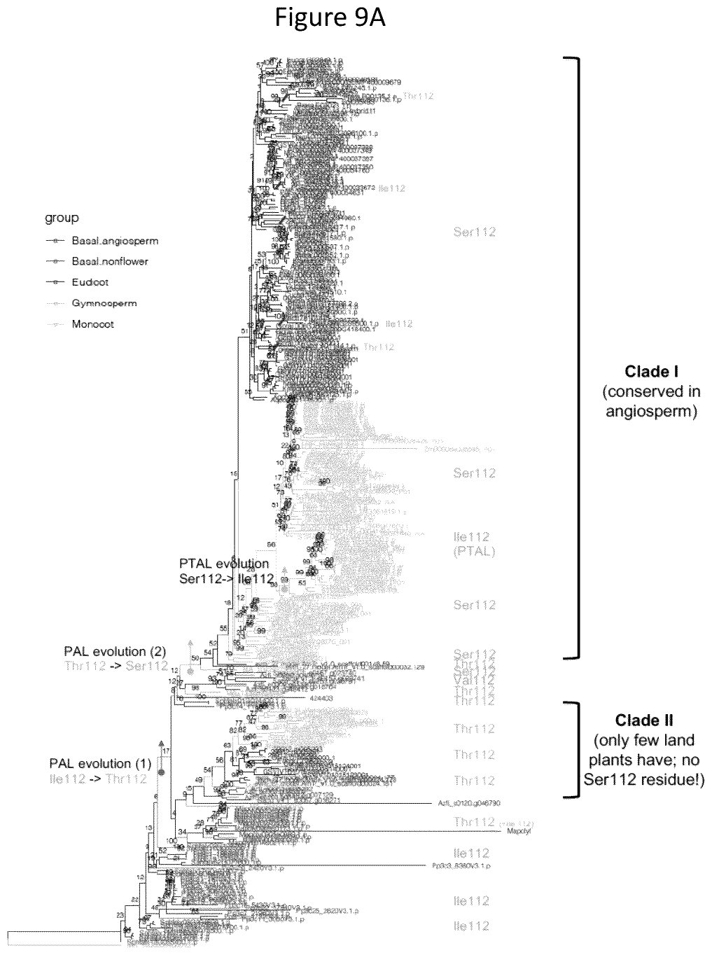

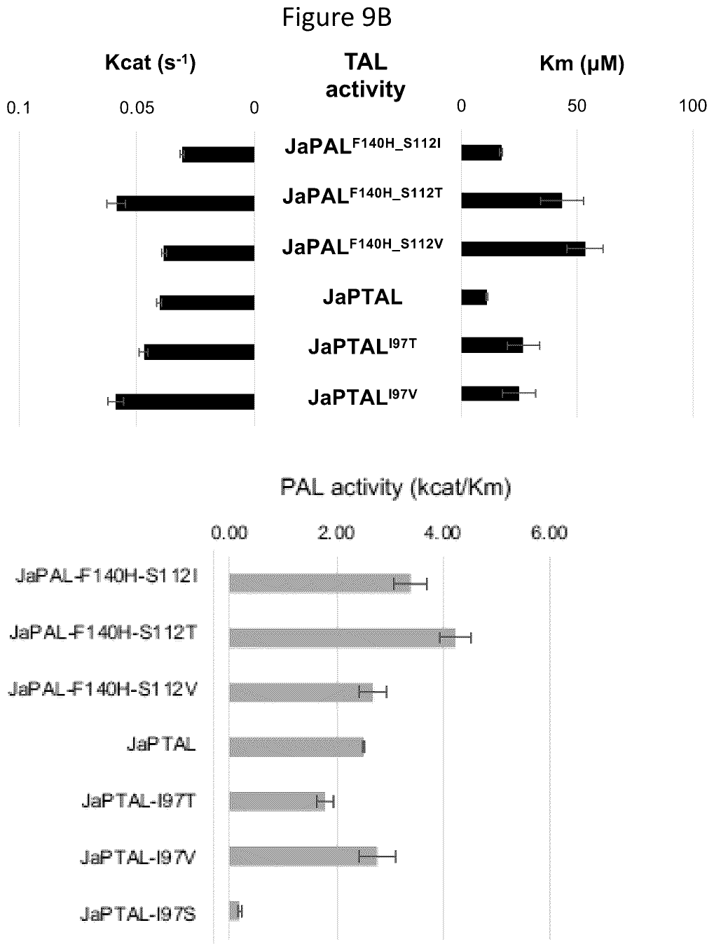

In some embodiments, the engineered PAL enzyme comprises a serine to isoleucine mutation at a position corresponding to residue 112 of SEQ ID NO: 28 (e.g., a S112I mutation). However, in Example 3, the inventors demonstrate that several different substitutions at position 112 retain the TAL activity of the JaPAL^{F140H_S112I} double mutant. Specifically, they show that substituting the Ile at this position with a valine or threonine retains strong TAL activity but substituting it with a serine does not (FIG. 9B). Thus, in some embodiments, the mutation is a serine to valine mutation or a serine to threonine mutation.

In Example 1, the inventors generated a JaPAL enzyme, referred to as JaPAL^{F140H_MUT8}, that has a PTAL-type substitution at residue 140 and at eight additional residues that are highly conserved within both PAL and PTAL enzymes but are distinct between these two groups (i.e., residues 102, 112, 121, 138, 267, 444, 448, and 500). Kinetic assays showed that the catalytic properties of TAL activity (especially tyrosine substrate affinity (Km)) of JaPAL^{F140H_MUT8} were significantly improved compared to those of wild-type JaPAL and were comparable with those of wild-type JaPTAL (FIG. 3C; Table 3). Thus, in some embodiments, the engineered PAL enzyme further comprises at least one additional mutation at a position corresponding to residue 102, 121, 138, 267, 444, 448, or 500 of SEQ ID NO: 28. In specific embodiments, the at least one additional mutation includes a valine to isoleucine mutation at a position corresponding to residue 102 of SEQ ID NO: 28, an alanine to glycine mutation at a position corresponding to residue 121 of SEQ ID NO: 28, an isoleucine to lysine mutation at a position corresponding to residue 138 of SEQ ID NO: 28, an alanine to serine mutation at a position corresponding to residue 267 of SEQ ID NO: 28, a proline to threonine mutation at a position corresponding to residue 444 of SEQ ID NO: 28, a serine to alanine mutation at a position corresponding to residue 448 of SEQ ID NO: 28, or an isoleucine to valine mutation at a position corresponding to residue 500 of SEQ ID NO: 28.

### Polynucleotides:

In a second aspect, the present invention provides polynucleotides encoding an engineered PAL enzyme described herein. The terms “polynucleotide,” “oligonucleotide,” and “nucleic acid” are used interchangeably to refer a polymer of DNA or RNA. A polynucleotide may be single-stranded or double-stranded and may represent the sense or the antisense strand. A polynucleotide may be synthesized or obtained from a natural source. A polynucleotide may contain natural, non-natural, or altered nucleotides, as well as natural, non-natural, or altered internucleotide linkages (e.g., phosphoroamidate linkages, phosphorothioate linkages). The term polynucleotide encompasses constructs, vectors, plasmids, and the like. In some embodiments, the polynucleotide is complementary DNA (cDNA; i.e., synthetic DNA that has been reverse transcribed from a messenger RNA) or genomic DNA (i.e., chromosomal DNA from an organism). Those of skill in the art understand that, due to degeneracy of the genetic code, a variety of polynucleotides can encode the same polypeptide.

While the polynucleotide sequences disclosed herein are derived from sequences found in plants, any polynucleotide sequence that encodes the desired engineered PAL enzyme may be used with the present invention. For example, in some embodiments, the polynucleotides are codon-optimized for expression in a particular cell (e.g., a plant cell, bacterial cell, or fungal cell). “Codon optimization” is a process used to increase expression of a polynucleotide in a particular host cell by altering the sequence of the polynucleotide to accommodate the codon bias of the host cell. Computer programs for generating codon-optimized sequences for use in a particular host cell are known in the art.

### Constructs:

In a third aspect, the present invention provides constructs comprising a promoter operably linked to one of the polynucleotides described herein. As used herein, the term “construct” refers to a recombinant polynucleotide, i.e., a polynucleotide that was formed by combining at least two polynucleotide components from different sources, natural or synthetic. For example, a construct may comprise the coding region of one gene operably linked to a promoter that is (1) associated with another gene found within the same genome, (2) from the genome of a different species, or (3) synthetic. Constructs can be generated using conventional recombinant DNA methods.

As used herein, the term “promoter” refers to a DNA sequence that defines where transcription of a polynucleotide beings. RNA polymerase and the necessary transcription factors bind to the promoter to initiate transcription. Promoters are typically located directly upstream (i.e., at the 5′ end) of the transcription start site. However, a promoter may also be located at the 3′ end, within a coding region, or within an intron of a gene that it regulates. Promoters may be derived in their entirety from a native or heterologous gene, may be composed of elements derived from multiple regulatory sequences found in nature, or may comprise synthetic DNA. A promoter is “operably linked” to a polynucleotide if the promoter is positioned such that it can affect transcription of the polynucleotide.

The promoter used in the constructs described herein may be a heterologous promoter (i.e., a promoter that is not naturally associated with the wild-type PAL enzyme), an endogenous promoter (i.e., a promoter that is naturally associated with the wild-type PAL enzyme), or a synthetic promoter that is designed to function in a desired manner in a particular host cell. Suitable promoters for use with the present invention include, but are not limited to, constitutive, inducible, temporally regulated, developmentally regulated, chemically regulated, tissue-preferred, and tissue-specific promoters. In some cases, it may be advantageous to use a tissue-specific promoter or a developmental stage-specific promoter to ensure that the construct will drive expression of the engineered enzyme in a particular tissue (e.g., roots, leaves) or during a particular developmental stage (e.g., leaf maturation, seed development, senescence).

In some embodiments, the promoter is a plant promoter, i.e., a promoter that is active in plant cells. Suitable plant promoters include, without limitation, the 35S promoter of the cauliflower mosaic virus, ubiquitin, the tCUP cryptic constitutive promoter, the Rsyn7 promoter, the maize In2-2 promoter, and the tobacco PR-la promoter.

### Vectors:

In a fourth aspect, the present invention provides vectors comprising one of the polynucleotides or constructs described herein. The term “vector” refers to a DNA molecule that is used to carry a particular DNA segment (i.e., a DNA segment included in the vector) into a host cell. Some vectors are capable of autonomous replication in a host cell (e.g., bacterial vectors that include an origin of replication and episomal mammalian vectors). Other vectors can be integrated into the genome of a host cell such that they are replicated along with the host genome (e.g., viral vectors and transposons). Vectors may include heterologous genetic elements that are necessary for propagation of the vector or for expression of an encoded gene product. Vectors may also include a reporter gene or a selectable marker gene. Suitable vectors include plasmids (i.e., circular double-stranded DNA molecules) and viral vectors.

### Cells:

In a fifth aspect, the present invention provides cells comprising one of the engineered enzymes, polynucleotides, constructs, or vectors described herein. The cells may be eukaryotic or prokaryotic. Preferably, the cell is a type of cell that can be used for large-scale production of phenylpropanoid-derived compounds or for carbon dioxide sequestration. In some embodiments, the cell is a plant cell, a bacterial cell, a fungal cell, or a protist cell.

### Seeds:

In a sixth aspect, the present invention provides seeds comprising one of the engineered enzymes, polynucleotides, constructs, vectors, or cells described herein. A “seed” is an embryonic plant enclosed in a protective outer covering. In embodiments in which the plant comprises a nucleic acid (i.e., a polynucleotide, construct, or vector) described herein, the nucleic acid may either be integrated into the genome of the seed or exist independently from the genome.

### Plants:

In a seventh aspect, the present invention provides plants grown from the seeds described herein and plants comprising one of the engineered PAL enzymes, polynucleotides, constructs, vectors, or cells described herein.

As used herein, the term “plant” includes both whole plants and plant parts. Examples of plant parts include, without limitation, embryos, pollen, ovules, flowers, glumes, panicles, roots, root tips, anthers, pistils, leaves, stems, seeds, pods, flowers, calli, clumps, cells, protoplasts, germplasm, asexual propagates, and tissue cultures. This term also includes chimeric plants in which only a subset of the plant's cells comprises the engineered PAL enzyme, polynucleotide, construct, or vector.

The inventors predict that engineering the native PAL enzymes of plants to introduce TAL activity will increase carbon flow into lignin/phenylpropanoid synthesis pathways. Thus, the inventors predict that the plants described herein will: (a) produce a greater quantity of lignin as compared to a control plant; (b) produce a greater quantity of phenylpropanoid-derived compounds as compared to a control plant; and/or (c) sequester a greater quantity of carbon dioxide (CO₂) into aromatic compounds as compared to a control plant.

Examples of phenylpropanoid compounds and derivatives thereof that could be produced in higher quantities by the plants of the present invention include flavonoids, anthocyanins, lignins, phenolic acids, stilbenes, coumarins, tannins, suberin, cutins, sporopollenin, lignans, and phenylpropenes. These compounds may be useful, for example, for making dyes, colorants, nutraceuticals, pharmaceuticals, and industrial materials. Lignin-derived aromatic monomers can be obtained from plants using microbial (Curr Opin Biotechnol 56: 179-186, 2019) or chemical (Angew Chem Int Ed 55: 8164-8215, 2016) lignin degradation methods.

“Carbon sequestration” is a process in which atmospheric CO₂ is captured and stored. It is one method for reducing the amount of CO₂ in the atmosphere (i.e., to reduce global climate change). In some embodiments, the methods further comprise harvesting part of the plant while leaving the roots of the plant in the soil such that the carbon contained in the roots is sequestered therein. Harvestable parts of plants include, without limitation, flowers, pollen, seedlings, tubers, leaves, stems, fruit, seeds, roots, cuttings, and the like.

As used herein, the term “control plant” refers to a comparable plant (e.g., of the same species, cultivar, and age) that was raised under the same or comparable conditions (e.g., water, sunlight, nutrients) but that does not express an engineered PAL enzyme described herein.

In some embodiments, the plant produces a greater quantity of lignin and/or phenylpropanoid-derived products or produces these products at a greater rate as compared to a control plant. Suitably, the plant produces at least 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold more lignin and/or phenylpropanoid-derived products as compared to the control plant. The amount of lignin produced by a plant may be measured using the thioglycolic acid method (J Agric Food Chem 60(4): 922-8, 2012), which is a standard method for estimating the total lignin content in plant biomass. The amount of a phenylpropanoid-derived product produced by a plant may be measured using liquid chromatography-mass spectrometry (LC-MS).

In some embodiments, the plant sequesters a greater quantity of CO₂ or sequesters CO₂ at a greater rate as compared to a control plant. Suitably, the CO₂ sequestration of the plant is at least 2%, 5%, 10%, 20%, 30%, 40%, 50%, or 60% greater than that of a control plant. CO₂ sequestration may be quantified by measuring the gas exchange activity of the plant. For example, CO₂ assimilation may be measured using an LI-6400XT photosynthesis system equipped with the 6400-40 leaf chamber (LI-COR). Alternatively, labeled ¹³CO₂ can be fed to plants and the rate of ¹³C incorporation into plants can be measured over time.

The plants of the present invention may be of any species. In some embodiments, the plant is a land plant that comprises a native PAL enzyme. PAL enzymes are expressed broadly in plants. In some embodiments, the plant is selected from Acorus americanus, Amborella trichopoda, Ananas comosus, Apostasia shenzhenica, Asparagus officinalis, Brachypodium distachyon, Calamus simplicifolius, Dendrobium catenatum, Ecdeiocolea monostachya, Elaeis guineensis, Flagellaria indica, Joinvillea ascendens, Musa acuminata, Oryza sativa, Panicum hallii, Panicum virgatum, Phalaenopsis equestris, Setaria italica, Setaria viridis, Sorghum bicolor, Spirodela polyrhiza, Streptochaeta angustifolia, Zea mays, and Zostera marina. Protein sequences of PAL enzymes found in these plants are provided as SEQ ID NO: 28-143, and these sequences are aligned in FIG. 8. In some embodiments, the plant is a bioenergy crop (i.e., a plant that can be used to produce bioenergy). In other embodiments, the plant is a plant that produces a useful phenylpropanoid-derived compound, such as a flavonoid, vanillin, lignan, stilbene, coumarin, or phenylpropene. For example, introducing the tyrosine-derived phenylpropanoid pathway in vanilla may result in increased production of vanillin and introducing this pathway in the legume Medicago truncatula may result in increased production of phenylpropanoids.

In some embodiments, the engineered PAL enzyme is encoded by the genome of the plant. In some embodiments, the plant is a plant that naturally expresses a PAL enzyme, and the gene encoding the native PAL enzyme was modified via gene editing to encode a mutation at a position corresponding to residue 112 of SEQ ID NO: 28. In other embodiments, a polynucleotide encoding an engineered version of a PAL enzyme that is not natively expressed by the plant is introduced into the genome of the plant. In other embodiments, the plant comprises a polynucleotide encoding an engineered PAL enzyme that exists independently of the genome. Methods of genetically engineering plants using recombinant biology or gene editing, such as CRISPR/Cas based gene editing, are known to those of skill in the art.

In some embodiments, the plants further comprise additional mutations that affect how they absorb and utilize atmospheric carbon. The inventors have previously identified mutations in Arabidopsis thaliana that deregulate the first step of the shikimate pathway, i.e., a pathway that connects central carbon metabolism to the pathway for aromatic amino acid biosynthesis in plants. See Yokoyama et al., Science Advances 8(23): eabo3416 (2022), which is hereby incorporated by reference in its entirety. These mutations map to genomic loci that encode the three Arabidopsis isoforms of the enzyme 3-deoxy-D-arabino-heptulosonate 7-phosphate synthase (DHS), which catalyzes the first reaction of the shikimate pathway. The inventors discovered that these mutations reduce inhibition by tyrosine/tryptophan-associated compounds and that plants that express DHS enzymes comprising these mutations produce greater quantities of aromatic amino acids and assimilate greater quantities of CO₂. Thus, in some embodiments, the plants of the present invention further comprise an engineered DHS enzyme that comprises one or more of these mutations, i.e., one or more mutation at a position corresponding to residue 109, 114, 159, 240, 244, 245, 247, 248, 319, 322, or 348 of the Arabidopsis thaliana DHS1 enzyme (SEQ ID NO: 152). Plants that further comprise such engineered DHS enzymes (i.e., in addition an engineered PAL enzyme) are expected to produce even higher levels of phenylpropanoids.

Additionally, the inventors have previously identified an active site residue (i.e., residue 220 of the Medicago truncatula PDH enzyme) that determines the substrate specificity (i.e., for prephenate or arogenate) and level of tyrosine feedback inhibition of TyrA family enzymes, which are the key regulatory enzymes of tyrosine biosynthesis. See U.S. Pat. No. 11,136,559, which is hereby incorporated by reference in its entirety. These mutations may be used to enhance the production of tyrosine and tyrosine-derived products in plants. Thus, in some embodiments, the plants of the present invention further comprise an engineered TyrA enzyme. In some embodiments, the engineered TyrA enzyme is an engineered arogenate dehydrogenase (ADH) enzyme comprising a non-acidic amino acid residue at a position corresponding to residue 220 of the Medicago truncatula ADH enzyme (e.g., SEQ ID NO: 153, which comprises a D220C mutation). These engineered ADH enzymes have increased prephenate dehydrogenase (PDH) activity and relaxed tyrosine sensitivity as compared to the corresponding wild-type ADH enzyme. In other embodiments, the engineered TyrA enzyme is an engineered PDH enzyme comprising an aspartic acid or glutamic acid at a position corresponding to residue 220 of the Medicago truncatula PDH enzyme (e.g., SEQ ID NO: 154, which comprises a C220D mutation). These engineered PDH enzymes have increased ADH activity and increased tyrosine sensitivity as compared to the corresponding wild-type PDH enzyme. Plants that further comprise such engineered TyrA enzymes (i.e., in addition an engineered PAL enzyme) are expected to produce even higher levels of phenylpropanoids.

### Methods for Making Plants:

In an eighth aspect, the present invention provides methods of making the plants described herein. In some embodiments, the methods comprise introducing one of the engineered PAL enzymes, polynucleotides, constructs, or vectors described herein into the plant. As used herein, “introducing” describes a process by which exogenous polypeptides or polynucleotides are introduced into a recipient cell. Suitable introduction methods include, without limitation, Agrobacterium-mediated transformation, the floral dip method, bacteriophage or viral infection, electroporation, heat shock, lipofection, microinjection, and particle bombardment.

In other embodiments, the plant comprises a native gene encoding a PAL enzyme, and the methods comprise editing the native gene to encode an engineered PAL enzyme described herein. “Gene editing” describes a process by which mutations (i.e., deletions, insertions, and substitutions) are introduced into a native gene within an organism's genome. Gene editing can be performed using several different nucleases, including zinc finger nucleases (ZFN), transcription activator-like effector nucleases (TALENs), and CRISPR/Cas endonucleases. Site-directed mutagenesis (e.g., homologous recombination) may also be used to edit a gene.

In specific embodiments, the methods comprise using a RNA-guided endonuclease (e.g., Cas9) to edit the native gene to have a mutation at a position corresponding to residue 112 of SEQ ID NO: 28. This can be accomplished by using the endonuclease to specifically edit the codon of the gene encoding the residue corresponding to residue 112 of SEQ ID NO: 28. In some embodiments, the methods further comprise using the endonuclease to edit the native gene to have a mutation at a position corresponding to residue 140 of SEQ ID NO: 28.

### Methods for Using Plants:

In a ninth aspect, the present invention provides methods for using the plants described herein to (1) produce a phenylpropanoid-derived product or (2) sequester CO₂. The methods comprise growing the plants described herein or plants genetically engineered to produce the engineered PAL enzymes described herein. The methods for producing phenylpropanoid-derived products further comprise purifying the phenylpropanoid-derived products produced by the plant.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1

In the following example, the inventors describe their discovery of a novel mutation that is necessary to convert monofunctional phenylalanine ammonia-lyase (PAL) enzymes into bifunctional phenylalanine tyrosine ammonia-lyase (PTAL) enzymes.

### BACKGROUND

Acquisition of the ability to synthesize lignin was one of the most important events that allowed vascular plants to migrate from water to land and adapt to the harsh environment. Lignin is essential in land plants for providing mechanical strength, facilitating water transportation, and strengthening the physical barrier against biotic and abiotic stresses. In addition to cellulose and hemicelluloses, lignin is one of the major components of plant secondary cell walls, and up to 30% of photosynthetically fixed carbon is utilized to produce lignin. Lignin hinders the efficient use of cell wall polysaccharides as a source of pulp, paper, and bioethanol. However, lignin is the only abundant, renewable feedstock that comprises aromatics. Thus, it has potential for use in the production of sustainable, value-added aromatic materials and high-energy-density solid fuels.

The monocot grass plant group is one of the most widely distributed plant groups on earth and contains 780 genera and about 12,000 species. These plants succeeded in expanding their habitat from forest to harsh open land by developing a series of morphological, physiological, and biochemical features. This plant group contains a substantial number of economically important crops. For example, grass cereal crops (e.g., rice, wheat, and corn) comprise a major portion of most people's diets, and grass straws are used as livestock feeds. This plant group also contains several crops with superior biomass productivity (e.g., switchgrass, sorghum, and Miscanthus) that have potential for use in the production of plant-based energy and materials. Grasses are classified as Poales, a large order of flowering, monocotyledonous plants that contains around 21,000 species of great diversity that evolved within a relatively short evolutionary timescale (Givnish et al., 2010; McKain et al., 2016) (FIG. 1A).

Although lignin is an indispensable component of vascular plants, the biosynthesis and structure of lignin differ not only among plant species but also across the organ and cell types of individual plants (Renault et al., 2019; Vanholme et al., 2019). In all vascular plants, lignin is composed of the monomeric units guaiacyl (G), syringyl unit (S), and p-hydroxyphenyl (H), which are produced via polymerization of coniferyl alcohol, sinapyl alcohol, and p-hydroxyphenyl alcohol, respectively. In addition to these three monomers, grass lignin uniquely incorporates γ-acylated (p-coumarylated and feruloylated) monomers and flavone tricin (FIG. 1B). The G/S/H lignin monomers are synthesized from the aromatic amino acid phenylalanine (L-Phe) through the phenylpropanoid pathway (FIG. 1B). In the first step of this pathway, L-Phe is deaminated by the enzyme phenylalanine ammonia-lyase (PAL) to produce cinnamic acid, which is then hydroxylated by the enzyme cinnamate 4-hydroxylase (C4H) to produce p-coumaric acid (FIG. 1B). In addition to this highly conserved PAL-C4H pathway, grasses possess an additional entry pathway that produces p-coumaric acid and lignin from tyrosine (L-Tyr) using the tyrosine ammonia-lyase (TAL) activity of the bifunctional enzyme phenylalanine tyrosine ammonia-lyase (PTAL) (Rosler et al., 1997; Barros et al., 2016). Since this TAL pathway does not require catalysis by the enzyme C4H, it is considered more efficient than the conserved PAL-C4H pathway (Maeda, 2016).

TAL activity has been detected in plant extracts of a wide range of grass species, including species classified in both the BOP and PACMAD clades (FIG. 1A), i.e., bamboo, rice, barley, wheat, sugarcane, maize, and oat (Young and Neish, 1966; Higuchi and Shimada, 1969; Havir et al., 1971; Jangaard, 1974). Although there are several reports suggesting that TAL activity is also present in other plant lineages such as legume (Jangaard, 1974; Beaudoin-Eagan and Thorpe, 1985; Giebel, 1973; Khan et al., 2003), the detection of TAL activity in grass extract is more consistent in the literature than in other lineages. Rosler et al. (1997) demonstrated that a PAL isoform from Zea mays can utilize both L-Tyr and L-Phe as a substrate by expressing it as a recombinant protein. Later, this bifunctional PTAL enzyme was also identified in Brachypodium distachyon via in vivo transgenic down-regulation (Cass et al., 2015; Barros et al., 2016) and in vitro enzyme assays (Barros et al., 2016). In these papers, eight PAL genes were identified in B. distachyon, and one of them was demonstrated to have bifunctional PTAL activity. The fact that PTAL genes are highly expressed in vascular organs (Cass et al., 2015; Barros et al., 2016) and that around half of all lignin is produced from L-Tyr (Barros et al., 2016) suggest that the PTAL pathway has a significant physiological role. However, the details regarding the evolutionary emergence of the PTAL enzyme are unknown.

The residue His 140, which is located in the substrate binding pocket of TAL enzymes, was previously proposed to be a key residue for the acquisition of TAL activity (Dixon and Barros, 2019). This residue was shown to be critical for recognition of the substrate tyrosine based on the crystal structure of the bacterial TAL enzyme (Watts et al. 2006). PAL enzymes have a highly conserved Phe 140 at this position (Louie et al. 2006; Watts et al. 2006). When a His 140 to Phe (H140F) mutation was introduced into the bacterial TAL enzyme, the TAL enzyme (which previously had a high substrate specificity for L-Tyr) was essentially converted into a PAL enzyme with a high specificity for L-Phe (Watts et al. 2006). However, in previous studies, introducing a Phe 140 to His (F140H) mutation into the Arabidopsis PAL enzyme failed to convert it into a bifunctional PTAL enzyme (Watts et al. 2006). Further, introducing a H140F mutation into the Sorghum bicolor PTAL enzyme produced an enzyme with kinetic properties that were noticeably different from other S. bicolor PAL enzymes (Jun et al., 2018). Thus, in addition to His140, other unidentified residue(s) are thought to be necessary for the acquisition of TAL activity (Barros and Dixon, 2020).

To elucidate the evolutionary history of the emergence of the PTAL enzyme in Poales, we obtained PAL/PTAL homolog sequences from 45 monocot species, including basal-grasses and non-grass graminids, whose genomes were sequenced only recently. We found that PAL orthologs from non-grass graminids nested directly into the grass PTAL clade and were distinct from the PAL clade. Biochemical characterization of recombinant PAL/PTAL homologs demonstrated that PTAL enzymes emerged in the common ancestor of the non-grass graminid Joinvillea ascendens and grasses, just before the emergence of grasses. A combined approach using phylogeny-guided sequence comparison and site-directed mutagenesis identified an additional mutation, Ser112 to Ile (S112I), that is essential for the transition from a monofunctional PAL enzyme to a bifunctional PTAL enzyme. We found that introduction of S112I and F140H mutations into PAL enzymes from J. ascendans and Arabidopsis thaliana conferred significant TAL activity to these enzymes.

### Results:

### PTAL Evolved in a Common Ancestor of Grasses and the Non-Grass Graminid Joinvillea

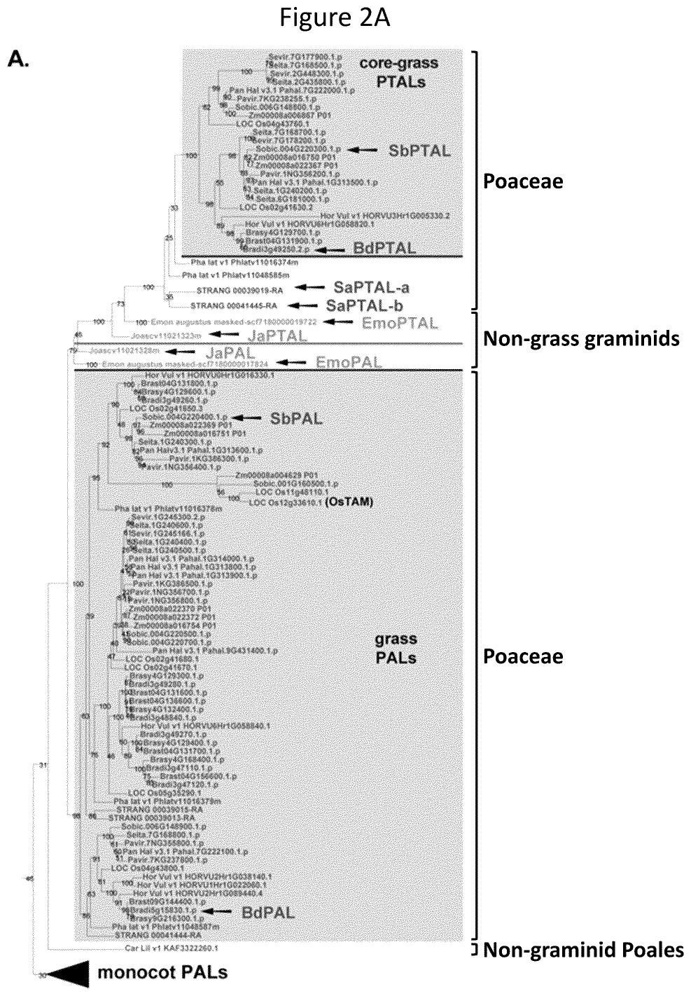

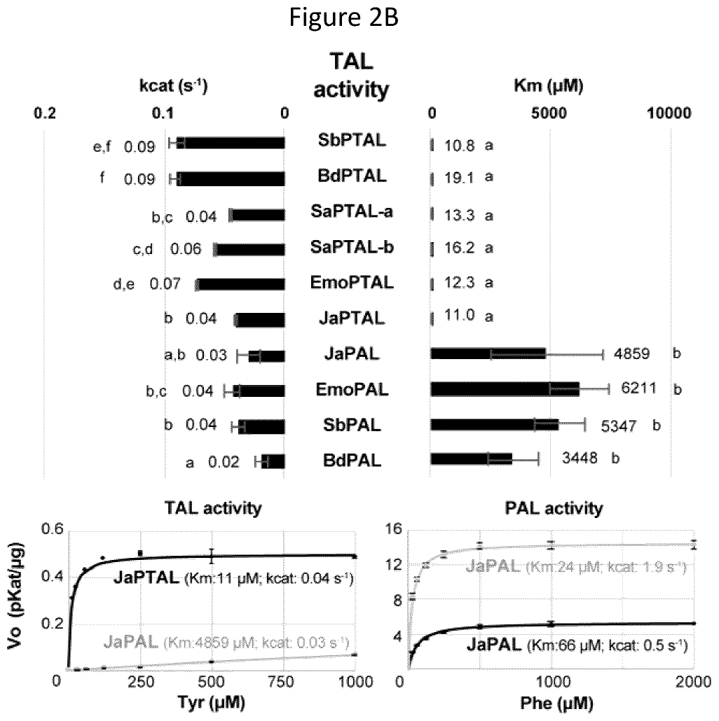

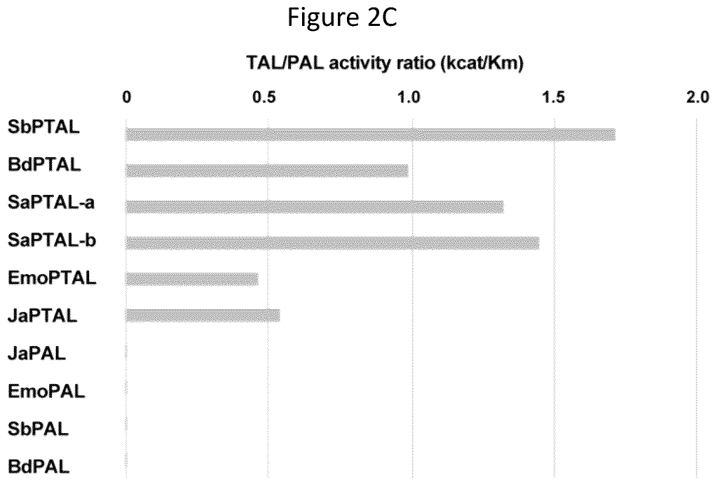

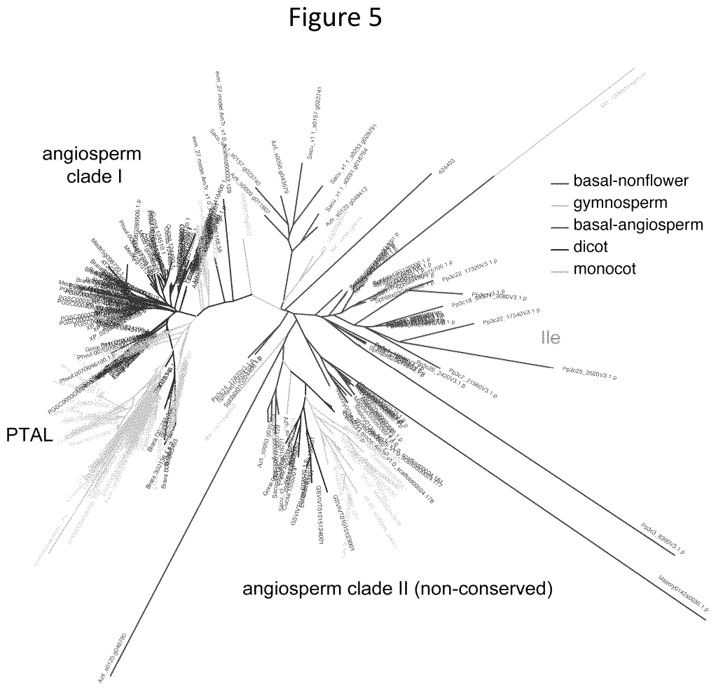

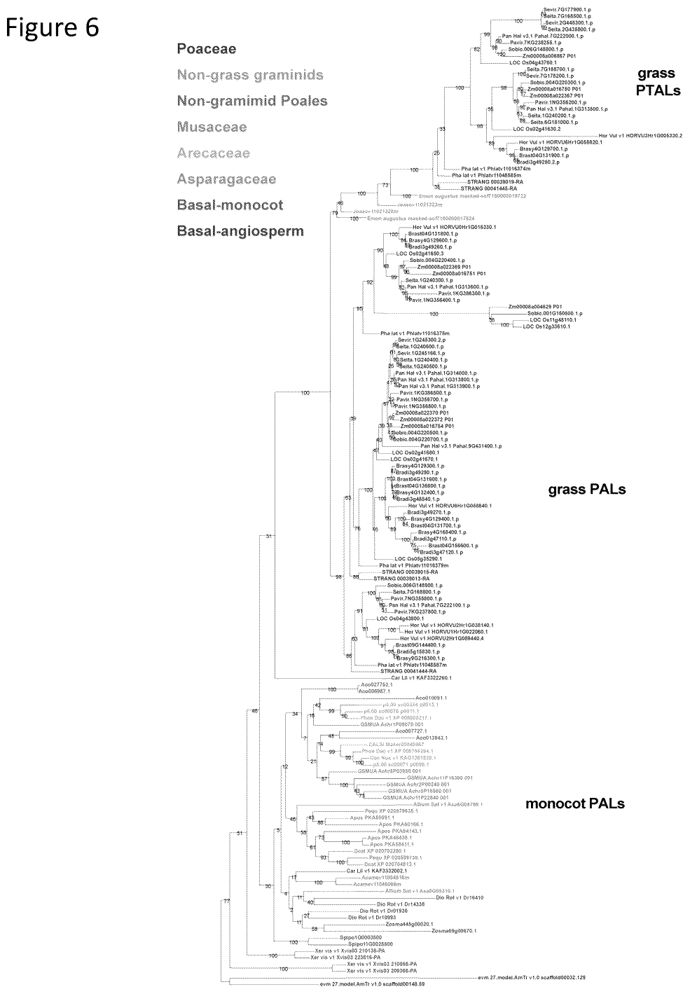

To determine when PTAL enzymes emerged in grasses, we obtained the genome sequences of 44 species of green plants, identified their PAL family enzymes using the PTAL orthogroup from OrthoFinder (Table 1), and generated a large-scale phylogenetic tree of plant PAL and PTAL enzymes. The angiosperm PAL family was divided into two distinct clades: clades I and II. Clade I includes well-characterized angiosperm PAL enzymes (e.g., from Arabidopsis thaliana, Cochrane et al., 2004) and both PAL and PTAL enzymes from grasses, such as Zea mays (Rosler et al., 1997), Sorghum bicolor (Jun et al., 2018), and Brachypodium distachyon (Barros et al., 2016) (FIG. 5). The clade II enzymes have not been characterized. We built a detailed phylogenetic tree of the clade I monocot PAL/PTAL family enzymes by identifying another orthogroup that includes 45 monocot species. In our analysis, we included several sister lineages to the core grasses, whose genome sequences became available only recently (FIG. 1B; Table 2), including a grass that diverged at the base of Poaceae (Streptochaeta angustifolia) and two non-grass graminid species (i.e., Joinvillea ascendens and Ecdeiocolea monostachya) (FIG. 1B). We found that PAL orthologs from S. angustifolia, J. ascendens, and E. monostachya nested directly into the PTAL clade of core grasses and were separate from the PAL clade of the remaining grasses (FIG. 2A; FIG. 6). This result suggests that monocot PAL enzymes diverged at a common ancestor of the non-grass graminids and that PTAL enzymes subsequently emerged under the selective pressure (FIG. 2A; FIG. 6).

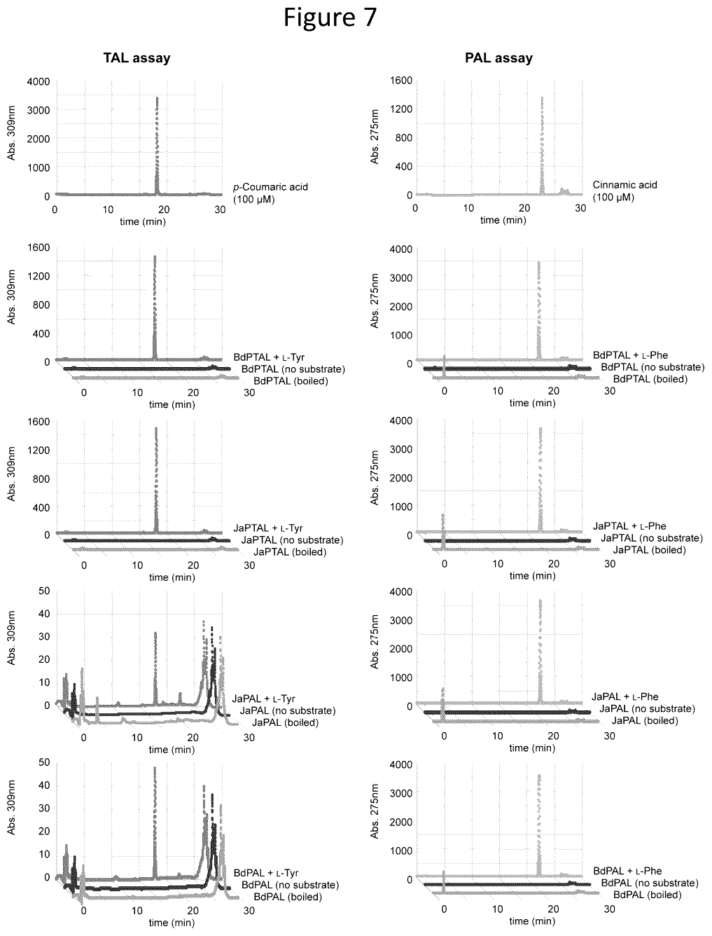

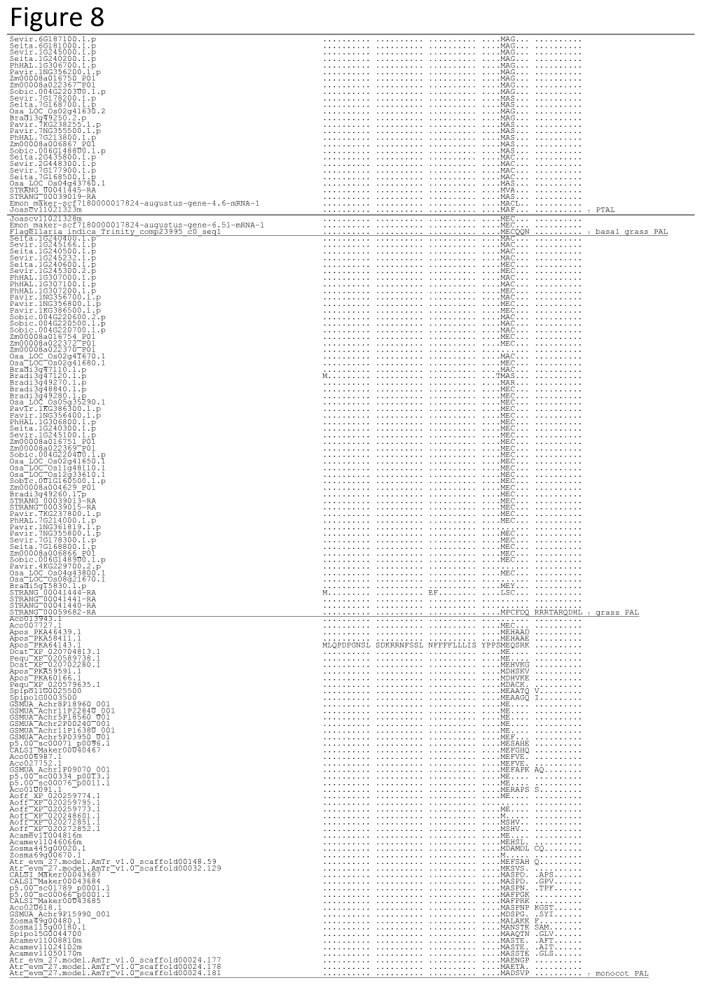

The residue His 140, which is located in the substrate binding pocket of TAL enzymes, was previously shown to be critical for the recognition of the substrate tyrosine based on the crystal structure of the bacterial enzyme (Watts et al. 2006). In contrast, PAL enzymes have highly conserved Phe 140 at this position (Louie et al. 2006, Watts et al. 2006). When the His residue of a bacterial TAL enzyme was mutated to Phe, the TAL enzyme was essentially converted to a PAL enzyme (Watts et al. 2006). To predict the functionality of the PAL/PTAL orthologs from S. angustifolia, J. ascendens, and E. monostachya (which are labeled in FIG. 2A), we compared their protein sequences to those of the PTAL enzymes from the core grass clade and PAL enzymes in the grass and monocot clades (FIG. 2A). Both of the S. angustifolia enzymes (i.e., STRANG_00039019-RA and STRANG_00041445-RA) and one of enzyme from each of J. ascendens (i.e., Joascv11021323m) and E. monostachya (i.e., Emon_augustus_masked-scf718000019722) possessed the His 140 residue that is critical for tyrosine recognition in the bacterial TAL enzyme (Watts et al. 2006) (FIG. 2B), suggesting that these proteins are bifunctional PTAL enzymes. To test this hypothesis, we cloned, expressed, and purified recombinant PAL/PTAL orthologs from S. angustifolia, J. ascendens, and E. monostachya as well as PAL and PTAL enzymes from Sorghum bicolor (i.e., SbPAL and SbPTAL) and Brachypodium distachyon (i.e., BdPAL and BdPTAL) as positive controls (Barros et al., 2016; Jun et al., 2018). These purified enzymes were mixed with the substrate, Phe or Tyr, at 1 mM and the production of cinnamic acid (CA) or p-coumaric acid (pCA) was analyzed by high-performance liquid chromatography to detect PAL or TAL activity. All ten of the tested enzymes showed detectable PAL and TAL activities as compared to negative controls (i.e., reactions that included boiled enzyme or no substrate) (FIG. 7). All enzymes produced similar levels of CA from Phe, whereas the production of pCA from Tyr was much higher (50-fold) in the reaction mixtures containing SbPTAL, BdPTAL, STRANG_00039019-RA, STRANG_00041445-RA, Emon_augustus_masked-scf718000019722, and Joascv11021323m than those containing Joascv11021328m, Emon_augustus_masked-scf718000017824, BdPAL, and SbPAL (FIG. 7). These results suggest that only the PAL/PTAL orthologs that comprise His 140 are bifunctional PTAL enzymes that have both TAL and PAL activity. Therefore, we tentatively named the enzymes with His 140 SaPTAL-a, SaPTAL-b, EmoPTAL, and JaPTAL, and named the enzymes with Phe140 EmoPAL and JaPAL.

To further examine the TAL activities of these PAL (i.e., JaPAL, EmoPAL, BdPAL, and SbPAL) and PTAL (i.e., SbPTAL, BdPTAL, SaPTAL-a, SaPTAL-b, EmoPTAL, and JaPTAL) enzymes, we determined the kinetic parameters of reactions using various concentrations of the substrate Tyr (FIG. 2B; Table 3). The apparent Km of the PAL enzymes ranged from 3449 to 6211 μM and the apparent Km of the PTAL enzymes ranged from 11 to 19 μM (Table 3). The kcat values of the PAL enzymes ranged from 0.02 to 0.04 s⁻¹ and the kcat values of the PTAL enzymes ranged from 0.04 to 0.09 s⁻¹ (Table 3). Consequently, the kcat/Km values of the PTAL enzymes (3.32 to 7.96 s⁻¹ μM⁻¹) were calculated to be much higher (485-fold on average) than those of the PAL enzymes (0.01 s⁻¹ μM⁻¹) (FIG. 2C). JaPTAL and JaPAL (which has a sequence similarity of 92.4%) were found to be distinct with regards to both the presence of TAL activity and the level of PAL activity. The PAL activity (kcal/Km) of JaPTAL (6.8 s⁻¹ μM⁻¹) was lower than that of JaPAL (78.8 s⁻¹ μM⁻¹) with significant differences in both k_{cat} (0.5 s⁻¹ and 1.9 s⁻¹) and Km (66 μM and 24 μM) (FIG. 2B; Table 3). The PAL/PTAL enzymes from other species showed similar kinetics to the PAL activity of JaPAL/JaPTAL, but higher Km values were observed with grass PTAL enzymes (150-227 μM) as compared to non-grass graminid PTAL enzymes (66-69 μM) (Table 3). Consequently, the TAL/PAL activity ratios (kcal/Km) for grass PTAL enzymes were higher than those of non-grass graminid PTAL enzymes (2.7-fold on average) (FIG. 2C). These quantitative data further support the hypothesis that S. angustifolia, E. monostachya, and J. ascendens have at least one enzyme having strong TAL activity. These results suggest that the bifunctional PTAL enzymes emerged within a common ancestor of grasses and the non-grass graminid J. ascendens, just before the emergence of grasses.

Additional Amino Acids are Involved in the Transition from PAL to PTAL

To experimentally test the role of His 140 in the acquisition of TAL activity, we next conducted site-directed mutagenesis on the PAL and PTAL enzymes of grasses and non-grass graminids characterized above and analyzed their effects on TAL activity. For the PAL enzymes, the residue corresponding to Phe 140 was converted to His to generate JaPAL^{F140H} EmoPAL^{F134H}, BdPAL^{F137H}, and SbPAL^{F135H}. A detailed kinetic analysis showed that, compared to the corresponding wild-type PAL enzymes, all these mutants exhibited increased overall TAL activity (kcat/Km; 9.7-fold on average) with significantly reduced Km values for Tyr (0.04-fold on average) (Table 3). For the PTAL enzymes, the residue corresponding to His 140 was converted to Phe to generate SbPTAL^{H123F}, BdPTAL^{H123F}, SaPTAL-a^{H118F}, SaPTAL-b^{H126F}, EmoPTAL^{H127F}, and JaPTAL^{H125F}. Compared to the corresponding wild-type PTAL enzymes, all these mutants exhibited decreased TAL activity (0.01-fold on average) and significantly increased Km for Tyr (13.2-fold on average) (FIG. 3A; Table 3). These results further support the role of His140 as a critical residue for the recognition of Tyr substrate in PTAL enzymes, consistent with prior studies (Watts et al., 2006; Louie et al., 2006; Jun et al., 2018). However, the Km values for TAL activity were still much higher in PAL^{F140H} mutants (222-450 μM) than in wild-type PTALs (11-19 μM) and lower in PTAL^{H140F} mutants (531-765 μM) than in wild-type PALs (3448-6211 μM) (Table 3). As a result, the TAL activity of the PAL^{F140H} mutants was much weaker (˜19% on average) than that of the wild-type PTAL enzymes, and PTAL^{H140F} mutants still showed higher TAL activity than that of the wild-type PAL enzymes (FIG. 3A). The PAL activity of the PAL^{F140H} and PTAL^{H140F} mutants showed much higher (35-fold on average) and lower (0.04-fold on average) Km values, respectively, toward Phe compared with the corresponding wild-type enzymes as expected, but an unexpected reduction in the kcat of the PTAL^{H140F} mutant was observed (0.25-fold on average) (Table 3). Thus, unlike in the bacterial TAL enzyme (Watts et al., 2006), other residues besides the His 140 are likely important for the acquisition of strong TAL activity in the PTAL enzymes of grasses and closely-related non-grass graminids.

Introduction of Eight Additional Mutations Besides F140H Converts PAL into PTAL

To identify the additional residues critical for the transition of PAL to PTAL in this plant lineage, we conducted a phylogeny-guided sequence comparison (Maeda, 2019) utilizing the phylogenetic distribution of the functional PAL and PTAL enzymes (FIG. 2A). In the amino acid sequence alignment of monocot PAL and PTAL enzymes (FIG. 3B, FIG. 8), we identified 16 residues that are highly conserved in PTAL enzymes. These highly conserved residues include 8 residues (denoted using circles in FIG. 3B) that are highly conserved within PAL and PTAL groups but are distinct between these two groups, as well as 8 residues (denoted using triangles in FIG. 3B) that are highly conserved among PTAL enzymes but are variable among PAL enzymes (FIG. 3B; Table 4). To determine the position of these residues within the PAL/PTAL protein structures, we generated a homology model of JaPAL from J. ascendens using the well-characterized parsley PAL structure as a template (PDB:6F6T, Bata et al., 2021). We found that most of the 16 highly conserved residues are located near the active center, with the exception of a few peripheral triangle residues (FIG. 3B).

To investigate the potential role of these residues in TAL activity, we generated two JaPAL mutant enzymes, one with PTAL-type substitutions in the 8 circle residues and the other with PTAL-type substitutions in both the circle and triangle residues (Table 4) in addition to the F140H mutation (JaPAL^{F140H_MUT8} and JaPAL^{F140H_MUT16}, respectively). Kinetic assays showed that the apparent Km value of JaPAL^{F140H_MUT8} (17.9 μM) was significantly improved compared to that of the JaPAL^{F140H} single mutant (222.7 μM) and closely approached that of wild-type JaPTAL with similar kcat values (FIG. 3C; Table 3). JaPAL^{F140H_MUT8} had a 2-fold higher Km for Phe as compared to wild-type JaPTAL with comparable kcat values (FIG. 3C). JaPAL^{F140H_MUT16} also showed significantly improved Km (42.2 μM) for TAL activity as compared to JaPAL^{F140H} (and wild-type JaPAL) but, unexpectedly, to a lesser extent than JaPAL^{F140H_MUT8} (FIG. 3C). Thus, these results demonstrate that some of the 8 circle residues are involved in TAL activity in PTAL enzymes from non-grass graminids and suggest that the overall configuration of the active site may be critical for the acquisition of bifunctional PTAL activity.

### S112I is Critical for Gaining the TAL Activity in Graminid PTALs

To determine which of the 8 circle residues are essential in the conversion of PAL enzymes to PTAL enzymes (FIG. 3C), we mutated, one by one, each one of these 8 residues back to the PAL type in JaPAL^{F140H_MUT8} and determined their effects on catalytic efficiency. The substitution of seven out of eight residues had no to minor impacts on the overall TAL and PAL activity of the mutant enzymes (FIG. 4A). In contrast, when the I112S substitution was introduced to JaPAL^{F140H_MUT8} (JaPAL^{F140H_MUT8_I112S}), both TAL and PAL activities were significantly decreased due to an increase of Km value and decrease of kcat value (FIG. 4A; Table 3). Therefore, the Ile 112 residue of the PTAL enzyme appears to be crucial for TAL activity.

We generated homology model structures of JaPAL and JaPTAL proteins using the parsley PAL and sorghum PTAL enzymes, respectively, as templates (FIG. 4B). We found that the Ser/Ile112 residue does not directly face the substrate but is located next to Tyr113/98 (PAL/PTAL), which is a critical proton acceptor for catalysis (Rother et al., 2002; Jun et al., 2018). These Ser/Ile 112-Tyr113 residues are in the ‘inner mobile loop’, which has been suggested to be important for substrate binding and catalysis (Rother et al., 2002; Dixon and Barros, 2019). Therefore, we hypothesize that a structural change in the inner-mobile loop affects the structure of the substrate binding pocket, resulting in the different catalytic activities of graminid PAL and PTAL enzymes.

Introduction of F140H and S112I is Sufficient to Change PAL into PTAL

To test this hypothesis further, the reciprocal S112I mutation was introduced into the JaPAL^{F140H} single mutant to generate the JaPAL^{F140H_S112I} double mutant. For comparison, a single mutant in which the residue corresponding to Ser112 was converted to Ile (i.e., JaPAL^{S112I}) was generated as well. While kcat was not drastically affected by these mutations, Km of the JaPAL^{F140H_S112}I mutant for TAL activity (17.5 μM) became significantly lower than those of wild-type JaPAL (4859 μM) and the single mutants JaPAL^{F140H} and JaPAL^{S112I} (223 μM and 354 μM, respectively) and reached to the level of wild-type JaPTAL (FIG. 4C). Thus, we identified an additional residue, Ile 112, which is essential for TAL activity, and our data demonstrate that the introduction of the S112I and F140H mutations is nearly enough to convert monofunctional PAL enzymes into bifunctional PTAL enzymes.

To test whether two amino acid substitutions equivalent to F140H and S112I can also confer TAL activity in distantly related PAL enzymes, we introduced these mutations into a recombinant Arabidopsis PAL1 enzyme that has higher PAL activity and weak TAL activity (Cochrane et al., 2004; Watts et al., 2006) (Table 3). AtPAL1^{F144H_S116I} showed a drastic reduction in its Km towards Tyr (20.2 μM) as compared to that of wild-type AtPAL1 (3070 μM) and its single mutants (AtPAL1^{F144H} and AtPAL1^{S116I}) (314 μM and 515 μM, respectively) (FIG. 4D). Overall, the kinetics behavior of the AtPALIF^{144H_S116I} and JaPAL^{H140F_I112S} double mutants were similar (FIGS. 4C-4D). Thus, these results demonstrate that conversion of monofunctional PAL enzymes into bifunctional PTAL enzymes can be achieved via introduction of two mutations in distantly related plant PAL enzymes.

The protein sequences of the JaPAL and AtPAL1 enzymes tested in this example are outlined in Table 6, and the DNA sequences of the JaPAL and AtPAL1 enzymes tested in this example are outlined in Table 7.

### Tables:

TABLE 1
List of sequence data used to build the green plant phylogenetic tree

| | | Gene starts | | Division/ | Common |
|---|---|---|---|---|---|
| File name | Species | with | Label | clade | name |
| Atrichopoda_291_v1.0.protein_{—} | Amborella | evm_27.model. | basal- | Angiosperms | Amborella |
| primaryTranscriptOnly.fa.mod.fa | trichopoda | AmTr_v1.0 | angiosperm | | |
| Ppatens_318_v3.3.protein_{—} | Physcomitrella | Pp | basal- | Bryophyta | moss |
| primaryTranscriptOnly.fa.mod.fa | patens | | nonflower | | |
| Sfallax_522_v1.1.protein_{—} | Sphagnum fallax | Sphfalx | basal- | Bryophyta | flat-topped |
| primaryTranscriptOnly.fa.mod.fa | | | nonflower | | bogmoss |
| Smoellendorffii_91_v1.0.protein_{—} | Selaginella | XXXXXX | basal- | Lycophytes | spike moss |
| primaryTranscriptOnly.fa.mod.fa | moellendorffii¬† | or XXXXX | nonflower | | |
| Mpolymorpha_320_v3.1.protein_{—} | Marchantia | Mapoly | basal- | Marchantiophyta | liverwort |
| primaryTranscriptOnly.fa.mod.fa | polymorpha¬† | | nonflower | | |
| Azolla_filiculoides.protein. | Azolla | Azfi_{—} | basal- | Polypodiophyta | fern |
| highconfidence_v1.1.fasta | filiculoides | | nonflower | | |
| Salvinia_cucullata.protein. | Salvinia | Sacu_v1.1 | basal- | Polypodiophyta | watermoss |
| highconfidence_v1.2.fasta | cucullata | | nonflower | | |
| Dcarota_388_v2.0.protein_{—} | Daucus carota | DCAR | dicot | Asterids | wild carrot |
| primaryTranscriptOnly.fa.mod.fa | | | | | |
| GCF_000188115.4_SL3.0_{—} | Solanum | NP_ or XP_{—} | dicot | Asterids | tomato |
| protein.faa.mod.fa | lycopersicum | | | | |
| Mguttatus_256_v2.0.protein_{—} | Mimulus guttatus | Migut | dicot | Asterids | monkey |
| primaryTranscriptOnly.fa.mod.fa | | | | | flower |
| Stuberosum_448_v4.03.protein_{—} | Solanum_tuberosum | PGSC | dicot | Asterids | potato |
| primaryTranscriptOnly.fa.mod.fa | | | | | |
| Ahypochondriacus_459_v2.1.protein_{—} | Amaranthus | AH | dicot | Eudicot | Prince-of- |
| primaryTranscriptOnly.fa.mod.fa | hypochondriacus | | | | Wales feather |
| Acoerulea_322_v3.1.protein_{—} | Aquilegia | Aqcoe | dicot | Eudicot | blue |
| primaryTranscriptOnly.fa.mod.fa | coerulea | | | | colombine |
| Athaliana_167_TAIR10.protein_{—} | Arabidopsis | AT | dicot | Rosid | Arabidopsis |
| primaryTranscriptOnly.fa.mod.fa | thaliana | | | | |
| Boleraceacapitata_446_v1.0.protein_{—} | Brassica | Bol | dicot | Rosid | cabbage |
| primaryTranscriptOnly.fa.mod.fa | oleracea capitata | | | | |
| BrapaFPsc_277_v1.3.protein_{—} | Brassica rapa | Brara | dicot | Rosid | turnip |
| primaryTranscriptOnly.fa.mod.fa | | | | | |
| Csativus_122_v1.0.protein_{—} | Cucumis sativus | Cucsa | dicot | Rosid | cucumber |
| primaryTranscriptOnly.fa.mod.fa | | | | | |
| Egrandis_297_v2.0.protein_{—} | Eucalyptus | Eucgr | dicot | Rosid | rose gum |
| primaryTranscriptOnly.fa.mod.fa | grandis | | | | |
| Fvesca_501_v2.0.a2.protein_{—} | Fragaria vesca | gene | dicot | Rosid | wild |
| primaryTranscriptOnly.fa.mod.fa | | | | | strawberry |
| Graimondii_221_v2.1.protein_{—} | Gossypium | Gorai | dicot | Rosid | cotton |
| primaryTranscriptOnly.fa.mod.fa | raimondii | | | | |
| Mtruncatula_285_Mt4.0v1.protein_{—} | Medicago | Medtr | dicot | Rosid | legume |
| primaryTranscriptOnly.fa.mod.fa | truncatula | | | | |
| Ptrichocarpa_210_v3.0.protein_{—} | Populus | Potri | dicot | Rosid | poplar/black |
| primaryTranscriptOnly.fa.mod.fa | trichocarpa | | | | cottonwood |
| Pvulgaris_442_v2.1.protein_{—} | Phaseolus | Phvul | dicot | Rosid | common bean |
| primaryTranscriptOnly.fa.mod.fa | vulgaris | | | | |
| Rcommunis_119_v0.1.protein_{—} | Ricinus | 2, 3, 4, 5, or | dicot | Rosid | castor bean |
| primaryTranscriptOnly.fa.mod.fa | communis | 6+ | | | |
| | | XXXX.mXXXXX | | | |
| Tcacao_233_v1.1.protein_{—} | Theobroma | Thecc | dicot | Rosid | cocoa |
| primaryTranscriptOnly.fa.mod.fa | cacao | | | | |
| Vvinifera_145 | Vitis vinifera | GSVIV | dicot | Rosid | grape |
| Kfedtschenkoi_382_v1.1.protein_{—} | Kalanchoe | Kaladp | dicot | Eudicot | formerly |
| primaryTranscriptOnly.fa.mod.fa | fedtschenkoi¬† | | | | Bryophyllum |
| | | | | | fedtschenkoi |
| Creinhardtii_281_v5.6.protein_{—} | Chlamydomonas | Cre | greenalgae | Chlorophyta | green algae |
| primaryTranscriptOnly.fa.mod.fa | reinhardtii | | | | |
| Pabies1.01.0-HC-pep.faa.mod.fa | Picea abies | MA_{—} | gymnosperm | Pinophyta | norway |
| | | | | | spruce |
| Aamericanusv1.1.primaryTrs.pep.fa.mod.fa | Acorus | Aca | monocot | Monocot | American |
| | americanus | | | | sweet |
| | | | | | flag/wetland |
| | | | | | plant |
| Spolyrhiza_290_v2.protein_{—} | Spirodela | Spipo | monocot | Monocot | duckweed |
| primaryTranscriptOnly.fa.mod.fa | polyrhiza | | | | |
| Zmarina_324_v2.2 | Zostera marina | Zosma | monocot | Monocot | sea grass |
| Jascendensv1.1.primaryTrs.pep.fa. | Joinvillea | Joasc | monocot | Commelinids | Joinvillea |
| mod.fa | ascendens | | | | |
| Macuminata_304_v1.protein_{—} | Musa acuminata | GSMUA | monocot | Commelinids | banana |
| primaryTranscriptOnly.fa.mod.fa | | | | | |
| proteome.all_transcripts.calsi.fasta. | Calamus | CALSI | monocot | Commelinids | rattan palm |
| mod.fa | simplicifolius | | | | |
| proteome.all_transcripts.egu.fasta. | Elaeis guineensis | p5.00_sc | monocot | Commelinids | oil palm |
| mod.fa | | | | | |
| Bdistachyon_556_v3.2.protein_{—} | Brachypodium | Bradi | monocot | Commelinids | purple false |
| primaryTranscriptOnly.fa.mod.fa | distachyon | | | | brome |
| Osativa_323_v7.0.protein_{—} | Oryza sativa | LOC_Os | monocot | Commelinids | rice |
| primaryTranscriptOnly.fa.mod.fa | | | | | |
| Pvirgatum_516_v5.1.protein_{—} | Panicum | Pavir | monocot | Commelinids | switchgrass |
| primaryTranscriptOnly.fa.mod.fa | virgatum | | | | |
| Sitalica_312_v2.2.protein_{—} | Setaria italica | Seita | monocot | Commelinids | fostail millet |
| primaryTranscriptOnly.fa.mod.fa | | | | | |
| Streptochaeta_maker_max_{—} | Streptochaeta | STRANG_{—} | monocot | Commelinids | Streptochaeta |
| proteins_V1.fasta.mod.fa | angustifolia | | | | |
| Sviridis_500_v2.1.protein_{—} | Setaria viridis | Sevir | monocot | Commelinids | green foxtail |
| primaryTranscriptOnly.fa.mod.fa | | | | | |
| ZmaysPH207_443_v1.1 | Zea mays | Zm | monocot | Commelinids | maize |
| Acomosus_321_v3.protein_{—} | Ananas comosus | Aco | monocot | Commelinids | pineapple |
| primaryTranscriptOnly.fa.mod.fa | | | | | |

TABLE 2
List of genome sequence data used to build the monocot phylogenetic tree

| | | Gene starts | | Common | |
|---|---|---|---|---|---|
| File name | Species | with | Clade | name | Ref |
| Atrichopoda_291_v1.0.protein_{—} | Amborella | evm_27.model. | Angiosperm | Amborella | ncbi |
| primaryTranscriptOnly.fa.mod.fa | trichopoda | AmTr_V1.0 | | | |
| Aamericanusv1.1.primaryTrs.pep | Acorus | Acame | monocot | wetland plant | phytozome |
| | americanus | | | | |
| Zmarina_324_v2.2 | Zostera marina | Zosma | monocot | sea grass | phytozome |
| Spolyrhiza_290_v2 | Spirodela | Spipo | monocot | duckweed | phytozome |
| | polyrhiza | | | | |
| GCA_002076135.1_ASM207613v1 | Xerophyta | Xer_vis_{—} | monocot | | ncbi |
| | viscosa | | | | |
| GCF_001876935.1_{—} | Asparagus | Aoff_{—} | monocot | asparagus | ncbi |
| Asparagusof.V1_protein.faa | officinalis | | | | |
| GCA_002786265.1_{—} | Apostasia | Apos_{—} | monocot | orchid | ncbi |
| ApostasiaASM278626v1_protein.faa | shenzhenica | | | | |
| GCF_001263595.1_Pequestris_{—} | Phalaenopsis | Pequ_{—} | monocot | | ncbi |
| ASM126359v1_protein.faa | equestris | | | | |
| GCF_001605985.2_Dendrobium_{—} | Dendrobium | Dcat_{—} | monocot | | ncbi |
| catASM160598v2_protein.faa | catenatum | | | | |
| Garlic.pep.fa.mod.fa | Allium sativum | Allium_Sat | monocot | garlic | ncbi |
| Dioscorea_rotundata_TDr96_F1_{—} | Dioscorea | Dio_Rot_v1 | monocot | white yam | DNA |
| v1.0.protein_20170801.fasta.mod.fa | rotundata | | | | Databank of |
| | | | | | Japan |
| | | | | | (DDBJ) |
| Macuminata_304_v1 | Musa acuminata | GSMUA_{—} | monocot | banana | phytozome |
| calsi_proteome.sel | Calamus | CALSI_{—} | monocot | rattan palm | plaza_v4.5_{—} |
| | simplicifolius | | | | monocots |
| egu_proteome.sel | Elaeis guineensis | p5.00_{—} | monocot | oil palm | ncbi |
| Cocos_GCA_008124465.1_{—} | Cocos nucifera | Coc_Nuc | monocot | coconut palm | Ncbi |
| ASM812446v1_protein.faa | | | | | |
| Phoenix_GCF_009389715.1_palm_55x_up_{—} | Phoenix | Phoe_Dac | monocot | date palm | Ncbi |
| 171113_PBpolish2nd_filt_p_protein.faa | dactylifera | | | | |
| Carex_littledalei_GCA_011114355.1 | Carex littledalei | Car_Lil | monocot | | Ncbi |
| ASM1111435v1_protein.faa.mod.fa | | | | | |
| Acomosus_321_v3 | Ananas comosus | Aco | monocot | pineapple | phytozome |
| Jascendensv1.1.primaryTrs.pep.fa.mod | Joinvillea | Joascv | monocot | | phytozome |
| | ascendens | | | | |
| Emo_MaSuRCA_v1_v0.all. | Ecdeiocolea | Emon_{—} | monocot | | Matthew |
| MERGE.proteins | monostachya | | | | Moscou |
| Streptochaeta | Streptochaeta | STRANG_{—} | monocot | basal grass | phytozome |
| | angustifolia | | | | |
| Platifoliusv1.1.primaryTrs.pep [coge | Pharus latifolius | Pha_lat | monocot | | |
| genome (not annotated)] | | | | | |
| Othomaeum_386_v1.0.protein_{—} | Oropetium | Oropetium | monocot | resurrection | phytozome |
| primaryTranscriptOnly.fa | thomaeum | | | plant | |
| Sbicolor_454_v3.1.1.protein_{—} | Sorghum bicolor | Sobic | monocot | cereal grass | phytozome |
| primaryTranscriptOnly.fa | | | | | |
| ZmaysPH207_443_v1.1 | Zea mays | Zm | monocot | maize | phytozome |
| Sviridis_500_v2.1 | Setaria viridis | Sevir | monocot | green foxtail | phytozome |
| Sitalica_312_v2.2.protein_{—} | Setaria italica | Seita | monocot | foxtail millet | phytozome |
| primaryTranscriptOnly.fa | | | | | |
| Pvirgatum_516_v5.1 | Panicum | Pavir | monocot | switchgrass | phytozome |
| | virgatum | | | | |
| PhalliiHAL_496_v2.1.protein_{—} | Panicum hallii | PhHAL | monocot | Hall's | phytozome |
| primaryTranscriptOnly.fa | | | | panicgrass | |
| Osativa_323_v7.0 | Oryza sativa | Osa_LOC_{—} | monocot | rice | phytozome |
| Bstacei_316_v1.1.protein_{—} | Brachypodium | Brast | monocot | grass | phytozome |
| primaryTranscriptOnly.fa | stacei | | | | |
| Bsylvaticum_490_v1.1.protein_{—} | Brachypodium | Brasy | monocot | grass | phytozome |
| primaryTranscriptOnly.fa | sylvaticum | | | | |
| Bdistachyon_556_v3.2 | Brachypodium | Bradi | monocot | grass | phytozome |
| | distachyon | | | | |
| Hvulgare_462_r1.protein_{—} | Hordeum vulgare | Hor_Vul | monocot | barley | JGI |
| primaryTranscriptOnly.fa.mod.fa | | | | | |

TABLE 3
Kinetic parameters of recombinant PTAL orthologs with or without mutations

| | TAL assay | | | PAL assay | | |
|---|---|---|---|---|---|---|
| | | | kcat/Km | | | kcat/Km |
| Protein | Km (μM) | kcat (s⁻¹) | (s⁻¹ mM⁻¹) | Km (μM) | kcat (s⁻¹) | (s⁻¹ mM⁻¹) |
| SbPTAL | 10.8 ± 2.2 | 0.09 ± 0.00 | 7.96 ± 1.18 | 150.1 ± 14.4 | 0.69 ± 0.01 | 4.63 ± 0.49 |
| BdPTAL | 19.1 ± 2.4 | 0.09 ± 0.00 | 4.78 ± 0.36 | 216.6 ± 10.3 | 1.05 ± 0.05 | 4.84 ± 0.11 |
| SaPTAL-a | 13.3 ± 1.0 | 0.04 ± 0.00 | 3.32 ± 0.13 | 154.5 ± 3.7 | 0.39 ± 0.01 | 2.51 ± 0.03 |
| SaPTAL-b | 16.2 ± 0.5 | 0.06 ± 0.00 | 3.57 ± 0.07 | 227.4 ± 1.5 | 0.56 ± 0.02 | 2.46 ± 0.10 |
| EmoPTAL | 16.3 ± 1.2 | 0.04 ± 0.0 | 2.55 ± 0.16 | 64.1 ± 3.1 | 0.48 ± 0.00 | 7.54 ± 0.39 |
| JaPTAL | 11.0 ± 0.4 | 0.04 ± 0.00 | 3.68 ± 0.22 | 65.6 ± 1.4 | 0.45 ± 0.02 | 6.80 ± 0.18 |
| JaPAL | 4859.1 ± 2350.1 | 0.03 ± 0.01 | 0.01 ± 0.00 | 24.4 ± 0.1 | 1.92 ± 0.01 | 78.60 ± 0.00 |
| EmoPAL | 4226.2 ± 150.6 | 0.03 ± 0.00 | 0.01 ± 0.00 | 27.7 ± 1.9 | 1.23 ± 0.02 | 44.70 ± 2.39 |
| BdPAL | 3448.6 ± 1045.4 | 0.02 ± 0.01 | 0.01 ± 0.00 | 21.3 ± 2.1 | 0.86 ± 0.03 | 40.74 ± 2.68 |
| SbPAL | 5347.8 ± 1284.4 | 0.04 ± 0.01 | 0.01 ± 0.00 | 43.9 ± 2.7 | 0.97 ± 0.02 | 22.05 ± 1.08 |
| SbPTAL^{H123F} | 750.7 ± 36.8 | 0.03 ± 0.00 | 0.04 ± 0.00 | 3.8 ± 2.0 | 0.10 ± 0.00 | 30.97 ± 13.70 |
| BdPTAL^{H123F} | 765.2 ± 65.8 | 0.03 ± 0.00 | 0.04 ± 0.00 | 6.0 ± 1.4 | 0.17 ± 0.00 | 29.92 ± 6.75 |
| SaPTAL-a^{H118F} | 531.0 ± 13.2 | 0.03 ± 0.00 | 0.06 ± 0.00 | 6.0 ± 0.8 | 0.20 ± 0.00 | 33.60 ± 5.10 |
| SaPTAL-b^{H126F} | 723.6 ± 54.8 | 0.05 ± 0.00 | 0.06 ± 0.01 | 6.3 ± 0.5 | 0.23 ± 0.00 | 37.05 ± 2.12 |
| EmoPTAL^{H127F} | 613.5 ± 18.1 | 0.04 ± 0.0 | 0.07 ± 0.01 | 3.6 ± 0.5 | 0.12 ± 0.00 | 32.62 ± 2.12 |
| JaPTAL^{H125F} | 535.4 ± 80.5 | 0.02 ± 0.00 | 0.04 ± 0.00 | 6.8 ± 0.7 | 0.08 ± 0.00 | 12.28 ± 0.71 |
| JaPAL^{F140H} | 222.7 ± 13.5 | 0.03 ± 0.00 | 0.13 ± 0.01 | 697.0 ± 169.2 | 0.60 ± 0.04 | 0.89 ± 0.18 |
| EmoPAL^{F134H} | 450.1 ± 14.2 | 0.02 ± 0.00 | 0.05 ± 0.00 | 1305.3 ± 25.6 | 0.42 ± 0.01 | 0.32 ± 0.01 |
| BdPAL^{F137H} | 371.3 ± 15.4 | 0.04 ± 0.00 | 0.11 ± 0.01 | 1082.0 ± 58.1 | 0.75 ± 0.01 | 0.70 ± 0.03 |
| SbPAL^{F135H} | 412.8 ± 6.5 | 0.04 ± 0.00 | 0.10 ± 0.00 | 1051.6 ± 58.5 | 0.88 ± 0.05 | 0.84 ± 0.02 |
| JaPAL^{F140H}^{_{—}}^{MUT8} | 17.9 ± 2.0 | 0.03 ± 0.00 | 1.81 ± 0.18 | 141.0 ± 6.3 | 0.60 ± 0.01 | 4.25 ± 0.22 |
| JaPAL^{F140H}^{_{—}}^{MUT16} | 42.2 ± 2.1 | 0.02 ± 0.00 | 0.56 ± 0.02 | 454.9 ± 4.6 | 0.46 ± 0.01 | 1.01 ± 0.0 |
| JaPAL^{F140H}^{_{—}}^{MUT8}^{_{—}}^{I102V} | 24.5 ± 1.4 | 0.04 ± 0.00 | 1.67 ± 0.08 | 231.7 ± 3.5 | 0.74 ± 0.01 | 3.19 ± 0.08 |
| JaPAL^{F140H}^{_{—}}^{MUT8}^{_{—}}^{I122S} | 282.3 ± 29.0 | 0.02 ± 0.00 | 0.07 ± 0.00 | 2290.9 ± 344.4 | 0.29 ± 0.04 | 0.31 ± 0.00 |
| JaPAL^{F140H}^{_{—}}^{MUT8}^{_{—}}^{G121A} | 12.6 ± 0.6 | 0.03 ± 0.00 | 2.12 ± 0.07 | 58.1 ± 3.4 | 0.57 ± 0.02 | 9.88 ± 0.31 |
| JaPAL^{F140H}^{_{—}}^{MUT8}^{_{—}}^{L138I} | 18.3 ± 0.6 | 0.05 ± 0.00 | 2.50 ± 0.02 | 74.3 ± 1.1 | 0.66 ± 0.01 | 8.86 ± 0.01 |
| JaPAL^{F140H}^{_{—}}^{MUT8}^{_{—}}^{S267A} | 43.1 ± 1.5 | 0.04 ± 0.00 | 0.96 ± 0.03 | 316.9 ± 6.6 | 0.85 ± 0.04 | 2.67 ± 0.06 |
| JaPAL^{F140H}^{_{—}}^{MUT8}^{_{—}}^{T444P} | 25.3 ± 2.4 | 0.04 ± 0.00 | 1.61 ± 0.09 | 191.9 ± 3.6 | 0.90 ± 0.05 | 4.70 ± 0.22 |
| JaPAL^{F140H}^{_{—}}^{MUT8}^{_{—}}^{A448S} | 23.3 ± 1.0 | 0.04 ± 0.00 | 1.62 ± 0.04 | 167.7 ± 11.3 | 0.80 ± 0.03 | 4.79 ± 0.43 |
| JaPAL^{F140H}^{_{—}}^{MUT8}^{_{—}}^{V500I} | 25.1 ± 3.2 | 0.05 ± 0.00 | 1.82 ± 0.14 | 150.7 ± 3.7 | 0.82 ± 0.02 | 5.45 ± 0.14 |
| JaPAL^{S112I} | 353.5 ± 45.0 | 0.05 ± 0.00 | 0.13 ± 0.01 | 2.6 ± 0.5 | 0.21 ± 0.04 | 79.80 ± 0.00 |
| JaPAL^{F140H}^{_{—}}^{S112I} | 17.2 ± 0.7 | 0.03 ± 0.00 | 1.79 ± 0.10 | 67.3 ± 2.5 | 0.77 ± 0.01 | 11.46 ± 0.21 |
| AtPAL1 | 3069.8 ± 433.4 | 0.05 ± 0.00 | 0.02 ± 0.00 | 52.2 ± 3.1 | 1.42 ± 0.07 | 27.31 ± 0.85 |
| AtPAL1^{S114I} | 515.4 ± 54.3 | 0.02 ± 0.00 | 0.04 ± 0.00 | 10.1 ± 1.9 | 0.23 ± 0.00 | 23.71 ± 4.63 |
| AtPAL1^{F144H} | 313.9 ± 13.9 | 0.01 ± 0.00 | 0.03 ± 0.00 | 1198.9 ± 21.1 | 1.58 ± 0.04 | 1.32 ± 0.02 |
| AtPAL1^{F144H}^{_{—}}^{S114I} | 20.2 ± 0.2 | 0.02 ± 0.00 | 1.05 ± 0.02 | 87.3 ± 2.9 | 0.88 ± 0.01 | 10.07 ± 0.44 |

| | | | | |
|---|---|---|---|---|
| JaPTAL^{H125F}^{_{—}}^{I97S} | Only trace activity detected. | 9.42 ± 0.8 | 0.02 ± 0.00 | 1.66 ± 0.12 |

TABLE 4
Residues potentially involved in the transition from PAL to PTAL in graminids.
Residue numbering is based on JaPAL (SEQ ID NO: 28).

| | Identity | Identity | Mutated | Mutated |
|---|---|---|---|---|
| Residue No. | in PAL | in PTAL | in JaPAL^{F140H}^{_{—}}^{MUT16} | in JaPAL^{F140H}^{_{—}}^{MUT8} |
| 70 | A (S) | G | x | |
| 102 | V | I | x | x |
| 110 | T (V/G) | G | x | |
| 112 | S | I | x | x |
| 121 | A | G | x | x |
| 129 | E (Q/K) | D | x | |
| 135 | R (K/Q/A) | V | x | |
| 138 | I | L | x | x |
| 267 | A | S | x | x |
| 271 | G (A) | A | x | |
| 279 | E (D) | D | x | |
| 334 | Y (F) | F | x | |
| 444 | P | T | x | x |
| 448 | S | A | x | x |
| 500 | I | V | x | x |
| 502 | S (A) | A | x | |

TABLE 5
Primers used in this study

| | | | |
|---|---|---|---|
| Sequence (5' to 3') | Purpose | Template | Lab ID |

Nested PCR and in-fusion cloning

| | | | |
|---|---|---|---|
| CGCGCGGCAGCCATATGATGGCGTTCCA | in-fusion cloning of | Joinvillea ascendens | pHM1810 |
| GAACGAC (SEQ ID NO: 155) | JaPTAL into pET28a | cDNA | |
| GCTCGAATTCGGATCCTCAGCAGATTGG | in-fusion cloning of | Joinvillea ascendens | pHM1811 |
| CAGGGG (SEQ ID NO: 156) | JaPTAL into pET28a | cDNA | |
| CAATTGCAGGGAGATCGAGC (SEQ ID | nested PCR for JaPAL | Joinvillea ascendens | pHM1869 |
| NO: 157) | | cDNA | |
| TGCTGTTGTAAGGTGGGGAT (SEQ ID NO: | nested PCR for JaPAL | Joinvillea ascendens | pHM1870 |
| 158) | | CDNA | |
| CGCGCGGCAGCCATATGATGGAGTGCGA | in-fusion cloning of | Joinvillea ascendens | pHM1812 |
| GAACGGC (SEQ ID NO: 159) | JaPAL into pET28a | CDNA | |
| GCTCGAATTCGGATCCTCAGCAGATTGG | in-fusion cloning of | Joinvillea ascendens | pHM1813 |
| CAGGGG (SEQ ID NO: 160) | JaPAL into pET28a | CDNA | |
| TCTTCTTCCACACCAAACG (SEQ ID NO: | nested PCR for SaPTAL- | Streptochaeta angustifolia | pHM1851 |
| 161) | a | cDNA | |
| GCACAAGAAGGATGCTAGAAAC (SEQ ID | nested PCR for SaPTAL- | Streptochaeta angustifolia | pHM1852 |
| NO: 162) | a | CDNA | |
| CGCGCGGCAGCCATATGATGGCGAGCCA | in-fusion cloning of | Streptochaeta angustifolia | pHM1814 |
| GAGGGAC (SEQ ID NO: 163) | SaPTAL-a into pET28a | CDNA | |
| GCTCGAATTCGGATCCTTAGCAGATGGG | in-fusion cloning of | Streptochaeta angustifolia | pHM1815 |
| CAGGGG (SEQ ID NO: 164) | SaPTAL-a into pET28a | cDNA | |
| ATGGTGGCCCAGAGCGAC (SEQ ID NO: | nested PCR for SaPTAL- | Streptochaeta angustifolia | pHM1841 |
| 165) | b | cDNA | |
| TTAGCAGATTGGAAGGGGC (SEQ ID NO: | nested PCR for SaPTAL- | Streptochaeta angustifolia | pHM1842 |
| 166) | b | CDNA | |
| CGCGCGGCAGCCATATGATGGTGGCCCA | in-fusion cloning of | Streptochaeta angustifolia | pHM1816 |
| GAGCGAC (SEQ ID NO: 167) | SaPTAL-b into pET28a | CDNA | |
| GCTCGAATTCGGATCCTTAGCAGATTGG | in-fusion cloning of | Streptochaeta angustifolia | pHM1817 |
| AAGGGGC (SEQ ID NO: 168) | SaPTAL-b into pET28a | CDNA | |
| CAAGAAGAGCACGCCAACTC (SEQ ID | nested PCR for SbPTAL | Sorghum bicolor RTx430 | pHM2009 |
| NO: 169) | | CDNA | |
| GCCACACACACATACGGATC (SEQ ID NO: | nested PCR for | Sorghum bicolor RTx430 | pHM2010 |
| 170) | SbPTAL | CDNA | |
| GCGCGGCAGCCATATGATGGCGGGCAAC | in-fusion cloning of | Sorghum bicolor RTx430 | pHM2011 |
| GGCGCC (SEQ ID NO: 171) | SbPTAL into pET28a | CDNA | |
| GCTCGAATTCGGATCCTTAGTTGACGAC | in-fusion cloning of | Sorghum bicolor RTx430 | pHM2012 |
| GTTGAT (SEQ ID NO: 172) | SbPTAL into pET28a | CDNA | |
| CCACTGTCAGTCACGCAATT (SEQ ID NO: | nested PCR for SbPAL | Sorghum bicolor RTx430 | pHM2066 |
| 173) | | CDNA | |
| TGCAACAGCCAAGAACATGC (SEQ ID | nested PCR for SbPAL | Sorghum bicolor RTx430 | pHM2067 |
| NO: 174) | | cDNA | |
| GCGCGGCAGCCATATGATGGAGTGCGAG | in-fusion cloning of | Sorghum bicolor RTx430 | pHM2068 |
| ACGGGT (SEQ ID NO: 175) | SbPAL into pET28a | cDNA | |
| GCTCGAATTCGGATCCTCAGCAGAGCGG | in-fusion cloning of | Sorghum bicolor RTx430 | pHM2069 |
| CAGTGG (SEQ ID NO: 176) | SbPAL into pET28a | cDNA | |
| CTCTGCAATTCGACGAGCTC (SEQ ID NO: | nested PCR for BdPAL | Brachypodium distachyon | pHM2072 |
| 177) | | BL31 cDNA | |
| AGTTCTACTGGCTGCCTACC (SEQ ID NO: | nested PCR for BdPAL | Brachypodium distachyon | pHM2073 |
| 178) | | BL31 cDNA | |
| GCGCGGCAGCCATATGATGGAGTACGAG | in-fusion cloning of | Brachypodium distachyon | pHM2074 |
| AACGGG (SEQ ID NO: 179) | BdPAL into pET28a | BL31 cDNA | |
| GCTCGAATTCGGATCCTCAGCAGAGAGG | in-fusion cloning of | Brachypodium distachyon | pHM2075 |
| CAGGGG (SEQ ID NO: 180) | BdPAL into pET28a | BL31 cDNA | |
| AGCTCCTATCTTCTTTCTTTCT (SEQ ID | nested PCR for AtPAL1 | Arabidopsis thaliana | pHM2536 |
| NO: 181) | | CDNA | |
| AACCACTTCACAGACAATCA (SEQ ID NO: | nested PCR for AtPAL1 | Arabidopsis thaliana | pHM2537 |
| 182) | | CDNA | |
| CGCGCGGCAGCCATATGATGGAGATTAA | in-fusion cloning of | Arabidopsis thaliana | pHM2522 |
| CGGGGCACAC (SEQ ID NO: 183) | AtPAL1 into pET28a | CDNA | |
| GCTCGAATTCGGATCCTTAACATATTGGA | in-fusion cloning of | Arabidopsis thaliana | pHM2523 |
| ATGGGAGCTCCG (SEQ ID NO: 184) | AtPAL1 into pET28a | cDNA | |

Sequencing analysis

| | | | |
|---|---|---|---|
| CGACTCACTATAGGGGAATTGTG (SEQ ID | sequencing of pET28a | All of the pET28a | pHM1826 |
| NO: 185) | vectors | construct generated | |
| GCTAGTTATTGCTCAGCGGTG (SEQ ID | sequencing of pET28a | All of the pET28a | pHM1827 |
| NO: 186) | vectors | construct generated | |
| CATTCAAGATCGCCGGCATC (SEQ ID NO: | sequencing of | JaPTAL-pET28a | pHM1828 |
| 187) | JaPTAL- | | |
| | pET28a | | |
| CTAACATCGAACTTGGCCGG (SEQ ID NO: | sequencing of JaPTAL- | JaPTAL-pET28a | pHM1829 |
| 188) | pET28a | | |
| TCTTCCTGGCAGAGACAAGG (SEQ ID NO: | sequencing of JaPTAL- | JaPTAL-pET28a | pHM1863 |
| 189) | pET28a | | |
| TTCCTCAATGCCGGAGTCTT (SEQ ID NO: | sequencing of JaPAL- | JaPAL-pET28a | pHM1830 |
| 190) | pET28a | | |
| CTTCTGCGAAGTCATGACCG (SEQ ID NO: | sequencing of | JaPAL-pET28a | pHM1831 |
| 191) | JaPAL- | | |
| | DET28a | | |
| CAACCCAGTGACCAACCATG (SEQ ID NO: | sequencing of | JaPAL-pET28a | pHM1832 |
| 192) | JaPAL- | | |
| | pET28a | | |
| CTACGACGCCAACATTCTCG (SEQ ID NO: | sequencing of | SaPTAL-a-pET28a | pHM1833 |
| 193) | SaPTAL- | | |
| | a-pET28a | | |
| ACATCGGCAAGCTCATGTTC (SEQ ID NO: | sequencing of SaPTAL- | SaPTAL-a-pET28a | pHM1834 |
| 194) | a-pET28a | | |
| TTGATGGCAGGAAGGTGGAT (SEQ ID NO: | sequencing of SaPTAL- | SaPTAL-b-pET28a | pHM1835 |
| 195) | b-pET28a | | |
| ATCGGAAAGCTCATGTTCGC (SEQ ID NO: | sequencing of SaPTAL- | SaPTAL-b-pET28a | pHM1836 |
| 196) | b-pET28a | | |
| CCCCAAGGAAGGTCTGGC (SEQ ID NO: | sequencing of | SbPTAL-pET28a | pHM2015 |
| 197) | SbPTAL- | | |
| | pET28a | | |
| ACATCGGCAAGCTCATGTTC (SEQ ID NO: | sequencing of SbPTAL- | SbPTAL-pET28a | pHM2016 |
| 198) | pET28a | | |
| CATCGTCAATGGCACCTCC (SEQ ID NO: | sequencing of BdPTAL- | BdPTAL^{H123F}-pET28a | pHM2026 |
| 199) | pET28a | | |
| CTCATGTTCGCGCAGTTCTC (SEQ ID NO: | sequencing of BdPTAL- | BdPTAL^{H123F}-pET28a | pHM2027 |
| 200) | pET28a | | |
| GTCTCGCCATGGTCAACG (SEQ ID NO: | sequencing of | SbPAL-pET28a | pHM2070 |
| 201) | SbPAL- | | |
| | pET28a | | |
| CCATCGGCAAGCTCATGTTC (SEQ ID NO: | sequencing of | SbPAL-pET28a | pHM2071 |
| 202) | SbPAL- | | |
| | pET28a | | |
| CCTTGCCATGGTGAACGG (SEQ ID NO: | sequencing of | BdPAL-pET28a | pHM2076 |
| 203) | BdPAL- | | |
| | pET28a | | |
| CAAGCTCATGTTTGCCCAGT (SEQ ID NO: | sequencing of | BdPAL-pET28a | pHM2077 |
| 204) | BdPAL- | | |
| | pET28a | | |

Site-directed mutagenesis (1)

| | | | |
|---|---|---|---|
| CTCAGGTTTCTGAACGCCGGGATCTTC | site-directed mutagenesis | BdPTAL-pET28a | pHM1894 |
| (SEQ ID NO: 205) | (H123F) | | |
| GTTCAGAAACCTGAGGAGCTCGACCTG | site-directed mutagenesis | BdPTAL-pET28a | pHM1895 |
| (SEQ ID NO: 206) | (H123F) | | |
| CTTAGATTCCTCAATGCCGGAATCTT | site-directed mutagenesis | JaPTAL-pET28a | pHM1896 |
| (SEQ ID NO: 207) | (^{F140H}) | | |
| ATTGAGGAATCTAAGGAGCTCTATTTG | site-directed mutagenesis | JaPTAL-pET28a | pHM1897 |
| (SEQ ID NO: 208) | (^{F140H}) | | |
| AATTAGACACCTCAATGCCGGAGTCTT | site-directed mutagenesis | JaPAL-pET28a | pHM1904 |
| (SEQ ID NO: 209) | (H128F) | | |
| TTGAGGTGTCTAATTAGCTCTCTTTGG | site-directed mutagenesis | JaPAL-pET28a | pHM1905 |
| (SEQ ID NO: 210) | (H128F) | | |
| CTCCGGTTTCTGAATGCTGGAATCTT | site-directed mutagenesis | SaPTAL-a-pET28a | pHM1900 |
| (SEQ ID NO: 211) | (H118F) | | |
| ATTCAGAAACCGGAGGAGCTCCACCTG | site-directed mutagenesis | SaPTAL-a-pET28a | pHM1901 |
| (SEQ ID NO: 212) | (H118F) | | |
| CTTCGGTTTCTCAATGCCGGAATCTT | site-directed mutagenesis | SaPTAL-b-pET28a | pHM1902 |
| (SEQ ID NO: 213) | (H127F) | | |
| ATTGAGAAACCGAAGGAGCTCCACCTG | site-directed mutagenesis | SaPTAL-b-pET28a | pHM1903 |
| (SEQ ID NO: 214) | (H127F) | | |
| CTCAGGTTTCTCAACGCCGGGATCTTCGG | site-directed mutagenesis | SbPTAL-pET28a | pHM2013 |
| CACC (SEQ ID NO: 215) | (H125F) | | |
| GTTGAGAAACCTGAGCAGCTCGACCTGG | site-directed mutagenesis | SbPTAL-pET28a | pHM2014 |
| AGCGC (SEQ ID NO: 216) | (H125F) | | |
| ATCAGACACCTCAATGCCGGCGCCTTCG | site-directed mutagenesis | SbPAL-pET28a | pHM2083 |
| GCACC (SEQ ID NO: 217) | (F135H) | | |
| ATTGAGGTGTCTGATGAGCTCCCTCTGGA | site-directed mutagenesis | SbPAL-pET28a | pHM2084 |
| GCGCG (SEQ ID NO: 218) | (F135H) | | |
| ATCCGACACCTTAATGCGGGAGCCTTCG | site-directed mutagenesis | BdPAL-pET28a | pHM2085 |
| GCACC (SEQ ID NO: 219) | (F138H) | | |
| ATTAAGGTGTCGGATGAGCTCTCTCTGCA | site-directed mutagenesis\| | BdPAL-pET28a | pHM2086 |
| GAGCGC (SEQ ID NO: 220) | (F138H) | | |
| CTTAGATTCCTCAATGCCGGAGTCTTCGG | site-directed | mutagenesis\| | pHM2232 |
| CACC (SEQ ID NO: 221) | (H140F) | JaPAL^{F140H}_MUT8-pET28a, | |
| | | JaPAL^{F140H}_MUT8-pET28a | |
| ATTGAGGAATCTAAGTAGCTCTCTTTGGA | site-directed mutagenesis | JaPAL^{F140H}_MUT8 | pHM2233 |
| GAGC (SEQ ID NO: 222) | (H140F) | -pET28a | |
| ATTGAGGAATCTAAGTAGCTCTACTTGG | site-directed mutagenesis | JaPAL^{F140H}_MUT16-pET28a | pHM2234 |
| AGAGC (SEQ ID NO: 223) | (H140F) | | |

Site-directed mutagenesis (2)

| | | | |
|---|---|---|---|
| GCGACTGGGTCATGAGCAGCATGATGAA | site-directed mutagenesis | JaPAL^{F140H}_MUT8-DET28a | pHM2354 |
| CGGC (SEQ ID NO: 224) | (I102V) | | |
| TCATGACCCAGTCGCTGCTGGCCTTGACG | site-directed mutagenesis | JaPAL^{F140H}_MUT8-pET28a | pHM2355 |
| (SEQ ID NO: 225) | (I102V) | | |
| ACCGACAGCTACGGTGTCACCACTGG | site-directed mutagenesis | JaPAL^{F140H}_MUT8-pET28a | pHM2328 |
| (SEQ ID NO: 226) | (I112S) | | |
| ACCGTAGCTGTCGGTGCCGTTCATCA | site-directed mutagenesis | JaPAL^{F140H}_MUT8-DET28a | pHM2329 |
| (SEQ ID NO: 227) | (I112S) | | |
| CTTTGGAGCCACCTCCCACAGGAGGACC | site-directed mutagenesis | JaPAL^{F140H}_MUT8-DET28a | pHM2356 |
| (SEQ ID NO: 228) | (G121A) | | |
| GAGGTGGCTCCAAAGCCAGTGGTGACAC | site-directed mutagenesis | JaPAL^{F140H}_MUT8-pET28a | pHM2357 |
| C (SEQ ID NO: 229) | (G121A) | | |
| GAGAGCTAATTAGACACCTCAATGCCGG | site-directed mutagenesis | JaPAL^{F140H}_MUT8-pET28a | pHM2385 |
| AGTC (SEQ ID NO: 230) | (L138I) | | |
| GTCTAATTAGCTCTCTTTGGAGAGCACCA | \|site-directed mutagenesis | JaPAL^{F140H}_MUT8-DET28a | pHM2386 |
| C (SEQ ID NO: 231) | (L138I) | | |
| CGGCACGGCCGTGGGTTCTGGTCTTG | site-directed mutagenesis | JaPAL^{F140H}_MUT8-DET28a | pHM2334 |
| (SEQ ID NO: 232) | (S267A) | | |
| CCCACGGCCGTGCCGTTCACCATGGC | site-directed mutagenesis | JaPAL^{F140H}_MUT8-pET28a | pHM2335 |
| (SEQ ID NO: 233) | (S267A) | | |
| TGGCCTGCCTTCCAACCTGGCCGGTG | site-directed mutagenesis | JaPAL^{F140H}_MUT8-pET28a | pHM2336 |
| (SEQ ID NO: 234) | (T444P) | | |
| TTGGAAGGCAGGCCATTGTTGTAGAAG | site-directed mutagenesis | JaPAL^{F140H}_MUT8-pET28a | pHM2337 |
| (SEQ ID NO: 235) | (T444P) | | |
| CAACCTGTCCGGTGGGCGCAACCCGA | site-directed mutagenesis | JaPAL^{F140H}_MUT8-pET28a | pHM2338 |
| (SEQ ID NO: 236) | (A448S) | | |
| CCACCGGACAGGTTGGAAGTCAGGCC | site-directed mutagenesis | JaPAL^{F140H}_MUT8-pET28a | pHM2339 |
| (SEQ ID NO: 237) | (A448S) | | |
| TGGCCTTATCTCATCCAGGAAGACCG | site-directed mutagenesis | JaPAL^{F140H}_MUT8-pET28a | pHM2340 |
| (SEQ ID NO: 238) | (V500I) | | |
| GATGAGATAAGGCCAAGCGAGTTGAC | site-directed mutagenesis | JaPAL^{F140H}_MUT8-DET28a | pHM2341 |
| (SEQ ID NO: 239) | (V500I) | | |

Site-directed mutagenesis (3)

| | | | |
|---|---|---|---|
| GGAGATAGCTATGGTGTCACCACTGGCT | site-directed mutagenesis | JaPTAL^{H128F}-pET28a | pHM2456 |
| TCG (SEQ ID NO: 240) | (197S) | | |
| ACCATAGCTATCTCCACCGTTCGCCACG | site-directed mutagenesis | JaPTAL^{H128F}-pET28a | pHM2457 |
| (SEQ ID NO: 241) | (197S) | | |
| ACCGACATATACGGTGTCACCACTGGCT | site-directed mutagenesis | JaPAL^{F140H}-pET28a | pHM2458 |
| (SEQ ID NO: 242) | (S112I) | | |
| ACCGTATATGTCGGTGCCGTTCATCA | site-directed mutagenesis | JaPAL^{F140H} pET28a | pHM2459 |
| (SEQ ID NO: 243) | (S112I) | | |
| CACCGACACCTACGGTGTCACCACTGGC | site-directed mutagenesis | JaPAL^{F140H}-pET28a | pHM2475 |
| T (SEQ ID NO: 244) | (S112T) | | |
| CCGTAGGTGTCGGTGCCGTTCATCA (SEQ | site-directed mutagenesis | JaPAL^{F140H}-pET28a | pHM2476 |
| ID NO: 245) | (S112T) | | |
| CACCGACGTCTACGGTGTCACCACTGGC | site-directed mutagenesis | JaPAL^{F140H}-pET28a | pHM2477 |
| (SEQ ID NO: 246) | (S112V) | | |
| CCGTAGACGTCGGTGCCGTTCATCATGC | site-directed mutagenesis | JaPAL^{F140H}-pET28a | pHM2478 |
| (SEQ ID NO: 247) | (S112V) | | |
| TGGAGATGTCTATGGTGTCACCACTGGCT | site-directed mutagenesis | JaPTAL^{H128F}-pET28a | pHM2479 |
| TCG (SEQ ID NO: 248) | (197V) | | |
| CCATAGACATCTCCACCGTTCGCCACG | site-directed mutagenesis | JaPTAL^{H128F}-pET28a | pHM2480 |
| (SEQ ID NO: 249) | (197V) | | |
| TGGAGATACCTATGGTGTCACCACTGGC | site-directed mutagenesis | JaPTAL^{H128F}-pET28a | pHM2481 |
| TTCG (SEQ ID NO: 250) | (197T) | | |
| CCATAGGTATCTCCACCGTTCGCCACG | site-directed mutagenesis | JaPTAL^{H128F}-pET28a | pHM2482 |
| (SEQ ID NO: 251) | (197T) | | |
| ACTGATATATATGGTGTTACTACTGGTTT | site-directed mutagenesis | AtPAL1-pET28a | pHM2524 |
| TGGTG (SEQ ID NO: 252) | (S116I) | | |
| ACCATATATATCAGTGCCTTTGTTCATAC | site-directed mutagenesis | AtPAL1-pET28a | pHM2525 |
| TCTC (SEQ ID NO: 253) | (S116I) | | |
| TATTAGACACCTTAACGCCGGAATATTC | site-directed mutagenesis | AtPAL1-pET28a | pHM2526 |
| G (SEQ ID NO: 254) | F144H) | | |
| TTAAGGTGTCTAATAAGTTCCTTCTGAAG | site-directed mutagenesis | AtPAL1-pET28a | pHM2527 |
| TGCG (SEQ ID NO: 255) | (F144H) | | |
| Site-directed mutagenesis (4) | | | |
| CATCGCCGCCATCGGCAAGCTCATGTTTG | site-directed mutagenesis | JaPTAL-pET28a | pHM2542 |
| (SEQ ID NO: 256) | (N407A) | | |
| CCGATGGCGGCGATGGCGAGGCGGGTG | site-directed mutagenesis | JaPTAL-pET28a | pHM2543 |
| (SEQ ID NO: 257) | (N407A) | | |

TABLE 6
Protein sequences of the JaPAL and AtPAL1 enzymes tested in Example 1

| Enzyme | Wild-type | S112I/F140H mutant | S112I mutant | F140H mutant |
|---|---|---|---|---|
| Joinvillea | JaPAL | JaPAL^{F140H}^{_{—}}^{S112I} | JaPAL^{S112I} | JaPAL^{F140H} |
| ascendens PAL | (SEQ ID NO: 28) | (SEQ ID NO: 145) | (SEQ ID NO: 258) | (SEQ ID NO: 259) |
| Arabidopsis | AtPAL1 | AtPAL1^{F144H}^{_{—}}^{S116I} | AtPAL1^{S116I} | AtPAL1^{F144H} |
| thaliana PAL1 | (SEQ ID NO: 144) | (SEQ ID NO: 146) | (SEQ ID NO: 260) | (SEQ ID NO: 261) |

TABLE 7
DNA sequences of the JaPAL and AtPAL1 enzymes tested in Example 1

| Enzyme | Wild-type | S1121/F140H mutant | S112I mutant | F140H mutant |
|---|---|---|---|---|
| Joinvillea | JaPAL | JaPAL^{F140H}^{_{—}}^{S112I} | JaPAL^{S112I} | JaPAL^{F140H} |
| ascendens PAL | (SEQ ID NO: 147) | (SEQ ID NO: 148) | (SEQ ID NO: 262) | (SEQ ID NO: 263) |
| Arabidopsis | AtPAL1 | AtPAL1^{F144H}^{_{—}}^{S116I} | AtPAL1^{S116I} | AtPAL1^{F144H} |
| thaliana PAL1 | (SEQ ID NO: 149) | (SEQ ID NO: 150) | (SEQ ID NO: 264) | (SEQ ID NO: 265) |

TABLE 8
PTAL and PAL protein sequences aligned FIG. 8

| Name | Organism | Sequence |
|---|---|---|
| Sevir.6G187100.1.p | Setaria viridis | SEQ ID NO: 1 |
| Seita.6G181000.1.p | Setaria italica | SEQ ID NO: 2 |
| Sevir.1G245000.1.p | Setaria viridis | SEQ ID NO: 3 |
| Seita.1G240200.1.p | Setaria italica | SEQ ID NO: 4 |
| PhHAL.1G306700.1.p | Panicum hallii | SEQ ID NO: 5 |
| Pavir.1NG356200.1.p | Panicum virgatum | SEQ ID NO: 6 |
| Zm00008a016750_P01 | Zea mays | SEQ ID NO: 7 |
| Zm00008a022367_P01 | Zea mays | SEQ ID NO: 8 |
| Sobic.004G220300.1.p | Sorghum bicolor | SEQ ID NO: 9 |
| Sevir.7G178200.1.p | Setaria viridis | SEQ ID NO: 10 |
| Seita.7G168700.1.p | Setaria italica | SEQ ID NO: 11 |
| Osa_LOC_Os02g41630.2 | Oryza sativa | SEQ ID NO: 12 |
| Bradi3g49250.2.p | Brachypodium distachyon | SEQ ID NO: 13 |
| Pavir.7KG238255.1.p | Panicum virgatum | SEQ ID NO: 14 |
| Pavir.7NG355500.1.p | Panicum virgatum | SEQ ID NO: 15 |
| PhHAL.7G213800.1.p | Panicum hallii | SEQ ID NO: 16 |
| Zm00008a006867_P01 | Zea mays | SEQ ID NO: 17 |
| Sobic.006G148800.1.p | Sorghum bicolor | SEQ ID NO: 18 |
| Seita.2G435800.1.p | Setaria italica | SEQ ID NO: 19 |
| Sevir.2G448300.1.p | Setaria viridis | SEQ ID NO: 20 |
| Sevir.7G177900.1.p | Setaria viridis | SEQ ID NO: 21 |
| Seita.7G168500.1.p | Setaria italica | SEQ ID NO: 22 |
| Osa_LOC_Os04g43760.1 | Oryza sativa | SEQ ID NO: 23 |
| STRANG_00041445-RA | Streptochaeta angustifolia | SEQ ID NO: 24 |
| STRANG_00039019-RA | Streptochaeta angustifolia | SEQ ID NO: 25 |
| Emon_maker-scf7180000017824- | Ecdeiocolea monostachya | SEQ ID NO: 26 |
| augustus-gene-4.6-mRNA-1 | | |
| Joascv11021323m | Joinvillea ascendens | SEQ ID NO: 27 |
| Joascv11021328m | Joinvillea ascendens | SEQ ID NO: 28 |
| Emon_maker-scf7180000017824- | Ecdeiocolea monostachya | SEQ ID NO: 29 |
| augustus-gene-6.51-mRNA-1 | | |
| Flagellaria_indica_Trinity_comp23995_c0_seq1 | Flagellaria indica | SEQ ID NO: 30 |
| Seita.1G240400.1.p | Setaria italica | SEQ ID NO: 31 |
| Sevir.1G245166.1.p | Setaria viridis | SEQ ID NO: 32 |
| Seita.1G240500.1.p | Setaria italica | SEQ ID NO: 33 |
| Sevir.1G245232.1.p | Setaria viridis | SEQ ID NO: 34 |
| Seita.1G240600.1.p | Setaria italica | SEQ ID NO: 35 |
| Sevir.1G245300.2.p | Setaria viridis | SEQ ID NO: 36 |
| PhHAL.1G307000.1.p | Panicum hallii | SEQ ID NO: 37 |
| PhHAL.1G307100.1.p | Panicum hallii | SEQ ID NO: 38 |
| PhHAL.1G307200.1.p | Panicum hallii | SEQ ID NO: 39 |
| Pavir.1NG356700.1.p | Panicum virgatum | SEQ ID NO: 40 |
| Pavir.1NG356800.1.p | Panicum virgatum | SEQ ID NO: 41 |
| Pavir.1KG386500.1.p | Panicum virgatum | SEQ ID NO: 42 |
| Sobic.004G220600.2.p | Sorghum bicolor | SEQ ID NO: 43 |
| Sobic.004G220500.1.p | Sorghum bicolor | SEQ ID NO: 44 |
| Sobic.004G220700.1.p | Sorghum bicolor | SEQ ID NO: 45 |
| Zm00008a016754_P01 | Zea mays | SEQ ID NO: 46 |
| Zm00008a022372_P01 | Zea mays | SEQ ID NO: 47 |
| Zm00008a022370_P01 | Zea mays | SEQ ID NO: 48 |
| Osa_LOC_Os02g41670.1 | Oryza sativa | SEQ ID NO: 49 |
| Osa_LOC_Os02g41680.1 | Oryza sativa | SEQ ID NO: 50 |
| Bradi3g47110.1.p | Brachypodium distachyon | SEQ ID NO: 51 |
| Bradi3g47120.1.p | Brachypodium distachyon | SEQ ID NO: 52 |
| Bradi3g49270.1.p | Brachypodium distachyon | SEQ ID NO: 53 |
| Bradi3g48840.1.p | Brachypodium distachyon | SEQ ID NO: 54 |
| Bradi3g49280.1.p | Brachypodium distachyon | SEQ ID NO: 55 |
| Osa_LOC_Os05g35290.1 | Oryza sativa | SEQ ID NO: 56 |
| Pavir.1KG386300.1.p | Panicum virgatum | SEQ ID NO: 57 |
| Pavir.1NG356400.1.p | Panicum virgatum | SEQ ID NO: 58 |
| PhHAL.1G306800.1.p | Panicum hallii | SEQ ID NO: 59 |
| Seita.1G240300.1.p | Setaria italica | SEQ ID NO: 60 |
| Sevir.1G245100.1.p | Setaria viridis | SEQ ID NO: 61 |
| Zm00008a016751_P01 | Zea mays | SEQ ID NO: 62 |
| Zm00008a022369_P01 | Zea mays | SEQ ID NO: 63 |
| Sobic.004G220400.1.p | Sorghum bicolor | SEQ ID NO: 64 |
| Osa_LOC_Os02g41650.1 | Oryza sativa | SEQ ID NO: 65 |
| Osa_LOC_Os11g48110.1 | Oryza sativa | SEQ ID NO: 66 |
| Osa_LOC_Os12g33610.1 | Oryza sativa | SEQ ID NO: 67 |
| Sobic.001G160500.1.p | Sorghum bicolor | SEQ ID NO: 68 |
| Zm00008a004629_P01 | Zea mays | SEQ ID NO: 69 |
| Bradi3g49260.1.p | Brachypodium distachyon | SEQ ID NO: 70 |
| STRANG_00039013-RA | Streptochaeta angustifolia | SEQ ID NO: 71 |
| STRANG_00039015-RA | Streptochaeta angustifolia | SEQ ID NO: 72 |
| Pavir.7KG237800.1.p | Panicum virgatum | SEQ ID NO: 73 |
| PhHAL.7G214000.1.p | Panicum hallii | SEQ ID NO: 74 |
| Pavir.1NG361819.1.p | Panicum virgatum | SEQ ID NO: 75 |
| Pavir.7NG355800.1.p | Panicum virgatum | SEQ ID NO: 76 |
| Sevir.7G178300.1.p | Setaria viridis | SEQ ID NO: 77 |
| Seita.7G168800.1.p | Setaria italica | SEQ ID NO: 78 |
| Zm00008a006866_P01 | Zea mays | SEQ ID NO: 79 |
| Sobic.006G148900.1.p | Sorghum bicolor | SEQ ID NO: 80 |
| Pavir.4KG229700.2.p | Panicum virgatum | SEQ ID NO: 81 |
| Osa_LOC_Os04g43800.1 | Oryza sativa | SEQ ID NO: 82 |
| Osa_LOC_Os08g21670.1 | Oryza sativa | SEQ ID NO: 83 |
| Bradi5g15830.1.p | Brachypodium distachyon | SEQ ID NO: 84 |
| STRANG_00041444-RA | Streptochaeta angustifolia | SEQ ID NO: 85 |
| STRANG_00041441-RA | Streptochaeta angustifolia | SEQ ID NO: 86 |
| STRANG_00041440-RA | Streptochaeta angustifolia | SEQ ID NO: 87 |
| STRANG_00059682-RA | Streptochaeta angustifolia | SEQ ID NO: 88 |
| Aco013943.1 | Ananas comosus | SEQ ID NO: 89 |
| Aco007727.1 | Ananas comosus | SEQ ID NO: 90 |
| Apos_PKA46439.1 | Apostasia shenzhenica | SEQ ID NO: 91 |
| Apos_PKA58411.1 | Apostasia shenzhenica | SEQ ID NO: 92 |
| Apos_PKA64143.1 | Apostasia shenzhenica | SEQ ID NO: 93 |
| Dcat_XP_020704813.1 | Dendrobium catenatum | SEQ ID NO: 94 |
| Pequ_XP_020589738.1 | Phalaenopsis equestris | SEQ ID NO: 95 |
| Dcat_XP_020702280.1 | Dendrobium catenatum | SEQ ID NO: 96 |
| Apos_PKA59591.1 | Apostasia shenzhenica | SEQ ID NO: 97 |
| Apos_PKA60166.1 | Apostasia shenzhenica | SEQ ID NO: 98 |
| Pequ_XP_020579635.1 | Phalaenopsis equestris | SEQ ID NO: 99 |
| Spipo11G0025500 | Spirodela polyrhiza | SEQ ID NO: 100 |
| Spipo1G0003500 | Spirodela polyrhiza | SEQ ID NO: 101 |
| GSMUA_Achr8P18960_001 | Musa acuminata | SEQ ID NO: 102 |
| GSMUA_Achr11P22840_001 | Musa acuminata | SEQ ID NO: 103 |
| GSMUA_Achr5P18560_001 | Musa acuminata | SEQ ID NO: 104 |
| GSMUA_Achr2P00240_001 | Musa acuminata | SEQ ID NO: 105 |
| GSMUA_Achr11P16380_001 | Musa acuminata | SEQ ID NO: 106 |
| GSMUA_Achr5P03950_001 | Musa acuminata | SEQ ID NO: 107 |
| p5.00_sc00071_p0096.1 | Elaeis guineensis | SEQ ID NO: 108 |
| CALSI_Maker00040467 | Calamus simplicifolius | SEQ ID NO: 109 |
| Aco006987.1 | Ananas comosus | SEQ ID NO: 110 |
| Aco027752.1 | Ananas comosus | SEQ ID NO: 111 |
| GSMUA_Achr1P09070_001 | Musa acuminata | SEQ ID NO: 112 |
| p5.00_sc00334_p0013.1 | Elaeis guineensis | SEQ ID NO: 113 |
| p5.00_sc00076_p0011.1 | Elaeis guineensis | SEQ ID NO: 114 |
| Aco010091.1 | Ananas comosus | SEQ ID NO: 115 |
| Aoff_XP_020259774.1 | Asparagus officinalis | SEQ ID NO: 116 |
| Aoff_XP_020259795.1 | Asparagus officinalis | SEQ ID NO: 117 |
| Aoff_XP_020259773.1 | Asparagus officinalis | SEQ ID NO: 118 |
| Aoff_XP_020248601.1 | Asparagus officinalis | SEQ ID NO: 119 |
| Aoff_XP_020272851.1 | Asparagus officinalis | SEQ ID NO: 120 |
| Aoff_XP_020272852.1 | Asparagus officinalis | SEQ ID NO: 121 |
| Acamev11004816m | Acorus americanus | SEQ ID NO: 122 |
| Acamev11046066m | Acorus americanus | SEQ ID NO: 123 |
| Zosma445g00020.1 | Zostera marina | SEQ ID NO: 124 |
| Zosma69g00670.1 | Zostera marina | SEQ ID NO: 125 |
| Atr_evm_27.model.AmTr_v1.0_scaffold00148.59 | Amborella trichopoda | SEQ ID NO: 126 |
| Atr_evm_27.model.AmTr_v1.0_scaffold00032.129 | Amborella trichopoda | SEQ ID NO: 127 |
| CALSI_Maker00043687 | Calamus simplicifolius | SEQ ID NO: 128 |
| CALSI_Maker00043684 | Calamus simplicifolius | SEQ ID NO: 129 |
| p5.00_sc01789_p0001.1 | Elaeis guineensis | SEQ ID NO: 130 |
| p5.00_sc00066_p0001.1 | Elaeis guineensis | SEQ ID NO: 131 |
| CALSI_Maker00043685 | Calamus simplicifolius | SEQ ID NO: 132 |
| Aco020618.1 | Ananas comosus | SEQ ID NO: 133 |
| GSMUA_Achr9P15990_001 | Musa acuminata | SEQ ID NO: 134 |
| Zosma49g00480.1 | Zostera marina | SEQ ID NO: 135 |
| Zosma115g00180.1 | Zostera marina | SEQ ID NO: 136 |
| Spipo15G0044700 | Spirodela polyrhiza | SEQ ID NO: 137 |
| Acamev11008810m | Acorus americanus | SEQ ID NO: 138 |
| Acamev11024102m | Acorus americanus | SEQ ID NO: 139 |
| Acamev11050170m | Acorus americanus | SEQ ID NO: 140 |
| Atr_evm_27.model.AmTr_v1.0_scaffold00024.177 | Amborella trichopoda | SEQ ID NO: 141 |
| Atr_evm_27.model.AmTr_v1.0_scaffold00024.178 | Amborella trichopoda | SEQ ID NO: 142 |
| Atr_evm_27.model.AmTr_v1.0_scaffold00024.181 | Amborella trichopoda | SEQ ID NO: 143 |

### Materials and Methods

### Dataset of Genome and Protein Sequences

We obtained the genome and protein sequence data listed in Table 1 and Table 2 from NCBI, DNA Databank of Japan (DDBJ), phytozome, JGI, and plaza_v4.5_monocots databases. The genome sequence of Streptochaeta angustifolia was downloaded from a publication (Seetharam et al., 2021). The genome sequence of Ecdeiocolea monostachya was provided by Dr. Matthew Moscou (University of Minnesota, MN).

Phylogenetic Tree Analysis and Identification of Residues Involved in the Transition from PAL to PTAL

To find PAL homologs, we used OrthoFinder with the protein sequence datasets for green plants (Table 1) and monocots (Table 2) with the options of an MCL inflation parameter of 1.5, DIAMOND for sequence alignment, FastME, MAFFT for multiple sequence alignment, and FastTree for gene trees (Emms and Kelly, 2015). Because many genome sequences had duplicated or truncated sequences annotated as genes, we then ran filter fasta script using the obtained orthogroup sequences to remove duplicate genes and genes shorter than 3× the standard deviation from the mean or a given length (less than 50 amino acids). Using the filtered sequence dataset, we generated an alignment using MAFFT v7.450 (Katoh and Standley, 2013). To determine the best evolutionary model for each PAL tree, we ran ModelTest-NG (Darriba et al. 2020). The best model was JTT+G4+F for the green plant dataset and JTT+I+G4+F for the monocot dataset. The maximum-likelihood phylogenetic tree was generated using RAXML-NG (Alexey et al., 2019).

### Cloning of PAL and PTAL Candidate Genes

Sequences encoding PAL and PTAL candidate enzymes from S. bicolor, B. distachyon, S. angustifolia, and J. ascendens were amplified from cDNA with gene specific primers and PrimeSTAR® MAX DNA polymerase (Takara Bio) and were cloned into the pET28a vector using the In-Fusion® HD Cloning Kit (Takara Bio). The resulting vectors were submitted for sequence analysis, which confirmed that the coding sequences matched the sequences in the database. Polynucleotides encoding BdPTAL1, EmoPTAL, EmoPAL, JaPAL-MUT9, and JaPAL-MUT17 were synthesized and cloned into pET28a vectors (SynbioTechnologies). For site-directed mutagenesis, 1:100 diluted plasmid was PCR amplified using PrimeSTAR® MAX DNA polymerase (Takara Bio) and mutagenesis primers. The primers used for cloning are shown in Table 5.

### Recombinant Protein Expression and Purification

For recombinant protein expression, the pET28a vectors were transformed into Rosetta-2 (DE3) E. coli and cultured in 3 ml of terrific broth (TB) medium containing kanamycin (50 μg/ml), chloramphenicol (34 μg/ml), and 0.1% glucose at 37° C. and 200 rpm overnight. Then, 500 μl of pre-culture solution was added to 50 ml TB medium containing the same antibiotics and further cultured at 27° C. and 200 rpm until the OD600 reached 0.5-0.7. The bacterial cultures were then cooled down on ice, isopropyl β-D-1-thiogalactopyranoside (IPTG, 0.5 mM final concentration) was added, and the cultures were incubated at 22° C. and 200 rpm. After 24 hours, the cultures were harvested by centrifugation (5000 g, 5 min, 4° C.) and the pellets were frozen at −30° C. The pellets were thawed and resuspended in lysis buffer containing 50 mM sodium phosphate buffer (pH 8.0), 300 mM NaCl, 10% glycerol, and 0.25 mg lysozyme. After a 30 min incubation on ice, the suspension was sonicated three times for 20 sec and the supernatant was recovered after centrifugation (12500 g, 20 min, 4° C.). The supernatants were added to a new tube containing 100 μl of Ni-NTA beads (Millipore) and the mixture was incubated at 25° C. for 30 min under constant inversion. After unbound proteins were washed away via three washes with washing buffer containing 50 mM sodium phosphate buffer (pH 8.0), 300 mM NaCl, 10% glycerol, and 10 mM imidazole, target proteins were eluted with elution buffer containing 50 mM sodium phosphate buffer (pH 8.0), 300 mM NaCl, 10% glycerol, and 300 mM imidazole. The purified enzyme solutions were desalted using a Sephadex G-50 column (GE Healthcare). The protein concentration was determined using the BioRad protein assay dye (BioRad). The purity was confirmed to be >90% using SDS-PAGE and ImageJ software.

### PAL and TAL Enzyme Assays

All substrate solutions were prepared with 0.01 N NaOH to increase the solubility of L-Tyr. A mixture containing 100 mM Tris-HCl (pH 8.5), 1% glycerol, and purified enzyme in a total volume of 50 μl was preincubated for 3 min at 30° C. PAL and TAL reactions were started by addition of 50 μl of 1 mM substrate (L-Phe or L-Tyr, respectively) and were incubated at 30° C. for 20 min unless otherwise noted. The reactions were terminated by addition of 6N acetic acid (10 μl).

The reaction products were analyzed using high-performance liquid chromatography (HPLC) (1200 Infinitely Series-Infinitely better, Agilent Technologies) to directly detect products produced by PAL and TAL activity, i.e., cinnamic acid and p-coumaric acid, respectively. Analytical conditions were as follows: column, Neptune T3 C18 column (3 μm, 2.1×150 mm, ES industries); solvent system, solvent A (water including 0.1%[v/v] formic acid) and solvent B (acetonitrile including 0.1%[v/v] formic acid); gradient program: 99% A/1% B at 0 min, 99% A/1% B at 4.5 min, 95% A/5% B at 7.5 min, 85% A/15% B at 12 min, 75% A/25% B at 16.5 min, 70% A/30% B at 21 min, 5% A/95% B at 23 min, 5% A/95% B at 26 min, 99% A/5% B at 26.5 min, and 99% A/5% B at 30 min; flow rate: 0.3 mL/min; DAD: 275 nm for cinnamic acid, 309 nm for p-coumaric acid.

The kinetic parameters of the recombinant enzymes were determined using HPLC. Reaction mixtures containing 100 mM Tris-HCl (pH 8.5), 1% glycerol, and purified enzyme (0.15 μg for PAL assay and 1 μg for TAL assay) in a 50 μl total volume were preincubated for 3 min at 30° C. PAL and TAL reactions were started by addition of 50 μl substrate solution prepared with 0-4 mM L-Phe and 0-2 mM L-Tyr. After 10 min and 20 min incubations for PAL and TAL assay, respectively, at 30° C., the reaction was terminated by addition of 6N acetic acid (10 μl). Analytical conditions were as follows: column, Atlantis T3 C18 column (3 μm, 2.1×150 mm, Waters); solvent system, solvent A (water including 0.1%[v/v] formic acid) and solvent B (acetonitrile including 0.1%[v/v] formic acid); gradient program: 85% A/15% B at 0 min, 85% A/15% B at 1 min, 70% A/30% B at 3 min, 15% A/95% B at 6.5 min, 15% A/95% B at 7.5 min, 85% A/15% B at 8.5 min, and 85% A/15% B at 10 min; flow rate: 0.4 mL/min; DAD: 275 nm for cinnamic acid, 309 nm for p-coumaric acid. The products were quantified using calibration curves generated using authentic standards. Non-linear hyperbolic regression analyses were conducted using the Excel Solver tool to calculate Km and Vmax values.

### Protein Modeling Analysis

The structures of JaPAL and JaPTAL were generated with SWISS-MODEL (Waterhouse et al., 2018) using a homo-tetrameric PAL structure from parsley 6F6T.pdb (Bata et al., 2021) and a homo-dimeric PTAL structure from sorghum 6AT7.pdb (Sun et al., 2018), respectively, as templates. The sequence identity against each template were 77.3% and 80.5% for JaPAL and JaPTAL, respectively.

### REFERENCES

Dixon, R. A. and Barros, J. (2019) Lignin biosynthesis: old roads revisited and new roads explored. Open Biol. 9, 190215.
Barros, J. and Dixon, R. A. (2020) Plant Phenylalanine/Tyrosine Ammonia-lyases. Trends Plant Sci., 25, 66-79.
Barros, J., Serrani-Yarce, J. C., Chen, F., Baxter, D., Venables, B. J. and Dixon, R. A. (2016) Role of bifunctional ammonia-lyase in grass cell wall biosynthesis. Nat. Plants, 2, 16050.
Bata, Z., Molnár, Z., Madaras, E., et al. (2021) Substrate Tunnel Engineering Aided by X-ray Crystallography and Functional Dynamics Swaps the Function of MIO-Enzymes. ACS Catal., 11, 4538-4549.
Beaudoin-Eagan, L. D. and Thorpe, T. A. (1985) Tyrosine and Phenylalanine Ammonia Lyase Activities during Shoot Initiation in Tobacco Callus Cultures 1. Plant Physiol., 78, 438-441.
Biasini, M., Bienert, S., Waterhouse, A., et al. (2014) SWISS-MODEL: modelling protein tertiary and quaternary structure using evolutionary information. Nucleic Acids Res., 42, W252-W258.
Cass, C. L., Peraldi, A., Dowd, P. F., et al. (2015) Effects of PHENYLALANINE AMMONIA LYASE (PAL) knockdown on cell wall composition, biomass digestibility, and biotic and abiotic stress responses in Brachypodium. J. Exp. Bot., 66, 4317-4335.
Cochrane, F. C., Davin, L. B. and Lewis, N. G. (2004) The Arabidopsis phenylalanine ammonia lyase gene family: kinetic characterization of the four PAL isoforms. Phytochemistry, 65, 1557-1564.
Darriba, D., Posada, D., Kozlov, A. M., Stamatakis, A., Morel, B. and Flouri, T. (2020) ModelTest-NG: A New and Scalable Tool for the Selection of DNA and Protein Evolutionary Models. Mol. Biol. Evol., 37, 291-294.
Emms, D. M. and Kelly, S. (2015) OrthoFinder: solving fundamental biases in whole genome comparisons dramatically improves orthogroup inference accuracy. Genome Biol., 16, 157.
Giebel, J. (1973) Phenylalanine and tyrosine ammonia-lyase activities in potato roots and their significance in potato resistance to Heterodera rostochiensis. Nematologica, 19, 3-6.
Givnish, T. J., Ames, M., McNeal, J. R., et al. (2010) Assembling the Tree of the Monocotyledons: Plastome Sequence Phylogeny and Evolution of Poales1. Ann. Mo. Bot. Gard., 97, 584-616.
Havir, E. A., Reid, P. D. and Marsh, H. V., Jr. (1971) 1-Phenylalanine Ammonia-Lyase (Maize): Evidence for a Common Catalytic Site for l-Phenylalanine and l-Tyrosine 1. Plant Physiol., 48, 130-136.
Higuchi, T. and Shimada, M. (1969) Metabolism of phenylalanine and tyrosine during lignification of bamboos. Phytochemistry, 8, 1185-1192.
Jangaard, N. O. (1974) The characterization of phenylalanine ammonia-lyase from several plant species. Phytochemistry, 13, 1765-1768.
Jun, S. Y., Sattler, S. A., Cortez, G. S., Vermerris, W., Sattler, S. E. and Kang, C. (2018) Biochemical and Structural Analysis of Substrate Specificity of a Phenylalanine Ammonia-Lyase. Plant Physiol., 176, 1452-1468.
Katoh, K. and Standley, D. M. (2013) MAFFT multiple sequence alignment software version 7: improvements in performance and usability. Mol. Biol. Evol., 30, 772-780.
Khan, W., Prithiviraj, B. and Smith, D. L. (2003) Chitosan and chitin oligomers increase phenylalanine ammonia-lyase and tyrosine ammonia-lyase activities in soybean leaves. J. Plant Physiol., 160, 859-863.
Kozlov, A. M., Darriba, D., Flouri, T., Morel, B. and Stamatakis, A. (2019) RAXML-NG: a fast, scalable and user-friendly tool for maximum likelihood phylogenetic inference. Bioinformatics, 35, 4453-4455.
Louie, G. V., Bowman, M. E., Moffitt, M. C., Baiga, T. J., Moore, B. S. and Noel, J. P. (2006) Structural Determinants and Modulation of Substrate Specificity in Phenylalanine-Tyrosine Ammonia-Lyases. Chem. Biol., 13, 1327-1338.
Maeda, H. A. (2019) Harnessing evolutionary diversification of primary metabolism for plant synthetic biology. J. Biol. Chem., 294, 16549-16566.
Maeda, H. A. (2016) Lignin biosynthesis: Tyrosine shortcut in grasses. Nat. Plants, 2, 1-2.
McKain, M. R., Tang, H., McNeal, J. R., et al. (2016) A Phylogenomic Assessment of Ancient Polyploidy and Genome Evolution across the Poales. Genome Biol. Evol., 8, 1150-1164.
Nishiyama, Y., Yun, C. S., Matsuda, F., Sasaki, T., Saito, K., Tozawa, Y. (2010) Expression of bacterial tyrosine ammonia-lyase creates a novel p-coumaric acid pathway in the biosynthesis of phenylpropanoids in Arabidopsis. Planta, 232, 209-218.
Renault, H., Werck-Reichhart, D. and Weng, J. K. (2019) Harnessing lignin evolution for biotechnological applications. Curr. Opin. Biotechnol., 56, 105-111.
Rosler, J., Krekel, F., Amrhein, N. and Schmid, J. (1997) Maize phenylalanine ammonia-lyase has tyrosine ammonia-lyase activity. Plant Physiol., 113, 175-179.
Seetharam, A. S., Yu, Y., Bélanger, S., Clark, L. G., Meyers, B. C., Kellogg, E. A. and Hufford, M. B. (2021) The Streptochacta Genome and the Evolution of the Grasses. Front. Plant Sci., 12.
Shadle, G. L., Wesley, S. V., Korth, K. L., Chen, F., Lamb, C., Dixon, R. (2003) Phenylpropanoid compounds and disease resistance in transgenic tobacco with altered expression of 1-phenylalanine ammonia-lyase. Phytochemistry, 64, 153-161.
Vanholme, R., De Meester, B., Ralph, J. and Boerjan, W. (2019) Lignin biosynthesis and its integration into metabolism. Curr. Opin. Biotechnol., 56, 230-239.
Watts, K. T., Lee, P. C. and Schmidt-Dannert, C. (2006) Biosynthesis of plant-specific stilbene polyketides in metabolically engineered Escherichia coli. BMC Biotechnol., 6, 22.
Young, M. R. and Neish, A. C. (1966) Properties of the ammonia-lyases deaminating phenylalanine and related compounds in Triticum aestivum and Pteridium aquilinum. Phytochemistry, 5, 1121-1132.

### Example 2

In the following example, the inventors describe experiments that demonstrate that several different amino acid substitutions at position 112 in JaPAL retain the TAL activity observed in the JaPAL^{F140H_S112I} double mutant.

A phylogenetic analysis revealed that, while the amino acids Ser and Ile are well conserved at positions corresponding to residue 112 in JaPAL in angiosperm PAL enzymes, basal non-flower PAL enzymes possess Ile, Thr, or Val at this position (FIG. 9A). Also, another group of angiosperm PAL enzymes (clade II in FIG. 9A), which is not conserved across angiosperms, possess Thr at the corresponding position. Accordingly, we tested the TAL activity of JaPAL and JaPTAL enzymes with and without mutations to these other amino acids at residue 112. We found that substituting the Ile at this position in JaPAL^{F140H_S112I} with Thr or Val retains strong TAL activity with comparable kcat and Km values but substituting it with Ser does not (FIG. 9B). Thus, replacing Ser112 with Ile, Val, or Thr together with the F140H mutation could potentially convert a PAL enzyme into a PTAL enzyme.

### Example 3

In the following example, the inventors describe future experiments in which engineered PAL enzymes will be tested in planta.

To test the effects of the F140H and S112I mutations in plants, we will transiently express recombinant PAL enzymes (e.g., Arabidopsis PAL_S112I-F140H) with and without the corresponding mutations in Nicotiana benthamiana using Agrobacterium-mediated transformation. Soluble metabolites will be extracted from the transformed Nicotiana leaves and quantified to determine if the production of any soluble phenylpropanoid compounds was affected by the presence of the recombinant PAL enzymes.

This experiment will also be conducted in plants that express deregulated TyrA enzymes that we previously discovered, such as Beta vulgaris TyrAalpha (Lopez-Nieves et al., Plant J 109: 844-855 (2021)). The presence of the deregulated TyrA enzymes should increase the availability of the tyrosine substrate for the TAL activity.

What is claimed:

1. An engineered phenylalanine ammonia-lyase (PAL) enzyme comprising a mutation relative to a wild-type PAL enzyme, wherein the mutation is at a position corresponding to residue 112 of SEQ ID NO: 28, and wherein the engineered PAL enzyme has increased TAL activity relative to the wild-type PAL enzyme.

2. The engineered PAL enzyme of claim 1, wherein the mutation is a serine to isoleucine mutation, a serine to valine mutation, or a serine to threonine mutation.

3. The engineered PAL enzyme of claim 1, further comprising a second mutation relative to the wild-type PAL enzyme, wherein the second mutation is at a position corresponding to residue 140 of SEQ ID NO: 28.

4. The engineered PAL enzyme of claim 3, wherein the second mutation is a phenylalanine to histidine mutation.

5. The engineered PAL enzyme of claim 1, wherein the wild-type PAL enzyme comprises a sequence selected from SEQ ID NO: 28-143 or a sequence having at least 90% identity to one of SEQ ID NO: 28-143.

6. The engineered PAL enzyme of claim 5, wherein the wild-type PAL enzyme comprises SEQ ID NO: 28 (JaPAL) or SEQ ID NO: 144 (AtPAL1).

7. The engineered PAL enzyme of claim 6, wherein the engineered PAL enzyme comprises SEQ ID NO: 145 (JaPAL^{F140H_S112I}), SEQ ID NO: 146 (AtPAL1^{F144H_S116I}), a sequence having at least 90% identity to SEQ ID NO: 145, or a sequence having at least 90% identity to SEQ ID NO: 146.

8. The engineered PAL enzyme of claim 1, wherein the engineered PAL enzyme further comprises at least one additional mutation relative to the wild-type enzyme at a position corresponding to residue 102, 121, 138, 267, 444, 448, or 500 of SEQ ID NO: 28.

9. A polynucleotide encoding the engineered PAL enzyme of claim 1.

10. A construct comprising a promoter operably linked to the polynucleotide of claim 9.

11. A vector comprising the polynucleotide of claim 9.

12. A cell comprising the polynucleotide of claim 9.

13. A seed comprising the polynucleotide of claim 9.

14. A plant comprising the engineered PAL enzyme of claim 1.

15. The plant of claim 14, wherein the plant:
a) produces a greater quantity of lignin as compared to a control plant;
b) produces a greater quantity of phenylpropanoid-derived compounds as compared to a control plant; and/or
c) assimilates a greater quantity of carbon dioxide (CO₂) as compared to a control plant.

16. The plant of claim 14, wherein the plant is a non-grass land plant.

17. The plant of claim 14, further comprising:
a) an engineered 3-deoxy-D-arabino-heptulosonate 7-phosphate synthase enzyme that comprises one or more mutation relative to a wild-type enzyme at a position corresponding to residue 109, 114, 159, 240, 244, 245, 247, 248, 319, 322, or 348 of SEQ ID NO: 152;
b) an engineered arogenate dehydrogenase enzyme comprising a non-acidic amino acid residue at a position corresponding to residue 220 of SEQ ID NO: 153; or
c) an engineered prephenate dehydrogenase enzyme comprising an aspartic acid (D) or glutamic acid (E) at a position corresponding to residue 220 of SEQ ID NO: 154.

18. A method of making the plant of claim 14, the method comprising: introducing a polynucleotide encoding the engineered PAL enzyme into plants and selecting a plant that expresses the engineered PAL enzyme.

19. A method of making the plant of claim 14, the method comprising: editing a gene encoding a wild-type PAL enzyme in the plant to have a mutation at a position corresponding to residue 112 of SEQ ID NO: 28.

20. The method of claim 19, wherein the method further comprises: editing the gene to have a mutation at a position corresponding to residue 140 of SEQ ID NO: 28.

21. A method for producing phenylpropanoid-derived products, the method comprising:
a) growing a plant genetically engineered to express the engineered PAL enzyme of claim 1; and
b) purifying phenylpropanoid-derived products produced by the plant.

22. A method for sequestering CO₂, the method comprising growing a plant genetically engineered to express the engineered PAL enzyme of claim 1.


SEQUENCE LISTING (265 sequences)

SEQ ID NO: 1 - protein, 698 aa, Setaria viridis
MAGNGLIVENDPLNWGAAAAELAGSHLDEVKRMVAQARQPVVKIEGSTLRVGQVAAVAAAKDASGVAVELDEEARPRVKASSEWILDCIAHGGDIYGVTTGFGGTSHRRTKDGPALQVELLRHLNAGIFGAGSDGHTLPSEVTRAAMLVRINTLLQGYSGIRFEILEAITKLLNSGVSPCLPLRGTITASGDLVPLSYIAGLITGRPNAQAVTVDGRKVDAAEAFKVAGIEGGFFRLNPKEGLAIVNGTSVGSALAATVLYDANLLAVLSEVLSAVFCEVMNGKPEYTDHLTHKLKHHPGSIEAAAIMEHILEGSAFMKHAKKVNELDPLLKPKQDRYALRTSPQWLGPQIEVIRAATKSIEREVNSVNDNPVIDVHRGKALHGGNFQGTPIGVSMDNARLAIANIGKLMFAQFSELVNEFYNNGLTSNLAGSRNPSLDYGFKGTEIAMASYCSELQYLGNPITNHVQSAEQHNQDVNSLGLVSARKTAEAIDILKLMSSTYIVALCQAIDLRHLEENIKTSVKNTVTQVAKKVLTMNPAGELSSARFSEKDLLTAIDREGVFTYAEDAASASLPLMQKLRAVLVDHAFSSGDEPSVFSKITKFEEELRAVLPQEVEAARVAVAEGTAAVENRIKDSRSFPLYRFVREELGCVFLTGEKLKSPGEECNKVFVGISQGKLVDPMLECLKEWDGKPLPIN

SEQ ID NO: 2 - protein, 698 aa, Setaria italica; identical to SEQ ID NO: 1

SEQ ID NO: 3 - protein, 698 aa, Setaria viridis
MAGNGLIVENDPLNWGAAAAELAGSHLDEVKRMVAQARQPVVKIEGSTLRVGQVAAVASARDASGVAVELDEEARPRVKASSEWILDCIAHGGDIYGVTTGFGGTSHRRTKDGPALQVELLRHLNAGIFGTGSDGHTLPSEVTRAAMLVRINTLLQGYSGIRFEILEAITKLLNSGVSPCLPLRGTITASGDLVPLSYIAGLITGRPNAQAVTADGRKVDAAEAFKIAGIEGGFFKLNPKEGLAIVNGTSVGSALAATVLYDANVLAVLSEVLSAVFCEVMNGKPEYTDHLTHKLKHHPGSIEAAAIMEHILEGSEFMKHAKKVNELDPLLKPKQDRYALRTSPQWLGPQIEVIRAATKSIEREVNSVNDNPVIDVHRGKALHGGNFQGTPIGVSMDNARLAIANIGKLMFAQFSELVNEFYNNGLTSNLAGSRNPSLDYGFKGTEIAMASYCSELQYLGNPITNHVQSAEQHNQDVNSLGLVSARKTAEAIDILKLMSSTYMVALCQAIDLRHLEENIKTSVKNTVTQVAKKVLTMNPTGDLSSARFSEKDLITAIDREGVFTYAEDAASASLPLMQKLRAVLVDHAFSSGDEPSMFSKITKFEEELRAVLPQEVEAARVAVAEGTAAVENRIKDSRSFPLYRFVREELGCVFLTGEKLKSPGEECNKVFIGISQGKLIDPMLECLKEWDGKPLPIN

SEQ ID NO: 4 - protein, 698 aa, Setaria italica; identical to SEQ ID NO: 3

SEQ ID NO: 5 - protein, 701 aa, Panicum hallii
MAGNGLIVENDPLNWGAAAAELAGSHLDEVKRMVAQARQPVVKIEGSTLRVGQVAAVAAARDASGVAVELDEEARPRVKASSEWILDCIAHGGDIYGVTTGFGGTSHRRTKDGPALQVELLRHLNAGIFGTGRDGHTLPSEVVRAAMLVRINTLLQGYSGIRFEILEAITKLLNTGVSPCLPLRGTITASGDLVPLSYIAGLITGRPNAQAVTVDGRKVDAAEAFKIAGIEGGFFKLNPKEGLAIVNGTSVGSALAATVLYDANVLAVLSEVLSAVFCEVMNGKPEYTDHLTHKLKHHPGSIEAAAIMEHILEGSSFMKQAKKVNELDPLLKPKQDRYALRTSPQWLGPQIEVIRAATKSIEREVNSVNDNPVIDVHRGKALHGGNFQGTPIGVSMDNARLAIANIGKLMFAQFSELVNEFYNNGLTSNLAGSRNPSLDYGFKGTEIAMASYCSELQYLGNPITNHVQSAEQHNQDVNSLGLVSARKTAEAIDILKLMSSTYIVALCQAVDLRHLEENIKTSVKNTVTQVAKKVLTMNPTGDLSAARFSEKDLITAIDREGVFTYAEDAASASLPLMQKLRAVLVDHALSSGDAEREPSVFSKITKFEEELRAVLPREVEAARVAVAEGTASVPNRIKDSRSFPLYRFVREELGCVFLTGEKLKSPGEECNKVFVGISEGKLIDPMLECLKEWDGKPLPIN

SEQ ID NO: 6 - protein, 701 aa, Panicum virgatum
MAGNGLIVENDPLNWGAAAAELAGSHLDEVKRMVAQARQPVVKIEGSTLRVGQVAAVAAARDASGVAVELDEEARPRVKASSEWILDCIAHGGDIYGVTTGFGGTSHRRTKDGPALQVELLRHLNAGIFGNDRDGHTLPSETVRAAMLVRINTLLQGYSGIRFEILEALAKLLNTGVSPCLPLRGTITASGDLVPLSYIAGLITGRPNAQAVTADGRKVDAAEAFKLAGIDGGFFRLNPKEGLAIVNGTSVGSALAATVLYDANVLAVLSEVLSAVFCEVMNGKPEYTDHLTHKLKHHPGSIEAAAIMEHILEGSSFMKQAKKVNELDPLLKPKQDRYALRTSPQWLGPQIEVIRAATKSIEREVNSVNDNPVIDVHRGKALHGGNFQGTPIGVSMDNARLAIANIGKLMFAQFSELVNEFYNNGLTSNLAGSRNPSLDYGFKGTEIAMASYCSELQYLGNPITNHVQSAEQHNQDVNSLGLVSARKTAEAVDILKLMSSTYIVALCQAVDLRHLEENIKTSVKNTVTQVAKKVLTMNPTGDLSTARFSEKDLITAIDREAVFTYAEDAASASLPLMQKLRAVLVDHALSSGDAEREPSVFSKITKFEEELRAVLPREVEAARVAVAEGAAAVPNRIKDSRSFPLYRFVREELGCVFLTGEKLKSPGEECNKVFIGISEGKLVDPMLECLKEWDGKPLPIN

SEQ ID NO: 7 - protein, 662 aa, Zea mays
MAGNGAIVESDPLNWGAAAAELAGSHLDEVKRMVAQARQPVVKIEGSTLRVGQVAAVASARDASGVAVELDEEARPRDGPALQVELLRHLNAGIFGTGSDGHTLPSEVTRAAMLVRINTLLQGYSGIRFEILEAITKLLNTGVSPCLPLRGTITASGDLVPLSYIAGLITGRPNAQAVTVDGRKVDAAEAFKIAGIEGGFFKLNPKEGLAIVNGTSVGSALAATVMYDANVLAVLSEXVMNGKPEYTDHLTHKLKHHPGSIEAAAIMEHILEGSSFMKHAKKVNELDPLLKPKQDRYALRTSPQWLGPQIEVIRAATKSIEREVNSVNDNPVIDVHRGKALHGGNFQGTPIGVSMDNARLAIANIGKLMFAQFSELVNEFYNNGLTSNLAGSRNPSLDYGFKGTEIAMASYCSELQYLGNPITNHVQSAEQHNQDVNSLGLVSARKTAEAVDILKLMSSTYIVALCQAVDLRHLEENIKTSVKNTVTQVAKKVLTMNPSGDLSSARFSEKELITAIDREAVFTYAEDAASASLPLMQKLRAVLVDHALSSGDAEREPSVFSRITKFEEELRAVLPHEVEAARVAVAEGTAPVANRIADSRSFPLYRFVRQELGCVFLTGEKLKSPGEECNKVFLGISQGKLVDPMLECLKEWDGKPLPININ

SEQ ID NO: 8 - protein, 644 aa, Zea mays
MAGNGAIVESDPLNWGAAAAELAGSHLDEVKRMVAQARQPVVKIEGSTLRVGQVAAVASAKDAHLNAGIFGTGSDGHTLPSEVTRAAMLVRINTLLQGYSGIRFEILEAITKLLNTGVSPCLPLRGTITASGDLVPLSYIAGLITGRPNAQAVTVDGRKVDAAEAFKIAGIEGGFFKLNPKEGLAIVNGTSVGSALAATVMYDANVLAVLSEVLSAVFCEVMNGKPEYTDHLTHKLKHHPGSIEAAAIMEHILEGSSFMKQAKKVNELDPLLKPKQDRYALRTSPQWLGPQIEVIRAATKSIEREVNSVNDNPVIDVHRGKALHGGNFQGTPIGVSMDNARLAIANIGKLMFAQFSELVNEFYNNGLTSNLAGSRNPSLDYGFKGTEIAMASYCSELQYLGNPITNHVQSAEQHNQDVNSLGLVSARKTAEAIDILKLMSSTYIVALCQAVDLRHLEENIKASVKNTVTQVAKKVLTMNPSGELSSARFSEKELISAIDREAVFTYAEDAASGSLPLMQKLRAVLVDHALSSGDAEREPSVFSKITRFEEELRAVLPQEVEAARVAVAEGTAPVANRIADSRSFPLYRFVREELGCVFLTGERLKSPGEECNKVFVGISQGKLVDPMLECLKEWDGKPLPINVK

SEQ ID NO: 9 - protein, 704 aa, Sorghum bicolor
MAGNGAIVESDPLNWGAAAAELSGSHLDEVKRMVAQARQPVVKIEGSTLRVGQVAAVASAKDASGVAVELDEEARPRVKASSEWILDCIAHGGDIYGVTTGFGGTSHRRTKDGPALQVELLRHLNAGIFGTGSDGHTLPSEVVRAAMLVRINTLLQGYSGIRFEILEAITKLLNTGVSPCLPLRGTITASGDLVPLSYIAGLITGRPNAQATTVDGRKVDAAEAFKIAGIEGGFFKLNPKEGLAIVNGTSVGSALAATVMYDANVLAVLSEVLSAIFCEVMNGKPEYTDHLTHKLKHHPGSIEAAAIMEHILDGSAFMKHAKKVNELDPLLKPKQDRYALRTSPQWLGPQIEVLRAATKSIEREVNSVNDNPVIDVHRGKALHGGNFQGTPIGVSMDNARLAIANIGKLMFAQFSELVNEFYNNGLTSNLAGSRNPSLDYGFKGTEIAMASYCSELQYLGNPITNHVQSAEQHNQDVNSLGLVSARKTAEAIDILKLMSSTYIVALCQAIDLRHLEENIKTSVKNTVTQVAKKVLTMNPSGDLSSARFSEKELITAIDREGVFTYAEDPASASLPLMTKLRAVLVDHALSSGDAEREPSVFSKITKFEEELRAVLPREVEAARVAVAEGTAPVANRIADSRSFPLYRFVREELGCVFLTGEKLKSPGEECTKVFNGINQGKLVDPMLECLKEWDGKPLPINVVN

SEQ ID NO: 10 - protein, 701 aa, Setaria viridis
MASNTAILESDPLNWGKAAAEMAGSHLDEVKRMVAQFREPLVKIEGSSLRVGQVAAVAAAKDASGVAVELDEDARPRVKASSEWILDCIAHGGDIYGVTTGFGGTSHRRTKDGPALQVELLRHLNAGIFGNGSDGHTLPSEVSRAAMLVRINTLLQGYSGIRFEILEAITKLINTGVSPCLPLRGTITASGDLVPLSYIAGLITGRPNAQAVTVDGRKVDAAEAFKVAGIEGGFFKLNPKEGLAIVNGTSVGSALAAMVCFDANVLAVLSEVLSAVFCEVMNGKPEYTDHLTHKLKHHPGSIEAAAIMEHILEGSSFMKHAKKVNELDPLLKPKQDRYALRTSPQWLGPQIEVIRAATKSIEREVNSVNDNPVIDVHRGKALHGGNFQGTPIGVSMDNARLAIANIGKLMFAQFSELVNEFYNNGLTSNLAGSRNPSLDYGFKGTEIAMASYCSELQYLGNPITNHVQSAEQHNQDVNSLGLVSARKTAEAIDILKLMSSTYMVALCQAIDLRHLEENIKTSVKNTVTQVAKKVLTMNPVGELSSARFSEKDIITAIDREGVFTYAEDAASASLPLMQKLRAVLVDHALSSGDAEREPSVFSKITKFEEELRAVLPREVEAARVAVAEGTAPVANRIKDSRSFPVYRFVREELGCVFLTGEKLKSPGEECNKVFIGISQGKLIDPMLECLKEWDGKPLPIC

SEQ ID NO: 11 - protein, 701 aa, Setaria italica
MASNTAILESDPLNWGKAAAEMAGSHLDEVKRMVAQFREPLVKIEGSSLRVGQVAAVAAAKDASGVAVELDEDARPRVKASSEWILDCIAHGGDIYGVTTGFGGTSHRRTKDGPALQVELLRHLNAGIFGNGSDGHTLPSEVSRAAMLVRINTLLQGYSGIRFEILEAITKLINTGVSPCLPLRGTITASGDLVPLSYIAGLITGRPNAQAVTVDGRKVDAAEAFKVAGIEGGFFKLNPKEGLAIVNGTSVGSALGAMVCFDANVLAVLSEVLSAVFCEVMNGKPEYTDHLTHKLKHHPGSIEAAAIMEHILEGSSFMKHAKKVNELDPLLKPKQDRYALRTSPQWLGPQIEVIRAATKSIEREVNSVNDNPVIDVHRGKALHGGNFQGTPIGVSMDNARLAIANIGKLMFAQFSELVNEFYNNGLTSNLAGSRNPSLDYGFKGTEIAMASYCSELQYLGNPITNHVQSAEQHNQDVNSLGLVSARKTAEAIDILKLMSSTYMVALCQAIDLRHLEENIKTSVKNTVTQVAKKVLTMNPVGELSSARFSEKDIITAIDREGVFTYAEDAASASLPLMQKLRAVLVDHALSSGDAEREPSVFSKITKFEEELRAVLPREVEAARVAVAEGTAPVANRIKDSRSFPVYRFVREELGCVFLTGEKLKSPGEECNKVFIGISQGKLIDPMLECLKEWDGKPLPIC

SEQ ID NO: 12 - protein, 701 aa, Oryza sativa
MAGNGPINKEDPLNWGAAAAEMAGSHLDEVKRMVAQFREPLVKIQGATLRVGQVAAVAQAKDAAGVAVELDEEARPRVKASSEWILNCIAHGGDIYGVTTGFGGTSHRRTKDGPALQVELLRHLNAGIFGTGSDGHTLPSETVRAAMLVRINTLLQGYSGIRFEILEAITKLLNTGVTPCLPLRGTITASGDLVPLSYIAGLITGRPNAQAISPDGRKVDAAEAFKLAGIEGGFFTLNPKEGLAIVNGTSVGSALAATVMFDANILAVLSEVLSAVFCEVMNGKPEYTDHLTHKLKHHPGSIEAAAIMEHILAGSSFMSHAKKVNEMDPLLKPKQDRYALRTSPQWLGPQIEVIRAATKSIEREVNSVNDNPVIDVHRGKALHGGNFQGTPIGVSMDNARLAIANIGKLMFAQFSELVNEFYNNGLTSNLAGSRNPSLDYGFKGTEIAMASYCSELQYLANPITNHVQSAEQHNQDVNSLGLVSARKTLEAVDILKLMTSTYIVALCQAVDLRHLEENIKSSVKNCVTQVAKKVLTMNPTGDLSSARFSEKNLLTAIDREAVFSYADDPCSANYPLMQKLRAVLVEHALTSGDAEPEASVFSKITKFEEELRSALPREIEAARVAVANGTAPVANRIVESRSFPLYRFVREELGCVFLTGEKLKSPGEECNKVFLGISQGKLIDPMLDCLKEWNGEPLPIN

SEQ ID NO: 13 - protein, 707 aa, Brachypodium distachyon
MAGNGAISEKDPLNWGAAAAELTGSHLDEVKRMVAQFREPVVKIEGASLRVGQVAAVAQAKDAAGVSVELDEEARPRVKASSEWILSCLAAGGDIYGVTTGFGGTSHRRTKDGPALQVELLRHLNAGIFGTGSDGHSLPAEVTRAAMLVRINTLLQGYSGIRFEILEAITKLLNTGVSPCLPLRGTITASGDLVPLSYIAGLITGRPNAQATTADGRKVDAAEAFKVAGIEGGFFTLNPKEGLAIVNGTSVGSALAATVLFDCNVLAVLSEVLSAVFCEVMNGKPEFTDHLTHKLKHHPGSIEAAAIMEHILAGSSFMSHAKKVNEIDPQLKPKQDRYALRTSPQWLGPQIEVIRSATKSIEREVNSVNDNPVIDVHRGKALHGGNFQGTPIGVSMDNTRLAIANIGKLMFAQFSELVNEFYNNGLTSNLAGSRNPSLDYGFKGTEIAMASYCSELQYLANPVTNHVQSAEQHNQDVNSLGLVSARKTAEAVDILKLMSSTYMVALCQAVDLRHLEENIKASVKNCVTQVSKKVLTMNPTGDLSSARFSEKSLLTAIDREAVFSYADDACSANYPLMQKLRAVLVDHALTSSGVDNAGESEATVFSKINKFEEELRAALPREIEAARVAFEKGTAPIPNLIKDSRSFPLYRFVREELGCVYLTGEKLLSPGEECNKVFIGISQGKLIDPMLECLKEWNGEPLPINVV

SEQ ID NO: 14 - protein, 703 aa, Panicum virgatum
MASNTAICESDPLNWGKAAAEMAGSHLDEVKRMVAQFREPLVKIEGSSLRVGQVAAVAVAKDASGVAVELDEAARPRVKASSEWILDCIAHGGDIYGVTTGFGGTSHRRTKDGPALQVELLRHLNAGIFGNGSDGHTLPSEVSRAAMLVRINTLLQGYSGIRFEILEALAKLINTGVSPCLPLRGTITASGDLVPLSYIAGLITGRPNAQAVTVDGRKVDAAEAFKVAGIEGGFFKLNPKEGLAIVNGTSVGSALAAVVCFDANVLAVLSEVLSAVFCEVMNGKPEYTDHLTHKLKHHPGSIEAAAIMEHILDGSSFMKHAKEVNAMDPLLKPKQDRYALRTSPQWLGPQIEVIRAATKSIEREVNSVNDNPVIDVHRGKALHGGNFQGTPIGVSMDNARLAIANIGKLMFAQFSELVNEFYNNGLTSNLAGSRNPSLDYGFKGTEIAMASYCSELQYLANPITNHVQSAEQHNQDVNSLGLVSARKTAEAIDILKLMSSTYMVALCQAVDLRHLEENLKSAVKSCVTAVARRVLTTRPDGELHSARFSEKSLLTAIDREAVYGYYDDPCGANSPLMKKIRAVLVDHALANGEAEKDASASVLSKINKLEEELREALPREMEAARVAFETGAAPIANRIEESRSYPLYRFIRQDLGAVYLTGEKLKSPGEECNKVFLALSEGKLIDPMLECLKEWDGKPLPIC

SEQ ID NO: 15 - protein, 771 aa, Panicum virgatum
MASNTAICESDPLNWGKAAAEMAGSHLDEVKRMVAQFREPLVKIEGSSLRVGQVAAVAAAKDASGVAVELDEEARPRVKASSEWILDCIAHGGDIYGVTTGFGGTSHRRTKDGPALQVELLRYSSRTKPHVFVCFFVPVQYAAPSDHVPAHWQRPVGGCKLQLVSTRLMSVSGSSLTTGARYVLFSRTCRHLNAGIFGNGSDGHTLPSEVSRAAMLVRINTLLQGYSGIRFEILEALTKLINTGVSPCLPLRGTITASGDLVPLSYIAGLITGRPNAQAVTADGRKVDAAEAFKVAGIEGGFFKLNPKEGLAIVNGTSVGSALAAMVCFDANVLAVLSEVLSAVFCEVMNGKPEYTDHLTHKLKHHPGSIEAAAIMEHILDGSSFMKHAKEVNAMDPLLKPKQDRYALRTSPQWLGPQIEVIRAATKSIEREVNSVNDNPVIDVHRGKALHGGNFQGTPIGVSMDNARLAIANIGKLMFAQFSELVNEFYNNGLTSNLAGSRNPSLDYGFKGTEIAMASYCSELQYLANPITNHVQSAEQHNQDVNSLGLVSSRKTAEAVDILKLMSSTYMVALCQAVDLRHLEENLKSAVKSCVTAVARKVLTTSPDGDLHNARFSEKSLLTAIDREAVYGYYDDPCSANSPLMKKIRAVLVDHALANGEAEKDASASVFSKINKLEEELREALPREMEAARVAFETGTAPIANRIEESRSYPLYRFIRQDLGAVYLTGEKLKSPGEECNKVFLALSEGKLIDPMLECLKEWDGKPLPIC

SEQ ID NO: 16 - protein, 703 aa, Panicum hallii
MASNTAICESDPLNWGKAAAEMAGSHLDEVKRMVAQFREPLVKIEGSSLRVGQVAAVAAAKDASGVAVELDEEARPRVKASSEWILDCIAHGGDIYGVTTGFGGTSHRRTKDGPALQVELLRHLNAGIFGKGSDGHTLPSEVSRAAMLVRINTLLQGYSGIRFEILEAITKLINTGVSPCLPLRGTITASGDLVPLSYIAGLITGRPNAQAVTVDGRKVDAAEAFKVAGIEGGFFKLNPKEGLAIVNGTSVGSALAAMVCFDANVLAVLSEVLSAVFCEVMNGKPEYTDHLTHKLKHHPGSIEAAAIMEHILDGSSFMKHAKEVNAMDPLLKPKQDRYALRTSPQWLGPQIEVIRSATKSIEREVNSVNDNPVIDVHRGKALHGGNFQGTPIGVSMDNARLAIANIGKLMFAQFSELVNEFYNNGLTSNLAGSRNPSLDYGFKGTEIAMASYCSELQYLANPITNHVQSAEQHNQDVNSLGLVSARKTAEAVDILKLMSSTYMVALCQAVDLRHLEENLKRAVKNCVTTVGRKVLTTSPDGDLHNARFSEKSLLTAIDREAVYGYYDDPCSANSSLMKKIRAVLVDHALANGEAEKDASASVFSKINKFEEELREALPREMEAARVAFETGTAPIANRIKESRSYPLYRFIRQDLGAVYLTGEKLKSPGEECNKVFLALSEGKLIDPMLECLKEWDGKPLPIC

SEQ ID NO: 17 - protein, 709 aa, Zea mays
MASNTAILESDPLSWGKAAAELTGSHLDEVKRMVAQFRDPVVKIEGSSLRVGQVAAVAAAKDASGVAVELDEEARPRVKASSEWILDCIAHGGDIXSTASPTAATSTASPPASAAPRTAAPRTGPRSRHLNAGIFGNGSDGHTLPSEVSRAAMLVRINTLLQGYSGIRFEILEAITKLINTGVSPCLPLRGTITASGDLVPLSYIAGLITGRPNAQAVTVDGRKVDAAEAFKAAGIEGGFFKLNPKEGLAIVNGTSVGSALAAMVCFDANVLAVLSEVLSAVFCEVMNGKPEYTDHLTHKLKHHPGSIEAAAIMEHILDGSSFMKHAKEVNAMDPLLKPKQDRYALRTSPQWLGPQIEVIRAATKSIEREVNSVNDNPVIDVHRGKALHGGNFQGTPIGVSMDNARLAVANIGKLMFAQFSELVNEFYNNGLTSNLAGSRNPSLDYGFKGTEIAIASYCSELQYLANPITNHVQSAEQHNQDVNSLGLVSARKTAEAVDILKLMSSTYMVALCQAVDLRHLEENLKSAVKSCVMAVARKVLTTSLGGDLHSARFSEKALLTAIDREAVYGYYDDPCSANSPLMKKIRAVLVDHALASGEAEKDASASVFSKINRFEEELREALPREMEAARVAFETGAAPIANRIKESRSYPLYRFIRQDLGAVYLTGEKLKSPGEECNKVFLALSEGKLIDPMLDCLKEWDGKPLPIC

SEQ ID NO: 18 - protein, 703 aa, Sorghum bicolor
MASNTAILESDPLSWGKAAAELTGSHLDEVKRMVAQFRDPVVKIEGSTLRVGQVAAVAAAKDASGVAVELDEEARPRVKASSEWILDCIAHGGDIYGVTTGFGGTSHRRTKDGPALQVELLRHLNAGIFGNGSDGHTLPSEVSRAAMLVRINTLLQGYSGIRFEILEAITKLLNTGVSPCLPLRGTITASGDLVPLSYIAGLITGRPNAQAVTVDGRKVDAAEAFKVAGIQGGFFKLNPKEGLAIVNGTSVGSALAAMVCFDANVLAVLSSVLSAVFCEVMNGKPEYTDHLTHKLKHHPGSIESAAIMEHILDGSSFMKHAKEVNAMDPLLKPKQDRYALRTSPQWLGPQIEVIRAATKSIEREVNSVNDNPVIDVHRGKALHGGNFQGTPIGVSMDNARLAIANIGKLMFAQFSELVNEFYNNGLTSNLAGSRNPSLDYGFKGTEIAMASYCSELQYLANPITNHVQSAEQHNQDVNSLGLVSARKTAEAVDILKLMSSTYMVALCQAVDLRHLEENLKSAVKNCVMAAARKVLTTSLDGDLHSARFSEKALLTAIDREAVYGYYDDPCSANSPLMKKIRAVLVDHALASGEAEKDASASVFSKINRFEEALREALPREMEAARVAFETGTAPIGNRIKDSRSYPLYRFIRQDLGAVYLTGEKLKSPGEECNKVFLALSEGKLIDPMLECLKEWDGKPLPIC

SEQ ID NO: 19 - protein, 703 aa, Setaria italica
MACNTAIVTSDPLNWGKAAAELTGSHLDEVRRMVAQSREPIVRVDGSRLHVGKVAAVAAAKDASGVAVELDEEARLRVRSSSEWVLSCIENGGDIYGVTTGFGGNSHRRTKDGHALQVELLRYLNAGIFGTGSDGHTLPSQVSRAAMLVRINALMQGYSGIRFEILEAIAKLINTGVSPCLPLRGSITASGDLVPLSYIAGLITGRPNAQAVTVDGRKVDAAEAFKVAGIKGGFFKLNPKEGLAMVNGTSVGSALAAMVCFDANVLAVLAVVLSAVFCEVMNGKPEYADHLTHKLKHHPGSIEAAAIMEHILDGSSLMKHAKEVNAMDPLLKPKQDRYALRTSPQWLGPQIEVIRAATKAIEREINSVSDNPVIDVNRGKALHGGNFQGTPIGVSMDNARLAVASIGRLMFAQFTELVIDFYNNGLPSNLAGSRNLSLDFGLKGVEIAMASYCSELQYLANPVTNHVQSAEQHTQDVNSLGLISARKTAEAVEILKLMSSTFMIALCQAVDLRHLEENLKSAVKNCVKTVALKVLTTSPDGEHCSARFSEKTLLAAIDRKAVYSYCDDPCSASSSLMMTIRAVLVDHALANGEAENEARAPIFSKITKFEEELREALPREMEKTRVAFETGTAPIGNRIKESRSYPLYRFIREDLGAVYLTGEKLKSAGEECNKVFLALSEGKLIDPMLGCLKEWNGEPLPIC

SEQ ID NO: 20 - protein, 703 aa, Setaria viridis
MACNTAIVTSDPLNWGKAAAELTGSHLDEVRRMVAQSREPIVRVDGSRLHVGKVAAVAAAKDASGVAVELDEEARLRVKASSEWVLSCIENGGDIYGVTTGFGGNSHRRTKDGHALQVELLRYLNAGIFGTGSDGHTLPSQVSRAAMLVRINALMQGYSGIRFEILEAIAKLINTGVSPCLPLRGSITASGDLVPLSYIAGLITGRPNAQAVTVDGRKVDAAEAFKVAGIKGGFFKLNPKEGLAMVNGTSVGSALAAMVCFDANVLAVLAVVLSAVFCEVMNGKPEYADHLIHKLKHHPGSIEAAAIMEHILDGSSLMKHAKEVNAMDPLLKPKQDRYALRTSPQWLGPQIEVIRAATKAIEREINSVSDNPVIDVNRGKALHGGNFQGTPIGVSMDNARLAVASIGRLMFAQFTELVIDFYNNGLPSNLAGSRNLSLDFGLKGAEIAMASYCSELQYLANPVTNHVQSAEQHTQDVNSLGLISARKTAEAVEILKLMSSTFMIALCQAVDLRHLEENLKSAVKNCVKTVALKVLTTSPDGEHCSARFSEKALLAAIDRKAVYSYYDDPCSASSSLMMTIRAVLVDHALANGEAENEARAPIFSKITKFEEELREALPREMEKTRVAFETGTAPIGNRIKESRSYPLYRFIREDLGAVYLTGEKLKSAGEECNKVFLALSEGKLIDPMLGCLKEWNGEPLPIC

SEQ ID NO: 21 - protein, 703 aa, Setaria viridis
MACNTAIVTSDPLNWGKAAAELTGSHLDEVRRMVAQSREPVVRVDGSRLHVGQVAAVAAAKDASGVAVELDEEARLRVKASSEWVLSCIENGGDIYGVTTGFGGNSHRRTKDGHALQVELLRYLNAGIFGTGSDGHTLPSQVSRAAMLVRINALMQGYSGIRFEILEAIAKLINTGVSPCLPLRGSITASGDLVPLSYIAGLITGRPNAQAVTVDGRKVDAAEAFKVAGIEGGFFKLNPKEGLAMVNGTSVGSALAAMVCFDANVLAVLAVVLSAVFCEVMNGKPEYADHLTHKLKHHPGSIEAAAIMEHILDGSSLMKHAKEVNAMDPLLKPKQDQYALRTSPQWLGPQIEVIRAATKAIEREINSVSDNPVIDVHRGKALHGGNFQGTPIGVSMDNARLAVASIGRLMFAQFTELVIDFYNNGLPSNLAGSRNLSLDFGLKGAEIAMASYCSELQYLANPVTNHVQSAEQHTQDVNSLGLISARKTAEAVEILKLMSSTFMIALCQAVDLRHLEENLKSAVKNCVKTVALKVLTTSPDGEHCSARFSEKALLAAIDRKAVYSYYDDPCSASSSLMMTIRAVLVDHALANGEAENEAGAPIFSKITKFEEELREALPREMEKTRVAFETGTAPIGNRIKESRSYPLYRFIREDLGAVYLTGEKLKSAGEECNKVFLALSEGKLIDPMLGCLKEWNGEPLPIC

SEQ ID NO: 22 - protein, 703 aa, Setaria italica
MACSTAIVTSDPLNWGKAAAELTGSHLDEVRRMVAQSREPVVRVDGSRLHVGQVAAVAAAKDASGVAVELDEEARLRVKASSEWVLSCIENGGDIYGVTTGFGGNSHRRTKDGHALQVELLRYLNAGIFGTGSDGHTLPSQVSRAAMLVRINALMQGYSGIRFEILEAIAKLINTGVSPCLPLRGSITASGDLVPLSYIAGLITGRPNAQAVTVDGRKVDAAEAFKVAGIEGGFFKLNPKEGLAMVNGTSVGSALAAMVCFDANVLAVLAVVLSAVFCEVMNGKPEYADHLTHKLKHHPGSIEAAAIMEHILDGSSLMKHAKEVNAMDPLLKPKQDQYALRTSPQWLGPQIEVIRAATKAIEREINSVSDNPVIDVHRGKALHGGNFQGTPIGVSMDNARLAVASIGRLMFAQFTELVIDFYNNGLPSNLAGSRNLSLDFGLKGAEIAMASYCSELQYLANPVTNHVQSAEQHTQDVNSLGLISARKTAEAVEILKLMSSTFMIALCQAVDLRHLEENLKSAVKNCVKTVALKVLTTSPDGEHCSARFSEKALLAAIDRKAVYSYYDDPCSASSSLMMTIRAVLVDHALANGEAENEARAPIFSKITKFEEELREALPREMEKTRVAFETGTAPIGNRIKESRSYPLYRFIREDLGAVYLTGEKLKSAGEECNKVFLALSEGKLIDPMLGCLKEWNGEPLPIC

SEQ ID NO: 23 - protein, 707 aa, Oryza sativa
MASQTADATGFVASDPLSWGKAALEMTGSHLDEVKRMVAQSREAVVKIEGSSLRVGQVAAVSAAKDASGVVVELDEEARPRVKASSEWILNCIAHGGDIYGVTTGFGGTSHRRTKDGQALQVELLRHLNAGIFGNGSDGNSLPSEVSRAAMLVRINTLLQGYSGIRFEILEAITKLINTGVSPCLPLRGTITASGDLVPLSYIAGLITGRPNAQAVTVDGKKVDAAEAFKIAGIQGGFFRLEPKEGLAIVNGTSVGSALAAMVLYDANVLAVLSEVLSAVFCEVMNGKPEYTDHLTHKLKHHPGSIEAAAIMEHILAGSAFMPHAQKVNEVDPLLKPKQDRYALRTSPQWLGPQIEVIRAATKSIEREVNSVNDNPVIDVHRGKALHGGNFQGTPIGVSMDNTRLAIANIGKLMFAQFSELVNEFYNNGLTSNLAGSRNPSLDYGFKGTEIAMASYCSELQFLANPVTNHVQSAEQHNQDVNSLGLVSARKTAEAVDILKLMSSTYLVALCQAVDLRHLEENLKSAVKNCVTTVAKKVLTTGPAGGLHSARFSEKALLTAIDREAVYSYADDPCSANYPLMTKIRAVLVEHALANGPAEKDDGSSVFSKITAFEEELREALPREMEAARVAFETGTAPITNRIKESRSFPLYRFVREELGCVYLTGEKLKSPGEECNKVFLAISERKLIDPMLECLKEWNGEPLPIC

SEQ ID NO: 24 - protein, 706 aa, Streptochaeta angustifolia
MVAQSDSAVSILRDPLNWGKAAEELTGSHFDEVKRMVAQFREPAVTIEGASLRVGQVASVAAARDGAGACVELDESARGRVKASSEWILDCIAHGGDIYGVTTGFGGTSHRRTKDGPALQVELLRHLNAGIFGTGSHGHTLPTEVVRAAMLVRTNTLLQGYSGIRFEILEAITKLLNSGVTPCLPLRGTITASGDLVPLSYIAGVLTGRPNAEAVTVDGRKVDAAEAFKVAGIEGGFFKLQPKEGLAIVNGTSVGAALAAMVLYDANILAVLSEVMSAVFCEVMNGKPEYTDHLTHKLKHHPGQIEAAAIMEHILDGSSFMKHAKKVNELDPLLKPKQDRYALRTSPQWLGPQIEVIRAATKSIEREVNSVNDNPVIDVSRGKALHGGNFQGTPIGVSMDNARLAIASIGKLMFAQFSELVNEFYNNGLTSNLAGSRNPSLDYGFKGTEIAMASYCSELQYLGNPITNHVQSAEQHNQDVNSLGLVSARKTAEAIDILKLMSSTYLVALCQAIDLRHLEENLKNAVKNCVMQVAKKVLTTNPAGDLHNARFSEKALLTAIDREAVFSYADDPCSANYPLMQKLRSVLVDHAIANGEDEKEPATSVFAKITKFEDELRTALPREMEAARVAVENGTAPTANRIKESRSYPLYRFIREDLGAVYLTGEKLRSPGEECNKVFVAINQGKLVDPLLECLKEWKGEPLPIC

SEQ ID NO: 25 - protein, 698 aa, Streptochaeta angustifolia
MASQRDPLNWAAAAAELTGSHLDEVKRMVAQFREPQVKIEGASLRVGQVAAVAAARDAAGARVELDEAARGRVKESSEWILNCIATGGDIYGVTTGFGGTSHRRTKDGPALQVELLRHLNAGIFGTGSDGHTLPSEVARAAMLVRINTLLQGYSGIRFEILEAIAKLLNTGVTPCLPLRGTITASGDLVPLSYIAGVITGRPNALAIAPDGRKVDAAEAFKIAGIEGGFFKLQPKEGLAIVNGTSVGAALAAMVLYDANILAVLSEVMSAVFCEVMNGKPEYTDHLTHKLKHHPGQIEAAAIMEHILAGSSFMKHAKKVNELDPLLKPKQDRYALRTAPQWLGPQIEVIRAATKSIEREVNSVNDNPVIDVKRGKALHGGNFQGTPIGVSMDNARLAIANIGKLMFAQFSELVNEFYNNGLTSNLAGSRNPSLDYGFKGTEIAMASYCSELQFLANPVTNHVQSAEQHNQDVNSLGLVSARKTAEAVDILKLMSSTYLVALCQAVDLRHLEENLKNAVKNCVTQVAKKVLTTNPTGDLSNARFSEKALITSIDREAVFSYADDPCSANYPLMQKIRAVLVDHAITNGDGENEPAAAVFARITKFEDELRSALPREMEAARVAFENGASQTPNRMKDSRSYPLYRFIREDLGAVYLTGEKLRSPGEECNKVFVAISQGKLVDPLLDCLKEWNGEPLPIC

SEQ ID NO: 26 - protein, 707 aa, Ecdeiocolea monostachya
MACLSSDNVVCIPKDALNWAKAAEELAGSHLEEVKRMVAEFRSPAVRIEGASLRVGQVAAVAGAEAAAAAKVELDESARARVKASSDWIVDSVANGGDIYGVTTGFGGTSHRRTRDGPALQVELLRHLNAGIFGTGKDGHVLPSEVTRAAMVVRINTLLQGYSGIRFEILEAITALLNSGVTPCLPLRGTITASGDLVPLSYIAGLITGRPNAMAVTPDGRKVDAAEAFKVAGIPHGFFKLQPKEGLAMVNGTSVGAALASMVLFDANILTVLAVVMSAVFCEVMTGKPEYTDHLTHKLKHHPGQIEAAAIMEHILAGSSFMKLAKKLGELDPLMKPKQDRYALRTSPQWLGPQIEVIRTATKSIEREINSVNDNPVIDVSRGKALHGGNFQGTPIGVSMDNARLAIANIGKLMFAQFSELVNEFYNNGLTSNLAGSRNPSLDYGFKGAEIAMASYCSELQFLANPVTNHVQSAEQHNQDVNSLGLVSARKTAEAIDILKLMSSTYLMALCQAIDLRHLEENLKSTVKNCVAQVAKKVLTMSTAGDLHSARFSEKGLLTAIDREAVFAYADDPCSPNYPLMQKVRAVLVEHGLANGEAERVTDTSVFAKITKFEDELRAALPKEVEAVRAAVENGNAPIPNRVKECRSYPLYRFLREELGTVYLTGEKLRSPGEECNKVFVAINEGKLIDPLLECLKEWNGEPLPIC

SEQ ID NO: 27 - protein, 705 aa, Joinvillea ascendens
MAFQNDNVLCIKKDPLNWGKAAEELTGSHLDEVKRMVAESRTPVVKIQGASLRIGQVAAVAVRETGVAKVELDESARGRVKASSDWIVNSVANGGDIYGVTTGFGGTSHRRTRDGPALQIELLRHLNAGIFGTGTDGHVLPSAVTRAAMFVRINTLLQGYSGIRFEILEAIAALLNAGVTPCLPLRGTITASGDLIPLSYIAGLITGRPNAVAIAPDGRKVDAAEAFKIAGIPHGFFKLQPKEGLAIVNGTSVGAALASTVLFDANILAILAEVLSAVFCEVMTGKPEYTDHLTHRLKHHPGQIEAAAIMEHILEGSSFMKLAKKLGELDPLLKPKQDRYALRTAPQWLGPQIEVIRAATKSIEREINSVNDNPIIDVSRGKALHGGNFQGTPIGVSMDNTRLAIANIGKLMFAQFSELVNDFYNNGLTSNLAGGRNPSLDYGFKGTEIAMASYCSELQFLANPVTNHVQSAEQHNQDVNSLGLVSARKTAEAVDILKLMSSTFLIALCQAIDLRHLEENLKSAVKNCVAQVAKKVLTVNTVGDLHNARFSEKDLLTAIDREAVFTYADDPCSPNYPLMQKVRAVLVEHALANGEAERVANTSVFAKITKFEEELRATLPKEVEAARVAVENRTAPTQNRIKESRSYPLYRFVREDLGTAYLTGEKLRSPGEECNKVFVAINQGKLIDPLLECLKKWNGEPLPIC

SEQ ID NO: 28 - protein, 720 aa, Joinvillea ascendens
MECENGNVAAVAAVNGGNGLCLQKPQHADPLNWGKAAGELMGSHLEEVKRMVAEFRAPVVKIEGASLRIAQVAAVAAGEAAAAKVELDESSRGRVKASSDWVMSSMMNGTDSYGVTTGFGATSHRRTKEGGALQRELIRFLNAGVFGTGSDGHVLPAAATRAAMLVRINTLLQGYSGIRFEILEAITALLNAGVTPCLPLRGTITASGDLVPLSYIAGLITGRPNSVAVAPDGRKVDAAEAFKIAGIQHGFFELQPKEGLAMVNGTAVGSGLASTVLFEANILTILAEVLSAVFCEVMTGKPEYTDHLTHKLKHHPGQIEAAAIMEHILEGSSYMKLAKKLGDLDPLMKPKQDRYALRTSPQWLGPQIEVIRASTKSIEREINSVNDNPLIDVSRGKALHGGNFQGTPIGVSMDNTRLAIAAIGKLMFAQFSELVNDFYNNGLPSNLSGGRNPSLDYGFKGAEIAMASYCSELQFLANPVTNHVQSAEQHNQDVNSLGLISSRKTAEAVDILKLMSSTFLIALCQAIDLRHLEENLKSAVKNCVAQVAKKALTLNTVGDLHNARFSEKDLLTAIDREALFAYADDPCNPNYPLMQKLRAVLVEHALANGEAEHVATTSVFAKITKFEEELRATLPKEVEAARVAVENGTAPTPNRIKECRSYPLYRFVREELGTEYLTGEKLRSPGEECNKVFVAINQGKLIDPLLECLKEWNGEPLPIC

SEQ ID NO: 29 - protein, 714 aa, Ecdeiocolea monostachya
MECINGNGAAANGSGLCLRAGGDPLNWGKAAEEMAGSHLEEVKRMVAEFRAPAVRIEGASLRIAQVAAVAAGGAAKARVELDEAARGRVQASSDWVMSSMMNGTDSYGVTTGFGATSHRRTKEGGALQRELIRFLNAGVFGEDSDGHVLPAPATRAAMLVRINTLLQGYSGIRFEILEAITALLNAGVTPCLPLRGTITASGDLVPLSYIAGLVTGRPNSVAVTPDGRKVNAAEAFKIAGIEHGFFELQPKEGLAMVNGTAVGSGLASIVLFETNILAVLAEVLSAVFCEVMTGKPEYTDHLTHKLKHHPGQIEAAAIMEHILDGSSYMKLAKKLGELDPLMKPKQDRYALRTSPQWLGPQIEVIRAATKSIEREINSVNDNPLIDVSRGKALHGGNFQGTPIGVSMDNTRLAIAAIGKLMFAQFSELVNDFYNNGLPSNLSGGRNPSLDYGFKGAEIAMASYCSELQFLANPVTNHVQSAEQHNQDVNSLGLISSRKTAEAVDILKIMSSTFLVALCQAIDLRHLEENLKSAIKNCVAQVAKKALTLNTAGDLHNARFSEKDLLTAIDREAVFAYADDPCSPNYPLMKKLRSVLVEHALANGEAELVAETSVFAKIAKFEEELRAALPSEVEAAWAAVENGTAATPNRIKECRSYPLYRFVREELGSGYLTGEKVRSPGEECNRVFVAINKGKLIDPLLDCLNEWNGEPLPIC

SEQ ID NO: 30 - protein, 725 aa, Flagellaria indica
MECQQNENGIAAAAVANGAAARETGFCLSDPLNWGKAAEEVKGSHLEEVKRMVAEFRAPVVRLEGASLRIAQVAAVAEGKKAAAGGARVELDEAARGRVKASSDWVMDSMTKGTDSYGVTTGFGATSHRRTKEGGALQRELIRFLNAGIFGSGADSGHVLPTPASRAAMLVRINTLLQGYSGIRFEILEAITALLNAGVTPCLPLRGTITASGDLVPLSYIAGLITGRPNAAAIAPDGRKVDAGEAFRIAGIPHGFFELQPKEGLALVNGTAVGSGLASMVLFEANVLALLAEVLSALFCEVMTGKPEYTDHLTHKLKHHPGQIEAAAIMEHILEGSSYMKLAKRLGELDPLMKPKQDRYALRTSPQWLGPQIEVIRAATKSIEREINSVNDNPLIDVSRSKALHGGNFQGTPIGVSMDNTRLAIAAIGKLMFAQFSELVNDFYNNGLPSNLSGGRNPSLDYGFKGAEIAMAAYCSELQFLANPVTNHVQSAEQHNQDVNSLGLISSRKTAEAVDILKLMSSTFMIALCQAVDLRHLEENLRSAVKNCVTQVAKKALTMNPVGDLHNARFCEKDLLTAFDREAVFAYADDPCSANYPLMQKLRAVLVEHALTNGEAELSQDTSVFMKIAKFEEELRAALPKEVEAARAAVESGNAAIPNRIKECRSCPLYQFVREEVGTEYLTGEKTRSPGEECNKVFVAINEGKVIDPLLECLKEWNGEPLPIC

SEQ ID NO: 31 - protein, 713 aa, Setaria italica
MACEKSNVAANGDGLCMATPRADPLNWGKAAEELMGSHLDEVKRMVADYRQPLVKIEGASLNIAQVAAVANGAGEARVELDESARERVKASSDWVMNSMMNGTDSYGVTTGFGATSHRRTKEGGALQRELIRFLNAGAFGTGTDGHVLPAEATRAAMLVRINTLLQGYSGIRFEILEAIVKLLNANVTPCLPLRGTVTASGDLVPLSYIAGLVTGRENSVAVAPDGTKVNAAEAFKIAGIQGGFFELQPKEGLAMVNGTAVGSGLASTVLFEANVLAVLAEVLSAVFCEVMNGKPEYTDHLTHKLKHHPGQIEAAAIMEHILEGSSYMKLAKKLGELDPLMKPKQDRYALRTSPQWLGPQIEVIRAATKSIEREINSVNDNPLIDVARSKALHGGNFQGTPIGVSMDNTRLAIAAIGKLMFAQFSELVNDYYNNGLPSNLSGGRNPSLDYGFKGAEIAMASYCSELQFLGNPVTNHVQSAEQHNQDVNSLGLISSRKTAEAIEILKIMSSTFLIALCQAVDLRHIEENVKSAVKSCVMTVAKKTLSTNSTGGLHVARFCEKDLLQEIEREAVFAYADDPCSANYPLMKKLRNVLVERALANGTAEFNAETSVLAKVAQFEEELRAALPKAVEAARAAVENGTAAIPIRITECRSYPLYRFVREELGAVYLTGEKTRSPGEELNKVLVAINLGKHIDPLLECLKEWNGEPLPIC

SEQ ID NO: 32 - protein, 713 aa, Setaria viridis
MACEKSNVAANGDGLCMATPRADPLNWGKAAEELMGSHLDEVKRMVADYRQPLVKIEGASLNIAQVAAVANGAGEARVELDESARERVKASSDWVMNSMMNGTDSYGVTTGFGATSHRRTKEGGALQRELIRFLNAGAFGTGTDGHVLPAEATRAAMLVRINTLLQGYSGIRFEILEAIVKLLNANVTPCLPLRGTVTASGDLVPLSYIAGLVTGRENSVAVAPDGTKVNAAEAFKIAGIQGGFFELQPKEGLAMVNGTAVGSGLASTVLFEANVLAVLAEVLSAVFCEVMNGKPEYTDHLTHKLKHHPGQIEAAAIMEHILEGSSYMKLAKKLGELDPLMKPKQDRYALRTSPQWLGPQIEVIRAATKSIEREINSVNDNPLIDVARSKALHGGNFQGTPIGVSMDNTRLAIAAIGKLMFAQFSELVNDYYNNGLPSNLSGGRNPSLDYGFKGAEIAMASYCSELQFLGNPVTNHVQSAEQHNQDVNSLGLISSRKTAEAIEILKLMTSTFLIALCQAVDLRHIEENVKSAVKSCVMTVAKKTLSTNSTGGLHVARFCEKDLLQEIEREAVFAYADDPCSANYPLMKKLRNVLVERALANGTAEFNAETSVFAKVAQFEEELRAALPKAVEAARAAVENGTAAIPIRITECRSYPLYRFVREELGAVYLTGEKTRSPGEELNKVLVAINQGKHIDPLLECLKEWNGEPLPIC

SEQ ID NO: 33 - protein, 713 aa, Setaria italica
MACEKSNVAANGDGLCMATPRADPLNWGKAAEELMGSHLDEVKRMVADYRQPLVKIEGASLNIAQVAAVANGAGEARVELDESARERVKASSDWVMNSMMNGTDSYGVTTGFGATSHRRTKEGGALQRELIRFLNAGAFGTGTDGHVLPAEATRAAMLVRINTLLQGYSGIRFEILEAIVKLLNANVTPCLPLRGTVTASGDLVPLSYIAGLVTGRENSVAVAPDGTKVNAAEAFKIAGIQGGFFELQPKEGLAMVNGTAVGSGLASTVLFEANVLAVLAEVLSAVFCEVMNGKPEYTDHLTHKLKHHPGQIEAAAIMEHILEGSSYMKLAKKLGELDPLMKPKQDRYALRTSPQWLGPQIEVIRAATKSIEREINSVNDNPLIDVARSKALHGGNFQGTPIGVSMDNTRLAIAAIGKLMFAQFSELVNDYYNNGLPSNLSGGRNPSLDYGFKGAEIAMASYCSELQFLGNPVTNHVQSAEQHNQDVNSLGLISSRKTAEAIEILKIMSSTFLIALCQAVDLRHIEENVKSAVKSCVMTVAKKTLSTNSTGGLHVARFCEKDLLQEIEREAVFAYADDPCSANYPLMKKLRNVLVERALANGTAEFNAETSVLAKVAQFEEELRAALPKAVEAARAAVENGTAAIPIRITECRSYPLYRFVREELGAVYLTGEKTRSPGEELNKVLVAINQGKHIDPLLECLKEWNGEPLPIC

SEQ ID NO: 34 - protein, 713 aa, Setaria viridis; identical to SEQ ID NO: 32

SEQ ID NO: 35 - protein, 714 aa, Setaria italica
MECENGRVAAANGDGLCMATPRADPLNWGKAAEELMGSHLDEVKRMVADYRQPLVKIEGASLNIAQVAAVANGAGEARVELDESARERVKASSDWVMNSMMNGTDSYGVTTGFGATSHRRTKEGGALQRELIRFLNAGAFGTGTDGHVLPAEATRAAMLVRINTLLQGYSGIRFEILEAIVKLLNANVTPCLPLRGTVTASGDLVPLSYIAGLVTGRENSVAVAPDGTKVNAAEAFKIAGIQGGFFELQPKEGLAMVNGTAVGSGLASTVLFEANVLAVLAEVLSAVFCEVMNGKPEYTDHLTHKLKHHPGQIEAAAIMEHILEGSSYMKLAKKLGELDPLMKPKQDRYALRTSPQWLGPQIEVIRAATKSIEREINSVNDNPLIDVARSKALHGGNFQGTPIGVSMDNTRLALAAIGKLMFAQFSELVNDYYNNGLPSNLSGGRNPSLDYGFKGAEIAMASYCSELQFLGNPVTNHVQSAEQHNQDVNSLGLISSRKTAEAIEILKLMTSTFLIALCQAVDLRHIEENVKSAVKGCVMTVAKKTLSTNSAGGLHVARFCEKDLLQEIEREAVFAYADDPCSANYPLMKKLRNVLVERALANGTAEFNAETSVLAKVAQFEEELRAALPKAVEAARAAVENGTAAIPNRITECRSYPLYRFVREELGAVYLTGEKTRSPGEELNKVLVAINQGKHIDPLLECLKEWNGEPLPIS

SEQ ID NO: 36 - protein, 714 aa, Setaria viridis
MECENGRVAAANGDGLCMATPRADPLNWGKAAEELMGSHLDEVKRMVADYRQPLVKIEGASLNIAQVAAVANGAGEARVELDESARERVKASSDWVMNSMMNGTDSYGVTTGFGATSHRRTKEGGALQRELIRFLNAGAFGTGTDGHVLPAEATRAAMLVRINTLLQGYSGIRFEILEAIVKLLNANVTPCLPLRGTVTASGDLVPLSYIAGLVTGRENSVAVAPDGTKVNAAEAFKIAGIQGGFFELQPKEGLAMVNGTAVGSGLASTVLFEANVLAVLAEVLSAVFCEVMNGKPEYTDHLTHKLKHHPGQIEAAAIMEHILEGSSYMKLAKKLGELDPLMKPKQDRYALRTSPQWLGPQIEVIRAATKSIEREINSVNDNPLIDVARSKALHGGNFQGTPIGVSMDNTRLALAAIGKLMFAQFSELVNDYYNNGLPSNLSGGRNPSLDYGFKGAEIAMASYCSELQFLGNPVTNHVQSAEQHNQDVNSLGLISSRKTAEAIEILKLMTSSFLIALCQAVDLRHIEENVKSAVKGCVMTVAKKTLSTNSAGGLHVARFCEKDLLQEIEREAVFAYADDPCSANYPLMKKLRNVLVERALANGTAEFNADTSVLAKVAQFEEELRAALPNAVEAARAAVENGTAAIPNRITECRSYPLYRFVREELGAVYLTGEKTRSPGEELNKVLVAINQGKHIDPLLECLKEWNGEPLPIS

SEQ ID NO: 37 - protein, 713 aa, Panicum hallii
MACEKSNVAANGDGLCMAKPRADPLNWGKAAEELMGSHLDEVKRMVAEYRQPLVKIEGASLRIAQVAAVANGAGEARVELDESARERVKASSDWVMNSMMNGTDSYGVTTGFGATSHRRTKEGGALQRELIRFLNAGAFGTGTDGHVLPGEATRAAMLVRINTLLQGYSGIRFEILEAIVKLLNANVTPCLPLRGTVTASGDLVPLSYIAGLVTGRENSVAVAPDGTKVNAAEAFKIAGIQGGFFELQPKEGLAMVNGTAVGSGLASTVLFEANILAVLAEVLSAVFCEVMNGKPEYTDHLTHKLKHHPGQIEAAAIMEHILEGSSYMKLAKKLGELDPLMKPKQDRYALRTSPQWLGPQIEVIRAATKSIEREINSVNDNPLIDVARSKALHGGNFQGTPIGVSMDNTRLALAAIGKLMFAQFSELVNDYYNNGLPSNLSGGRNPSLDYGFKGAEIAMASYCSELQFLGNPVTNHVQSAEQHNQDVNSLGLISSRKTAEAIEILKLMSSTFLIALCQAVDLRHIEENVKSAVKSCVMTVAKKTLSTNATGGLHAARFCEKDLLQEIEREAVFAYADDPCSANYPLMKKLRNVLVERALANGTAEFNAETSVFAKVAQFEEELRAALPKAVEAARAAVENGTAAIPNRIAECRSYPLYRFVREELGAVYLTGEKTRSPGEELNKVLVAINQGKHIDPLLECLKEWNGEPLPIC

SEQ ID NO: 38 - protein, 713 aa, Panicum hallii
MACEQSNVAANGDGLCMAKPRADPLNWGKAAEELMGSHLDEVKRMVAEYRQPLVKIEGASLRIAQVAAVANGAGEARVELDESARERVKASSDWVMNSMMNGTDSYGVTTGFGATSHRRTKEGGALQRELIRFLNAGAFGTGTDGHVLPGEATRAAMLVRINTLLQGYSGIRFEILEAIVKLLNANVTPCLPLRGTVTASGDLVPLSYIAGLVTGRENSVAVAPDGTKVNAAEAFKIAGIQGGFFELQPKEGLAMVNGTAVGSGLASTVLFEANILAVLAEVLSAVFCEVMNGKPEYTDHLTHKLKHHPGQIEAAAIMEHILEGSSYMKLAKKLGELDPLMKPKQDRYALRTSPQWLGPQIEVIRAATKSIEREINSVNDNPLIDVARSKALHGGNFQGTPIGVSMDNTRLALAAIGKLMFAQFSELVNDYYNNGLPSNLSGGRNPSLDYGFKGAEIAMASYCSELQFLGNPVTNHVQSAEQHNQDVNSLGLISSRKTAEAIEILKLMSSTFLIALCQAVDLRHIEENVKSAVKSCVMTVAKKTLSTNATGGLHAARFCEKDLLQEIEREAVFAYADDPCSANYPLMKKLRNVLVERALANGTAEFNAETSVFAKVAQFEEELRAALPKAVEAARAAVENGTAAIPNRIAECRSYPLYRFVREELGAVYLTGEKTRSPGEELNKVLVAINQGKHIDPLLECLKEWNGEPLPIC

SEQ ID NO: 39 - protein, 712 aa, Panicum hallii
MECENGRVANGDSLCMATPRGDPLNWGKAAEELMGSHLDEVKRMVAEYRQPLVKIEGASLRIAQVAAVANGAGEARVELDESARERVKASSDWVMNSMMNGTDSYGVTTGFGATSHRRTKEGGALQRELIRFLNAGAFGTGTDGHVLPGEATRAAMLVRINTLLQGYSGIRFEILEAIVKLLNANVTPCLPLRGTVTASGDLVPLSYIAGLVTGRENSVAVAPDGTKVNAAEAFKIAGIQGGFFELQPKEGLAMVNGTAVGSGLASTVLFEANILAVLAEVLSAVFCEVMNGKPEYTDHLTHKLKHHPGQIEAAAIMEHILEGSSYMKLAKKLGELDPLMKPKQDRYALRTSPQWLGPQIEVIRAATKSIEREINSVNDNPLIDVARSKALHGGNFQGTPIGVSMDNTRLALAAIGKLMFAQFSELVNDYYNNGLPSNLSGGRNPSLDYGFKGAEIAMASYCSELQFLGNPVTNHVQSAEQHNQDVNSLGLISSRKTAEAIEILKLMSSTFLIALCQAVDLRHIEENVKSAVKSCVMTVAKKTLSTNATGGLHAARFCEKDLLQEIEREAVFAYADDPCSANYPLMKKLRNVLVERALANGTAEFNAETSVFAKVAQFEEELRAALPKAVEAARAAVENGTAAIPNRIAECRSYPLYRFVREELGAVYLTGEKTRSPGEELNKVLVAINQGKHIDPLLECLKEWNGEPLPIC

SEQ ID NO: 40 - protein, 713 aa, Panicum virgatum
MACEKSNVATNGDGLCMATPRADPLNWGKAAEELMGSHLDEVKRMVAEYRQPLVKIEGASLRIAQVAAVANGAGEARVELDESARDRVKASSDWVMNSMMNGTDSYGVTTGFGATSHRRTKEGGALQRELIRFLNAGAFGTGTDGHVLPAEATRAAMLVRINTLLQGYSGIRFEILEAIVKLLNANVTPCLPLRGTVTASGDLVPLSYIAGLVTGRENSVAVAPDGTKVNAAEAFKIAGIQGGFFELQPKEGLAMVNGTAVGSGLASTVLFEANILAILAEVLSAVFCEIMNGKPEFTDHLTHKLKHHPGQIEAAAIMEHILEGSSYMKLAKKLGELDPLMKPKQDRYALRTSPQWLGPQIEVIRAATKSIEREINSVNDNPLIDVARSKALHGGNFQGTPIGVSMDNTRLALAAIGKLMFAQFSELVNDYYNNGLPSNLSGGRNPSLDYGFKGAEIAMASYCSELQFLGNPVTNHVQSAEQHNQDVNSLGLISSRKTAEAIEILKLMSSTFLIALCQAVDLRHIEENVKSAVKSCVMTVAKKTLSTNATGGLHVARFCEKDLLQEIEREAVFAYADDPCSANYPLMKKLRNVLVERALANGAAEFSAETSVFAKVAQFEEELRAALPKAVEAARAAVENGTAAIPNRIAECRSYPLYRFVREELGAVYLTGEKTRSPGEELNKVLVAINQGKHIDPLLECLKEWNGEPLPIC

SEQ ID NO: 41 - protein, 712 aa, Panicum virgatum
MECENGRGANGDTLCMATPRADPLNWGKAAEELMGSHLDEVKRMVAEYRQPLVKIEGASLRIAQVAAVANGAGEARVELDESARDRVKASSDWVMNSMMNGTDSYGVTTGFGATSHRRTKEGGALQRELIRFLNAGAFGTGTDGHVLPAEATRAAMLVRINTLLQGYSGIRFEILEAIVKLLNANVTPCLPLRGTVTASGDLVPLSYIAGLVTGRENSVAVAPDGTKVNAAEAFKIAGIQGGFFELQPKEGLAMVNGTAVGSGLASTVLFEANILAILAEVLSAVFCEVMNGKPEFTDHLTHKLKHHPGQIEAAAIMEHVLEGSSYMKLAKKLGELDPLMKPKQDRYALRTSPQWLGPQIEVIRAATKSIEREINSVNDNPLIDVARSKALHGGNFQGTPIGVSMDNTRLALAAIGKLMFAQFSELVNDYYNNGLPSNLSGGRNPSLDYGFKGAEIAMASYCSELQFLGNPVTNHVQSAEQHNQDVNSLGLISSRKTAEAIEILKLMSSTFLIALCQAVDLRHIEENVKSAVKSCVMTVAKKTLSTNSTGGLHVARFCEKDLLQEIEREAVFAYADDPCSANYPLMKKLRNVLVERALANGAAEFSAETSVFAKVAQFEEELRAALPKAVEAARAAVENGTAAIPNRIAECRSYPLYRFVREELGAVYLTGEKTRSPGEELNKVLVAINLGKHIDPLLECLKEWNGEPLPIC

SEQ ID NO: 42 - protein, 712 aa, Panicum virgatum
MECKNGRVANGDSLCVATPRADPLNWGKAAEELMGSHLDEVKRMVAEYRQPLVKIEGASLRIAQVAAVANGAGEARVELDESARDRVKASSDWVMNSMMNGTDSYGVTTGFGATSHRRTKEGGALQRELIRFLNAGAFGTGTDGHVLPAEATRAAMLVRINTLLQGYSGIRFEILEAIAKLLNANVTPCLPLRGTITASGDLVPLSYIAGLVTGRENSVAVAPDGSKVNAAEAFKIAGIQGGFFELQPKEGLAMVNGTAVGSGLASTVLFEANILAILAEVLSAVFCEVMNGKPEFTDHLTHKLKHHPGQIEAAAIMEHILEGSSYMKHAKKLGELDPLMKPKQDRYALRTSPQWLGPQIEVIRAATKSIEREINSVNDNPLIDVSRGKALHGGNFQGTPIGVSMDNTRLALAAIGKLMFAQFSELVNDYYNNGLPSNLSGGRNPSLDYGFKGAEIAMASYCSELQFLGNPVTNHVQSAEQHNQDVNSLGLISSRKTAEAIEILKLMSSTFLIALCQAVDLRHIEENVKSAVKSCVMTVAKRTLSTNSSGGLHVARFCEKDLLQEIEREAVFAYADDPCSANYPLMKKLRNVLVERALANGAAEFNAETSVFAKVAQFEEELRAALPKAVEAARASVENGTAAIPNRIAECRSYPLYRFVREELGAVYLTGEKARSPGEELNKVLVAINQGKHIDPLLECLKEWNGEPLPIC

SEQ ID NO: 43 - protein, 714 aa, Sorghum bicolor
MACENGRVAATNGDGLCMATPRADPLNWGKAAEELMGSHLDEVKRMVAEYRQPLVKIEGASLRIAQVAAVAAGAGEARVELDESARGRVKASSDWVMNSMMNGTDSYGVTTGFGATSHRRTKEGGALQRELIRFLNAGAFGTGTDGHVLPAEATRAAMLVRINTLLQGYSGIRFEILEAIVKLLNANVTPCLPLRGTVTASGDLVPLSYIAGLVTGRENSVAVAPDGTKVNAAEAFKIAGIQGGFFELQPKEGLAMVNGTAVGSGLASTVLFEANVLAILAEVLSAVFCEVMNGKPEYTDHLTHKLKHHPGQIEAAAIMEHILEGSSYMKLAKKLGELDPLMKPKQDRYALRTSPQWLGPQIEVIRAATKSIEREINSVNDNPLIDVARSKALHGGNFQGTPIGVSMDNTRLAIAAIGKLMFAQFSELVNDYYNNGLPSNLSGGRNPSLDYGFKGAEIAMASYCSELQFLGNPVTNHVQSAEQHNQDVNSLGLISSRKTAEAIEILKLMSSTFLIALCQAVDLRHIEENVKSAVKSCVMTVAKKTLSTNSTGGLHVARFCEKDLLQEIEREAVFAYADDPCSANYPLMKKLRNVLVERALANGAAEFDAETSVFAKVAQFEEELRAALPKAVEAARAAVENGTAAIPNRITECRSYPLYRFVRQEVGAVYLTGEKTRSPGEELNKVLVAINQGKHIDPLLECLKEWNGEPLPIC

SEQ ID NO: 44 - protein, 714 aa, Sorghum bicolor; identical to SEQ ID NO: 43

SEQ ID NO: 45 - protein, 714 aa, Sorghum bicolor
MACDNGRVAATNGDGLCMATPRADPLNWGKAAEELMGSHLDEVKRMVAEYRQPLVKIEGASLRIAQVAAVAAGAGEARVELDESARGRVKASSDWVMNSMMNGTDSYGVTTGFGATSHRRTKEGGALQRELIRFLNAGAFGTGTDGHVLPAEATRAAMLVRINTLLQGYSGIRFEILEAIVKLLNANVTPCLPLRGTVTASGDLVPLSYIAGLVTGRENSVAVAPDGTKVNAAEAFKIAGIQGGFFELQPKEGLAMVNGTAVGSGLASTVLFEANVLAILAEVLSAVFCEVMNGKPEYTDHLTHKLKHHPGQIEAAAIMEHILEGSSYMKLAKKLGELDPLMKPKQDRYALRTSPQWLGPQIEVIRAATKSIEREINSVNDNPLIDVARSKALHGGNFQGTPIGVSMDNTRLAIAAIGKLMFAQFSELVNDYYNNGLPSNLSGGRNPSLDYGFKGAEIAMASYCSELQFLGNPVTNHVQSAEQHNQDVNSLGLISSRKTAEAIEILKLMSSTFLIALCQAVDLRHIEENVKSAVKSCVMTVAKKTLSTNSTGGLHVARFCEKDLLQEIEREAVFAYADDPCSANYPLMKKLRNVLVERALANGAAEFDAETSVFAKVAQFEEELRAALPKAVEAARAAVENGTAAIPNRITECRSYPLYRFVRQEVGAVYLTGEKTRSPGEELNKVLVAINQGKHIDPLLECLKEWNGEPLPIC

SEQ ID NO: 46 - protein, 716 aa, Zea mays
MECDNGRVAATNGDSLCMALPRAADPLNWGKAAEEMMGSHLDEVKRMVAEYRQPLVKIEGASLRIAQVAAVAAGAGEARVELDESARGRVKASSDWVRDSMMNGTDSYGVTTGFGATSHRRTKEGGALQRELIRFLNAGAFGIGTDAGHVLPAEATRAAMLVRINTLLQGYSGIRFEILEAIVKLLNANVTPCLPLRGTVTASGDLVPLSYIAGLVTGRENAVAVAPDGTKVNAAEAFRIAGIQSGFFELQPKEGLAMVNGTAVGSGLASTVLFEANVLAVLAEVLSAVFCEVMNGKPEYTDHLTHKLKHHPGQIEAAAIMEHILEGSSYMKLAKKLGELDPLMKPKQDRYALRTSPQWLGPQIEVIRASTKSIEREINSVNDNPLIDVARSKALHGGNFQGTPIGVSMDNTRLAVAAIGKLMFAQFSELVNDYYNNGLPSNLSGGRNPSLDYGFKGAEIAMASYCSELQFLGNPVTNHVQSAEQHNQDVNSLGLISSRKTAEAIEILKLMSSTFLIALCQAVDLRHIEENVKSAVKSCVMTVAKKTLSTNSTGGLHVARFCEKDLLQEIEREAVFAYADDPCSANYPLMKKLRNVLVERALANGTAEFDAETSVFAKVAQFEEELRTALPSAVEAARAAVENGTAAIPNRITECRSYPLYRFVREELGAVYLTGEKTRSPGEELNKVLVAINQGKHIDPLLECLKEWNGEPLPIC

SEQ ID NO: 47 - protein, 714 aa, Zea mays
MECENGRVAATNSDSLCMAMPRADPLNWGKAAEELMGSHLDEVKRMVAEYRQPLVKIEGASLSIAQVAAVATGAGEARVELDESARSRVKASSDWVMSSMMNGTDSYGVTTGFGATSHRRTKEGGALQRELIRFLNAGAFGTGADGHVLPAEATRAAMLVRINTLLQGYSGIRFEILEAIVKLLNANVTPCLPLRGTVTASGDLVPLSYIAGLVTGRENSVALAPDGSKINAAEAFKIAGIQGGFFELQPKEGLAMVNGTAVGSGLASTVLFEANILAILAEVLSAVFCEVMNGKPEYTDHLTHKLKHHPGQIEAAAIMEHILEGSSYMKLAKKLGELDPLMKPKQDRYALRTSPQWLGPQIEVIRAATKSIEREINSVNDNPLIDVARSKALHGGNFQGTPIGVSMDNTRLAIAAIGKLMFAQFSELVNDYYNNGLPSNLSGGRNPSLDYGFKGAEIAMASYCSELQFLGNPVTNHVQSAEQHNQDVNSLGLISSRKTAEAIEILKLMSSTFLIALCQAVDLRHIEENVKSAVKSCVMTVARKTLSTNSTGGLHVARFCEKDLLQEIEREAVFAYADDPCSANYPLMKKLRNVLVERALANGAAEFNAETSVFAKVAQFEDDLRAALPKAVEAARAAVENGTAGIPNRIADCRSYPLYRFVREELGAVYLTGEKTRSPGEELNKVLVAINQGKHIDPLLECLKEWNGEPLPIC

SEQ ID NO: 48 - protein, 714 aa, Zea mays
MATPRADPLNWGKAAEELMGSHLDEVKRMVAEYRQPLVKIEGASLSIAQVAAVAAGAGEARVELDESARGRVKASSDWVMSSMMNGTDSYGVTTGFGATSHRRTKEGGALQRELIRFLNAGAFGTGADGHVLPADATRAAMLVRINTLLQGYSGIRFEILEAIVKLLNANVTPCLPLRGTVTASGDLVPLSYIAGLVTGRENSVAVAPDGSKVNAAEAFKIAGIQGGFFELQPKEGLAMVNGTAVGSGLASTVLFEANILAILAEVLSAVFCEVMNGKPEYTDHLTHKLKHHPGQIEAAAIMEHILEGSSYMKLAKKLGELDPLMKPKQDRYALRTSPQWLGPQIEVIRAATKSIEREINSVNDNPLIDVARSKALHGGNFQGTPIGVSMDNTRLAIAAIGKLMFAQFSELVNDYYNNGLPSNLSGGRNPSLDYGFKGAEIAMASYCSELQFLGNPVTNHVQSAEQHNQDVNSLGLISSRKTAEAIEILKLMSSTFLIALCQAVDLRHIEENVKSAVKSCVMTVAKKTLSTDSTGGLHVARFCEKDLLQEIEREAVFAYADDPCSANYPLMKKLRNVLVERALANGAAEFNAETSVFAKVAQFEEDLRAALPKAVEAARAAVENGTAAIPNRITDCRSYPLYRFVREELGAVYLXYPLYRFVREELGAVYLTGEKTRSPGEELNKVLIAINQGKHIDPLLECLKEWNGEPLPIC

SEQ ID NO: 49 - protein, 713 aa, Oryza sativa
MACENGQVAADGINGLCMAAPRADPLNWGKAAEEMSGSHLDEVKRMVAEYRQPLVKIEGASLRIAQVAAVAAAGEARVELDESARERVKASSDWVMNSMMNGTDSYGVTTGFGATSHRRTKEGGALQRELIRFLNAGAFGTGTDGHVLSAEATRAAMLVRINTLLQGYSGIRFEILEAITKLLNANVTPCLPLRGTVTASGDLVPLSYIAGLVTGRENSVAVAPDGSKVNAAEAFKIAGIQGGFFELQPKEGLAMVNGTAVGSGLASTVLFEANILAVLAEVLSAVFCEVMNGKPEYTDHLTHKLKHHPGQIEAAAIMEHILEGSSYMKLAKKLGELDPLMKPKQDRYALRTSPQWLGPQIEVIRAATKSIEREINSVNDNPLIDVSRDKALHGGNFQGTPIGVSMDNTRLAIAAIGKLMFAQFSELVNDYYNNGLPSNLSGGRNPSLDYGFKGAEIAMASYCSELQFLGNPVTNHVQSAEQHNQDVNSLGLISSRKTAEAVEILKLMSSTFLVALCQAIDLRQIEENVKSAVKSCVMTVARKTLSTSATGGLHAARFCEKDLLQEIDREAVFAYADDPCSANYPLMKKLRNVLVERALANGAAEFNAETSVFAKVAQFEEELRAALPAAVLAARAAVENGTAATPNRITECRSYPLYRFVREELGTAYLTGEKTRSPGEELNKVLVAINQGKHIDPLLECLKEWNGAPLPIC

SEQ ID NO: 50 - protein, 713 aa, Oryza sativa
MECENGRVSANGMSGLCVAAPRADPLNWGKATEEMTGSHLDEVKRMVAEYRQPLVKIEGASLRIAQVAAVAAAGEARVELDESARERVKASSDWVMNSMMNGTDSYGVTTGFGATSHRRTKEGGALQRELIRFLNAGAFGTGTDGHVLPAEATRAAMLVRINTLLQGYSGIRFEILEAIAKLLNANVTPCLPLRGTITASGDLVPLSYIAGLVTGRENAVAVAPDGSKVNAAEAFKIAGIQGGFFELQPKEGLAMVNGTAVGSGLASTVLFEANILAILAEVLSAVFCEVMNGKPEYTDHLTHKLKHHPGQIEAAAIMEHILEGSSYMKHAKKLGELDPLMKPKQDRYALRTSPQWLGPQIEVIRAATKSIEREINSVNDNPLIDVSRGKALHGGNFQGTPIGVSMDNTRLAIAAIGKLMFAQFSELVNDFYNNGLPSNLSGGRNPSLDYGFKGAEIAMASYCSELQFLGNPVTNHVQSAEQHNQDVNSLGLISSRKTDEAIDILKLMSSTFLIALCQAVDLRHIEENVKSAVKSCVMTVAKKTLSTNSTGDLHVARFCEKDLLKEIDREAVFAYADDPCSHNYPLMKKLRNVLVERALANGAAEFNADTSVFAKVAQFEEELRATLPGAIEAARAAVENGTAAIPSRITECRSYPLYRFVREELGTKYLTGEKTRSPGEELNKVLVAINEGKHIDPLLECLKEWNGEPLPIC

SEQ ID NO: 51 - protein, 710 aa, Brachypodium distachyon
MACENGQVAANGICTAIQHADPLNWGKAAEALTGSHLEEVKRMVAEYRQPVVTIEGASLSIAKVAAVAAAGEAQVQLDESARERVKASSDWVMDSMANGVDSYGVTTGFGATSHRRTKEGGALQRELIRFLNAGAFGTGSDGHVLPAGATRAAMLVRINTLLQGYSGIRFEILEAIAKLLNANVTPCLPLRGTITASGDLVPLSYIAGLVTGRENSVAVAPDGSKVNAAEAFKIAGIHGGFFELQPKEGLAMVNGTAVGSGLASTVLFDANVLAVMAEVISAVFCEVMNGKPEFTDHLTHKLKHHPGQIEAAAIMEHILEGSSYMKLAKKLGDLDPLMKPKQDRYALRTSPQWLGPQIEVIRAATKSIEREINSVNDNPLIDVSRGKAIHGGNFQGTPIGVSMDNTRLALAAIGKLMFAQFSELVNDFYNNGLPSNLSGGRNPSLDYGFKGAEIAMASYCSELQFLGNPVTNHVQSAEQHNQDVNSLGLISARKTAEAIEILKLMTSTFLVALCQAIDLRHIEENVKSAVTSCVRAVAKKTLSTNSAGGLHVARFSEKDLIQEIDREAVFAYADDPCNPNYPLMKKLRGVLVERALANGVAEFDAETSVFAKVARFEEELRAALPVAVEAARAAVESGTAEAPNRIAECRSYPLYRFVRQELGTVYLTGEKTRSPGEELNKVLVAINQGKHIDPLLECLKEWNGEPLPIC

SEQ ID NO: 52 - protein, 716 aa, Brachypodium distachyon
MTMASKNVHVSADGYLILCPATSQHADPLNWGKAAEALTGSHLEEVKRMVAEYRQPVVTIEGASLSIAKVAAVAAAGEAQVQLDESARERVKASSDWVMDSMANGVDSYGVTTGFGATSHRRTKEGGALQRELIRFLNAGAFGTGSDGHVLPAGATRAAMLVRINTLLQGYSGIRFEILEAIAKLLNANVTPCLPLRGTITASGDLVPLSYIAGLVTGRENSVAVAPDGSKVNAAEAFKIAGIHGGFFELQPKEGLAMVNGTAVGSGLASTVLFDANVLAVMAEVISAVFCEVMNGKPEFTDHLTHKLKHHPGQIEAAAIMEHILEGSSYMKLAKKLGDLDPLMKPKQDRYALRTSPQWLGPQIEVIRAATKSIEREINSVNDNPLIDVSRGKAIHGGNFQGTPIGVSMDNTRLALAAIGKLMFAQFSELVNDFYNNGLPSNLSGGRNPSLDYGFKGAEIAMASYCSELQFLGNPVTNHVQSAEQHNQDVNSLGLISARKTAEAIEILKLMTSTFLVALCQAIDLRHIEENVKSAVTSCVRAVAKKTLSTNSAGGLHVARFSEKDLIQEIDREAVFAYADDPCNPNYPLMKKLRGVLVERALANGVAEFDAETSVFAKVARFEEELRAALPVAVEAARAAVESGTAEAPNRIAECRSYPLYRFVRQELGTVYLTGEKTRSPGEELNKVLVAINQGKHIDPLLECLKEWNGEPLPIC

SEQ ID NO: 53 - protein, 711 aa, Brachypodium distachyon
MARENARVAAANGICTAIQHADPLNWGKAAEELTGSHLDEVKRMVVEYREPVVTIEGASLSIAKVAAVAAAGEAKVELDESARERVKASSDWVMNSMANGVDSYGVTTGFGATSHRRTKEGGALQRELIRFLNAGAFGTGSDGHVLPAGATRAAMLVRINTLLQGYSGIRFEILEAIAKLLNANVTPCLPLRGTITASGDLVPLSYIAGLVTGRENSVAVAPDGRKVNAAEAFKIAGIHGGFFELQPKEGLAMVNGTAVGSGLASTVLFEANILAVLAEVISAVFCEVMNGKPEFTDHLTHKLKHHPGQIEAAAIMEHILEGSSYMKLAKKQGELDPLMKPKQDRYALRTSPQWLGPQIEVIRAATKSIEREINSVNDNPLIDVSRGKAIHGGNFQGTPIGVSMDNTRLALAAIGKLMFAQFSELVNDLYNNGLPSNLSGGRNPSLDYGLKGAEIAMASYCSELQFLGNPVTNHVQSAEQHNQDVNSLGLISSRKTAEAIEILKLMSSTFLVALCQAIDLRHIEENVKSAVKSCVMTVAKKTLSTNSTGDLHVSRFCEKDMLQEIDREAVFAYADDPCNHNYPLMKKLRGVLVESALANGVAEYNVETSVFAKVAQFEEELRAALPKAVEAARAAVESGTAATPNRITECRSYPLYRFVREELGTVFLTGEKTRSPGEELNKVLMAINQGKHIDPLLECLKEWNGEPLPIC

SEQ ID NO: 54 - protein, 712 aa, Brachypodium distachyon
MECENGQFAANGTGLCMATPSADPLNWGKAAEELTGSHLDEVKRMVAEYRKPVVTIEGATLSIAKVAAVAAAGEAKVELDESARERIKASSDWVMNSMMNGTDSYGVTTGFGATSHRRTKEGGALQRELIRFLNAGAFGTGSDGHVLPAGATRAAMLVRINTLLQGYSGIRFEILEAIAKLLNANVTPCLPLRGTITASGDLVPLSYIAGLVTGRENSVAVTPDGRKVNAAEAFKLAGIHGGFFELQPKEGLAMVNGTAVGSGLASTVLFEANILAVLAEVISAVFCEVMNGKPEFTDHLTHKLKHHPGQIEAAAIMEHILEGSSYMKLAKKLGDLDPLMKPKQDRYALRTSPQWLGPQIEVIRAATKSIEREINSVNDNPLIDVSRGKAIHGGNFQGTPIGVSMDNTRLALAAIGKLMFAQFSELVNDFYNNGLPSNLSGSRNPSLDYGFKGAEIAMASYCSELQFLGNPVTNHVQSAEQHNQDVNSLGLISSRKTAEAVEILKLMSSTFLVALCQAIDLRHIEENVKIAVKSCVMTVAKKTLSTNSTGGLHVARFCEKDLLQEIEREAVFAYADDPCSANYPLMKKLRSVLVERALSNGMAEFNAETSVFAKVAQFEEELRTVLPKAVEAARAAVESGTAATPNRIKECRSYPLYRFVREELGTAYLTGEKTRSPGEELNKVLVAINQGKHIDPLLECLKEWNGEPLPIC

SEQ ID NO: 55 - protein, 712 aa, Brachypodium distachyon
MECENGQFAANGTGLCMATPRADPLNWGKAAEELTGSHLDEVKRMVAEYRKPVVTIEGATLSIAKVAAVAAAGEAKVELDESARERVKASSDWVMNSMANGVDSYGVTTGFGATSHRRTKEGGALQRELIRFLNAGAFGTGSDGHVLPAGATRAAMLVRINTLLQGYSGIRFEILEAIAKLLNANVTPCLPLRGTITASGDLVPLSYIAGLVTGRENSVAVTPDGRKVNAAEAFKIAGIHGGFFELQPKEGLAMVNGTAVGSGLASTVLFEANILAVLAEVISAVFCEVMNGKPEFTDHLTHKLKHHPGQIEAAAIMEHILEGSSYMKLAKKLGDLDPLMKPKQDRYALRTSPQWLGPQIEVIRAATKSIEREINSVNDNPLIDVSRGKAIHGGNFQGTPIGVSMDNTRLALAAIGKLMFAQFSELVNDFYNNGLPSNLSGSRNPSLDYGFKGAEIAMASYCSELQFLGNPVTNHVQSAEQHNQDVNSLGLISSRKTAEAVEILKLMSSTFLVALCQAIDLRHIEENVKSAVKSCVMTVAKRTLSTNSTGGLHVARFCEKDLLQEIEREAVFAYADDPCSANYPLMKKLRGVLVERALSNGKAEFNAETSVFAKVAQFEEELRTALPKAVEAARSAVESGTAATPNRIKECRSYPLYRFVREELGTAYLTGEKTRSPGEELNKVLVAINQGKHIDPLLECLKEWNGEPLPIC

SEQ ID NO: 56 - protein, 716 aa, Oryza sativa
MECETGYVAAAADGGLCMEVPRADPLNWGKAAEEMAGSHLDEVKRMVAEYRQPVVRIEGASLRIAQVAAVAGAGDGEAAMVELDESARERVKASSDWVMNSMANGTDSYGVTTGFGATSHRRTKEGGALQRELIRFLNAGAFGTGADGHVLPAGATRAAMLVRINTLLQGYSGIRFEILEAVAKLLNANVTPCLPLRGTITASGDLVPLSYIAGLVTGRENSVAVAPDGRKVNAAEAFKIAGIQGGFFELQPKEGLAMVNGTAVGSGLASTVLFEANVLAILAEVLSAVFCEVMNGKPEYTDHLTHKLKHHPGQIEAAAIMEHILEGSSYMKEAKRLGELDPLMKPKQDRYALRTSPQWLGPQIEVIRAATKSIEREINSVNDNPLIDVSRGKALHGGNFQGTPIGVSMDNTRLAIAAIGKLMFAQFSELVNDFYNNGLPSNLSGGRNPSLDYGFKGAEIAMASYCSELQFLGNPVTNHVQSAEQHNQDVNSLGLISSRKTAEAIDILKLMSSTFLVALCQAIDLRHIEENVKGAVKTCVMTVAKKTLSTNSTGGLHVARFCEKDLLSEIDREAVFAYADDPCSANYPLMKKLRSVLVERALANGAAEFDAETSVLAKVARFEEELRAALPRAVEAARAAVESGTAAAPNRIAECRSYPLYRFVREELGTAYLTGEKTRSPGEELNKVLVAINQGKHIDPLLECLKEWNGAPLPIC

SEQ ID NO: 57 - protein, 717 aa, Panicum virgatum
MECETGLVRSLHGDGLCAPAPAPRAADPLNWGKAAEDLSGSHLGEVQRMVAEFREPLVRIQGASLSIAQVAAVAAGTGEARVELDESARGRVKASSDWVMSSMMNGTDSYGVTTGFGATSHRRTKEGGALQRELIRFLNAGAFGTGADGHVLPAEATRAAMLVRINTLLQGYSGIRFEILEAIAKLLNANVTPCLPLRGTITASGDLVPLSYIAGLITGRQNSVAVAPDGRKVDAAEAFKIAGIEHGFFELQPKEGLAMVNGTAVGSGLASTVLFEANVLAVMAEVISAVFCEVMTGKPEFTDHLTHKLKHHPGQIEAAAIMEHILEGSSYMKLAKKLGELDPLMKPKQDRYALRTSPQWLGPQIEVIRFATKSIEREINSVNDNPLIDVSRGKALHGGNFQGTPIGVSMDNTRLALAAIGKLMFAQFSELVNDYYNNGLPSNLSGGRNPSLDYGFKGAEIAMASYCSELQFLGNPVTNHVQSAEQHNQDVNSLGLISSRKTAEAIDILKLMTSTFLIALCQAIDLRHLEENMKAAVKNCVTQVAKKTLSMNAMGGLHIARFCEKDLQTAIDREAVFAYADDPCSPNYPLMQKLRAVLIEHALANGDAERVAETSIFAKITKFEEELRAVLPREVEAARVAVAEGAAPLPNRIKDSRSFPLYRFVREELGCVFLTGEKLKSPGEECNKVFVGISEGKLVDPMLECLKEWDGKPLPIN

SEQ ID NO: 58 - protein, 717 aa, Panicum virgatum
MECETGLVRSLHGDGLCVPAPAAARADPLNWGKAAEDLSGSHLGEVQRMVAEFREPLVRIQGASLSIAQVAAVAAGAGEARVELDESARGRVKASSDWVMSSMMNGTDSYGVTTGFGATSHRRTKEGGALQRELIRFLNAGAFGTGADGHVLPAEATRAAMLVRINTLLQGYSGIRFEILEAIAKLLNANVTPCLPLRGTITASGDLVPLSYIAGLITGRQNSVAVAPDGRKVDAAEAFKIAGIEHGFFELQPKEGLAMVNGTAVGSGLASTVLFEANVLAVMAEVISAVFCEVMTGKPEFTDHLTHKLKHHPGQIEAAAIMEHILEGSSYMKLAKKLGELDPLMKPKQDRYALRTSPQWLGPQIEVIRFATKSIEREINSVNDNPLIDVSRGKALHGGNFQGTPIGVSMDNTRLALAAIGKLMFAQFSELVNDFYNNGLPSNLSGGRNPSLDYGFKGAEIAMASYCSELQFLGNPVTNHVQSAEQHNQDVNSLGLISSRKTAEAIDILKLMTSTFLIALCQAIDLRHLEENMKAAVKNCVTQVAKKTLSMNAMGGLHIARFCEKDLQTAIDREAVFAYADDPCSPNYPLMQKLRAVLIEHALANGDAERVVETSIFAKVAEFEQQVRAALPKGVEAARAAVESGNPMVPNRIKECRSYPLYRFVREELGTEYLTGEKTRSPGEELNKVLVAINQRKHIDPLLECLKEWNGEPLPLC

SEQ ID NO: 59 - protein, 718 aa, Panicum hallii
MECETGIVRSLHGDGLCVPAPAAAARADPLNWGKAAEDLSGSHLGEVQRMVAEFREPLVRIQGASLSIAQVAAVAAGAGEARVELDESARERVKASSDWVMNSMMNGTDSYGVTTGFGATSHRRTKEGGALQRELIRFLNAGAFGTGADGHVLPAEATRAAMLVRINTLLQGYSGIRFEILEAIAKLLNANVTPCLPLRGTITASGDLVPLSYIAGLITGRQNSVAVAPDGRKVDAAEAFKIAGIEHGFFELQPKEGLAMVNGTAVGSGLASTVLFEANVLAIMAEVISAVFCEVMTGKPEFTDHLTHKLKHHPGQIEAAAIMEHILEGSSYMKLAKKLGELDPLMKPKQDRYALRTSPQWLGPQIEVIRFATKSIEREINSVNDNPLIDVSRGKALHGGNFQGTPIGVSMDNTRLALAAIGKLMFAQFSELVNDFYNNGLPSNLSGGRNPSLDYGFKGAEIAMASYCSELQFLGNPVTNHVQSAEQHNQDVNSLGLISSRKTAEAIDILKLMTSTFLIALCQAIDLRHLEENMKAAVKNCVTQVAKKTLSMNAMGGLHIARFCEKDLQTAIDREAVFAYADDPCSANYPLMQKLRAVLIEHALANGDAERVVETSIFAKVAEFEQQVRAALPKEVEAARAAVESGNPMVPNRIKECRSYPLYRFVREELGTEYLTGEKTRSPGEELNKVLVAINQRKHIDPLLECLKEWNGEPLPLC

SEQ ID NO: 60 - protein, 718 aa, Setaria italica
MECETGLVRSLHGDGLCMSAQAAAPRADPLNWGKAAEDLSGSHLDEVKRMVAEFREPLVKIQGASLSIAQVAAVAAGAGEARVELDESARERVKASSDWVMNSMMNGTDSYGVTTGFGATSHRRTKEGGALQRELIRFLNAGAFGTGADGHVLPAEATRAAMLVRINTLLQGYSGIRFEILETIAKLLNANVTPCLPLRGTITASGDLVPLSYIAGLITGRQNSVAVAPDGRKVDAAEAFKIAGIEHGFFELQPKEGLAMVNGTAVGSGLASTVLFEANVLAIMAEVISAVFCEVMTGKPEFTDHLTHKLKHHPGQIEAAAIMEHILEGSSYMKLAKKLGELDPLMKPKQDRYALRTSPQWLGPQIEVIRFATKSIEREINSVNDNPLIDVSRGKALHGGNFQGTPIGVSMDNTRLALAAIGKLMFAQFSELVNDFYNNGLPSNLSGGRNPSLDYGFKGAEIAMASYCSELQFLGNPVTNHVQSAEQHNQDVNSLGLISSRKTAEAIEILKLMTSTFLIALCQAIDLRHLEENMKAAVKNCVTQVAKKTLSMNAMGGLHIARFCEKDLQTAIDREAVFAYADDPCSPNYPLMQKLRAVLIEHALANGDAERVVETSIFAKVAEFEQQVRAALPKEVEAARAAVESGRPMVPNRIKECRSYPLYRFVREELGAEYLTGEKTRSPGEELNKVLVAINERKHIDPLLECLKEWNGEPLPLC

SEQ ID NO: 61 - protein, 718 aa, Setaria viridis; identical to SEQ ID NO: 60

SEQ ID NO: 62 - protein, 728 aa, Zea mays
MECEAGLLVRSSLNGEGLCMPAPRADPLNWGKAAEGLSGSHLDEVKRMVAEFREPLVKIQGASLSVAQVAAVAVGAGGGEARVELDESARERVRASSDWVMGSMMNGTDSYGVTTGFGATSHRRTKEGGALQRELIRFLNAGAFGTGADGHVLPAEATRAAMLVRVNTLLQGYSGIRFEILEAIAKLLNANVTPCLPLRGTIXRATSSRSPTSPASSRAARTPWRKVGAAEAFKIAGIEHGFFELQPKEGLAMVNGTAVGSGLASTVLFEANVLAVLAEVISAVFCEVMTGKPEFTDHLTHKLKHHPGQIEAAAVMEHILEGSSYMKLAKRLGELDPLMKPKQDRYALRTSPQWLGPQIEVIRFATKSIEREINSVNDNPLIDVSRGKALHGGNFQGTPIGVSMDNTRLALAAIGKLMFAQFSELVNDYYNNGLPSNLSGGRNPSLDYGFKGAEIAMASYCSELQFLGNPVTNHVQSAEQHNQDVNSLGLISSRKTAEAIDILKLMSSTFLIALCQAIDLRHLEENVKAAVKNCVTQVAKKSLSLNARGGLHNARFCEKDLQTAIDREAVFAYADDPCSPNYALMQKLRAVLVEHALANGDAERDVDTSIFAKVAEFEQQVRAALPKEVEAARAAVENGSPLVPNRIKECRSYPLYRFVREEVGTEYLTGEKTRSPGEELNKVLVAINQRKHIDPLLECLKXAINQRKHIDPLLECLKEWNGEPLPLC

SEQ ID NO: 63 - protein, 721 aa, Zea mays
MECETGLVRSLNGEGLCMSSSSAAAAPRADALNWGKAAEDLSGSHLDEVKRMVVEFREPLVRIQGASLSIGQVAAVAAGAGGEARVELDESARGRVKASSDWVMSSMMNGTDSYGVTTGFGATSHRRTKEGGALQRELIRFLNAGAFGTGADGHVLPAEATRAAMLVRINTLLQGYSGIRFEILEAIAKLLNANVTPCLPLRGTITASGDLVPLSYIAGLITGRQNSVAVGPDGRKVGAAEAFRIAGIEHGFFELQPKEGLAMVNGTAVGSGLASTVLFEANVLAVMAEVISAVFCEVMTGKPEFTDHLTHKLKHHPGQIEAAAIMEHVLEGSSYMKLAKKLGELDPLMKPKQDRYALRTSPQWLGPQIEVIRFATKSIEREINSVNDNPLIDVSRGKALHGGNFQGTPIGVSMDNTRLALAAIGKLMFAQFSELVNDYYNNGLPSNLSGGRNPSLDYGFKGAEIAMASYCSELQFLGNPVTNHVQSAEQHNQDVNSLGLISSRKTAEAVDILKLMTSTFLIALCQAIDLRHLEENVKAAVKNCVTQVAKKSLSLNARGGLHNARFCEKDLQTAIDREAVFAYADDPCSPNYPLMQKLRAVLVEHALANGDAERAAETSIFAKVAEFEQQVRAALPKEVEAARAAVESGNPLVPNRIRECRSYPLYRFVREEVGTEYLTGERTRSPGEELNKVLVAINQRKHIDPLLECLKEWNGAPLPLC

SEQ ID NO: 64 - protein, 718 aa, Sorghum bicolor
MECETGLVRSLNGDGLCMSAQAAPRGADPLNWGKAAEDLSGSHLEEVKRMVAEFRDPVVKIQGASLSIAQVAAVAAGAGEARVELDESARERVKASSDWVMSSMMNGTDSYGVTTGFGATSHRRTKEGGALQRELIRFLNAGAFGTGADGHVLPAEATRAAMLVRINTLLQGYSGIRFEILEAIAKLLNANVTPCLPLRGTITASGDLVPLSYIAGLITGRQNSVAVAPDGRKVDAAEAFKIAGIEHGFFELQPKEGLAMVNGTAVGSGLASTVLFEANVLAIMAEVISAVFCEVMTGKPEFTDHLTHKLKHHPGQIEAAAIMEHILEGSSYMKLAKKLGELDPLMKPKQDRYALRTSPQWLGPQIEVIRFATKSIEREINSVNDNPLIDVSRGKALHGGNFQGTPIGVSMDNTRLALAAIGKLMFAQFSELVNDYYNNGLPSNLSGGRNPSLDYGFKGAEIAMASYCSELQFLGNPVTNHVQSAEQHNQDVNSLGLISSRKTAEAIDILKLMTSTFLIALCQAIDLRHIEENVKAAVKNCVTQVAKKSLSLNARGGLHNARFCEKDLQTAIDREAVFAYADDPCSPNYPLMQKLRAVLIEHALANGDAERVVETSIFAKVAEFEQQVRAALPKEVEAARAAVESGNPLVPNRIKECRSYPLYRFVREEVGTQYLTGEKTRSPGEELNKVLVAINQRKHVDPLLECLKEWNGEPLPLC

SEQ ID NO: 65 - protein, 718 aa, Oryza sativa
MECETGLVRSLNGDGLCMSSVSAPPRADPLNWGKAADELAGSHLDEVKRMVEDFRQPLVKIEGASLTIAQVAAVAAGAGDARVELDESARGRVKASSDWVMNSMMNGTDSYGVTTGFGATSHRRTKEGGALQRELIRFLNAGAFGTGTDGHVLPAEATRAAMLVRINTLLQGYSGIRFEILEAITKLLNANVTPCLPLRGTITASGDLVPLSYIAGLITGRQNSVAVAPDGRKVTAAEAFKIAGIEHGFFELQPKEGLAMVNGTAVGSGLASTVLFEANVLAILAEVLSAVFCEVMTGKPEYTDHLTHKLKHHPGQIEAAAIMEHILEGSSYMKLAKKLGELDPLMKPKQDRYALRTSPQWLGPQIEVIRFATKSIEREINSVNDNPLIDVSRGKALHGGNFQGTPIGVSMDNTRLALAAIGKLMFAQFSELVNDFYNNGLPSNLSGGRNPSLDYGFKGAEIAMASYCSELQFLGNPVTNHVQSAEQHNQDVNSLGLISSRKTAEAIDILKLMSSTFLIALCQAIDLRHLEENMKTAVKNCVMQVAKKSLSMNHMGGLHIARFCEKDLLTAIDREAVFAYADDPCSANYPLMQKLRAVLIEHALANGDAERVLETSIFAKVAEFEQHVRAALPKEVEAARAAVENGTPLVPNRIKECRSYPLYRFVREEVGTEYLTGEKTRSPGEELNKVLVAINERKHIDPLLECLKEWNGAPLPLC

SEQ ID NO: 66 - protein, 700 aa, Oryza sativa
MECETGVNGSGPRGSDPLYWGKAAEGVAGSHLEEVKRMVVEYRAPLVKIDGAMLSVAKVAAVAGEAARVQVVLDESARPRLEASREWVFESTMNGTDTYGVTTGFGGAAHRRTKEFAALQKELIRYLNAGVFGASRSDGYTLPTEVTRAAMLVRINTLTQGYSGIRFEILEAIAKLLNANVTPCLPLRGTITASGDLVPLSYIAGLITGRHNSAAVAPDGTKVDAAEAFRMAGIEHGFFALQPKEGLAIVNGTAVGSGLAAIVLFEANVLAVLAEVLSAVYCEVMAGNPEYTDHLIHALKHHPGQIEAAAIMEHILEGSSYMKKLAKDKEQGQGELDRLTKLKQDRYAIRTAPQWLGPQVEVIRFATKSIEREINSVNDNPVIDVARRKALHGGNFQGTPVGVSMDNTRLAIAAIGRLMFSQFSELVSSFYSNGLPSNLSGGRNPSLDYGFNGAEVAMASYCSELQFLANPVTNHVQTAEQHNQSVNSLGLISSRMTAEAVTILKLMSSTFLIALCQAVDLRQLEESIKAAVNKCVTNVAKKSLTLAMDDDDHLLALLGAAIDRVAVFTYAEDPCRSSLPLMQKLRAVLMEHALANNGDILLAKVAEFEQQLRAVLPDEVEAARAAVESGTAPNRISDCRSYPLYRFVRKELGAEYLTGEKTRSPGEEVDKVVIAMNQHKHIHPLLECLSEWKGAPLPLS

SEQ ID NO: 67 - protein, 680 aa, Oryza sativa
MECETGLVDRPLNGDPLYWGKAAEGLAGSHLDEVKRMVVEYRAPLVKIDGAMLSVAKVAAVAGEAARVQVVLDESARPRLEASREWVFDSTMNGTDTYGVTTGFGGAAHRRTKEFAALQKELIRRSDGYTLPTEVTRAAMLVRINTLTQGYSGIRFEILEAIAKLLNANVTPCLPLRGTITASGDLVPLSYIAGLITGRQNSAAVAPDGSKVDAAEAFRIAGIEHGFFALQPKEGLAIVNGTAVGSGLAAIVLFEANVLAVLAEVLSAVYCEVMAGNPEYTDHLIHALKHHPGQIEAAAIMEHILEGSSYMRLAKEQGELDRLTKLRQDRYAIRTAPQWLGPQVEVIRFATKSIEREINSVNDNPVIDVARRKALHGGNFQGTPIGVSMDNTRLAIAAIGRLMFSQFSELASSFYSNGLPSNLSGGRNPSLDYGFNGAEVAMASYCSELQFLANPVTNHVQTAEQHNQSVNSLGLISSRMTAEAVTILKLMSSTFLIALCQAVDLRQLEESIKAAVNKCVTNVAKKSLAMDDDDLLALLGAAIDRVAVFTYAEDPCRSSLPLIQKLRAVLMDHALANGDNQLAKVAEFEQQLRAVLHDEVEAARAAVESGTAPNRITQCRSYPLYRFVRKELGAEYLTGEKTRSPGEEVDKVVIAMNQHKHINPLLECLSEWKGAPLPLN

SEQ ID NO: 68 - protein, 709 aa, Sorghum bicolor
MECETGLVDRPRRNGSAGPHVVDDDPLKWGKAAEDLTGSHLDDVRRMVEEYRMPLVKIDGAMLSVAKVAAVAGEATRRVQVVLDESARPRLEASREWVFDSTMNGTDTYGVTTGFGGASHRRTKEFRALQTELIRYLNAGVFGASSSDGYTLPAEVTRAAMLVRINTLTQGYSGIRFEILEAIAKLLNANVTPCLPLRGSITASGDLVPLSYIAGLITGRGNSAAVAPDGTKVGAAEAFRIAGIDHGFFALEPKEGLAIVNGTAVGSGLAAIVLFEANVLAVLAEVLSAVYCEVMAGNPEYTDHLIHALKHHPGQIEAAAIMEHVLEGSSYMKLAREQAELDPLTKPKQDRYAIRTTPQWLGPQVEVIRFATKSIEREINSVNDNPVIDVARGKALHGGNFQGTPVGVSMDNTRLAVAAIGKLMFAQFTELVSSFYSNGLPSNLSGGRDPSLDYGFNGAEVAMASYCSELQFLANPVTNHVQTAEQHNQSVNSLGLISSRMTAEAVTILKLMSSTFLIALCQAIDLRQLEESIKAAVKKCVARVAKKCLAMDDDAAAAAAQLLGAVDRVAVFTYAEDPCRPSLPLMQKLRVVLMEHALGAAEGAVETTSILAKVAEFEQQLRAVLPDEVEAARAAVESGTAPKRIAECRSYPLYRFVREELGAEYLTGEKTRSPGEEVDKVVIAMNQRKHIDPLLECLSDWKGAPLHAT

SEQ ID NO: 69 - protein, 698 aa, Zea mays
MECETGLVDRPLNSSAPRTGDPLKWGKAAEGLTGSHLDEVKRMVEEYRVPLVKIDGAVLSVAKVAAVAGRVQVALDESARPRLEASREWVFESTMNGTDTYGVTTGFGGACHRRTKEFAALQKELIRYLNAGVFGASSSDGYTLPPEVTRAAMLVRINTLTQGYSGIRFEILEAIAKLLNANVTPCLPLRGSITASGDLVPLSYIAGLITGRKNSVAVAPDGTKVDAVEAFRIAGIEHGFFALQPKEGLAIVNGTAVGSGLAAIVLFEANILAVLAEVLSALYCEVMAGNPEYTDHLIHALKHHPGQIEAAAIMEHILEGSSYMKLAKEQGELDPLTKMKQDRYAIRTTPQWLGPQVEVIRFATKSIEREINSVNDNPIIDVARRKALHGGNFQGTPVGVSMDNTRLAVAAIGKLMFAQFSELVSSFYSNGLPSNLSGGRNPSLDYGFNGAEVAMASYCSELQFLANPVTNHVQTAEQHNQSVNSLGLISSRMTAEAVTILKLMSSTFLIALCQAVDLRQLEESIKAAVKKCVSHVAKKSLAIDDHDLLVAIDRVAVFTYAEDPCRPSYPLMQKLRAVLMEHALINGDSNTERPMETSILAKVAEFEKQLSAVLPDEVEAARAAVESGTTPNRITECRSYPLYRFVREELGAEYLTGEKTRSPGEEVDKVVIAMNQRKHIDPLLQCLSEWKGAPLPII

SEQ ID NO: 70 - protein, 717 aa, Brachypodium distachyon
MECENGLVGSLNGEGLCMSAPPRAAADPLNWAKTAEELAGSHLEEVKKMVAQFRMPLVKIEGATLGIAQVAAVAAGAGEARVELDESARGRVKESSDWVMNSMMNGTDSYGVTTGFGATSHRRTKEGGALQRELIRFLNAGAFGTGTDGHVLPAEATRAAMLVRINTLLQGYSGIRFEILEAITKLLNANVTPCLPLRGTITASGDLVPLSYIAGLITGRQNSVAVAPDGSKVSAAEAFKIAGIEHGFFELQPKEGLAMVNGTAVGSGLASTVLFEANIQAILAEVLSAVFCEVMTGKPEFTDHLTHKLKHHPGQIEAAAIMEHILEGSSYMKEAKKQGELDPLMKPKQDRYALRTSPQWLGPQIEVIRFATKSIEREINSVNDNPLIDVSRGKAIHGGNFQGTPIGVSMDNTRLAIAAIGKLMFAQFSELVNDFYNNGLPSNLSGGRNPSLDYGFKGAEIAMASYCSELQFLGNPVTNHVQSAEQHNQDVNSLGLISSRKTAEAIDILKIMSSTFLIALCQAIDLRHLEENMKTAVRNCVMQVAKKTLSMNAMGGLHIARFCEKDLLTAIDREAVFAYADDPCSPNYPLMQKLRAVLIEHALANGDGERALETSIFAKVAEFEQNLRAALPKEVEAARASVENGTPLAPNRIKDCRSYPLYRFVREELGTEYLTGEKTRSPGEELNKVLVAMNQRKHIDPLLECLKEWNGEPLPLC

SEQ ID NO: 71 - protein, 716 aa, Streptochaeta angustifolia
MECENSQVAANGNCLCLAKPQADPLNWGKAAEELTGSHLDEVKRMVAEYREPVVRIEGAGLRIAQVAAVAAAADGGEAAKVELDESARGRVKASSDWVMNSMTKGTDSYGVTTGFGATSHRRTKEGGALQRELIRFLNAGAFGTGTDGHVLPAAATRAAMLVRINTLLQGYSGIRFEILEAIAALLNVNVTPCLPLRGTITASGDLVPLSYIAGLVTGRQNSVAVTTDGRKVNAAEAFKIAGIQHGFFELQPKEGLAMVNGTAVGSGLASTVLFEANILAILSEVLSAVFCEVMNGKPEYTDHLTHKLKHHPGQIEAAAIMEHILEGSSYMKLAKKLGELDPLMKPKQDRYALRTSPQWLGPQIEVIRAATKSIEREINSVNDNPLIDVSRGKALHGGNFQGTPIGVSMDNTRLAIAAIGKLMFAQFSELVNDFYNNGLPSNLSGGRNPSLDYGFKGAEIAMASYCSELQFLGNPVTNHVQSAEQHNQDVNSLGLISSRKTAEAIDILKLMSSTFLIALCQAIDLRHIEETVKSAVKNCVMQVAKKVLSMNAAGDLHIARFCEKDLLTAIDREAVFAYADDPCSGTYPLMKNLRAVLVEHALANGEAERNAETSVFAKVAKFEEELRAALPKAVEAARASVENGTGPTPNRIKACRSYPLYRFVREELGTEYLSGEKTRSPGEELDKVLVAINQGKHIDPLLECLKEWNGKPLPIC

SEQ ID NO: 72 - protein, 716 aa, Streptochaeta angustifolia
MECENVHVAANGNGLCLAKPHADPLNWGAAASELTGSHLDEVKRMVAHFREPVVNIEGAGLRIGQVAAVAAAADGVAAAMVELDESARGRVKASSDWVMNSMTEGTDSYGVTTGFGATSHRRTKEGGALQRELIRFLNAGAFGTGADGYVLPAPATRAAMLVRINTLLQGYSGIRFEILEAIAKLLNANVTPCLPLRGTITASGDLVPLSYIAGLVTGRQNSVAVTLDGRKVNAAEAFKIAGIQHGFFELQPKEGLAMVNGTAVGSGLASTVLFEANILAILAEVLSAVFCEAMNGKPEYTDHLTHKLKHHPGQIEAAAIMEHILEGSSYMKLAKKLGELDPLMKPKQDRYALRTSPQWLGPQIEVIRAATKSIEREINSVNDNPLIDVSRGKALHGGNFQGTPIGVSMDNTRLAIAAIGKLMFAQFSELVNDFYNNGLPSNLSGGRNPSLDYGFKGAEIAMASYCSELQFLGNPVTNHVQSAEQHNQDVNSLGLISSRKTAEAIDILKLMSSTFLIALCQAIDLRHIEENVKSAVKNCVMQVSKRVLSLNATGNLHIARFCEKDLLTAIDREAVFAYADDPCNASYPLMKNLRAVLVEHALANGEAERNVDTSVFTKVAEFEQQLRAALPKEVEAARAAVENGAATVPNRIKECRSYPLYRFAREELGTDYLTGEKARSPGEEVDKVLVAINQGKHIDPLLECLKEWNGEPLPIC

SEQ ID NO: 73 - protein, 714 aa, Panicum virgatum
MECENGRVAAATGNGVCLPVPPRADPLNWGKAAENLAGSHLEAVKRMVEEYRRPLVRIEGASLTVAQVAAVAAAGEARVELDESARGRVKASSDWVMSSMMNGTDSYGVTTGFGATSHRRTKEGGALQRELIRFLNAGAFGTGDDGHVLPASATRAAMLVRINTLLQGYSGIRFEILEAIAALLNANVTPCLPLRGTITASGDLVPLSYIAGLVTGRPNSVAVTPDGRKVDAAEAFKAAGIQHGFFELQPKEGLAMVNGTAVGSGLASPVLFEANVLAVLAEVLSAVFCEVMNGKPEYTDHLTHKLKHHPGQIEAAAIMEHILEGSSYMALAKKLGELDPLMKPKQDRYALRTSPQWLGPQIEVIRAATKSIEREINSVNDNPLIDVSRGKALHGGNFQGTPIGVSMDNTRLAVAAIGKLMFAQFSELVNDFYNNGLPSNLSGGRNPSLDYGFKGAEIAMASYCSELQFLANPVTNHVQSAEQHNQDVNSLGLISSRKTAEAVDILKLMSSTFLVALCQAIDLRHLEENLKSAVKSCVMTVAKKTLSTGATGALHSARFCEKDLLTAIDREAVFAYADDPCSANYPLMQKMRSVLVEHALANGEAEHDPDTSVFAKLATFEEELRAALPREVDAARAAVENGTAAIPNRIAECRSYPLYRFVRQELGTEYLTGEKTRSPGEEVNKVFVAMNAGKHIDAVLECLKGWNGEPLPIC

SEQ ID NO: 74 - protein, 713 aa, Panicum hallii
MECENGHVAASGNGVCLPVPPRADPLNWGKAAEDLAGSHLEAVKRMVEEYRRPLVKIEGASLTVAQVAAVAAAGEARVELDESARGRVKASSDWVMNSMMNGTDSYGVTTGFGATSHRRTKEGGALQRELIRFLNAGAFGTGDDGHVLPASATRAAMLVRINTLLQGYSGIRFEILETIAALLNANVTPCLPLRGTITASGDLVPLSYIAGLVTGRPNSVAVTPDGRKVDAAEAFKVAGIQHGFFELQPKEGLAMVNGTAVGSGLASMVLFEANVLAVLAEVLSAVFCEVMNGKPEYTDHLTHKLKHHPGQIEAAAIMEYILDGSSYMMLAKKLGELDPLMKPKQDRYALRTSPQWLGPQIEVIRAATKSIEREINSVNDNPLIDVSRGKALHGGNFQGTPIGVSMDNTRLAIAAIGKLMFAQFSELVNDFYNNGLPSNLSGGRNPSLDYGFKGAEIAMASYCSELQFLANPVTNHVQSAEQHNQDVNSLGLISSRKTAEAIDILKLMSSTFLIALCQAIDLRHLEENLKSAVKGCVMTVAKKTLSTSATGALHSARFCEKDLLTAIDREAVFAYADDPCSANYPLMQKMRSVLVEHALANGEAERDPDTSVFAKVTAFEEELRAALPREVDAARAAVESGTAAIPNRIAECRSYPLYRFVREELGTEYLTGEKTRSPGEEVNKVFVAMNAGKHIDAVLECLKGWNGEPLPIC

SEQ ID NO: 75 - protein, 80 aa, Panicum virgatum
MTVFAKVATFEEELRAALSREVDAARGAVIPNRIAECRSYLKTRSFDEEVNKVFVAVNAGKHIDAMLECLKEWNGEPMPM

SEQ ID NO: 76 - protein, 713 aa, Panicum virgatum
MECENGHVAANGNGVCLPVPPRADPLNWGKAAEDLAGSHLEAVRRMVEEYRRPLVKIEGASLTVAQVAAVAAAAEARVELDESARGRVKASSDWVMNSMMNGTDSYGVTTGFGATSHRRTKEGGALQRELVRFLNAGAFGTGDDGHVLPASATRAAMLVRINTLLQGYSGIRFEILETIAALLNANVTPCLPLRGTITASGDLVPLSYIAGLVTGRPNSVAVTPDGRKVDAAEAFKVAGIQHGFFELQPKEGLAMVNGTAVGSGLASIVLFEANVLAVLAEVLSAVFCEVMNGKPEYTDHLTHKLKHHPGQIEAAAIMEHILDGSSYMMLAKKLGELDPLMKPKQDRYALRTSPQWLGPQIEVIRAATKSIEREINSVNDNPLIDVSRGKALHGGNFQGTPIGVSMDNTRLAIAAIGKLMFAQFSELVNDFYNNGLPSNLSGGRNPSLDYGFKGAEIAMASYCSELQFLANPVTNHVQSAEQHNQDVNSLGLISSRKTAEAVDILKLMSSTFLIALCQAIDLRHLEENLKSAVKSCVMTVAKKTLSTSATGALHSARFCEKDLLTAIDREAVFAYADDPCSTNYPLMQKMRSVLVEHALANGEAERDPDTSVFGKVATFEEELRAALPPEVEASRAGVEKGAAAIPNRIAECRSYPLYRFVRQELGTEYLTGEKTRSPGEEVNKVFVAMNAGKHIDAVLECLKEWNGEPLPIC

SEQ ID NO: 77 - protein, 715 aa, Setaria viridis
MECENGHVVAANGNGVCLPVPPRADPLNWGKAAEDLSGSHLEAVKRMVEEYRRPLVRIEGSSLTVAQVAAVANGAGEARVELDESARGRVKASSDWVMNSMMNGTDSYGVTTGFGATSHRRTKEGGALQRELIRFLNAGAFGTGDDGHVLPASATRAAMLVRINTLLQGYSGIRFEILETIASLLNANVTPCLPLRGTITASGDLVPLSYIAGLVTGRPNSVAVTADGKKVGAAEAFKAAGIQHGFFELQPKEGLAMVNGTAVGSGLASMVLFEANVLAVLAEVLSAVFCEVMNGKPEYTDHLTHKLKHHPGQIEAAAIMEHILEGSSYMKLAKKLGELDPLMKPKQDRYALRTSPQWLGPQIEVIRAATKSIEREINSVNDNPLIDVSRGKALHGGNFQGTPIGVSMDNTRLAIAAIGKLMFAQFSELVNDFYNNGLPSNLSGGRNPSLDYGFKGAEIAMASYCSELQFLGNPVTNHVQSAEQHNQDVNSLGLISSRKTAEAVDILKLMSSTFLIALCQAIDLRHLEENLKGAVKSCVMTVAKKTLSTGATGALHNARFCEKDLLTAIDREAVFAYADDSCSANYPLMQKMRSVLVEHALANGEAERDPETSVFAKVATFEEEIRAALPREVEAARAAVENGTAAIPNRIAECRSYPLYRFVREELGTQYLTGEKTRSPGEEVDKVFVAMNLGKHIDAVLECLKEWNGEPLSIC

SEQ ID NO: 78 - protein, 715 aa, Setaria italica; identical to SEQ ID NO: 77

SEQ ID NO: 79 - protein, 824 aa, Zea mays
MECENGHVAASGNGGVCLAAPRADPLNWVKAAEDLAGSHLDAVKRMVEEYRRPLVKIEGASLTVAQVAAVAAAGEARVELDESARGRVKASSDWVMDSMMNGTDSYGVTTGFGATSHRRTKEGGALQRELIRFLNAGAFGTGDDGHVLPASATRAAMLVRINTLLQGYSGIRFEILEAIAALLNANVTPCLPLRGTITASGDLVPLSYIAGLVTGRPNSTAVAPDGRKVGAAEAFEIAGIQHGFFELQPKEGLAMVNGTAVGSGLASMVLFEANVLAVLAEVMSAVFCEVMNGKPEYTDHLTHKLKHHPGQIESAAIMEHILEGSSYMALAKKLGELDPLMKPKQDRYALRTSPQWLGPQIEVIRAATKSIEREINSVNDNPLIDALHGGNFQGTPIGVSMDNTRLAVAAIGKLMFAQFSELVNDFYNNGLPSNLSGGRNPSLDYGFKGAEIAMASYCSELQFLANPVTNHVQSAEQHNQDVNSLGLISSRKTAEAVDVLKLMSSTFLVALCQAIDLRHLEENLRSAVKRCVTVVARKTLSTGATGALHDARFCEKDLLTAIEREAVFAYADDPCSATYPLMQKMRSVLVEHALANGEAERDPDTSVFAKVATFEEELRAALPREVDAARAAVESGTAAIRNRIAECRSYPLYRFVREELGTEYLTGEKARSPGEEVDKVFVAMNLGKHIDAVLECLKEWNEIRLHDKLWRRPYPPAHAPQTVVGPLQALLVFLPRRCTDESSPGTVCIPPQAPGGRVIPRRCSSSSLGARRPYPPAPAPQTATGPLQALLVFLPGATRLTPPQALDDGGLQTYNCKQHGSVQH

SEQ ID NO: 80 - protein, 714 aa, Sorghum bicolor
MECENGHAAGASGNGVCLATPRAADPLNWGKAAEDLTGSHLEAVKRMVEEYRRPLVKIEGASLTVAQVAAVAAAGEARVELDESARGRVKASSDWVMNSMMNGTDSYGVTTGFGATSHRRTKEGGALQRELIRFLNAGAFGTGDDGHVLPAAATRAAMLVRINTLLQGYSGIRFEILETIAALLNANVTPCLPLRGTITASGDLVPLSYIAGLVTGRPNSTAVAPDGRKVGAAEAFKIAGIQHGFFELQPKEGLAMVNGTAVGSGLASMVLFEANVLAVLAEVMSAVFCEVMNGKPEYTDHLTHKLKHHPGQIESAAIMEHILDGSSYMMLAKKMGELDPLMKPKQDRYALRTSPQWLGPQIEVIRAATKSIEREINSVNDNPLIDVSRGKALHGGNFQGTPIGVSMDNTRLAVAAIGKLMFAQFSELVNDFYNNGLPSNLSGGRNPSLDYGFKGAEIAMASYCSELQFLANPVTNHVQSAEQHNQDVNSLGLISSRKTAEAVDVLKLMSSTFLIALCQAVDLRHLEENLKSAVKSCVTTVAKKTLSTSATGGLHNARFCEKDLLTAIDREAVFAYADDPCSANYPLMQKMRSVLVEHALANGEAERDPETSVFAKVATFEEELRAALPREVDAARAAVENGTAAVPNRIAECRSYPLYRFVREELGTEYLTGEKTRSPGEEVNKVFVAMNLGKHIDAVLECLKEWNGEPLPIC

SEQ ID NO: 81 - protein, 418 aa, Panicum virgatum
MNVTTGFGATSGFGATSHRRTKEGSALQRELIRFLNAGAFGTGDDGHVLPAAATHAAMLVRINTLLQGYSGIRFQILEAIAALLNANVTPCFPLRGTITASGDLVPLSYIAGLVTGRPNSVAVAPDGRKVGAAEAFGIAGIQHGFFELQPKEGLAMVNGTAVGSGLASIMLFEANVLAVLAEVLSAVFCEAMNGKSEYTDRLTHKLKHHPGQIEAAAIMEHILEGSSYMMLAKKLGELDPLMKPNQDRYALRTSPQWLGPQIEVIRAATKSIEREINSVNDNPLIDVSRGKALHGGNFQGTPIGVSMDNTRLAIAAIGKLMFAQFSELVNDFYNNGLPSNLSGGRNPSLDYGFKGAEIAMASYCSDLQFLANPVTNHVQSAEQHNQDVNSLGLISSRKTAEAVEILKLMSSTFLIALC

SEQ ID NO: 82 - protein, 714 aa, Oryza sativa
MECENGHVAAAANGSSLCVAKPRADPLNWGKAAEELSGSHLDAVKRMVEEYRRPVVTIEGASLTIAQVAAVASAGAARVELDESARGRVKASSDWVMNSMMNGTDSYGVTTGFGATSHRRTKEGGALQRELIRFLNAGAFGNGDDGHVLPAAATRAAMLVRINTLLQGYSGIRFEILETIATLLNANVTPCLPLRGTITASGDLVPLSYIAGLVTGRPNSVAVTPDGRKVDAAEAFKIAGIQHGFFELQPKEGLAMVNGTAVGSGLASMVLFEANVLGVLAEVLSAVFCEVMNGKPEYTDHLTHKLKHHPGQIEAAAIMEHILEGSSYMMLAKKLGELDPLMKPKQDRYALRTSPQWLGPQIEVIRAATKSIEREINSVNDNPLIDVSRGKALHGGNFQGTPIGVSMDNTRLAIAAIGKLMFAQFSELVNDFYNNGLPSNLSGGRNPSLDYGFKGAEIAMASYCSELQFLANPVTNHVQSAEQHNQDVNSLGLISSRKTAEAIDVLKLMSSTFLIALCQAIDLRHLEENVRSAVKGCVTTVARKTLSTSATGDLHKARFCEKDLLQAIDREAVFAYADDPCSANYPLMQKMRAVLIEHALANGEAERNVDTSVFAKVATFEEELRVALPREVEAARAAVENGTAAKANRITECRSYPLYRFVREELGTEYLTGEKTRSPGEEVNKVFVAMNQGKHIDALLECLKEWNGEPLPIC

SEQ ID NO: 83 - protein, 189 aa, Oryza sativa
MQKMRTVLIEHALANSEAERNVNTSVFAKVAMFEEKLRAALPREVEATRAAVENGTAAKANRITECKSYSLYRFVCKELRTEYLTGKKIDAVARSQCSRRSSARRCPGKWRLPRAAVENGTATKANRITECRSYSLYRFVRKELGIEYLTGEKIDAVTWRGCEQGVRRHEPGSGGRSEKPVFSGASMGR

SEQ ID NO: 84 - protein, 715 aa, Brachypodium distachyon
MEYENGHAATYGDGLCVAAPLAPRADPLNWGKAAEELSGSHLDAVKRMVEEYRRPVVKMEGASLTIAQVAAVAAGAEARVELDESARGRVKESSDWVMNSMMNGTDSYGVTTGFGATSHRRTKEGGALQRELIRFLNAGAFGTGEDGHVLPAAATRAAMLVRVNTLLQGYSGIRFEILETIATLLNANVTPCLPLRGTITASGDLVPLSYIAGLVTGRPNSVATAPDGRKVNAAEAFKIAGIQHGFFELQPKEGLAMVNGTAVGSGLASMVLFEANILGVLAEVLSAVFCEVMNGKPEFTDHLTHKLKHHPGQIEAAAIMEHILEGSSYMMLAKKLGELDPLMKPKQDRYALRTSPQWLGPQIEVIRAATKSIEREINSVNDNPLIDVSRGKAIHGGNFQGTPIGVSMDNTRLAIAAIGKLMFAQFSELVNDFYNNGLPSNLSGGRNPSLDYGFKGAEIAMASYCSELQFLGNPVTNHVQSAEQHNQDVNSLGLISSRKTAEAIDILKLMSSTFLVALCQAIDLRHLEENVRSAVKNCVTTVARKTLSTNVNGHLHNARFCEKDLLLTIDREAVFAYADDPCSANYPLMQKMRAVLVEHALANGEAERDVETSVFAKLAAFEQELRAVLPKEVEAARAAVENGTATKQNRIAECRSYPLYRFVREELGTEYLTGEKTRSPGEEVDKVFVAMNQGKHIDALLECLKEWNGEPLPLC

SEQ ID NO: 85 - protein, 715 aa, Streptochaeta angustifolia
MEFLSCVAANGNGLCLATPQADPLNWGKAAAELTGSHLDEVKRMVAEYRKPVVRIEGANLSIAQVVAVALAGGAAAAAKVELDESARGRVKASSDWVMNSMMNDTDSYGVTTGFGATSHRRTKQGGALQRELIRFLNAGAFGTGEDGHVLPTAATRAAMLVRINTLLQGYSGIRFEILEAVAALLNANVTPCVPLRGTITASGDLVPLSYIAGLVTGRPNSVAVAPDGRKVDAAEAFKIAGIQHGFFELQPKEGLAMVNGTAVGSGLASTVLFEANILAVLAEVLSAVFCEVMNGKPEYTDHLTHKLKHHPGQIEAAAIMEHILEGSSYMMLAKKLGELDPLMKPKQDRYALRTSPQWLGSQIEVIRAATKSIEREINSVNDNPLIDVSRGKALHGGNFQGTPIGVSMDNARLAIAAIGKLMFAQFSELVNDIYNNGLPSNLSGGRNPSLDYGFKGAEIAMASYCSELQFLANPVTNHVQSAEQHNQDVNSLGLISSRKTAEAIDILKLMSSTYLIALCQAVDLRHLEENIKSAVKKCVTAAAKKALNTSTTGDLHIARFCEKDLLTAIDREAVFAYADDPCSNNYPLMQKLREVLVEHALANGEAERNVETSVFAKIAMFEEELRAVLPREVEAARAAVENGAAAKPNRITECRSYPLYRFVREELGAKYLTGEKTRSPGEEVDKVFVAINQGKHIDPLLECLKEWNGEPLPIC

SEQ ID NO: 86 - protein, 245 aa, Streptochaeta angustifolia
MASYCSKLQFLANPVTDHVKSTEQHTQDVNSLGLISSRKTAEAIDIPKLMSSTFLIALCQAVDLRHVEENIKSAIKNCVTMVAKKALNISTTGDLHITRFCKDLLTVIEREAVFTYAGDPCSENYRLMQKLCAMHVEHILANGEAKHNGETTFLAKITMFEEVLPGEVKAIREAMENRATVMPNRTIECWLYPLYQFMREKLGVEDLTGEMTWSPDGEVDKVFVAINQGKHIDPLLECLKEWNIE

SEQ ID NO: 87 - protein, 436 aa, Streptochaeta angustifolia
LNWGKAAEELTGSHLDEVKHLVAQYRKPMVRIKGTSLSIAQVKAVASAGGDAAAAKLELDESTRGRVKASSDWAMSSMTNDTDTYGVTTAFGANSHRTTKESGALQRALIRYLHAGVFGTGTGDNVLPAAATRGAMLVRINSLLQGYSGIRFEIVEAIVALLNANVTPCLPLRGTITAKVGAAEACTIAGIQHGFFELQPKEGIALVNGTAVGCGLASAVLFEANILAVLAVVLCAVFCEVMIGKPEYTDHLTHKLKHHLGQIEAVANMEHILEGSSYKMLAKKLDELDPLMKPKKDRYALRTSPQWLGSHVEVIRATTKSIEREINSVNDNPLIDVSRSKALHGGNFQGTPIGVSTDMSLTFAAIGKLMFSQLSELVNDCTTMACLPTCPADPKLAAPLALLMPCRRSGTTARGSACVLPTMPPSAPTCRAPRGY

SEQ ID NO: 88 - protein, 906 aa, Streptochaeta angustifolia
MPCFDQRRRTARQDHLQRRPPTVKWGAAKWNSREVTENEVQRQKIEHLVCVTVHAVGDRAELEDGRCDGVMGTLAKAGRGRGGGGVVGRGGAGGRGRGGAGVVGRGGAAGRGRGGAGRCGRIGVVRGENMLGEGGAAGRLGGGELVREKKNLQHGGDVAWGEKVLGRASGNEWQVWGRPSMTWVVHRSFGSWRGERESGRVEAYCNGLRLARPYADPLNWGTAAVELTGSHLEEVKRMVAESRLPVVTIEGAGLRIAHVAAVATACGGGAAAARVELDESARGHVKASSDWVMNSMVNGVDTYGITTGFGGTSYRRTKEGGSLQRELIRFLNAGVFGTGADGHVLPAATTRAAMLVRINTLLQGYSGIRFEILEAIAALLNANVTPRLPLRGTLTASGDVIPLSYIAGLITGRQNSVAAAPDGRNMDAAEAFKIAGIQHGFFELQPKEGLALVNGTAVGSGLASMVLFEANVLALLAEVLAAVFCEVMHGKPEYTDHLTHKLKHHPGQIEAAAIMEHILEGSSYMKLAKELAELDPLTKPKQDRYALRSSPQWLGPQVEVIRAATKSIEREINSVSDNPLIDVSRDKALHCGNFQGTPIGVSMDNTRLAVAAISRLMFAQFSELVNDFYNNGLPANLSGGRDPSLDFGFKGADIAIASYCSELQFLANPVTSHVQSAERHNQDINSLGFISARMTAEAIDLLKLMSSTFLVALCQAVDLRHLEENIKSTVKNCVTTAATKTLGTTHTVRFSEDLLTAIDREAVFAYADDPCSPKYPLMKNLRAVLLERALANGEAERDVETSVLAKLASFEEELHAELPKAVEAARVAVENGGAPTPNRVKECRSYPLYRFVRDEAGAEYLTGEKARSPGEEVDKVFVAMNLGKHIDPLLDCLKEWNGEPLSICRT

SEQ ID NO: 89 - protein, 649 aa, Ananas comosus
MGGGSRPRAARGRQADGIAVAAVAGAPPAAGAAWPRAPTAYRRHHGFGATSHRRTKEGAASRGQLIRFLNAGIFGSGRESGQTLPAAATRAAMLVRINTLLQGYSGIRFEILESITALLNSHVTPCLPLRGTITASGDLVPLSYIAGILTGRPNSVAVAPDGRSVDAAEAFRLAGIPHGFFELQPKEGLALVNGTAVGSGLASIVLFEANILAVLSEVMSAVFCEVMQGKPEFTDHLTHKLKHHPGQIEAAAIMEHVLEGSSYMKMARKLHELDPLQKPKQDRYALRTSPQWLGPQIEVIRAATISIEREINSVNDNPLIDVSRNKALHGGNFQGTPIGVSMDNTRLAIAAIGKLMFAQLSELVNDFYNNGLPSNLSGGRNPSLDYGFKGAEIAMAAYCSELQFLANPVTNHVQSAEQHNQDVNSLGLISARKTAEAVEILKLMSSTFLIALCQAIDLRHLEENLKNAIKNAVSQIAKRVLTMGPNGELHPSRFCEKDLITVIDREHVFSYADDPCSATYPLMQKLRQVLVEHALGNGEKEKATETSIFQKIFAFEEELKAQLPKEVEAVRAAVESGSSAIANRIKECRSYPLYRFVREELGTEYLTGEKVRSPGEEFDKVFVAINEGKLIDPLLECLKEWNGEPLPIC

SEQ ID NO: 90 - protein, 752 aa, Ananas comosus
MECDANGVVTDPLGWAAAAAAAAESGSHLEEVKRMVEEFRAPAVTLKGVELKISQVAAVAMAKAVAPGVRVELAEAARGGVEASSEWVMESMARGTDSYGVAAVAMAKAVAPGVRVELAEAARGGVEASSEWVMESMARGTDSYGVTTGFGATSHRRTKQGGALQKELIRFLNAGIFGSGPKSEQTLPASATRAAMLVRINTLLQGYSGIRFEILEAIAAFLNSHITPCLPLRGTITASGDLVPLSYIAGLLTGRPNSIAVGPDGRSLDASEAFRLAGIMQGFFDLQPKEGLALVNGTAVGSGLASIVLYEANILAVLSEVLSAIFCEVMQGKPEFTDHLTHKLKHHPGQIEAAAIMEHILDGSSYMKAAKKLHELDPLQKPKQDRYALRTSPQWLGPQIEVIRAATKSIEREINSVNDNPLIDVSRNKALHGGNFQGTPIGVSMDNTRLAIAAIGKLMFAQFSELVNDFYNNGLPSNLSGGRNPSLDYGFKGAEIAMAAYCSELQFLANPVTNHVQSAEQHNQDVNSLGLISSRKTAEAVEILKLMSSTYLIALCQAIDLRHLEENLMHAVKNAVSQVAKRVLTMDTAGELHPARFCVKDLIKVVDCEAAFSYADDPCSPSYLLMQKLRQVLVEHALKNGEKEKNSNSSIFQKITAFEEELKANLPKEVDAARVAFETGSSAIPNRVKECRSYPLYRFIREELGTEYLTGERVRSPGEECDKVFAAINGGLLIDPLLECLKDWDGAPLPIC

SEQ ID NO: 91 - protein, 714 aa, Apostasia shenzhenica
MEHAADNGHLNGKVDDGTFCIQGRRDPLGWRAAAEAVEKSHLDEVKRMVEEFLRPSVKLEGTALTISQVAAVAAGAATTVELAESSRSGVKASNDWVLESMSRGVDSYGITTGFGATSHRRTKEVASLQKELIRFLNAGIFGSGQDNTLPPAATRAAMLVRINTLLQGYSGIRFEVLEAITALLNSNITPCLPLRGTVTASGDLVPLSYIAGILIGRPNAKAITADGDNVGAAEALRLAGISGGFFELQPKEGLAIVNGTAVGSGLASIVLFEANILAVMAEVLSALFCEVMQGKPEFTDHLTHKLKHHPGQIEAAAIMEHVLDGSSYMKMAKKIHEMDPLQKPKQDRYALRTSPQWLGPQIEVIRAATKSIEREINSVNDNPLIDVSRNRALHGGNFQGTPVGVSMDNTRLAIAAIGKLMFAQFSELVNDFYNNGLPSNLSGGRNPSLDYGFKGAEIAMAAYCSELQFLGNPVTNHVESAEQHNQDVNSLGLISSRKTAEAVEILKLMTSTFLVGLCQAVDLRHLEENLKNVVKNSVTQAAKRVLTMGVNGELHPSRFCEKDLIKMIDREYVFAYADDPCSSTYPLMQKLRQVLVEHALTNGENEKDPNSSIFQKITAFEEEIKSSLPKELEAARANVENGSPAIANRIMECRSYPLYRFVREELGSEFLTGEKVRSPGEEFDKVFNAICQGKAINPLLECLKEWNGIPLPIC

SEQ ID NO: 92 - protein, 713 aa, Apostasia shenzhenica
MEHAAENGHLNGKVDDGFCIQGRRDPLGWRAAAEAVEKSHLDEVKRMVEEFLRPSVKLEGTALTISQVAAVAAGAAATVELAESTRSGVKASNDWVLGSMSRGVDSYGVTTGFGATSHRRTKEVASLQKELIRFLNAGIFGSGQDNTLPPAATRAAMLVRINTLLQGYSGIRFEILEAITALLNSNITPCLPLRGTVTASGDLVPLSYIAGILIGRPNAKAITADGDNVGAAEALRLAGISGGFFELQPKEGLAIVNGTAVGSGLASIVLFEANILAVMAEVLSALFCEVMQGKPEFTDHLTHKLKHHPGQIEAAAIMEHVLDGSSYMKMEKKIHEMDPLLKPKHGRYALRTSPQWLGPQIEVIRAATKSIEREINSVNDNPLIDVSRNRALHGGNFQGTPVGVSMDNTRLAIAAIGKLMFAQFSELVNDFYNNGLPSNLSGGRNPSLDYGFKGAEIAMAAYCSELQFLGNPVTNHVESTEQHNQDVNSLGLISSRKTAEAVEILKLMTSTFLVGLCQAVDLRHLEENLKNVVKNSVSQAAKRVLTMGVNGELHPSRFCEKDLIKMIDREYVFAYADDPCSSTYPLMQKLRQMLVEHALTNGEKEKDPNFSIFQKITAFEEEIKSSLPKELEAARANVENGSPAIANRIMLCRSYPLYRFVREELGSEFLTGEKVRSPGEEFDKVFNAICQGKAINPLLECLKEWNGVPLPIC

SEQ ID NO: 93 - protein, 737 aa, Apostasia shenzhenica
MLQPDPGNSLSDKRRNFSSLNFFFFLLLISYPPSMEQSRKNEHLKGWPDALGWVAAAAAMERSHLSEVKRMVDEFRQPTVKLVGAELKISQVAAVASGTMSAVELADSTRAGVKASSDWVMESMNRGTDSYGVTTGFGSTSHRRTKQGAALQKELIRFLNAGIFGSRTENTLPPAAARAAMLVRINTLLQGYSGIRFEILEAITALLNSNITPCLPLRGTITASGDLVPLSYIAGILTGRPNAKALTSDGSTVDAAEAFRLAGISGGFFELQPKEGLALVNGTAVGSGLASIVLFEANILSLMAVVLSALFCEVMQGKPEFTDHLTHKLKHHPGQIEAAAIMEYVLEGSSYVKMAKKLHEMDPLLKPKQDRYALRTSPQWLGPQIEVIRSATNSIEREINSVNDNPLIDVSRNKALHGGNFQGTPVGVSMDNTRLAIAAIGKLMFAQFSELVNDFYSNGLPSNLSGGRNPSLDYGFKGAEIAMAAYCSELQFLANPVTNHVQSAEQHNQDINSLGLISARKTMEAVEILKLMSSTFLVGICQAVDLRHLEENLKNAVKNAVSQAAKKVLTTGVNGELHPSRFCEKDLIKVIDREYVFNYADDPCSSSYPLMQKLRQVLVDHALNNGDKEKDPTSSIFQKIAAFEEEVKNAVTKDVETARAAVENGSPAISNRIKDCRSYPIYRFVREELGTGFLSGNSVKSPGEEFDKVFDAICEGKAIDPLLECLKDWNGSPLPIS

SEQ ID NO: 94 - protein, 709 aa, Dendrobium catenatum
MENGHANGIAVMEDLCLKGSDPLGWVAAAKAAEGSHLDEVKRMVEDFRRPVVMLEGAELKISQVAAVASGAATSVELAESARAGVKASSDWVLESMNLGGDHYGVTTGFGATSHRRTKQGGALQKELIRFLNAGIFGSNPDNTLSPSATRAAMLVRINTLLQGYSGIRFEILEAIASLLNKNITPCLPLRGTITASGDLVPLSYIAGVLTGRPNAKAITSDGAAIDASEAFRLAGISGGFFELQPKEGLALVNGTAVGSGLASIVLFEANILAVMAEVLSALFCEVMQGKPEYTDHLTHKLKHHPGQIEAAAIMEHVLEGSSYMKMAKKLHEMDPLQKPKQDRYALRTSPQWLGPQIEVIRAATKSIEREINSVNDNPLIDVSRNKALHGGNFQGTPIGVSMDNTRLAIAAIGKLMFAQISELVNDFYNNGLPSNLSGGRNPSLDYGFKGAEIAMAAYCSELQYLANPVTNHVQSAEQHNQDVNSLGLISSRKTAEAVEILKLMTSTFLVAICQAIDLRHLEENLKSAVKNAVSLAAKKTLTMGINGELHPSRFCEKDLITVIEREYVFAYADDPCCPTYPLMKNLRQVLVEHALNNGEKEKDANSSIFQKITAFEEEIKAVIPKEVEATRVAFEKGSSTIGNRIKECRSYPLYRFVREELGASFLTGEKVTSPGEEFDKVFEAISKGKAIDPLFECLKEWNGAPLPIC

SEQ ID NO: 95 - protein, 709 aa, Phalaenopsis equestris
MENGHANGVAVMEGLCVKGRDPLGWAAAAKAAEGSHLDEVKRMVEEFRQPLVLLGGAELSISQVAAVASGAAPAVELAESARAGVKASSDWVLDNTNHGGDYYGVTTGFGATSHRRTKQAGALQKELIRFLNAGIFGSNPNNTLSPATTRAAMLVRINTLLQGYSGIRFEILEAIASLLNNKITPCLPLRGTITASGDLVPLSYIAGVLTGRPNAKAVTADGVAIDAAEAFRLAGISGVFFKLQPKEGLALVNGTAVGSGLASIVLFEANILAVMAEVLSALFCEVMQGKPEYTDHLTHKLKHHPGQIEAAAIMEHVLEGSSYMKVAKKLHEMDPLQKPKQDRYALRTSPQWLGPQIEVIRSATKSIEREINSVNDNPLIDVSRNKALHGGNFQGTPIGVSMDNTRLAIAAIGKLMFAQISELVNDFYNNGLPSNLSGGRNPSLDYGFKGAEIAMAAYCSELQYLANPVTNHVQSAEQHNQDVNSLGLISSRKTAEAVEILKLMTSTFLVAICQAIDLRHLEENLKSTVKNAVSLAAKKTLTTGVNGELHPSRFCEKDLIKVIDREYVFAYADDPCSSTYPLMKNLRQVLVEHALNNGEKEKDSTSSIFQKIAAFEEEIKATVPKEVEAARAAFEKGSSAIGNRIKECRSYPLYRFVREELGAGFLTGEKVRSPGEEFDKVFDAISEGKAIDPLLECLKEWNGAPLPIC

SEQ ID NO: 96 - protein, 713 aa, Dendrobium catenatum
MEHVKGNGYANGAKAMEGLCLKGRDQLGWAAAAKALEGSHLDEVKRMVKEFRSPVVRLEGAELKISQVAAVAAGAASTVELAESARAGVKASSDWVMESMDSGGDTYGVTTGFGATSHRRTKQGGALQKELIRFLNAGIFGSGKDNTLPPAATRAAMLVRINTLLQGYSGIRFEILEAITNLLNNKITPCLPLRGTITASGDLVPLSYIAAILTGRPNVKAITAEGATIDAAEAFRLAGISGGFFELQPKEGLALVNGTAVGSGLASTVLFEANILSVMAEVLSAVFCEVMQGKPEYTDHLTHKLKHHPGQIEAAAIMEHVLEGSSYMKVAKKLHEMDPLQKPKQDRYALRTSPQWLGPQIEVIRAATKSIEREINSVNDNPLIDVSRNIALHGGNFQGTPIGVSMDNTRLAIAAIGKLMFAQISELVNDFYNNGLPSNLSGGRNPSLDYGFKGAEIAMASYCSELQYLANPVTNHVQSAEQHNQDVNSLGLISSRKTAEAVEILKLMTSTFLVALCQAIDLRHLEENLKSAVKNTVSQVAKRVLTIGVKGELHPSRFCEKDLIKVIDREYVFAYADDPCSSTYPLMQNLRQVLVEHALNNGEKEKEADSSIFQKITAFEEELKAVLPKEVEATRLAFENGTSTIGNRIKDCRSYPIYRFVREELGTSFLTGEEVRSPGEEFDKVFNAICEGKAIDPLLECLQEWDGAPLPIC

SEQ ID NO: 97 - protein, 710 aa, Apostasia shenzhenica
MDHSKVNGHANGAMEGLCLRRPDPLNWGAAAEALEGSHLDEVKRMVEEFRRPEVKLEGADLKISQVAAVAAGDVASVELAESARAGVKASSDWVLDSVNRGTDSYGVTTGFGATSHRRTKQGGALQKELIRFLNAGIFGSGKENTLPATATRAAMLVRINTLLQGYSGIRFEILEAITSLLNSNVTPCLPLRGTVTASGDLVPLSYIAGILTGRQNAKAVAADGSVVDAAGAFRLAGISGGFFELQPKEGLALVNGTAVGSGLASIVLFESNILVLMAEVLSALFCEVMQGKPEYTDHLTHKLKHHPGQIEAAAIMEHILDGSSYMKMAKKIHEMDPLQKPKQDRYALRTSPQWLGPQIEVIRAATKSIEREINSVNDNPLIDVSRNKALHGGNFQGTPIGVSMDNTRLAIAAIGKLMFAQFSELVNDFYNNGLPSNLSGGRNPSLDYGFKGAEIAMASYCSELQFLANPVTNHVQSAEQHNQDVNSLGLISSRKTMEAVQILQLMTSTFLVGLCQAVDLRHLEENLKSSVKNAVSQAAKRVLTMGVNGELHPSRFCEKDLIKVIDREYVFAYADDPCSFSYPLMQKLRQVLVDHAIGNGEKERDPNTSIFQKILAFEEELKAVLPKEAEAARAAFDNGSPAIANRIRDCRSYPLYRFVKEVGAGFLTGEKTRSPGEAFDKVFVAISEGKVIDPLLECLKEWDGAPLPIC

SEQ ID NO: 98 - protein, 711 aa, Apostasia shenzhenica
MDHVKENGHGNGELNGLCLRAPADPLNWGSAAAAMEGSHLDEVKRMVEEFRRPAVRLEGAALKISQVAAVAAGSGVAVVLSESARAGVKASSEWVLESMNRGTDSYGVTTGFGATSHRRTKQGGALQKELIRFLNAGIFGSGEENQLPAPATRAAMLVRINTLLQGYSGIRFEILEAITSLLNSNVTPCLPLRGSITASGDLVPLSYIAGILIGRPNAKALTPEGSAVDAAEAFRLAGIPNGFFELQPKEGLALVNGTAVGSGLAALVLFEANILALMAEVLSALFCEVMQGMPEFADHLTHKLKHHPGQIEAAAIMEHILAGSSYMKAAKKIHETDPLQKPKQDRYALRTSPQWLGPQIEVIRSATKSIEREINSVNDNPLIDVSRNRALHGGNFQGTPIGVSMDNTRLAVAAIGKLMFAQFSELVNDSYNNGLPSNLSGGRNPSLDYGFKGAEVAMAAYCSELQFLANPVTNHVQSAEQHNQDVNSLGLISARKTMEAVQNLKLMTSTFLVALCQAVDLRHLEENLKNAVKNAVGQAAKRVLTMGVTGELHPARFCEKDLIKAIDREHVFAYADDPCSASYPLMQRLRQVLVEHAIANGEKEKDSSASIFQKIVDFEEEVKATVPREVEAARAAVESGSPAIENRIRECRSYPLYRFVKETGAGFLTGEKARSPGEEFDKVFGAFCEGKVIDPLLECLKEWDGAPLPIC

SEQ ID NO: 99 - protein, 701 aa, Phalaenopsis equestris
MDACKVNGLCVQGHDPLNWGAAAAELQGSHLDEVKKMVEEFRRPVVQLEGEXISQAAAVAIGGGGATVELAESARAGVKASSDWVLESVDKGTDSYGVTTGFGATSHRRTKQGGALQKELIRFLNAGIFGSGNSNTLPSSTTRAAMLVRINTLLQGYSGIRFEILEAIATLLNTNITPCLPLRGTITASGDLIPLSYIAGILTGRPNSKALTPNGSTVDAVTAFHLAGISTGFFELQPKEGLALVNGTAVGSGLASMVLFETNILAIMAEVLSALFCEVMQGKPEYTDHLTHKLKHHPGQIEAAAIMEHILEGSSYMKMAKKLHEMDPLQKPKQDRYALRTSPQWLGPQIEVIRAATKSIEREINSVNDNPLIDVSRNKALHGGNFQGTPIGVSMDNTRLAIAAIGKLMFAQFSELVNDFYNNGLPSNLTSGRNPSLDYGFKGAEIAMASYCSELQALANPVTNHVQSAEQHNQDVNSLGLISSRKTAESVDILKLMTTTFLVGLCQAVDLRHLEENLKNAVKNTVSQVAKRTLTMGVNGELHPSRFCEKDLIKVIDREYVFSYADDPCSSTYPLMPKLRAVLVEHALNNGDKEKNSSTSIFQKISSFEAELKAAMPKEVEAARAAFENGSPAIENRIKDCRSYPLYRFVKQVGAGFLTGEKIVSPGEEFDKVYNAICEGKAIDPLLECLKEWDGAPLPIC

SEQ ID NO: 100 - protein, 731 aa, Spirodela polyrhiza
MEAATQVHCNGNGNVKLEAFCLKPAAAAAAPAVAPVSGDPLNWGAAADSLRGSHLDEVKRMVEDFRRPLVRLEGADLKISQVAAVASGRGTLRVELAESARERVKASSDWVMDSMNKGTDSYGVTTGFGATSHRRTKQGGALQRELIRFLNAGIFGSGDDSANTTLPQSATRAAMLVRINTLLQGYSGIRFEIMEAITALLNHHVTPCLPLRGTITASGDLVPLSYIAGMLTGRPNARAVNAAGEKVSAQEAFQQAGISGGFFELQPKEGLALVNGTAVGSGLASVVLFEANILAVLAEVLSAVFCEVMQGKPEFTDHLTHKLKHHPGQIEAAAIMEHILEGSSYMKMAKKIHELDPLQKPKQDRYALRTSPQWLGPQVEVLRHATKSIEREINSVNDNPLVDVSRNKALHGGNFQGTPIGVSMDNARLALAAIGKLMFAQLSELVNDFYNNGLPSNLSGGRNPSLDYGFKGAEIAMASYCSELQFLGNPVTNHVQSAEQHNQDVNSLGLISARKTAEAVEILKLMTSTYLVALCQAVDLRHLEENLKAAVKNTVSQVAKRVLTMGVNGELHPSRFCEKDLLKVVDREYVFAYADDPCLSTYPLMQKLRQVLVEHAIANGEKEKDSATSIFQKVTAFEEELKMALPKEVEAAREAVEKGAPAIPNRIEDCRSYPLYKFVREELGTAMLTGEKVRSPGEEFDKVFTAISQGKVIDPLFNCLSGWNGSPVPIC

SEQ ID NO: 101 - protein, 723 aa, Spirodela polyrhiza
MEAAGQINSNGHSGVLEGFCLKPTIAAGDPLNWGAAAESLSGSHLEEVKRMVEEFRQPLVQLEGADLKISQVAAVAAAGGGASLRVELADSARPRVKASSDWVMDSMNRGTDSYGVTTGFGATSHRRTKQGAALQKELIRFLNAGIFGSGADSGNTTLPPAATRAAMLVRVNTLLQGYSGIRFEILEAITALLNNGVTPCLPLRGTITASGDLVPLSYIAGMLTGRPNAKAVTAEGKQVSAEEAFRLAGIAGGFFELQPKEGLALVNGTAVGSGLASMVLFEANILAVLAEVLSAVFCEVMQGKPEFTDHLTHKLKHHPGQIEAAAIMEHILEGSSYMKMAKKLHELDPLQKPKQDRYALRTSPQWLGPQVEVLRQATKSIEREINSVNDNPLIDVSRGKALHGGNFQGTPIGVSMDNSRLAIAAIGKLMFAQISELVNDFYNNGLPSNLTAGRNPSLDYGFKGAEIAMAAYCSELQFLANPVTNHVQSAEQHNQDVNSLGLISARKTAEAVEILKLMTSTYLVALCQAVDLRHLEENLKNAVKNTVSQVAKRVLTIGVNGELHPSRFCEKDLIRAVDREHVFTYADDPCSATYPLMQKLRQVLVEHALTNGEKEKDSNTSIFQKVAAFEEELKMTLPKEVEAVRAAFDNGTAVIPNRIKECRSYPLYKFVREELGTSLLTGEKVRSPGEEFDKVYVAISQGKVVDPLFECLRDWNGAPVPIC

SEQ ID NO: 102 - protein, 601 aa, Musa acuminata
MEVENGVGNGPCIKADPLNWGAAAEAMTRSHLDDVKRMVEEFRQPLVRLEGATLNISQVAAVAMAAHSSAVRVELSESARDRVSASSEWVMDSMTKGTDSYGVTTGFGATCHRRTREGGALQRELIRFLNAGIFGSGWESGHTLPPSAARAAMLVRINTLLQGYSGIRFEILEAMASLLNSGITPCIPLRGTITASGDLLPLSYIAALLTGRPNAKAFCPGGETVDAAEAFRRAGIPRGFFELQPKEGLALVNGTAVGSGLASVVLYEANVLAVLAEVLSAIFCEVMQGKPEFTDHLTHKLRHHPGQIEAAAIMEHILDGSSYMKMAKNLHEQDPLQKPKQDRYALRTSPQWLGPQIEVIRSATKSIEREINSVNDNPLIDVSRNKALHGGNFQGTPIGVSMDNARLALATIGKLLFAQFSELDNDLYNNGLPSNLSGGRNPSLDYGFKGAEIAMAAYCSELQFLANPVTNHVQSAEQHNQDVNSLGLISSRKTAEAVDILKLMSATYLVALCQAIDLRHLEENLKKAVTNTVSQVAKRCRSYPLYRFVREELGTAYLTGEKVRSPGEEFDKVFAAINRGQLIDPLLECLKEWNGAPLPIC

SEQ ID NO: 103 - protein, 655 aa, Musa acuminata
MEVENGVHGNGFADGLCVKADPLNWGAAAEALTGSHLDEVKRMVEEFRRPLVRLEGATLKISQVAAVAMAPHSAVRVELSESAREGVRASSQWVTDSMTNGTDSYGVTTGFGATSHRRTKEGGALQKELIRFLNAGIFGSGTESAHTLPTPAARAAMLVRVNTLLQGYSGIRFEILEAMASLLNSGITPCLPLRGTITASGDLVPLSYIAGVLTGRPNAKAFAPGGEAVDAAEAFRRSGIPHGFFELQPKEGLALVNGTAVGSGLASVVLYEANVLAVLAEVLSAVFCEVMQGKPEFTDHLTHKLKHHPGQIEAAAIMEHILDGSSYMKMAKKLHEQDPLQKPKQDRYALRTSPQWLGPQIEVIRSATKSIEREINSVNDNPLIDVSRNKALHGGNFQGTPIGVSMDNARLALAAIGKLMFAQFSELVNDFYNNGLPSNLSGGRNPSLDYGFKGAEIAMAAYCSELQFLANPVTNHVQSAEQHNQDVNSLGLISSRKTAEAVDILKLMSTTYLVALCQAIDLRHLEENLKQAVKNTVSQVAKRVLTTAANGELHPSRFCEKDLITVIDREHVFSYIDDPCSSTYALMPKLRMECRSYPLYRFVRKELGAAYLTGEKVRSPGEEFDKVFAAINKGLLIDPLMECLKEWNGAPLPIC

SEQ ID NO: 104 - protein, 652 aa, Musa acuminata
MENGGVHANGSLCIRTDPLNWGAAAEAVTGSHLEEVKRMVGEFRQPLVRLEGATLKISQVAAVATEPHSAVRVELSESAREGVRASSQWVMDSMTKGTDSYGVTTGFGATSHRRTKQGGALQQELIRFLNAGIFGSGAESGHTLPTPAARAAMLVRINTLLQGYSGIRFEILEAMASLLNSGITPCLPLRGTITASGDLVPLSYIAGLLTGRGNAKALAPGGEWVDAAEAFRRAGIPHGFFELQPKEGLAMVNGTAVGSGLASLVLYEVNVLAVLAEALSAVFCEVMQGKPEFTDHLTHKLKHHPGQIEAAAIMEHILDGSSYMKMAKKLHEQDPLQKPKQDRYALRTSPQWLGPQIEVIRASTKSIEREINSVNDNPLIDVSRNKALHGGNFQGTPIGVSMDNTRLAVAAIGKLMFAQFSELVNDFYNNGLPSNLSGGRNPSLDYGFKGAEIAMAAYCSELQFLANPVTNHVQSAEQHNQDVNSLGLISSRKTAEAVDILKLMSATYLVALCQAIDLRHLEENLKHAVKNTVGQVAKRVLTTGANGELHPSRFCEKDLIMVVDRENVFSYIDDPCSSTYALMPKLRMVLCRSYPLYRLVREELGVAYLTGEKARSPGEEFDKVFMAVNEGLLIDPLLECLKEWNGAPLPIC

SEQ ID NO: 105 - protein, 653 aa, Musa acuminata
MEVENGAYTNSTTDGLCIKADALNWATAAEALSGNHLDEVRRMVEESRRPLVRLEGATLTISQVAAVATAAAVRVELSESARDGVRASSQWVVDSMTKGTDSYGVTTGFGATSHRRTKEGGALQKELIRFLNAGIFGSGEESRHTLPASAARAAMLVRVNTLLQGYSGIRFEILEAMASLLNCGITPCLPLRGTITASGDLVPLSYIAGLLTGRPNAKALDPAGEAIDAAEAFRRAGIPHGFFELQPKEGLALVNGTAVGSGLASVVLYEANVLAVLAEVLSAVFCEVMLGKPEFTDHLTHKLKHHPGQIEAAAIMEHILDGSSFMKIAKKLHEQDPLQKPKQDRYALRTSPQWLGPQIEVIRSSTKSIEREINSVNDNPLIDVSRNKALHGGNFQGTPIGVSMDNTRLALAAIGKLMFAQFSELVNDFYNNGLPSNLSGGRTPSLDYGFKGAEIAMAAYCSELQFLANPVTNHVQSAEQHNQDVNSLGLISSRKTADAVDILKLMSATYLVALCQAIDLRHLEENLKNAVKNTVSQVAKRVLTAGVNGELQPSRFCEKDLITIVDREHVFSYIDDPCSSTYALMPKLRMECRSYPLYRFVREELGTACLTGEKVRSPGEEFDKVFVAINGGSLIDPLLECLKEWNGAPLPIC

SEQ ID NO: 106 - protein, 643 aa, Musa acuminata
MEVENGAAGGLRVETDPLNWAAAAEALAGSHLDEVRRMTWEFRQPLVKLEGSTLRISQVAAIAAGAHSAVRVELSESARDEVCASSRWVVDSTAKGTNSNGMTAGFGAASHRRTKEGGALQKELIRFLNVGIFGSGAESGHTLPPSATRAAMLVRINTLLQGYSGIRFEILEAMASLLNSGITPCLPLRGTITASGDLVPLSYIAGVLTGRPNAKACDPAGETMDAAEAFRRAGIPHEFFELQPKEGLALVNGTTVGSGLASVVLYEANVLALLAEVLSAVFCEVMQGNPEFTDHLTHRLKHHPGQIEAAAIMEHILDGSSYMKMAKKLHEQDPLQKPKKDRYALRTSPQWLGPQIEVIRSSTKSIEREINSVNDNPLIDVARNKTIYGGNFQGTPIGVSMDNARLALAAVGKLMFAQFSELVNDLYNNGLPSNLSGGRNPSLDYGFKGAEIAMAAYCSELQFLANPVTNHVQSAEQHNQDVNSLGFISSRKAAEAVDILKLMSATYLLALCQAIDLRHLEENLKNAVKNTVSQVAKRVLTTGAFCEKNLITVADREHIFGYIDDPCSSTYALMPKLRMALCRSYPLYRFVREELGTEYLTGEKVRSPGEEFDKVFVAIDRGLVIDPLLECLKEWNGVPLPMC

SEQ ID NO: 107 - protein, 651 aa, Musa acuminata
MEFERNGHHANGGLCIKDPLNWAAAAEALTGSHLDEVKKMVAESRQPLVSLEGTTLKIAHVAAVAGQSGARVELSESARGRVRASSDWVMDSMTKGTDSYGVTTGFGATSHRRTKQGGALQKELIRFLNAGIFGSGSDQSGHTLPPPATRAAMLVRINTLLQGYSGIRFEILEAITSLLNSNITPCLPLRGTITASGDLVPLSYIAGILTGRPNSKAISPAGEAIDAAEAFRLAGIPHGFFELQPKEGLALVNGTAVGSGLASMVLFDANILAVLSEVLSAIFCEVMQGKPEFTDHLTHKLKHHPGQIEAAAIMEHVLEGSSYMKMAKKLHEQDPLQKPKQDRYALRTSPQWLGPQIEVIRSSTKSIEREINSVNDNPLIDVSRNKALHGGNFQGTPIGVSMDNTRLAIAAIGKLMFAQFSELVNDFYNNGLPSNLSGGRNPSLDYGFKGAEIAMAAYCSELQFLANPVTNHVQSAEQHNQDVNSLGLISSRKTAEAVDILKLMSSTYLVALCQAIDLRHLEENLKNAVKNTVIQVAKKTLTTGVNGELHPSRFCEKDLITIIDREHVFSYVDDPCSSTYPLMQKLRQECRSYPLYRFVRAELSTGYLTGEKVRSPGEDFDKIFAAINKGMVIDPLLECLKEWNGAPLPIC

SEQ ID NO: 108 - protein, 711 aa, Elaeis guineensis
MESAHESGNANGAVKGLCIKDPLNWVAAAEALTGSHFDEVKRMVEEFRQPMVRLEGASLKISQVAAVAMAESAARVELSEASREGVQASSQWVMDSMNNGTDSYGVTTGFGATSHRRTKQGGALQKELIRFLNAGIFGSSPQSVHTLPPSATRAAMLVRINTLLQGYSGIRFEILEAMNRLLNSNITPCVPLRASISASGDLVPLSYIAGILSGRPNAVAISPTGQPIDAAEAFRLAGIPHGFFELQPKEGLALVNGTAVGSGLASLVLFEANILALLSEVLSAIFCEVMNGKPEFTDHLTHKLKHHPGQIEAAAIMEHVLEGSSYMKLAKKLHELDPLQKPKQDRYALRTSPQWLGPQIEVIRSSTKSVEREINSVNDNPLIDVSRKKALHGGNFQATPVGVSMDNTRLAIAAIGKLMFAQFSELVNDFYNNGLPSNLSGGRDPSLDYGFKGAEIAMAAYCSELQFLGNPVTNHVQSAEQHNQDVNSLGLISARKTQEAVDILKLMSSTYLVAICQAIDLRHVEENLKNTVKNTVGQVAKRVLTMGVNGELHPSRFCEKDLIKVIDREYVFSYADDPCSSTYTLMEKLRQVLVEHALNNGEKEKDATTSIFQKITAFEQELKAVLPKEVEATRVAFENGNLPIENRIKDCRSYPLYRFVRELGTGFLTGEKVRSPGEEFDKVFVAINRGMVIDPLLECLKGWDGAPLPLH

SEQ ID NO: 109 - protein, 713 aa, Calamus simplicifolius
MEFGHQNGNGNGAVDGFCIKDPLNWIAAAESLTGSHLDEVKRMVEEFRQPSVKLEGATLKISQVAAVAVAESAVRVELSEASREGVQASSQWVLDSMDKGTDSYGVTTGFGATSHRRTKQGGALQRELIRFLNAGIFGSGLESSDHTLPSSATRAAMLVRINTLLQGYSGIRFEILEAITSLLNSNITPCVPLRGSISASGDLIPLSYIAGVLSGRPNAVAISPSGQRIDATEAFRLAGIPHNFFELQPKEGLAMVNGTAVGSGLASIVLFEANILAVLSEVLSAIFCEVMNGKPEFTDHLTHKLKHHPGQIEAAAIMEHILDGSFYMKMAKKLHELDPLQKPKQDRYALRTSPQWLGPQIEVIRSSTKSIEREINSVNDNPLIDVSRNKALHGGNFQGTPIGVSMDNTRLAIAAIGKLMFAQFSELVNDYYNNGLPSNLSGGPNPSLDYGFKGAEIAMAAYCSELQFLGNPVTNHVQSAEQHNQDVNSLGLISARKTQEAVEILKLMSSTYLVALCQAIDLRHLEENLTATVKNAVIQVAKRVLTMGVNGELHPSRFCEKDLIKVIDREYVFSYADDPCSSTYTLMPKLRQVLVEHALNNGERERDASTSIFQKITAFEEELKATLPKEVEAARVAFENGSLPIENRIKECRSYPLYRFVREELGAGYLTGEKARSPGEEFNKVFVAISRGLVIDPLFECLKEWNGAPLPLC

SEQ ID NO: 110 - protein, 725 aa, Ananas comosus
MEFVEGYLCADEKKQRDPLNWGRAAEAMSGSHLEEVKRMVEEFRRPLVRLEGADLKISQVAAVAMGDFAQAQAQAQPVPVPVPVPVRVELSEGARDAVRASSDWVLASMDKGTDSYGVTTGFGATSHRRTKQGGALQRELIRFLNAGIFGSGRESGQTLPASATRAAMLVRINTLLQGYSGIRFEILEAITALLNSRVTPCLPLRGTITASGDLVPLSYIAGILTGRPNAKAVGPDGEAELSAAEAFEVAGIGGGFFELQPKEGLALVNGTAVGSGLASIVLFEANILAVLSEVLSAVFCEVMQGKPEFTDHLTHKLKHHPGQIEAAAIMEHVLEGSSYMRMAKKLHELDPLQKPKQDRYALRTSPQWLGPQIEVIRSSTRSIEREINSVNDNPLIDVARNKALHGGNFQGTPIGVSMDNTRLALAAIGKLMFAQFSELVNDFYNNGLPSNLSGGRNPSLDYGFKGAEIAMAAYCSELQFLGNPVTNHVQSAEQHNQDVNSLGLISARKTAEAVEILKLMTSTYLVALCQAIDLRHLEENLKSAVKNAVGQAAKRVLTVGAGGELHPARFCEKDLIKVVDRDHVFAYADDPCSSTYPLMQKLRQVLVEQALGNGEKEKDPNNSIFQKIAAFEEELKAQLPKEVEAARVAFENGTSAIPNRIQECRSYPLYRFVREELGAGYLTGEKVRSPGEEFNKVFNAICKGKAIDPMLECLKEWNGAPLPLC

SEQ ID NO: 111 - protein, 725 aa, Ananas comosus
MEFVEGYLCADEKKQRDPLNWGRAAEAMSGSHLEEVKRMVEEFRRPLVRLEGADLKISQVAAVAMGDFAQAQAQAQAQPVPVPVPVRVELSEGARDAVRASSDWVLASMDKGTDSYGVTTGFGATSHRRTKQGGALQRELIRFLNAGIFGSGRESGQTLPASATRAAMLVRINTLLQGYSGIRFEILEAITALLNSRVTPCLPLRGTITASGDLVPLSYIAGILTGRPNAKAVGPDGEAELSAAEAFEVAGIGGGFFELQPKEGLALVNGTAVGSGLASIVLFEANVLAVLSEVLSAVFCEVMQGKPEFTDHLTHKLKHHPGQIEAAAIMEHVLEGSSYMRMAKKLHELDPLQKPKQDRYALRTSPQWLGPQIEVIRSSTRSIEREINSVNDNPLIDVARNKALHGGNFQGTPIGVSMDNTRLALAAIGKLMFAQFSELVNDFYNNGLPSNLSGGRNPSLDYGFKGAEIAMAAYCSELQFLGNPVTNHVQSAEQHNQDVNSLGLISARKTAEAVEILKLMTSTYLVALCQAIDLRHLEENLKSAVKNAVGQAAKRVLTVGAGGELHPARFCEKDLIKVVDRDHVFAYADDPCSSTYPLMQKLRQVLVEQALGNGEKEKDPNNSIFQKIAAFEEELKAQLPKEVEAARVAFENGTSAIANRIQECRSYPLYRFVREELGAGYLTGEKVRSPGEEFNKVFNAICKGKAIDPMLECLKEWNGAPLPLC

SEQ ID NO: 112 - protein, 654 aa, Musa acuminata
MEFAPKAQVVENGEAFCLKADPLNWIKAAESLTGSHLDEVKRMVEEFRKPQVRLEGATLTISQVAAVAAARSPVRVELSEEARDGVRASSEWVMESMNKGTDSYGVTTGFGATSHRRTKQGGALQKELIRFLNAGIFGSGPESGNTLPSSAAKAAMLVRVNTLLQGYSGIRFEILEAIASLLNNGITPCLPLRGTITASGDLVPLSYIAGILTGRPNAKAVGPDGKVIGAAEAFRLAGIADGFFELQPKEGLALVNGTAVGSGLASMVLFEANVLAVLSEVLSAVFCEVMQGKPEFTDHLTHKLKHHPGQIEAAAIMEHILEGSSYMKMAQKLHEQDPLQKPKQDRYALRTSPQWLGPQIEVIRASTKSIEREINSVNDNPLIDVSRSKALHGGNFQGTPIGVSMDNTRLAVAAIGKLMFAQFSELVNDFYNNGLPSNLSGGRNPSLDYGFKGAEIAMAAYCSELQFLANPVTNHVQSAEQHNQDVNSLGLISARKTAEAVDILKLMSTTYLVALCQAVDLRHLEENLKSAVKSTVSQVAKRVLTMGANGELHPARFCEKELIKVVDREHVFTYVDDPCSATYPLMQKLRQECRSYPLYRLVREELKTGFLTGEKVTSPGEEFDKVFDAICQGKVIDPLLECLKEWDGAPIPIC

SEQ ID NO: 113 - protein, 704 aa, Elaeis guineensis
MENCNANGFCLPDPLRWAEAAEELSGSHLDEVKLMVEEYRKPLVRLEGATLRISQVAAVATSKSPVTVELSEAARDGVKASSVWVMESMSKGTDSYGVTTGFGATYHRRTKQGGALQMELIRFLNAGIFGAGPESGHALPVSATRAAMLVRINTLLQGYSGIRFEILEAMARLLNCSVTPCLPLRGTITASGDLVPLSYIAGLLTGRPNSKATTPDGTAIDASEGFQLAGIAGGFFKLQPKEGLALVNGTAVGSGLASMVLFEANILAVLSEVVSAMFCEVMQGKPEFTDHLTHKLKHHPGQMEAAAIMEHILEGSSYVKMAEKLHERDPLQKPKQDRYALRTSPQWLGPQIEVIRMSTKSIEREINSVNDNPLIDVSRSKALHGGNFQGTPVGVSMDNTRLAIAAIGKLMFAQFSELVNDSYSNGLPSNLSAGRNPSLDYGFKGAEIAMAAYCSELQFLGNPVTNHVQSAELHNQDVNSLGLISARKTAEAVEILKLMSSTYLVALCQAIDLRHLEENLKSTVKNTVAQVSKGVLTMGADGELHPSRFCEKDLIRVIDGEYVFSYVDDPCSSTYPLIQKLRQVLVEHALNNGEKEKDPSSSIFQKIAAFEEELRTVLPREVESARVALENGRSAIANRIQECRSYPLYRFVREELDTGLLTGEKVRSPGEEFDKVFDAICEAKVIEPLLECLKDWNGAPLPTC

SEQ ID NO: 114 - protein, 704 aa, Elaeis guineensis
MENGGAKSFCLPDPLHWAKAAEALTGSHLDEVKHMVEEYRKPLVRLEGATLKISQVAAVATSESPVTVELSEATRDGVKASSQWVMDSMNKGTDSYGVTTGFGATSHRRTKQGGALQKELIRFLNAGIFGGGAESGHTLPVSASRAAMLVRINTLLQGYSGIRFEILEAMTNLLNGRITPCLPLRGSISASGDLVPLSYIAGLLTGRPNSKATTPDGTTVDASEAFQLAGIPSGFFELQPKEGLAIVNGTAVGSGLASMVLFETNILAVLSEVLSAIFCEVMQGKPEFTDHLTHKLKHHPGQIEAAALMEYILEGSSYMKMAKKLHELDPLQKPKQDRYALRTSPQWLGPQIEVIRMSTKSVEREINSVNDNPLIDVSRSKALHGGNFQGTPVGVSMDNTRLAIAAIGKLMFAQFSELVNDFYNNGLPSNLSGGRNPSLDYGFKGAEIAMAAYCSELQFLGNPVTNHVQSAEQHNQDVNSLGLISARKTAEAVEILKLMSSTYLVALCQAIDLRHLEENLKNTVKNTVTQVAKRVLTIGANGELHPSRFCEKDLIKVIDREYVFAYIDDPCSFTYPLMQKLRQVLVEHALNNGEKEKDPSSSIFQKISVFEEELKTLLPKEVEGARVAFENGNSAIANRIKECRSYPLYRFVRDELDAGFLTGEKVRSPGEEFNKVFNAICEGKVIDPLLECLKDWNGAPLPIC

SEQ ID NO: 115 - protein, 709 aa, Ananas comosus
MERAPSSTKKDLSMEDPLNWAGAAEPTKVSHFDEVKRMVEEFRKPLVRLAGANLTISQVAAVAASEATVTVELSEAARAGVKASSEWVMESIDKGKDSTGITAGFGAASHRRTEQGAALQNELIRFLNVGIFGDGLESGHMLPRAATRAAMLVRINSLLQGYSGIRFEILEAIASLLNANVTPCVPLRGSITASGDLVPLAYIAGLLSGRPNSKAIGPDGMCIDAAEAFQLARIIGGFFELRPKEGIALVNGTTVGSGLASIVLFEANILAVLSEVLSAVFCEVMQGKPEFTDHLTHKLKHHPGQIEAAAIMEHILEGSSYMKKAKELHELDPLQKPKKDRYAVCTSPQWLGPQIEVIRSSTKSIEREINSVNDNPLIDVSRDKALHGGNFQGTPIGVSMDNTRLAVAAIGKLMFAQFSELVNDLYNNGLPSNLSGGRNPSLDYGFKGAEIAMAAYCSELQFLANPVTTHVQSAEQHNQDVNSLGLISARKTAEAVDVLKLMSSTYVVALCQAIDLRHLEENLKAAVKHTVSQAAKRVLMMGADGGLQPSMFCVEGLIKVIDREHVFSYIDDPCSSAYPLVQKLRQALVEHALSRGEKEKDNDSSTFFLHRITAFEEELSAHLPKDVEAAWVAFGSGKSAIQNRIKECRSYPLYKFVKEELGTECLTGEKVRSPGEEFDKVFDAICQARMIDPLLECLKDWNGAPLPIS

SEQ ID NO: 116 - protein, 701 aa, Asparagus officinalis
MENICMKTKQADPLNWGAAAEALTGSHLDEVKRMVEDFRNPVVKLEGADLKISQVAAVAMGEGRVELAEAAREGVKASSDWVMESMGKGTDSDGVTTGFGATSHRRTKQGGALQKELIRFLNAGIFGSGRESGNMLSTSATRAAMLVRINTLLQGYSGIRFEILEAITSFLNANITPCLPLRGTISSSGDLVPLSYIAGMLTGRPNSKAIGPNGSEIDAAEAFKLAGIPSGFFELQPKEGLALVNGTAVGSALASTVLFEANILAVLAEIISAVFCEVMQGKPEFTDHLTHKLKHHPGQIEAAAIMEHILEGSSYMQMAKKVHELDPLQKPKQDRYALRTSPQWLGPQIEVIRSSTKSIEREINSVNDNPLIDVSRNKALHGGNFQGTPIGVSMDNTRLAIAAIGKLMFAQVSELVNDFYNNGLPSNLSGGRNPSLDYGFKGAEIAMASYCSELQYLANPVTNHVESAEQHNQDVNSLGLISSRKTAEAVEILKLMTSTFLVALCQAIDLRHLEENLKQAVKNTVSQVSKRVLTTGVNGELHPSRFCEKELIKVIDREYVFAYIDDPCSYTYPLMQKVRQVLVEHALNNGEKERDLNTSIFQKIAVFEEELKAVLPKEVEAARVAVENGSAAIANRIKECRSYPLYRFVREELGTELLTGEKVRSPGEEFDKVFVAISQGKMIDAVLACLKDWNGAPLPIC

SEQ ID NO: 117 - protein, 567 aa, Asparagus officinalis
MLSTSATRAAMLVRINTLLQGYSGIRFEILEAITSFLNANITPCLPLRGTISSSGDLVPLSYIAGMLTGRPNSKAIGPNGSEIDAAEAFKLAGIPSGFFELQPKEGLALVNGTAVGSALASTVLFEANILAVLAEIISAVFCEVMQGKPEFTDHLTHKLKHHPGQIEAAAIMEHILEGSSYMQMAKKVHELDPLQKPKQDRYALRTSPQWLGPQIEVIRSSTKSIEREINSVNDNPLIDVSRNKALHGGNFQGTPIGVSMDNTRLAIAAIGKLMFAQVSELVNDFYNNGLPSNLSGGRNPSLDYGFKGAEIAMASYCSELQYLANPVTNHVESAEQHNQDVNSLGLISSRKTAEAVEILKLMTSTFLVALCQAIDLRHLEENLKQAVKNTVSQVSKRVLTTGVNGELHPSRFCEKELIKVIDREYVFAYIDDPCSYTYPLMQKVRQVLVEHALNNGEKERDLNTSIFQKIAVFEEELKAVLPKEVEAARVAVENGSAAIANRIKECRSYPLYRFVREELGTELLTGEKVRSPGEEFDKVFVAISQGKMIDAVLACLKDWNGAPLPIC

SEQ ID NO: 118 - protein, 701 aa, Asparagus officinalis
MENICMKTKQADPLNWGAAAEALTGSHLDEVKRMVEDFRNPVVKLEGADLKISQVAAVAMGEGRVELAEAAREGVKASSDWVMESMGKGTDSYGVTTGFGATSHRRTKQGGALQKELIRFLNAGIFGSGRESGNMLSTSATRAAMLVRINTLLQGYSGIRFEILEAITSFLNANITPCLPLRGTISSSGDLVPLSYIAGMLTGRPNSKAIGPNGSEIDAAEAFKLAGIPSGFFELQPKEGLALVNGTAVGSALASTVLFEANILAVLAEIISAVFCEVMQGKPEFTDHLTHKLKHHPGQIEAAAIMEHILEGSSYMQMAKKVHELDPLQKPKQDRYALRTSPQWLGPQIEVIRSSTKSIEREINSVNDNPLIDVSRNKALHGGNFQGTPIGVSMDNTRLAIAAIGKLMFAQVSELVNDFYNNGLPSNLSGGRNPSLDYGFKGAEIAMASYCSELQYLANPVTNHVESAEQHNQDVNSLGLISSRKTAEAVEILKLMTSTFLVALCQAIDLRHLEENLKQAVKNTVSQVSKRVLTTGVNGELHPSRFCEKELIKVIDREYVFAYIDDPCSYTYPLMQKVRQVLVEHALNNGEKERDLNTSIFQKIAVFEEELKAVLPKEVEAARVAVENGSAAIANRIKECRSYPLYRFVREELGTELLTGEKVRSPGEEFDKVFVAISQGKMIDAVLACLKDWNGAPLPIC

SEQ ID NO: 119 - protein, 613 aa, Asparagus officinalis
MGEGRVELAEAAREGVKASSDWVMESMGKGTDSYGVTTGFGATSHRRTKQGGALQKELIRFLNAGIFSSGKXNPTIEAITSFLNTNITPCLPLRGTISSSGDLVPLSYIAGMLTGRPNSKAISPDGTHIDAAEAFKLAGIPSGFFELQPKEGLALVNGTAVGSGLASTVLFEANILAVLSEIISAVFCEVMQGKPEFTDHLTHKLKHHPGQIEAAAIMEHILDGSSYMQMAKKVHELDPLQKPKQDRYALRTSPQWLGPQIEVIRSSTKSIEREINSVNDNPLIDVSRNKALHGGNFQGTPIGVSMDNTRLAIAAIGKLMFAQLSELVNDFYNNGLPSNLSGGRNPSLDYGFKGAEIAMASYCSELQYLANPVTNHVQSAEQHNQDVNSLGLISSRKTAEAVEILKLMTSTFLVALCQAIDLRHLEENLKQAVKNTVSQVSKRVLTTGVNGELHPSRFCELELIKMIDREYVFAYIDEPCSYNYPLMQKLRQVLVEHALNNGEKEKDVKTSIFQKIAAFEEELKAVLPKEVEAARVAVEAGNAQIGNRIKECRSYPLYKFVREELGTGLLTGEKVRSPGEEFDKVFVAISEGKVIDPMLECLRDWNGAPLPIC

SEQ ID NO: 120 - protein, 705 aa, Asparagus officinalis
MSHVNGNGVCVAADPLNWGAAAQSLSGSHLDEVKKMVKDYREGPVRIEGATLKVAHVAAVAAGEATARVELDESARARVKASSDWVMESMGKGTDSYGVTTGFGATSHRRTKEGGALQKELIRFLNAGIFGSGPESGNTLPAPATRAAMLVRINTLLQGYSGIRFEILEAITRLLNSNITPVLPLRGTISSSGDLVPLSYIAGMLTGRPNSKAVAPDGRTVDATEAFKLAGIDGGFFELQPKEGLALVNGTAVGSGLASTVLFDANVLLVLSEVLSAMFCEVMQGKPEYTDHLTHKLKHHPGQIEAAAIMEHILDGSSYMKMAKKLHEQDPLQKPKQDRYALRTSPQWLGPLTEVIRAATKSIEREINSVNDNPLIDVSRNKALHGGNFQGTPIGVSMDNTRLAIAAIGKLMFAQFSELVNDFYNNGLPSNLSGGRNPSLDYGFKGAEIAMASYCSELQFLANPVTNHVESAEQHNQDVNSLGLISSRKTAEAVQILKLMSSTFLVGLCQAINLRHLEENLRSTVKNTVSQVAKRILTVGVNGELHPSRFCELDLIKVVEREPVFAYIDDPCSATYPLMQKLRQVLVEHALTNGDKEKPVNTSIFHRIEEFEDELKALLPKEVEGVRMAFENGRLSIPNRIKECRSYPLYRLVREELGAEYLTGERVRSPGEEFDKVFNAICGGKLIDPLLECLSEWNGAPIPIC

SEQ ID NO: 121 - protein, 705 aa, Asparagus officinalis; identical to SEQ ID NO: 120

SEQ ID NO: 122 - protein, 707 aa, Acorus americanus
MENGFCMKKEEEVVDPLNWAKSAEPLMGSHLEEVKRMVEEFRNPLVCLEGADLKISQVAAVAAEGGGHVRVELSEAARPRVKASSDWVMESMNKGTDSYGVTTGFGATSHRRTKQGGALQKELIRFLNAGIFGSSNDSSNTLPPPATRAAMLVRTNTLLQGYSGIRFEILESITALLNTGVTPCLPLRGTITASGDLVPLSYIAGMLTGRPNAKAIGPDGRTIDATEAFRAAGIQTGFFELQPKEGLALVNGTAVGSGLASMVLYEANVLSLLAEVLSAVFCEVMQGKPEYTDHLTHKLKHHPGQIEAAAIMEHILDGSSYMKLAQKLHEQDPLQKPKQDRYALRTSPQWLGPQIEVIRAATQSIQREINSVNDNPLIDVSRNKALHGGNFQGTPIGVSMDNTRLAIAAIGKLMFAQFSELVNDFYNNGLPSNLSGGRNPSLDYGFKGAEIAMAAYCSELQFLGNPVTNHVQSAEQHNQDVNSLGLISSRKTAEAVEILKLMSATYLVGLCQAIDLRHLEEILRNTVRNTVMQVAKRVLTMGGNGELHPSRFCEKDLIKVVDRESVYAYVDEPCSATYPLMQKLRQVLVEHAINNGEKEREVGTSIFQKIGVFEEEIKAVLPKEVEAARVAYEAGNASVGNRIAECRSYPLYRFVREELKTGLLTGEKVRSPGEEFDKVFVAISEGRVIDPLLECLSDWNGAPIPIC

SEQ ID NO: 123 - protein, 720 aa, Acorus americanus
MEHSLNGFTNGSVNQNGLCLKKEAASTDPLNWAKSTEPLMGSHLEEVKKMVEEFRNPLVSLEGADLKISQVAAVAAGGGEGVRVQLSETARPRVKASSDWVMDSMSKGTDSYGVTTGFGATSHRRTKQGGALQKELIRFLNAGIFGASNDTGNTLPPPATRAAMLVRINTLLQGYSGIRFEILESITAFLNTGITPCLPLRGTITASGDLVPLSYIAGMLTGRPNSKAIGPDGQTIDATEAFRLSGIQTGFFELQPKEGLALVNGTAVGSGLASMVLYEANILSVLAEVISAVFCEVMQGKPEYTDHLTHKLKHHPGQIEAAAIMEHILDGSSYMKLAQKLHEQDPLQKPKQDRYALRTSPQWLGPQIEVIRSATQSIQREINSVNDNPLIDVSRNKALHGGNFQGTPIGVSMDNTRLAIAAIGKLMFAQFSELVNDFYNNGLPSNLSGGRNPSLDYGFKGAEIAMAAYCSELQFLGNPVTNHVQSAEQHNQDVNSLGLISSRKTAEAVEILKLMSATYLVGLCQAIDLRHLEEIMRNTVRNTVSQVAKRTLTMGVNGELHPSRFCEKDLIKVIDREYVFAYVDDPCSATYPLMQKLRQVLVEHALNNGEKEKEWSTSIFQKISLFEEELKAVLPKEVEAARAAYEAGNSSVSNRIEECRSYPLYKFVREELKTGLLTGEKVRSPGEEFDKVFVAISQGKVIDPLLECLSGWNGAPIPIC

SEQ ID NO: 124 - protein, 712 aa, Zostera marina
MDAMDLCQVKLSNGATNGITDADPLNWGAAAEELTGSHLQEVKRMVEDFNNPLVKIEGATLKISQVAAVATASAEVQLSETARERVTASSHWVMDSMNKGTDSYGVTTGFGATSHRRTKQGGALQKELIRFLNAGIFGGSKDTANTLPSATTRAAMLVRANTLLQGYSGIRFEILEAMTKFLNGNITPCLPLRGTITASGDLVPLSYIAGFLTGRTNSKAIGPDGKQITSSEAFAIAGIQEGFFQLQPKEGLALVNGTAVGSGLASMVLFDANILALSAEVLSAVFCEVMQGKPEFTDHLTHKLKHHPGQIEAAAIMEHILDGSSYMKMAAKIHEQDPLQKPKQDRYALRTSPQWLGPQVEVIRASTKSIEREINSVNDNPLIDVSRNKALHGGNFQGTPIGVSMDNTRLAIAAIGKLMFAQFSELVNDFYNNGLPSNLSGGRNPSLDYGFKGGEIAMASYCSELQFLANPVTNHVQSAEQHNQDVNSLGLISARKTAESIEILKLMTSTFLVGICQAIDLRHMEENLKASVKNTVSQVAKRVLTMTANGELHPSRFCEKDLLKVVDREYVFSYIDDPCSATYPLMQKLRSVLVDHALNNGDKEKDEAMSIFQKIAVFEEELIAALPKEVEAARLAVEAGKPAIANRIEECRSYPLYKFVREELRTGLLTGEKVRSPGEEFDKVFVAINEGELVGPLMECLKEWNGAPIPIC

SEQ ID NO: 125 - protein, 694 aa, Zostera marina
MGADPLKWEEAAEGMKGSHVAEVKQMIEDFRNPLVVLEGKNLKIAHVAAVAGDSSGSVGVVVSQENRVVVAASSDWIMESMNKGTDSYGVTTGFGATSHRRTKQGGALQAELIRFLNAGIFSSGNSDSCNMLPASTTRAAMLVRINSLLQGYSGIRFEILEALTDFLNHNITPCLPLRGSITASGDLIPLSYIVGLLIGRPNSKAIAPDGTQITAEKAFSLAGIHSGFFELQPKEGLALVNGTTVGSGLASTVLFDANILSILAEVLSAVFCEVMAGKPEFTDHLTHKLKHHPGQIEAAAIMEHILDGSSYMKNVNEHDPLQKPKQDRYALRTSPQWLGPQIEVIRASTKSIEREINSVNDNPLIDVKRGKAIHGGNFQGTPIGVSMDNTRLAIAAIGKLVFAQFSELVNHFYNNGLPSNLSGGSNPSLDYGFKGGEIAMASYCSELQFLGNPVTNHVQSAEQHNQDVNSLGLISARKTAESVEILKMMSATFLVALVQAVNLRHLEENMKRTVKNVVGQVAKRVLTLGVNSELHPSRFCEKDLIKVIDREQVFDYIDDPCNATYPLMEKLRGVLVDHALGNWEKETDVSTSIFQKIPTFEAELKLTLPKEVDAARLAHENGNSVVGNRIKDCRSYPLYRFVTEELGVGLLTGEKIRSPGEEFDKVFAAINRGELIDPLFHCLADWNGSPLPIS

SEQ ID NO: 126 - protein, 710 aa, Amborella trichopoda
MEFSAHQSHSNGKLDGLCVSDPLNWTAMAETFKGSHLEEVKKMVSEFQKPLVKLEGHSLSIAKVAAVARKEHDVSVELSHEARPRVQASSNWVMDSMTKGTDSYGVTTGFGATSHRRTRNGEALQKELIRFLNAGIFGDNSDLTLPRSTTRAAMLVRVNTLLQGFSGIRFEILEAIAALLNKGISPCLPLRGTITASGDLVPLSYIAGLLTGRPNAKAIGPNGQVMSSEEALNLAGVQSGPFELQPKEGLALVNGTAVGSGLASTVLFDANILAILAEVLSAIFCEVMQGKLEFTDHLTHKLKHHPGQIEAAALMEHILAGSYYMKEAARIHEFDPLQKPKQDRYALRTSPQWLGPQIEVIRAATKSIEREINSVNDNPIIDVSRNKALHGGNFQGTPIGVSMDNTRLALAAIGKLMFAQFSELVNDFYNNGLPSNLSGGRNPSLDYGFKGAEIAMASYTSELQFLANPVTNHVQSAEQHNQDVNSLGLISSRKTAEAIEILQLMSATFISALCQAIDLRQLEENLKAAVKLAVGQAVKKVLTMRENGELLPSRFCEKDLLNVVEREYVFAYADDPCSSNYPLMQKLRGVLVEHALASGEKEKEMGSSIFERIGLFEQELKAILPKEVETARASVEKGDSAIPKRIKECRSYPIYKFVRSVLGTELLSGERQRSPGEEFDKVFEAIRGGRIIEPLLDCVKEWDGTPLPIC

SEQ ID NO: 127 - protein, 690 aa, Amborella trichopoda
MKSVSTDPLNWGSFAEGSHLSLVKKLVDEFRCRTISLSGTDLSIAHVAAVSSGAPNSGPRVDLSSESMSNVSASCQWITESSTKGADTYGVTTGFGASSHRRTNQADSLQRELIRFLNAGIINADNPALPVSATRAAMIVHTNTLMKGYSGIRWKILRALVALLNSGVTPLVPLRGTITASGDLVPLSYIAGVLICRPNAQAIAPDGSIVGAKQALEMANTSDGPFELGPKEGVALVNGTSVGAGLASLVLFDANILAVLSEVLSAIFCEAMGGKPEFCDHLVHKLKHHPGQIEAGAIMEHILAGSSYMNRVAKFHGTSPSQKPKKDRYAMVTSPQWLGPQIEVIRAATETIQKEINSVNDNPLIDVSRNKTIQGGNFQGTAIGVAMDNTRLALAAIGKLMFAQFTELVNDFYNNGLPSNLSGGPNPSLDFGFKGAEVAMASYMSELGFLANPVTNHVQSAEQHNQDVNSLGLISARKTNEAVEILKLMSSTYLVALCQAIDLRHIEENLQGAVNRAVSRVARQVLDATIEGELSGSNLCEELLRVVEAEYAFSYIDDPCNPNYPLIQKLRGVLFGRVMAAKKEVGILVLQKIEDFELELKNHLPKEVELTREAIEKGNSMIPNRIRECRSYPLYRFVRSELEVGFLSGERTVSPGEEFNKVSIAINKGKLIDPLLECLHGWDGAPLPIC

SEQ ID NO: 128 - protein, 737 aa, Calamus simplicifolius
MASPDAPSHFIRDLRPPTLPIPIDGVNPIAVSGTSYVPHPPHWKKAAEALDCAHFNEVHRMISQFSSVSSVKIEGTTLTIADVAAVARRAAVFVELDAAAARTRVDCSVNWVAETVKRGTDTYGVTTGFGATSHYRTKKFAELQTELIRFLNAGVIGREPLSSTYAKAAMLVRINTLMQGYSGIRWELLEAITKLMNHNLIPKLPLRGTITASGDLVPLSYIAGLLTGRHNSRVITPEGDEITSTEALKRVGIDRPFDLQAKEGLALVNGTAVGSAVAATVCYDANILAILSVILSAMFCEAMQGNPEFADPLTHELKHHPGQIESAAIMEYLLDGSDYMKEALLRHEKEPLTKPKQDRYALRTSPQWLGPQIEVIRMATHSIEREINSVNDNPLIDVARDIALHGGNFQGTPVGVSMDNLRIALAAIGKLVFAHSPSSLDYGLKGAEIAMAAYCSELQYLANPVTTHVQSAEQHNQDVNSLGLISARKSAEAVEILKLMMATYMVGLCQAIDLRQLEENLKEVVKHAVMQAARKALFVAEDGSLLETRFCEKDLIQVVEKQPVFSYIDDPANPAYALMLQLREVLVEKALEGSEEGMVMFKRTPKFQEELKERLREEVGRARERFEKGDFIVGNRIRKCRTYPVYQLVREELGTELLTGKKKVSPGEHIEKVYQAIVEGKVGSVVVKCVEGWRGSAGPFTPRPDVSSPVHRRPEFWAWFEQGRSPSATSGRGYWNL

SEQ ID NO: 129 - protein, 729 aa, Calamus simplicifolius
MASPDGPVRSIRNLRPPTLPIPADGINPIAVSGTSYVPHPPHWKKAAEALDCAHFDEVHRMISQFSSVSSVKIEGTALTVADVAAVARCAAVSVELDAAAARARVDRSAAWVAEKIDSGTDTYGVTTGFGATSHRRTKKTADLQTELIRFLNAGVIGTEALPSKYAKAAMLVRTNTLMQGYSGIRWELLETIVKFMNHNLIPKLPLRGTITASGDLVPLSYIAGLLTGRHNSRVITPEGDEIASTEALKRVGIDRPFDLQAKEGLALVNGTAVGSAVAATVCYDANILVILSVILSAMFCEAMQGKPEFTDPLTHELKHHPGQIESAAIMEYLLDGSDYMKEALLRHEKEPLTKPKQDRYALRTSPQWLGPQIEVIRMATHSIEREINSVNDNPLIDVARDIALHGGNFQGTPIGVSMDNLRIALAAIGKLVFAQFSELVCDYYNDGLPSNLCAGADPSLDYGLKGAEIAMAAYCSELQYLANPVTTHVQSAEQHNQDVNSLGHRFEAVGGEPERGGEARGDASCEESPVRGRGRELLETRFCEKDLIQVVEKQPVFSYIDDPANPAYALMLQLREVLVEKALEGSEEGMVMFKRVPKFQEELKERLREEVGRARERFEKGDFIVGNRIRKCRTYPVYQLVREELGTELLTGEKKVSPGEYIEKVYQAIGEGKVGSVVVKCVEGWRGSAGPFTPRPNVSSPVHKKPEFWAWFEQGRSPSATSGRGYWNL

SEQ ID NO: 130 - protein, 757 aa, Elaeis guineensis
MASPNTPFRSIRALRPPTLPVPADGINPIAVSGTSYVPNPPHWKKAAEALDCAHFDEVHRMISQFSSLSSVKIEGTTLTVAAVAAIARRAAVSVELDAASAQSRVDRSAAWVAENIERGTDTYGVTTGFGATSHRRTKKTADLQTELIRFLNAGVIGTEPLPSSYAKAAMLVRTNTLLQGYSGIRWELLETIAKLMNHNLIPRLPLRGTITASGDLVPLSYIAGVLTGRHNSRVITPEGDEIAATEALKRVDINRPFDLQAKEGLALVNGTAVGAAVAATVCYDANILAVLSVILSAMFCEAMQGKPEFTDPLTHELKHHPGQIESAAIMEYLLDGSDYMKEAQLRHEREPLTKPKQDRYALRTSPQWLGPQIEVIRMATHSIEREINSVNDNPLIDVARGIALHGGNFQGTPIGVSMDNLRIALAAIGKLVFAQFSELVCDYYNDGLPSNLSAGADPSLDYGLKGAEIAMAAYCSELQYLANPVTTHVQSAEQHNQDVNSLGLISARKSAEAVEILKLMIATYMVALCQAIDLRQLEENLKEVVKHSVMQAARKALFVAEDGSLLETRFCEKDLIQVVEKQPVFSYIDDPGNPAYALMLQLREVLVEKALEGSEAGMAMFKRIPEFQEELRQRLREEVGKARERFGKGDFMVGNRIKKCRTCPVYRFVREELGTELLTGEKKVSPGEYIEKVYQAIGEGKVGSVVVECVEGWRGSAGPFTPRPEVSSPVHKKPEYWGWFEQGRSPSATSGRGYWNL

SEQ ID NO: 131 - protein, 698 aa, Elaeis guineensis
MAFPGKPSEPNVCIHSHPIADSASAYISDPYHWKKAAETLSSAYFDEVRRMITQFSELTSICLQGTTSLTVAQVAAITRRSSVTVTLDAATARARVDRSAAWVAEHSNTAGFGASSHRRTNKTGDLQAELIRFLNAGVIGADPFPSSYTKAAMLVRTNTLMQGYSGVRWELLEAMSKLMNQNLIPKIPLRGSICELLPLSYIAGVLIGRDNSLVTTLEGREITSMEALKRVGIDRPFELHSREGIALVNGTALGSAVAAAACYDANILAILAVILSAIFCEAMRGRPEFGDPLTHELKHHLGQIESAAIMKYLLEGSDYMKEAELRRGREPPTKPKQDRYALRTSPQWLGPQIELIRMATHSIEREINSVDDNPLVDISRGVFLRGGNFQGTPIDVSMDSLRIALAAIGKLIFAQFSELVCDYYNDGLPSNLSAGADPSLDYGFKGAEIAMAAYCSELQYLANPVTAHVQSAEQNNQDVNSLGLIAARKSVEAVEILRLMMATYMVALCQAIDLRQLEENLREVVKQAVVEAARKVLDSAEDGSLSEARFWEKDLIQVVERLPVYSYIDDPGNAKNALMQQLREALVEKALEGNEEGRVVFKRISEFQEEARQRSREEVERARERFRKGDFVIGSRINKCRTYPMYKFVREEVGTELLTGEKKVSPGEYIEKVYEAMKERRFFYYHIVYIMSVIHPLS

SEQ ID NO: 132 - protein, 661 aa, Calamus simplicifolius
MAFPRKPSESTVCDSQATFISDPDPWKKAAETLSSTYFDEASRMITQFSELTSICLQGTPLTVAQVAAITSRPSVAVTLDAATAHARVDDSAAWVAERSNTTAGFGASSHRGTNKTADLQAELIRFLNAGVVGADPLPASYTKAAMLVRTNTLMQGYSGVRWELLEAMTKLMNHNLIPKIPLRGSICELLPLSYIAGVLIGRHNSLVITPEGDEITSVEALKRAGIDRPFELHSREGIALVNGTALGSAVAAAVCFDANILALLSVVLSAMFCEAMQGRPEFAHPLTHELKHHPGQIESAAIMKYLLDGSDYMKEAELRRDREPITKPKQDRYALRTSPQWLGPHIELIRMATHSIEREINSVDDNPLIDVSRGVFLRGGNFQGTPIDVSMDSLRIALAAIGKLVFAQFSELVCDYYNNGLPSNLSAGADPSLDYGLKGRRSRWQPRKSVEAVEILRLMMATYIVALCQAIDLRQWEENLREVVKQAVVQAASKALDSAEDGRSSEARFWEKDLIQVVERLPVYSYIDDPANAKYALMQQLREVLVEKALQGGEEERVVFKSIPKFQEEVRERLKEEVGRARERFGKGDFVIGNRISKCRTYPVYKFVREEVGTELLTGEKKVSPGEYIEKVCKAMKEKRVGEVIVECIEGWSGLAANKNR

SEQ ID NO: 133 - protein, 686 aa, Ananas comosus
MASPNPKGSTRPIRRPRPPTLPVPTDGINPIAVSGTSYVPDPPHWAAAADALRCTHFDDVHRMVAQFSSVASVDLEGTALTVAQVAAVARRPAVSVALDAAAARARGYSGIRWELLEAMAKLMNHNLIPKMPLRGTITASGDLVPLSYIAGVLTGRHNVRVLAPDGGEISAAEALARAGVGPAFELQAKEGLALVNSTAVGAAVAATVCYDANVLAVLSVVLSAMFCEAMNGKPEFADPLTHELKHHPGQIESAAIMEFLLDGSDYVSEAKLRHEREPLTKPKQDRYALRTSPQWLGPQIEVIRTATHSVEREINSVNDNPLIDVARDMALHGGNFQGTPIGVSMDNLRIALAAVGKLVFAQFSELVCDYYNDGLPSNLSAGEDPSLDYGLKGAEIAMAAYCSELQYLANPVTTHVQSAEQHNQDVNSLGLISARKSAEAVDILKLMMATHMVGLCQAVDLRRLEELLLEAVKRAVARAARKALYVADDGALSEARFCEKELLQVVERQPVFSYIDDPANPNYALMLQLREVLVEKALGGSEEGMAAFKRIPQFQEELKRVLKEEVGKTREEFTSKKDETLKSRIEKCRTYPVYRFVRAEVGTEMLTGEKRASPGEHIEKVYAAMGEKGRIGEVVVECVGAWRGTPGPFTPRPEVASPANRNPENWGWFDPVRSPSATSGRGYWNL

SEQ ID NO: 134 - protein, 703 aa, Musa acuminata
MDSPGSYIRPIRCFRPPTLPVPTDGINPIAVSGTSYVPDPPHWKKAAEALSCAHFDEVHRMITQFSTLSSVDLEGTTLTVAQVAAVSRRLTVSVVLDADTARERVDRSAAWTELIRFLNAGVVGKEFLPSSYAKAAMLVRTNTLMQGYSGIRWELLEAIAKLMNCNMIPKLPLRGTITASGDLVPLSYIAGLLTGRHNSRVVTPEGDEITSVEALKRVGIGAPFELQAKEGLALVNGTAVGSAVAATVCYDANILALLSVILSAMFCEAMQGKPEFTDPLTHELKHHPGQIESAAIMEFLLDGSDYMKEAKLRHEREPLTKPKQDRYALRTSPQWLGPQIEVIRTATHSIEREINSVNDNPLIDVARDIALHGGNFQGTPIGVSMDNLRVALAAIGKLVFAQFSELVCDYYNDGLPSNLSAGADPSLDYGLKGAEIAMAAYCSELQYLANPVTTHVQSAEQHNQDINSLGLISARKSAEAVEILKLMMATYMIALCQAIDLRHLEENLREVVKHVLLQSARKSLYVAEDGRLTEARFCEKELIQVAERQPVFSYIDDPANPSYALMLQLREVLVEKALEGTEEGMAAFKRIPKFQEELGSALNEEIAKARERFGRGDFIVGSRIKKCRTYPVYNFVREEVGTELLTGEKKVVVKCVGEWRGSAGPFTPRPEASSPALRNPENWSWFEQVRSPSATSGRGYWNL

SEQ ID NO: 135 - protein, 741 aa, Zostera marina
MALAKKFSINVPATQTDEASKTSHWTKASEAIQSNHFDEVRRMISQFSNSESVNIEGASLTVAQVSAIARNSSRPVTLDASAARERVDRSANWVADNIARGTDTYGVTTGFGSTSHRRTKKTSDLQAELIRFLNAGVIGKDYLPINFAKAAMLVRTNTLMQGYSGIRWEVLEAMEKLMNKNLIPKLPLRGTITASGDLVPLSYIAGFLTGRHNSSAVTSDGEVISSQEALKRIGIDEPFELQAKEGLAICNGTAVGSAVAATVCYDANILGYLAVTLSAMFCEAMQGKSEFTDPLTHKLKHHPGQIEAAAIMEYILDGSDLLKEAKARNDREPLTKPKQDRYALRTSPQWLGPQLEVIRMATHSIEREINSVNDNPIIDVDRDIALHGGNFQGTPIGVSMDNIRIAIAAIGKLMFAQFSELVCDYYNNGLPSNLPGGKDPSLDYGLKGAEIAMASYCSEIQYLANPVTTHVQSAEQHNQDVNSLGLISARKSAEAIEILKLMSSTYMVGLCQAIDLRHLEENLREVVKHSVLQTARKTLYTTEDGSLSSTRFCEKELLQVVECQPVFSYIDDPANTSYALLLQLREVLVEKSLTSEEDKDGYFVFKKITEFTNDLKVRVAKDVEVAREKFEKGEYLVGKRIGKCRTNGVYQLVRNVAQTEILTGEKKVSPGECIEKVYDCILQGKLEDVLVKCLSMWKGMAGPFSPRPDLMSPVYKNPDTWGWFDNMRSPSSTSGRGFWSK

SEQ ID NO: 136 - protein, 744 aa, Zostera marina
MANSTKSAMIRSITVPAVSENAPAQHWKKASEAIQSNHFDEVRQMITQFSQSQSVNIEGTSLTVAQVAAITRRADVRVTLDADAARARVSRSATWVSDNIARGTDTYGVTTGFGSTSHRRTNKTSDLQAELIRFLNAGVIGKDHLPLSYSKAAMLVRTNTLMQGYSGIRWEVLQGMEKLMNKNVIPKLPLRGTITASGDLVPLSYIAGLLTGRHNSRAVTAEGEVLTSLEALKHAGIDGPFELQAKEGLALVNGTAVGSAVAATVCYDANIIGYLAVILSAMFCEAMQGKPEFTDPLTHALKHHPGQIEAAAIMEFILDGSDFMKEAKARNEREPLTKPKQDRYALRTSPQWLGPQLEVIRMATHSIEREINSVNDNPIIDVDRDIALHGGNFQGTPIGVSMDNIRIALAAIGKLMFAQFSEMVCDYYNNGLPSNLPGGKDPSLDYGLKGAEIAMASYCSELQYLANPVTTHVQSAEQHNQDVNSLGLISARKSAEAIEILKLMSSTYMVGLCQAIDLRQMEENLREVVKHSILQSIKKTLYMSEGDSDLARTRFCEKELLQVVECQPVFSYIDDPSNSSYALLLQLREVLVEESLKSSKEEVGYSAFKKITVFEEDLKERVVKDVAVAREEIEKGVYMVGNRIRKCRTKSVYDLVRKVAQTEILTGEKKISPGECIEKVYEVFEQGKVEDALLQCLDIWKGNTGPFTPRPELMSPVHKNPEAWGWFDNLRSPSSTSGRGYWSK

SEQ ID NO: 137 - protein, 758 aa, Spirodela polyrhiza
MAAQTNGLVRPTEAFRPPTLPLPKDGINPIFVSGDSYIPDPPHWKKAAEALQCTHFEEVRRMISQFQSTQTVDLQGTTLTVAQVAAVSRRQEVAVRLNEPVARDRVDRSAKWVADNIARGTDTYGVTTGFGATSHRRTKKVTDLQTELIRFLNAGVIGKDFLPTSYSKAAMLVRTNTLMQGYSGIRWEVLEAVTKLMNQNVIPKLPLRGTITASGDLVPLSYIAGLLTGRHNSRAVTPEGEELTSSEALKRVGILCPFELQAKEGLALVNGTAVGSAVAATVCFDANVLALLSVVLSALFCEAMQGKPEFADPLTHELKHHPGQIESAAIMEFLLDGSDYMREAKLRNEREPLTKPKQDRYALRTSPQWLGPQIEVIRMATHAIEREINSVNDNPLIDVARDIALHGGNFQGTPVGVSMDNLRIAIAAIGKLVFAQFSELVCDYYNNGLPSNLSGGEDPSLDYGLKGAEIAMAAYCSELQYLANPVTTHVQSAEQHNQDVNSLGLISARKSAEAVEILKLMMSTYMVGLCQALDLRHLEENLREVVKHAVTEAARKTLYTAEDGTMLESRFCEKDLLQVVECQPIFSYIDDPANPSYALLLQLREVLVEKSLNKSEDGYAVFTRIPVFQEELKARLSEEVPQARQRFEKGDFLVPHRARRCRTYPIYKLVRSVAGAEFLTGEKKISPGECIEKVVDAIDEGKLGDALSACLAMWRGSAGPFTPRPEVTSPAHCNPEFWSWFDKMRSPSATSGRGYWNN

SEQ ID NO: 138 - protein, 762 aa, Acorus americanus
MASTEAFTRPIESLRPPTLPLPTAGKNPISVSGNSYVSNPPHWQKASEALLCTHFDEVRKMVDQYRNSPSVDLTGTSLTVAQVAAITRRPEVSVALDAAAAKARVARSAQWVADNIARGTDTYGVTTGFGATSHRRTNKTADLQAELIRFLNAGVIGKEHLPASYAKAAMLVRTNTLMQGYSGIRWELLEAMEKLMNHNLIPKMPLRGTITASGDLVPLSYIAGLLTGRHNSVAVATETGELVKATEALKRIGVNTPFELQAKEGLALVNGTAVGSAVAATVCYDSNVLALLAEVLSAAFCEAMHGKPEFTDPLTHELKHHPGQIESAAIMEYLLDGSDLIKAAQARHEKDPLTKPKQDRYALRTSPQWLGPQIEVIRAATHSIAREINSVNDNPIIDCDRDMALHGGNFQGTPIGVSMDNLRIAIAAIGKLVFAQFSELVCDYYNNGLPSNLSGGPNPSLDYGLKGAEIAMASYCSELQYLANPVTTHVQSAEQHNQDVNSLGLISARKSAEAVDILKLMMSTYMVGLCQAFDLRHLEENMREVVKHVVLKTAKKTLYCEESDGTLLESRFCEKELLMVVEHQPVFSYIDDPANPSYALLLQLREVLVEKALKDLKDDTSSVVFKRIPLFQEELKKRLTEEVLKARERFDEGDFPVPNRAKKGRTSAVYEFVRGEVGTELLTGEKKTSPGEQIEKVYEAMMEGKVEGVLMRCLSFWRGSAGPFTPRPAAGSPAQHNPEYWGWFENMRSPSATSGRGYWNNM

SEQ ID NO: 139 - protein, 730 aa, Acorus americanus
MASTEAITRPIESLRPPILPLPTAGDNPISVSGNSYVSNPPHWKKASEALLCTHFDEVRKMVDQYRNSPDVSLTGTSLTVAQVAAVTRRPTVSVTLDGAAAKSRVARSAQWVADNSLRDTNGITTGFGATSHRRTNKTADLQEELIRFLNAGVIGKDNLPASYAKAAMLVRTNTLMQGYSGIRWELLEAMEKLMNHNLIPKLPLRGTITASGDLIPLAYIAGLLTGRHNSVAVATDTGELVKATEALKRIGVESPFELQAKEGLALVNGTAVGSAVAATVCYDANVLALLAEVLSAAFCEVMHGKPEFTDPLTHELKHHPGQIESAAIMEYLFDGSDLIKAGQARHENDPLTKPDRYALRTSPQWLGPQIEVIRAATHSIEREINSVNDNPIIDCDRDMALHGGNFQGTPIGVSMDNLRIAIAAIGKLVFAQFSELVCDYHNNGLPSNLSGGPNPSLDYGLKGAEIAMASYCSELQYLANPVTTHVQSAEQHNQDVNSLGLISARKSAEAVDILKLKMSTYMVGLCQAFDLRHLEENMREVVKHIVLKTAKTLYGEECEKELLMVVEHQPVFSYIEDPVNPSYALLLQLREVLVEKALKDLNDPSSVVFKRIPLFEEELKKRLSEEVAKARERFDEGDFLVPNRAKKGRTYPVYEFVRGEVGTELLTGEKKTSPGEQIEKVYEATMEGKVEEVLMRCLSFWRGSAGPFTPRPTAGSPAHHNPCTGAGLIT

SEQ ID NO: 140 - protein, 763 aa, Acorus americanus
MASSTEGLSRPIESLRPPTLPLPTGGKNPISVSGNSYVSNPPHWKKAAEALLCTHFDEVRKMVHQYRNSSSVDLTGTTLTVAQVAAIARRPKVSVALDAASAKARVARSAQWVADNIARGTDTYGITTGFGATSHRRTNKAEDLQAELIRFLNAGVIGKEYLPTSYAKAAMLVRTNTLMQGYSGIRWELLEAMESLMNHNLIPKMPLRGTITASGDLVPLSYIAGLLTGRHNSVAVATETGELVKATEALKRIGVGSPFELQAKEGLALVNGTAVGSAVAATVCYDANVLALLSEVLSAAFCEAMQGKPEFTDPLTHELKHHPGQIESAAIMEYLLDGSDHIKACAARQEKDPLTKPKQDRYALRTSPQWLGPQIEVIRAATHSIAREINSVNDNPIIDCDRDLALHGGNFQGTPIGVSMDNLRIAIAAIGKLVFAQFSELVCDYYNNGLPSNLSGGPNPSLDYGLKGAEIAMASYCSELQYLANPVTTHVQSAEQHNQDVNSLGLISARKSAEAVDILKLMLSTYMIGLCQAFDLRHLEENMREVVKHVVLQAAKKTLYTEADGTLLDTRFCEKELLQVVEHQPVFSYIDDPTNPSYALLLQLREVLVERALKNINEENGHGKVLKMIPVFQEELKRRLSEEVPKARERFDKGDFPVPNRANKCRTYPLYQFVRGEVGTELLTGEKKTSPGEYIEKVYEAMKEGKVEDVLMKCLTFWRGSAGPFTPRPAGGSPAQHNPEYWSWFDNLRSPSATSGRGYWNNM

SEQ ID NO: 141 - protein, 736 aa, Amborella trichopoda
MAENGPQAIESCRPATLPLPIKSTSTICSNGFHSTVTPPPHWKKASEALEISHFQEVSKMISQFNQAQTVTLQGTSFTVAQVTAITRRAHVSVDLDEHTARARVAESANWVRENIARGTDTYGVTTGFGATSHRRTTKTSELQTELIRFLNAGVIGKEHLPVTYARAAMAVRTNTLMQGYSGIRWEILEAMAKLISLNLIPKIPLRGTITASEGGEEIAALEALRRAGIENPFELQAKEGLALVNGTAVGAAVASSVCFDANLLALLAEVLSALFCEVMQGKPEFTDPLTHQLKHHPGQIEAAAVMEFLLDQSDYMNEAKIKHEKDPLSKPKQDRYALRTSPQWLGPQIEVIRMATHSIEREINSVNDNPLIDVARDMALHGGNFQGTPIGVSMDNLRIAIAAIGKLMFAQFSELVCDYYNNGLPSNLSGGPNPSLDYGMKGAEIAMAAYTSELQYLANPVTTHVQSAEQHNQDVNSLGLISARKSAEAIEILKLMSATFLVALCQAVDLRHLEENMREIVKHVVMQTARKTLYMEEDSGSLLDTRFYEKELLQVVDHQPVFSYLDDPANPSYALLLQLREVLVEKSLESEKEGDGFSIFKRIGVFQEELKARLREEIPKARERFDAGDFPVPNRIKGCRTYPVYKFVRSEMGTALLSGTTKVSPGEEIEKVYEGISEGKLGNVLLQCLSFWNGLAGPFTPHSVPASPAQGNPTYWAWFENTRSPSAISGRGFWNM

SEQ ID NO: 142 - protein, 755 aa, Amborella trichopoda
MAETAESCRPPSLLLPMKTTSIICSNGLHSNVTPPPHWKKASEALETSHFHEVSEMISQFNQAHTVTLQGTSLTVAQVTAITRRANISVHLDEPTARARVAESSNWVRENIARGTHINGVTSGFGATSHSRTNKTTELQAELIRFLNVGVMGKEHLSVTYARAAMAVRTNTLMQGYSGIRWEILEAMAKLTSLNLIPKIPLRGTITASGDLVPLSYIAGLLTNRPNSRVLTEGGEEIAALEALRRAGIENPFDLQAKEGLALVNGTAVGAAVASSVCFDANLLALLAEVLSALFCEVMQGKPEFTDPLTHQLKHHPGQIEAAAVMEFLLDQSDYMNEAKIKHKKDPLSKPKQDRYALRTSPQWLGPQIEVIRMATHSIEREINSVNDNPLIDVARDMALHGGNFQGTPIGVSMDNLRIAIAAIGKLMFAQFSELVCDYYNNGLPSHLSGGPNPSLDYGMKGAEIAMAAYTSELQYLANPVTTHVQSAEQHNQDVNSLGLISARKSAEAIEILKLMSATFLVALCQAVDLRHLEENMREIVKHVVMQTARKTLYMEEDSGSLLDTRFDEKELLQVVDHQPVFSYLDDPANPSYALLLQLREVLVEKSLKSEKEGDGFSIFKRIGVFQEELKARLTEEIPKARERFDAGDFPVPNRIKRCRTYPVYKFVRSEMGTALLRGTKKVSPGEEIEKVYEGISEGKLGNVLLQCLSFWNGLAGPFTPHSVPASPAQCNPTYWTWFENTRSPSATSGRGYWNM

SEQ ID NO: 143 - protein, 759 aa, Amborella trichopoda
MADSVPRSIESCRPPTLPLPNKTREPICSKGLHSTVTPPPHWEKASEAFKTSHFEEVFRMISQFNQAQTVTLQGTSLTVAQVTAITRQTGVSVNLDEHTARARVARSADWVRENIARGTDTYGVTTGFGATSHRRTSKTTDLQTELIRFLNAGVISKEHLPVAYARASMAVRTNTLMQGYSGIRWEILEALAKLISLNLIPKIPLRGTITASGDLVPLSYIAGLLTNRPNSRVLTKDGEEISALEALKRSGIETPFELQAKEGLALVNGTAVGAAVASSVCFDANLLALLAEVLSALFCEVMQGKPEFTDPLTHQLKHHPGQIEAAAVMEFLLDQSDYMNEAKIKHEKDPLTKPKQDRYALRTSPQWLGPQIEVIRMATHSIEREINSVNDNPLIDVDRDMALHGGNFQGTPIGVSMDNLRIAIAAIGKLMFAQFSELVCDYYNNGLPSNLSGGPNPSLDYGMKGAEIAMAAYTSELQYLANPVTTHVQSAEQHNQDVNSLGLISARKSAEAIEILKLMSATFLVALCQAVDLRHLEENMREIVKHVVLQTAKKTLYMEEDNGSLLDTRFYEKELLQVVDHQPVFSYLDDPSNPSYALMLQLREVLVEKSLKSNTEADGFSIFKRIGVFQEELKVRLSEEIPKARERFDAGDFPVPNRIKKCRTYPIYKFVRNEMGTALLSGTKQISPGEEIEKVYEGISEGKLGKVLLQCLSLWNGLAGPFTPRPVPASPAHCNPTYWAWFENTRSPSATSGRGYWNL

SEQ ID NO: 144 - protein, 725 aa, Arabidopsis thaliana
MEINGAHKSNGGGVDAMLCGGDIKTKNMVINAEDPLNWGAAAEQMKGSHLDEVKRMVAEFRKPVVNLGGETLTIGQVAAISTIGNSVKVELSETARAGVNASSDWVMESMNKGTDSYGVTTGFGATSHRRTKNGVALQKELIRFLNAGIFGSTKETSHTLPHSATRAAMLVRINTLLQGFSGIRFEILEAITSFLNNNITPSLPLRGTITASGDLVPLSYIAGLLTGRPNSKATGPNGEALTAEEAFKLAGISSGFFDLQPKEGLALVNGTAVGSGMASMVLFETNVLSVLAEILSAVFAEVMSGKPEFTDHLTHRLKHHPGQIEAAAIMEHILDGSSYMKLAQKLHEMDPLQKPKQDRYALRTSPQWLGPQIEVIRYATKSIEREINSVNDNPLIDVSRNKAIHGGNFQGTPIGVSMDNTRLAIAAIGKLMFAQFSELVNDFYNNGLPSNLTASRNPSLDYGFKGAEIAMASYCSELQYLANPVTSHVQSAEQHNQDVNSLGLISSRKTSEAVDILKLMSTTFLVAICQAVDLRHLEENLRQTVKNTVSQVAKKVLTTGVNGELHPSRFCEKDLLKVVDREQVYTYADDPCSATYPLIQKLRQVIVDHALINGESEKNAVTSIFHKIGAFEEELKAVLPKEVEAARAAYDNGTSAIPNRIKECRSYPLYRFVREELGTELLTGEKVTSPGEEFDKVFTAICEGKIIDPMMECLNEWNGAPIPIC

SEQ ID NO: 145 - protein, 720 aa, synthetic construct
MECENGNVAAVAAVNGGNGLCLQKPQHADPLNWGKAAGELMGSHLEEVKRMVAEFRAPVVKIEGASLRIAQVAAVAAGEAAAAKVELDESSRGRVKASSDWVMSSMMNGTDIYGVTTGFGATSHRRTKEGGALQRELIRHLNAGVFGTGSDGHVLPAAATRAAMLVRINTLLQGYSGIRFEILEAITALLNAGVTPCLPLRGTITASGDLVPLSYIAGLITGRPNSVAVAPDGRKVDAAEAFKIAGIQHGFFELQPKEGLAMVNGTAVGSGLASTVLFEANILTILAEVLSAVFCEVMTGKPEYTDHLTHKLKHHPGQIEAAAIMEHILEGSSYMKLAKKLGDLDPLMKPKQDRYALRTSPQWLGPQIEVIRASTKSIEREINSVNDNPLIDVSRGKALHGGNFQGTPIGVSMDNTRLAIAAIGKLMFAQFSELVNDFYNNGLPSNLSGGRNPSLDYGFKGAEIAMASYCSELQFLANPVTNHVQSAEQHNQDVNSLGLISSRKTAEAVDILKLMSSTFLIALCQAIDLRHLEENLKSAVKNCVAQVAKKALTLNTVGDLHNARFSEKDLLTAIDREALFAYADDPCNPNYPLMQKLRAVLVEHALANGEAEHVATTSVFAKITKFEEELRATLPKEVEAARVAVENGTAPTPNRIKECRSYPLYRFVREELGTEYLTGEKLRSPGEECNKVFVAINQGKLIDPLLECLKEWNGEPLPIC

SEQ ID NO: 146 - protein, 725 aa, synthetic construct
MEINGAHKSNGGGVDAMLCGGDIKTKNMVINAEDPLNWGAAAEQMKGSHLDEVKRMVAEFRKPVVNLGGETLTIGQVAAISTIGNSVKVELSETARAGVNASSDWVMESMNKGTDIYGVTTGFGATSHRRTKNGVALQKELIRHLNAGIFGSTKETSHTLPHSATRAAMLVRINTLLQGFSGIRFEILEAITSFLNNNITPSLPLRGTITASGDLVPLSYIAGLLTGRPNSKATGPNGEALTAEEAFKLAGISSGFFDLQPKEGLALVNGTAVGSGMASMVLFETNVLSVLAEILSAVFAEVMSGKPEFTDHLTHRLKHHPGQIEAAAIMEHILDGSSYMKLAQKLHEMDPLQKPKQDRYALRTSPQWLGPQIEVIRYATKSIEREINSVNDNPLIDVSRNKAIHGGNFQGTPIGVSMDNTRLAIAAIGKLMFAQFSELVNDFYNNGLPSNLTASRNPSLDYGFKGAEIAMASYCSELQYLANPVTSHVQSAEQHNQDVNSLGLISSRKTSEAVDILKLMSTTFLVAICQAVDLRHLEENLRQTVKNTVSQVAKKVLTTGVNGELHPSRFCEKDLLKVVDREQVYTYADDPCSATYPLIQKLRQVIVDHALINGESEKNAVTSIFHKIGAFEEELKAVLPKEVEAARAAYDNGTSAIPNRIKECRSYPLYRFVREELGTELLTGEKVTSPGEEFDKVFTAICEGKIIDPMMECLNEWNGAPIPIC

SEQ ID NO: 147 - DNA, 2163 bp, Joinvillea ascendens
  source 1..2163: mol_type = genomic DNA
atggagtgcgagaacggcaatgttgcagctgttgccgccgtcaacggcggcaatggcctgtgcttgcagaagccgcagcacgccgaccccctgaactgggggaaggcggcgggggagctgatggggagccatcttgaggaggtgaagaggatggtggcggagttccgggcgccggtggtgaagatcgagggcgccagcctgaggatcgcgcaggtggcggccgttgcggccggcgaggcggccgcggccaaggtggagctcgatgagtcctcccgcggccgcgtcaaggccagcagcgactgggtcatgagcagcatgatgaacggcaccgacagctacggtgtcaccactggctttggagccacctcccacaggaggaccaaggagggtggtgctctccaaagagagctaattagattcctcaatgccggagtcttcggcaccggctccgacggccacgtgctgccggccgcggcaacaagggcggcaatgctcgtccgcatcaacaccctcctccagggctactctggaatccgcttcgagatccttgaggcgatcactgcgctgctcaacgccggcgtcacgccgtgcctgcctctccgtggcacgatcaccgcgtcgggagacctggtcccgctgtcctacattgccggcctcattaccggccgcccgaactccgtggcagtcgcccccgacggcaggaaggtggacgccgctgaggcgttcaagatcgctggcatccagcacggcttcttcgagctgcagccaaaggaaggtctggccatggtgaacggcacggccgtgggttctggtcttgcatccacagtgctcttcgaggcgaacattcttaccattcttgcggaggtcttgtcggcagtcttctgcgaagtcatgaccggcaagccggagtacaccgaccacctgacacacaagctgaagcaccaccccggacagatagaggccgcggccatcatggagcacatcttggaaggaagctcgtacatgaagctggcgaagaagctcggcgaccttgacccgctgatgaagccgaagcaggaccggtacgcactccgcacatcgccacagtggctcggcccccagatcgaggtcatccgtgcctccaccaagtcaattgagcgtgagatcaactccgtcaacgacaacccgctcatcgatgtctcccgtggcaaggcgctccacggcggcaacttccagggcactcccatcggtgtgtccatggacaatacccgcctcgccatcgcggccattggcaagctcatgtttgcgcagttctcagagctcgttaacgacttctacaacaatggcctgccttccaacctgtccggtgggcgcaacccgagcttggactatggtttcaagggtgccgagatcgccatggcctcatactgctctgagctccagttcttggccaacccagtgaccaaccatgtccagagcgctgagcagcacaaccaggatgtcaactcgcttggccttatctcatccaggaagaccgccgaggcagtggacatactgaagctcatgtcgtcgacgttcttgatcgcattgtgccaggccattgacctgcgccaccttgaggagaacctcaagagcgcagtcaagaactgcgtggcgcaggttgccaagaaggcgctgaccttgaacacagtcggggacctccacaacgcacgcttcagcgagaaggacctactcacagcgatcgaccgtgaggcgttgttcgcgtacgcggacgacccgtgcaaccccaactacccgctgatgcagaaactgcgcgcggtactcgtcgagcatgccctcgccaacggcgaggctgagcacgtggccaccacctccgtgtttgccaagatcaccaagttcgaggaggaactgcgtgcgacactgcccaaggaggtcgaggcagcccgggtggccgtggagaacggcaccgcgcctacaccgaaccggatcaaggaatgccggtcataccctctgtaccggttcgtgcgcgaggagcttgggacagagtacttgaccggggagaaactgcggtctcctggtgaggagtgcaacaaggtgtttgtggccatcaaccagggcaagctcattgacccactgctggagtgcctcaaggagtggaacggtgagcccctgccaatctgctga

SEQ ID NO: 148 - DNA, 2163 bp, synthetic construct
  source 1..2163: mol_type = other DNA
atggagtgcgagaacggcaatgttgcagctgttgccgccgtcaacggcggcaatggcctgtgcttgcagaagccgcagcacgccgaccccctgaactgggggaaggcggcgggggagctgatggggagccatcttgaggaggtgaagaggatggtggcggagttccgggcgccggtggtgaagatcgagggcgccagcctgaggatcgcgcaggtggcggccgttgcggccggcgaggcggccgcggccaaggtggagctcgatgagtcctcccgcggccgcgtcaaggccagcagcgactgggtcatgagcagcatgatgaacggcaccgacatatacggtgtcaccactggctttggagccacctcccacaggaggaccaaggagggtggtgctctccaaagagagctaattagacacctcaatgccggagtcttcggcaccggctccgacggccacgtgctgccggccgcggcaacaagggcggcaatgctcgtccgcatcaacaccctcctccagggctactctggaatccgcttcgagatccttgaggcgatcactgcgctgctcaacgccggcgtcacgccgtgcctgcctctccgtggcacgatcaccgcgtcgggagacctggtcccgctgtcctacattgccggcctcattaccggccgcccgaactccgtggcagtcgcccccgacggcaggaaggtggacgccgctgaggcgttcaagatcgctggcatccagcacggcttcttcgagctgcagccaaaggaaggtctggccatggtgaacggcacggccgtgggttctggtcttgcatccacagtgctcttcgaggcgaacattcttaccattcttgcggaggtcttgtcggcagtcttctgcgaagtcatgaccggcaagccggagtacaccgaccacctgacacacaagctgaagcaccaccccggacagatagaggccgcggccatcatggagcacatcttggaaggaagctcgtacatgaagctggcgaagaagctcggcgaccttgacccgctgatgaagccgaagcaggaccggtacgcactccgcacatcgccacagtggctcggcccccagatcgaggtcatccgtgcctccaccaagtcaattgagcgtgagatcaactccgtcaacgacaacccgctcatcgatgtctcccgtggcaaggcgctccacggcggcaacttccagggcactcccatcggtgtgtccatggacaatacccgcctcgccatcgcggccattggcaagctcatgtttgcgcagttctcagagctcgttaacgacttctacaacaatggcctgccttccaacctgtccggtgggcgcaacccgagcttggactatggtttcaagggtgccgagatcgccatggcctcatactgctctgagctccagttcttggccaacccagtgaccaaccatgtccagagcgctgagcagcacaaccaggatgtcaactcgcttggccttatctcatccaggaagaccgccgaggcagtggacatactgaagctcatgtcgtcgacgttcttgatcgcattgtgccaggccattgacctgcgccaccttgaggagaacctcaagagcgcagtcaagaactgcgtggcgcaggttgccaagaaggcgctgaccttgaacacagtcggggacctccacaacgcacgcttcagcgagaaggacctactcacagcgatcgaccgtgaggcgttgttcgcgtacgcggacgacccgtgcaaccccaactacccgctgatgcagaaactgcgcgcggtactcgtcgagcatgccctcgccaacggcgaggctgagcacgtggccaccacctccgtgtttgccaagatcaccaagttcgaggaggaactgcgtgcgacactgcccaaggaggtcgaggcagcccgggtggccgtggagaacggcaccgcgcctacaccgaaccggatcaaggaatgccggtcataccctctgtaccggttcgtgcgcgaggagcttgggacagagtacttgaccggggagaaactgcggtctcctggtgaggagtgcaacaaggtgtttgtggccatcaaccagggcaagctcattgacccactgctggagtgcctcaaggagtggaacggtgagcccctgccaatctgctga

SEQ ID NO: 149 - DNA, 2178 bp, Arabidopsis thaliana
  source 1..2178: mol_type = genomic DNA
atggagattaacggggcacacaagagcaacggaggaggagtggacgctatgttatgcggcggagacatcaagacaaagaacatggtgatcaacgcggaggatcctctcaactggggagctgcagcggagcaaatgaaaggtagccatttggatgaagtgaagagaatggttgctgagtttaggaagccagttgtgaatcttggtggtgagactctgaccattggacaagtggctgcgatctcaactattggtaacagtgtgaaggtggagctatcggagacagctagagccggtgtgaatgctagtagtgattgggttatggagagtatgaacaaaggcactgatagttatggtgttactactggttttggtgctacttctcatcggagaaccaaaaacggtgtcgcacttcagaaggaacttattagattccttaacgccggaatattcggaagcacgaaagaaacaagccacacattgccacactccgccacaagagccgccatgcttgtacgaatcaacactctcctccaaggattttccggtatccgatttgagattctcgaagcaattaccagtttcctcaacaacaacatcactccatctctccccctccgtggtacaatcaccgcctccggagatctcgttcctctctcctacatcgccggacttctcaccggtcgtcccaattccaaagctactggtcccaacggtgaagctttaacagcagaggaagctttcaaattagcaggaatcagctccggattctttgatctccagcctaaggaaggtctcgcgctagtcaatggcacggcggttggatctggaatggcgtcaatggtgttattcgaaacgaatgttctctctgttttggctgagattttgtcggcggttttcgcagaggtgatgagtggtaagcctgagttcaccgatcatctcactcacagacttaaacatcatcccggtcaaatcgaagcggcggcgataatggagcatatcctcgacggaagctcgtacatgaaattagctcagaagcttcacgagatggatccgttacagaaacctaaacaagatcgttacgctcttcgtacttctcctcaatggttaggtcctcaaatcgaagtgatccgttacgcaacgaaatcgatcgagcgtgagattaactccgtcaacgataatccgttgatcgatgtttcgaggaacaaggcgattcacggtggtaacttccaaggaacaccaatcggagtttcaatggataacacgagattggcgatagcagcgattggtaaactcatgtttgctcaattctcagagcttgtgaatgatttctacaacaatggtttaccctcgaatctaaccgcttcgaggaatccaagtttggattatggattcaagggagctgagattgcaatggcttcttattgttcagagcttcaatacttagctaatcctgtgactagccatgttcaatcagcagagcaacataaccaagatgtcaactctttgggactaatctcgtctcgcaaaacttctgaagctgttgatattctcaagcttatgtcaacaacgttcctcgttgcgatttgtcaagctgtggatttgagacatttggaggagaatttgagacagactgtgaagaacactgtctctcaagtggcgaagaaagttcttactactggagtcaatggtgagcttcatccttctcgcttctgcgaaaaggatttactcaaagttgtagaccgtgaacaagtctacacatacgcggatgatccttgtagcgcaacgtacccgttgattcagaagctgagacaagttattgttgaccatgctttgatcaatggtgagagtgagaagaatgcagtgacttcaatcttccataagattggagctttcgaggaggagcttaaggcagtgctaccgaaagaagtggaagcagcaagagcagcctacgataacggaacatcggctatcccgaacaggatcaaggaatgtaggtcgtatccattgtatagattcgtgagggaagagcttggaacagagcttttgaccggagagaaagtgacgtcgcctggagaagagttcgacaaggttttcacggcgatttgtgaaggtaaaatcattgatccgatgatggaatgtctcaacgagtggaacggagctcccattccaatatgttaa

SEQ ID NO: 150 - DNA, 2178 bp, synthetic construct
  source 1..2178: mol_type = other DNA
atggagattaacggggcacacaagagcaacggaggaggagtggacgctatgttatgcggcggagacatcaagacaaagaacatggtgatcaacgcggaggatcctctcaactggggagctgcagcggagcaaatgaaaggtagccatttggatgaagtgaagagaatggttgctgagtttaggaagccagttgtgaatcttggtggtgagactctgaccattggacaagtggctgcgatctcaactattggtaacagtgtgaaggtggagctatcggagacagctagagccggtgtgaatgctagtagtgattgggttatggagagtatgaacaaaggcactgatatatatggtgttactactggttttggtgctacttctcatcggagaaccaaaaacggtgtcgcacttcagaaggaacttattagacaccttaacgccggaatattcggaagcacgaaagaaacaagccacacattgccacactccgccacaagagccgccatgcttgtacgaatcaacactctcctccaaggattttccggtatccgatttgagattctcgaagcaattaccagtttcctcaacaacaacatcactccatctctccccctccgtggtacaatcaccgcctccggagatctcgttcctctctcctacatcgccggacttctcaccggtcgtcccaattccaaagctactggtcccaacggtgaagctttaacagcagaggaagctttcaaattagcaggaatcagctccggattctttgatctccagcctaaggaaggtctcgcgctagtcaatggcacggcggttggatctggaatggcgtcaatggtgttattcgaaacgaatgttctctctgttttggctgagattttgtcggcggttttcgcagaggtgatgagtggtaagcctgagttcaccgatcatctcactcacagacttaaacatcatcccggtcaaatcgaagcggcggcgataatggagcatatcctcgacggaagctcgtacatgaaattagctcagaagcttcacgagatggatccgttacagaaacctaaacaagatcgttacgctcttcgtacttctcctcaatggttaggtcctcaaatcgaagtgatccgttacgcaacgaaatcgatcgagcgtgagattaactccgtcaacgataatccgttgatcgatgtttcgaggaacaaggcgattcacggtggtaacttccaaggaacaccaatcggagtttcaatggataacacgagattggcgatagcagcgattggtaaactcatgtttgctcaattctcagagcttgtgaatgatttctacaacaatggtttaccctcgaatctaaccgcttcgaggaatccaagtttggattatggattcaagggagctgagattgcaatggcttcttattgttcagagcttcaatacttagctaatcctgtgactagccatgttcaatcagcagagcaacataaccaagatgtcaactctttgggactaatctcgtctcgcaaaacttctgaagctgttgatattctcaagcttatgtcaacaacgttcctcgttgcgatttgtcaagctgtggatttgagacatttggaggagaatttgagacagactgtgaagaacactgtctctcaagtggcgaagaaagttcttactactggagtcaatggtgagcttcatccttctcgcttctgcgaaaaggatttactcaaagttgtagaccgtgaacaagtctacacatacgcggatgatccttgtagcgcaacgtacccgttgattcagaagctgagacaagttattgttgaccatgctttgatcaatggtgagagtgagaagaatgcagtgacttcaatcttccataagattggagctttcgaggaggagcttaaggcagtgctaccgaaagaagtggaagcagcaagagcagcctacgataacggaacatcggctatcccgaacaggatcaaggaatgtaggtcgtatccattgtatagattcgtgagggaagagcttggaacagagcttttgaccggagagaaagtgacgtcgcctggagaagagttcgacaaggttttcacggcgatttgtgaaggtaaaatcattgatccgatgatggaatgtctcaacgagtggaacggagctcccattccaatatgttaa

SEQ ID NO: 151 - DNA, 2118 bp, Joinvillea ascendens
  source 1..2118: mol_type = genomic DNA
atggcgttccagaacgacaatgtcctttgcataaagaaggaccccctaaactgggggaaagcggcggaggagctgacggggagccacctcgacgaggtgaagaggatggtggcggagtcccggacgccagtggttaagatccagggcgccagcctgaggatcgggcaggtggcggcggtggcggtccgcgagacgggcgtcgccaaggtggagcttgacgagtcagcccgcggccgcgtcaaggccagcagcgactggatcgtgaacagcgtggcgaacggtggagatatatatggtgtcaccactggcttcggaggcacctctcacaggaggaccagggacggtcctgcgctccaaatagagctccttagacacctcaatgccggaatcttcggcaccggcaccgacggccacgtcctgccgtcggcggtgacacgtgctgccatgttcgtccgtatcaacactttgctccagggctactccggcatccgctttgagatcctcgaggcgatcgctgcactgctgaatgctggcgtcacgccgtgcctgccactccgcggcaccatcaccgcgtccggagacctcatccccttgtcctacattgcgggcctcattaccggccgaccgaacgcagtggcgatcgcccccgatggcaggaaggtggacgccgccgaggcattcaagatcgccggcatcccgcacggcttcttcaagctgcagcccaaggaaggtcttgccatagtgaacggcacatccgtaggggctgccctggcatccacggtgctcttcgacgcgaatattcttgccatccttgccgaggtcttgtctgctgtgttctgcgaggtcatgaccggcaagccggagtacaccgaccacctgacacacaggctgaagcaccacccaggacagatagaggccgcggccatcatggagcacatcttggaaggcagctcgttcatgaagctggcgaagaagctcggggagctcgaccctctgctgaagccgaagcaggaccggtacgcgctacgcacggcgccgcagtggctcggcccccagatcgaggtcatacgcgccgccaccaagtcgatcgagcgcgagatcaactctgtcaatgacaacccaatcatcgacgtctcccgcggcaaggcgctccatggcggcaacttccaaggcacccccatcggcgtgtccatggacaacacccgcctcgccatcgccaacatcggcaagctcatgtttgcgcagttctccgagctcgtgaacgacttctacaacaatggcctaacatcgaacttggccggcgggcgcaacccgagcttggactacggtttcaagggtaccgagatcgccatggcctcgtactgctctgagctccagttcttggccaaccccgtgactaaccatgtccagagtgccgagcagcacaaccaggatgtcaactcgcttggccttgtctctgccaggaagactgccgaggcagtggacatactgaagctcatgtcgtcgacgttcttgattgcactgtgccaggccatcgacctgcgccacctcgaggagaacctcaagagtgcagtcaagaactgcgtggcgcaggtggccaagaaggtgctgaccgtgaacacagtcggggacctgcacaatgcgcgcttcagcgagaaggacctgctcacggcgatcgaccgcgaggcagtgttcacttacgcagatgacccctgcagccccaactatccgctgatgcagaaggtgcgcgctgtgctcgttgagcacgccctcgccaatggcgaggctgagcgcgtggccaacacgtccgtgtttgccaagatcaccaagttcgaggaggagttgcgagcgacactgcccaaggaagtcgaggcggcccgggtggccgtggagaatcgcactgcgccaacccagaaccggatcaaggaatcacggtcatacccactgtaccgtttcgtgcgtgaggacctcgggacggcgtacttgaccggagagaagctgcggtctcctggcgaggagtgcaacaaggtgtttgtggccatcaaccagggcaagcttattgacccactgctggagtgcctcaagaagtggaacggtgagcccctgccaatctgctga

SEQ ID NO: 152 - protein, 525 aa, Arabidopsis thaliana
MALSNASSLSTRSIYGGDLSHRPSNRQSSFTFHPAVNTKPKSVNLVTAVHAAEPARNAVSVKESVASSSSGALKWTPESWKLKKALQLPDYPNANELESVLKTIEAFPPIVFAGEARNLEERLADAAVGKAFLLQGGDCAESFKEFNATNIRDTFRVLLQMSIVLTFGGQVPVIKVGRMAGQFAKPRSDAFEEKDGVKLPSYKGDNINGDTFDEKSRIPDPNRMIRAYTQSAATLNLLRAFATGGYAAIQRVTQWNLDFVEQSEQADRYQELANRVDEALGFMSACGLGTDHPLMTTTDFYTSHECLLLPYEQSLTRLDSTSGLYYDCSAHMVWCGERTRQLDGAHVEFLRGIANPLGIKVSNKMDPFELVKLVEILNPNNKPGRITVIVRMGAENMRVKLPHLIRAVRRSGQIVTWVCDPMHGNTIKAPCGLKTRAFDSILAEVRAFLDVHEQEGSHAGGIHLEMTGQNVTECIGGSRTVTYDDLSSRYHTHCDPRLNASQSLELAFIVAERLRKRRTGSQRVS

SEQ ID NO: 153 - protein, 266 aa, synthetic construct
MSNSPSLKIGIVGFGSFGQFLAKTMIKQGHTLTATSRTDYSHTCLQLGIQFFRDVGTFIEANNDVILICTSIMSFTKVLSSMPLACLKKPTTLFVDVLSVKEHPREVLLRVLPEESDILCTHPMFGPESGKNGWKDLNFMYDKVRIHDEATCSNFLHIFASEGCKMLQMSCEEHDKIAAKSQFITHTIGRTLAEMDIESTPIDTKGFQTLTQLKNTTMRCSFDLYSGLFVHNRFAKQELENLQRALDRVKEMLVQRMREELGPEKD

SEQ ID NO: 154 - protein, 268 aa, synthetic construct
MSSSSKSLKIGIVGFGTFGQFLANTMIKQGHTLTATSRTDYSQLCDQMGIHFFRDITAFLDADMDVILLCTSISSLSEVVGSMPLACLKRPTLFVDVLSVKEHPKNLLLKVLPEESDILCTHPMFGPVSGKNGWQNLTFMFDKVRIKDEVTCSKFLQIFASEGCKMVEMSCEEHDKAAAKSQFITHTIGRTLAEMDIKSTPIDTKGFQTLVELKKPVMGDSFDLYSGLFVYNRFARQELENLEHALHKVKEMLVQRIDEGQNPERTES

SEQ ID NO: 155 - DNA, 35 bp, synthetic construct
  source 1..35: mol_type = other DNA
cgcgcggcagccatatgatggcgttccagaacgac

SEQ ID NO: 156 - DNA, 34 bp, synthetic construct
  source 1..34: mol_type = other DNA
gctcgaattcggatcctcagcagattggcagggg

SEQ ID NO: 157 - DNA, 20 bp, synthetic construct
  source 1..20: mol_type = other DNA
caattgcagggagatcgagc

SEQ ID NO: 158 - DNA, 20 bp, synthetic construct
  source 1..20: mol_type = other DNA
tgctgttgtaaggtggggat

SEQ ID NO: 159 - DNA, 35 bp, synthetic construct
  source 1..35: mol_type = other DNA
cgcgcggcagccatatgatggagtgcgagaacggc

SEQ ID NO: 160 - DNA, 34 bp, synthetic construct; identical to SEQ ID NO: 156
  source 1..34: mol_type = other DNA

SEQ ID NO: 161 - DNA, 19 bp, synthetic construct
  source 1..19: mol_type = other DNA
tcttcttccacaccaaacg

SEQ ID NO: 162 - DNA, 22 bp, synthetic construct
  source 1..22: mol_type = other DNA
gcacaagaaggatgctagaaac

SEQ ID NO: 163 - DNA, 35 bp, synthetic construct
  source 1..35: mol_type = other DNA
cgcgcggcagccatatgatggcgagccagagggac

SEQ ID NO: 164 - DNA, 34 bp, synthetic construct
  source 1..34: mol_type = other DNA
gctcgaattcggatccttagcagatgggcagggg

SEQ ID NO: 165 - DNA, 18 bp, synthetic construct
  source 1..18: mol_type = other DNA
atggtggcccagagcgac

SEQ ID NO: 166 - DNA, 19 bp, synthetic construct
  source 1..19: mol_type = other DNA
ttagcagattggaaggggc

SEQ ID NO: 167 - DNA, 35 bp, synthetic construct
  source 1..35: mol_type = other DNA
cgcgcggcagccatatgatggtggcccagagcgac

SEQ ID NO: 168 - DNA, 35 bp, synthetic construct
  source 1..35: mol_type = other DNA
gctcgaattcggatccttagcagattggaaggggc

SEQ ID NO: 169 - DNA, 20 bp, synthetic construct
  source 1..20: mol_type = other DNA
caagaagagcacgccaactc

SEQ ID NO: 170 - DNA, 20 bp, synthetic construct
  source 1..20: mol_type = other DNA
gccacacacacatacggatc

SEQ ID NO: 171 - DNA, 34 bp, synthetic construct
  source 1..34: mol_type = other DNA
gcgcggcagccatatgatggcgggcaacggcgcc

SEQ ID NO: 172 - DNA, 34 bp, synthetic construct
  source 1..34: mol_type = other DNA
gctcgaattcggatccttagttgacgacgttgat

SEQ ID NO: 173 - DNA, 20 bp, synthetic construct
  source 1..20: mol_type = other DNA
ccactgtcagtcacgcaatt

SEQ ID NO: 174 - DNA, 20 bp, synthetic construct
  source 1..20: mol_type = other DNA
tgcaacagccaagaacatgc

SEQ ID NO: 175 - DNA, 34 bp, synthetic construct
  source 1..34: mol_type = other DNA
gcgcggcagccatatgatggagtgcgagacgggt

SEQ ID NO: 176 - DNA, 34 bp, synthetic construct
  source 1..34: mol_type = other DNA
gctcgaattcggatcctcagcagagcggcagtgg

SEQ ID NO: 177 - DNA, 20 bp, synthetic construct
  source 1..20: mol_type = other DNA
ctctgcaattcgacgagctc

SEQ ID NO: 178 - DNA, 20 bp, synthetic construct
  source 1..20: mol_type = other DNA
agttctactggctgcctacc

SEQ ID NO: 179 - DNA, 34 bp, synthetic construct
  source 1..34: mol_type = other DNA
gcgcggcagccatatgatggagtacgagaacggg

SEQ ID NO: 180 - DNA, 34 bp, synthetic construct
  source 1..34: mol_type = other DNA
gctcgaattcggatcctcagcagagaggcagggg

SEQ ID NO: 181 - DNA, 22 bp, synthetic construct
  source 1..22: mol_type = other DNA
agctcctatcttctttctttct

SEQ ID NO: 182 - DNA, 20 bp, synthetic construct
  source 1..20: mol_type = other DNA
aaccacttcacagacaatca

SEQ ID NO: 183 - DNA, 38 bp, synthetic construct
  source 1..38: mol_type = other DNA
cgcgcggcagccatatgatggagattaacggggcacac

SEQ ID NO: 184 - DNA, 41 bp, synthetic construct
  source 1..41: mol_type = other DNA
gctcgaattcggatccttaacatattggaatgggagctccg

SEQ ID NO: 185 - DNA, 23 bp, synthetic construct
  source 1..23: mol_type = other DNA
cgactcactataggggaattgtg

SEQ ID NO: 186 - DNA, 21 bp, synthetic construct
  source 1..21: mol_type = other DNA
gctagttattgctcagcggtg

SEQ ID NO: 187 - DNA, 20 bp, synthetic construct
  source 1..20: mol_type = other DNA
cattcaagatcgccggcatc

SEQ ID NO: 188 - DNA, 20 bp, synthetic construct
  source 1..20: mol_type = other DNA
ctaacatcgaacttggccgg

SEQ ID NO: 189 - DNA, 20 bp, synthetic construct
  source 1..20: mol_type = other DNA
tcttcctggcagagacaagg

SEQ ID NO: 190 - DNA, 20 bp, synthetic construct
  source 1..20: mol_type = other DNA
ttcctcaatgccggagtctt

SEQ ID NO: 191 - DNA, 20 bp, synthetic construct
  source 1..20: mol_type = other DNA
cttctgcgaagtcatgaccg

SEQ ID NO: 192 - DNA, 20 bp, synthetic construct
  source 1..20: mol_type = other DNA
caacccagtgaccaaccatg

SEQ ID NO: 193 - DNA, 20 bp, synthetic construct
  source 1..20: mol_type = other DNA
ctacgacgccaacattctcg

SEQ ID NO: 194 - DNA, 20 bp, synthetic construct
  source 1..20: mol_type = other DNA
acatcggcaagctcatgttc

SEQ ID NO: 195 - DNA, 20 bp, synthetic construct
  source 1..20: mol_type = other DNA
ttgatggcaggaaggtggat

SEQ ID NO: 196 - DNA, 20 bp, synthetic construct
  source 1..20: mol_type = other DNA
atcggaaagctcatgttcgc

SEQ ID NO: 197 - DNA, 18 bp, synthetic construct
  source 1..18: mol_type = other DNA
ccccaaggaaggtctggc

SEQ ID NO: 198 - DNA, 20 bp, synthetic construct
  source 1..20: mol_type = other DNA
acatcggcaagctcatgttc

SEQ ID NO: 199 - DNA, 19 bp, synthetic construct
  source 1..19: mol_type = other DNA
catcgtcaatggcacctcc

SEQ ID NO: 200 - DNA, 20 bp, synthetic construct
  source 1..20: mol_type = other DNA
ctcatgttcgcgcagttctc

SEQ ID NO: 201 - DNA, 18 bp, synthetic construct
  source 1..18: mol_type = other DNA
gtctcgccatggtcaacg

SEQ ID NO: 202 - DNA, 20 bp, synthetic construct
  source 1..20: mol_type = other DNA
ccatcggcaagctcatgttc

SEQ ID NO: 203 - DNA, 18 bp, synthetic construct
  source 1..18: mol_type = other DNA
ccttgccatggtgaacgg

SEQ ID NO: 204 - DNA, 20 bp, synthetic construct
  source 1..20: mol_type = other DNA
caagctcatgtttgcccagt

SEQ ID NO: 205 - DNA, 27 bp, synthetic construct
  source 1..27: mol_type = other DNA
ctcaggtttctgaacgccgggatcttc

SEQ ID NO: 206 - DNA, 27 bp, synthetic construct
  source 1..27: mol_type = other DNA
gttcagaaacctgaggagctcgacctg

SEQ ID NO: 207 - DNA, 26 bp, synthetic construct
  source 1..26: mol_type = other DNA
cttagattcctcaatgccggaatctt

SEQ ID NO: 208 - DNA, 27 bp, synthetic construct
  source 1..27: mol_type = other DNA
attgaggaatctaaggagctctatttg

SEQ ID NO: 209 - DNA, 27 bp, synthetic construct
  source 1..27: mol_type = other DNA
aattagacacctcaatgccggagtctt

SEQ ID NO: 210 - DNA, 27 bp, synthetic construct
  source 1..27: mol_type = other DNA
ttgaggtgtctaattagctctctttgg

SEQ ID NO: 211 - DNA, 26 bp, synthetic construct
  source 1..26: mol_type = other DNA
ctccggtttctgaatgctggaatctt

SEQ ID NO: 212 - DNA, 27 bp, synthetic construct
  source 1..27: mol_type = other DNA
attcagaaaccggaggagctccacctg

SEQ ID NO: 213 - DNA, 26 bp, synthetic construct
  source 1..26: mol_type = other DNA
cttcggtttctcaatgccggaatctt

SEQ ID NO: 214 - DNA, 27 bp, synthetic construct
  source 1..27: mol_type = other DNA
attgagaaaccgaaggagctccacctg

SEQ ID NO: 215 - DNA, 33 bp, synthetic construct
  source 1..33: mol_type = other DNA
ctcaggtttctcaacgccgggatcttcggcacc

SEQ ID NO: 216 - DNA, 33 bp, synthetic construct
  source 1..33: mol_type = other DNA
gttgagaaacctgagcagctcgacctggagcgc

SEQ ID NO: 217 - DNA, 33 bp, synthetic construct
  source 1..33: mol_type = other DNA
atcagacacctcaatgccggcgccttcggcacc

SEQ ID NO: 218 - DNA, 34 bp, synthetic construct
  source 1..34: mol_type = other DNA
attgaggtgtctgatgagctccctctggagcgcg

SEQ ID NO: 219 - DNA, 33 bp, synthetic construct
  source 1..33: mol_type = other DNA
atccgacaccttaatgcgggagccttcggcacc

SEQ ID NO: 220 - DNA, 35 bp, synthetic construct
  source 1..35: mol_type = other DNA
attaaggtgtcggatgagctctctctgcagagcgc

SEQ ID NO: 221 - DNA, 33 bp, synthetic construct
  source 1..33: mol_type = other DNA
cttagattcctcaatgccggagtcttcggcacc

SEQ ID NO: 222 - DNA, 33 bp, synthetic construct
  source 1..33: mol_type = other DNA
attgaggaatctaagtagctctctttggagagc

SEQ ID NO: 223 - DNA, 33 bp, synthetic construct
  source 1..33: mol_type = other DNA
attgaggaatctaagtagctctacttggagagc

SEQ ID NO: 224 - DNA, 32 bp, synthetic construct
  source 1..32: mol_type = other DNA
gcgactgggtcatgagcagcatgatgaacggc

SEQ ID NO: 225 - DNA, 29 bp, synthetic construct
  source 1..29: mol_type = other DNA
tcatgacccagtcgctgctggccttgacg

SEQ ID NO: 226 - DNA, 26 bp, synthetic construct
  source 1..26: mol_type = other DNA
accgacagctacggtgtcaccactgg

SEQ ID NO: 227 - DNA, 26 bp, synthetic construct
  source 1..26: mol_type = other DNA
accgtagctgtcggtgccgttcatca

SEQ ID NO: 228 - DNA, 28 bp, synthetic construct
  source 1..28: mol_type = other DNA
ctttggagccacctcccacaggaggacc

SEQ ID NO: 229 - DNA, 29 bp, synthetic construct
  source 1..29: mol_type = other DNA
gaggtggctccaaagccagtggtgacacc

SEQ ID NO: 230 - DNA, 32 bp, synthetic construct
  source 1..32: mol_type = other DNA
gagagctaattagacacctcaatgccggagtc

SEQ ID NO: 231 - DNA, 30 bp, synthetic construct
  source 1..30: mol_type = other DNA
gtctaattagctctctttggagagcaccac

SEQ ID NO: 232 - DNA, 26 bp, synthetic construct
  source 1..26: mol_type = other DNA
cggcacggccgtgggttctggtcttg

SEQ ID NO: 233 - DNA, 26 bp, synthetic construct
  source 1..26: mol_type = other DNA
cccacggccgtgccgttcaccatggc

SEQ ID NO: 234 - DNA, 26 bp, synthetic construct
  source 1..26: mol_type = other DNA
tggcctgccttccaacctggccggtg

SEQ ID NO: 235 - DNA, 27 bp, synthetic construct
  source 1..27: mol_type = other DNA
ttggaaggcaggccattgttgtagaag

SEQ ID NO: 236 - DNA, 26 bp, synthetic construct
  source 1..26: mol_type = other DNA
caacctgtccggtgggcgcaacccga

SEQ ID NO: 237 - DNA, 26 bp, synthetic construct
  source 1..26: mol_type = other DNA
ccaccggacaggttggaagtcaggcc

SEQ ID NO: 238 - DNA, 26 bp, synthetic construct
  source 1..26: mol_type = other DNA
tggccttatctcatccaggaagaccg

SEQ ID NO: 239 - DNA, 26 bp, synthetic construct
  source 1..26: mol_type = other DNA
gatgagataaggccaagcgagttgac

SEQ ID NO: 240 - DNA, 31 bp, synthetic construct
  source 1..31: mol_type = other DNA
ggagatagctatggtgtcaccactggcttcg

SEQ ID NO: 241 - DNA, 28 bp, synthetic construct
  source 1..28: mol_type = other DNA
accatagctatctccaccgttcgccacg

SEQ ID NO: 242 - DNA, 28 bp, synthetic construct
  source 1..28: mol_type = other DNA
accgacatatacggtgtcaccactggct

SEQ ID NO: 243 - DNA, 26 bp, synthetic construct
  source 1..26: mol_type = other DNA
accgtatatgtcggtgccgttcatca

SEQ ID NO: 244 - DNA, 29 bp, synthetic construct
  source 1..29: mol_type = other DNA
caccgacacctacggtgtcaccactggct

SEQ ID NO: 245 - DNA, 25 bp, synthetic construct
  source 1..25: mol_type = other DNA
ccgtaggtgtcggtgccgttcatca

SEQ ID NO: 246 - DNA, 28 bp, synthetic construct
  source 1..28: mol_type = other DNA
caccgacgtctacggtgtcaccactggc

SEQ ID NO: 247 - DNA, 28 bp, synthetic construct
  source 1..28: mol_type = other DNA
ccgtagacgtcggtgccgttcatcatgc

SEQ ID NO: 248 - DNA, 32 bp, synthetic construct
  source 1..32: mol_type = other DNA
tggagatgtctatggtgtcaccactggcttcg

SEQ ID NO: 249 - DNA, 27 bp, synthetic construct
  source 1..27: mol_type = other DNA
ccatagacatctccaccgttcgccacg

SEQ ID NO: 250 - DNA, 32 bp, synthetic construct
  source 1..32: mol_type = other DNA
tggagatacctatggtgtcaccactggcttcg

SEQ ID NO: 251 - DNA, 27 bp, synthetic construct
  source 1..27: mol_type = other DNA
ccataggtatctccaccgttcgccacg

SEQ ID NO: 252 - DNA, 34 bp, synthetic construct
  source 1..34: mol_type = other DNA
actgatatatatggtgttactactggttttggtg

SEQ ID NO: 253 - DNA, 33 bp, synthetic construct
  source 1..33: mol_type = other DNA
accatatatatcagtgcctttgttcatactctc

SEQ ID NO: 254 - DNA, 29 bp, synthetic construct
  source 1..29: mol_type = other DNA
tattagacaccttaacgccggaatattcg

SEQ ID NO: 255 - DNA, 33 bp, synthetic construct
  source 1..33: mol_type = other DNA
ttaaggtgtctaataagttccttctgaagtgcg

SEQ ID NO: 256 - DNA, 29 bp, synthetic construct
  source 1..29: mol_type = other DNA
catcgccgccatcggcaagctcatgtttg

SEQ ID NO: 257 - DNA, 27 bp, synthetic construct
  source 1..27: mol_type = other DNA
ccgatggcggcgatggcgaggcgggtg

SEQ ID NO: 258 - protein, 720 aa, synthetic construct
MECENGNVAAVAAVNGGNGLCLQKPQHADPLNWGKAAGELMGSHLEEVKRMVAEFRAPVVKIEGASLRIAQVAAVAAGEAAAAKVELDESSRGRVKASSDWVMSSMMNGTDIYGVTTGFGATSHRRTKEGGALQRELIRFLNAGVFGTGSDGHVLPAAATRAAMLVRINTLLQGYSGIRFEILEAITALLNAGVTPCLPLRGTITASGDLVPLSYIAGLITGRPNSVAVAPDGRKVDAAEAFKIAGIQHGFFELQPKEGLAMVNGTAVGSGLASTVLFEANILTILAEVLSAVFCEVMTGKPEYTDHLTHKLKHHPGQIEAAAIMEHILEGSSYMKLAKKLGDLDPLMKPKQDRYALRTSPQWLGPQIEVIRASTKSIEREINSVNDNPLIDVSRGKALHGGNFQGTPIGVSMDNTRLAIAAIGKLMFAQFSELVNDFYNNGLPSNLSGGRNPSLDYGFKGAEIAMASYCSELQFLANPVTNHVQSAEQHNQDVNSLGLISSRKTAEAVDILKLMSSTFLIALCQAIDLRHLEENLKSAVKNCVAQVAKKALTLNTVGDLHNARFSEKDLLTAIDREALFAYADDPCNPNYPLMQKLRAVLVEHALANGEAEHVATTSVFAKITKFEEELRATLPKEVEAARVAVENGTAPTPNRIKECRSYPLYRFVREELGTEYLTGEKLRSPGEECNKVFVAINQGKLIDPLLECLKEWNGEPLPIC

SEQ ID NO: 259 - protein, 720 aa, synthetic construct
MECENGNVAAVAAVNGGNGLCLQKPQHADPLNWGKAAGELMGSHLEEVKRMVAEFRAPVVKIEGASLRIAQVAAVAAGEAAAAKVELDESSRGRVKASSDWVMSSMMNGTDSYGVTTGFGATSHRRTKEGGALQRELIRHLNAGVFGTGSDGHVLPAAATRAAMLVRINTLLQGYSGIRFEILEAITALLNAGVTPCLPLRGTITASGDLVPLSYIAGLITGRPNSVAVAPDGRKVDAAEAFKIAGIQHGFFELQPKEGLAMVNGTAVGSGLASTVLFEANILTILAEVLSAVFCEVMTGKPEYTDHLTHKLKHHPGQIEAAAIMEHILEGSSYMKLAKKLGDLDPLMKPKQDRYALRTSPQWLGPQIEVIRASTKSIEREINSVNDNPLIDVSRGKALHGGNFQGTPIGVSMDNTRLAIAAIGKLMFAQFSELVNDFYNNGLPSNLSGGRNPSLDYGFKGAEIAMASYCSELQFLANPVTNHVQSAEQHNQDVNSLGLISSRKTAEAVDILKLMSSTFLIALCQAIDLRHLEENLKSAVKNCVAQVAKKALTLNTVGDLHNARFSEKDLLTAIDREALFAYADDPCNPNYPLMQKLRAVLVEHALANGEAEHVATTSVFAKITKFEEELRATLPKEVEAARVAVENGTAPTPNRIKECRSYPLYRFVREELGTEYLTGEKLRSPGEECNKVFVAINQGKLIDPLLECLKEWNGEPLPIC

SEQ ID NO: 260 - protein, 725 aa, synthetic construct
MEINGAHKSNGGGVDAMLCGGDIKTKNMVINAEDPLNWGAAAEQMKGSHLDEVKRMVAEFRKPVVNLGGETLTIGQVAAISTIGNSVKVELSETARAGVNASSDWVMESMNKGTDIYGVTTGFGATSHRRTKNGVALQKELIRFLNAGIFGSTKETSHTLPHSATRAAMLVRINTLLQGFSGIRFEILEAITSFLNNNITPSLPLRGTITASGDLVPLSYIAGLLTGRPNSKATGPNGEALTAEEAFKLAGISSGFFDLQPKEGLALVNGTAVGSGMASMVLFETNVLSVLAEILSAVFAEVMSGKPEFTDHLTHRLKHHPGQIEAAAIMEHILDGSSYMKLAQKLHEMDPLQKPKQDRYALRTSPQWLGPQIEVIRYATKSIEREINSVNDNPLIDVSRNKAIHGGNFQGTPIGVSMDNTRLAIAAIGKLMFAQFSELVNDFYNNGLPSNLTASRNPSLDYGFKGAEIAMASYCSELQYLANPVTSHVQSAEQHNQDVNSLGLISSRKTSEAVDILKLMSTTFLVAICQAVDLRHLEENLRQTVKNTVSQVAKKVLTTGVNGELHPSRFCEKDLLKVVDREQVYTYADDPCSATYPLIQKLRQVIVDHALINGESEKNAVTSIFHKIGAFEEELKAVLPKEVEAARAAYDNGTSAIPNRIKECRSYPLYRFVREELGTELLTGEKVTSPGEEFDKVFTAICEGKIIDPMMECLNEWNGAPIPIC

SEQ ID NO: 261 - protein, 725 aa, synthetic construct
MEINGAHKSNGGGVDAMLCGGDIKTKNMVINAEDPLNWGAAAEQMKGSHLDEVKRMVAEFRKPVVNLGGETLTIGQVAAISTIGNSVKVELSETARAGVNASSDWVMESMNKGTDSYGVTTGFGATSHRRTKNGVALQKELIRHLNAGIFGSTKETSHTLPHSATRAAMLVRINTLLQGFSGIRFEILEAITSFLNNNITPSLPLRGTITASGDLVPLSYIAGLLTGRPNSKATGPNGEALTAEEAFKLAGISSGFFDLQPKEGLALVNGTAVGSGMASMVLFETNVLSVLAEILSAVFAEVMSGKPEFTDHLTHRLKHHPGQIEAAAIMEHILDGSSYMKLAQKLHEMDPLQKPKQDRYALRTSPQWLGPQIEVIRYATKSIEREINSVNDNPLIDVSRNKAIHGGNFQGTPIGVSMDNTRLAIAAIGKLMFAQFSELVNDFYNNGLPSNLTASRNPSLDYGFKGAEIAMASYCSELQYLANPVTSHVQSAEQHNQDVNSLGLISSRKTSEAVDILKLMSTTFLVAICQAVDLRHLEENLRQTVKNTVSQVAKKVLTTGVNGELHPSRFCEKDLLKVVDREQVYTYADDPCSATYPLIQKLRQVIVDHALINGESEKNAVTSIFHKIGAFEEELKAVLPKEVEAARAAYDNGTSAIPNRIKECRSYPLYRFVREELGTELLTGEKVTSPGEEFDKVFTAICEGKIIDPMMECLNEWNGAPIPIC

SEQ ID NO: 262 - DNA, 2163 bp, synthetic construct
  source 1..2163: mol_type = other DNA
atggagtgcgagaacggcaatgttgcagctgttgccgccgtcaacggcggcaatggcctgtgcttgcagaagccgcagcacgccgaccccctgaactgggggaaggcggcgggggagctgatggggagccatcttgaggaggtgaagaggatggtggcggagttccgggcgccggtggtgaagatcgagggcgccagcctgaggatcgcgcaggtggcggccgttgcggccggcgaggcggccgcggccaaggtggagctcgatgagtcctcccgcggccgcgtcaaggccagcagcgactgggtcatgagcagcatgatgaacggcaccgacatatacggtgtcaccactggctttggagccacctcccacaggaggaccaaggagggtggtgctctccaaagagagctaattagattcctcaatgccggagtcttcggcaccggctccgacggccacgtgctgccggccgcggcaacaagggcggcaatgctcgtccgcatcaacaccctcctccagggctactctggaatccgcttcgagatccttgaggcgatcactgcgctgctcaacgccggcgtcacgccgtgcctgcctctccgtggcacgatcaccgcgtcgggagacctggtcccgctgtcctacattgccggcctcattaccggccgcccgaactccgtggcagtcgcccccgacggcaggaaggtggacgccgctgaggcgttcaagatcgctggcatccagcacggcttcttcgagctgcagccaaaggaaggtctggccatggtgaacggcacggccgtgggttctggtcttgcatccacagtgctcttcgaggcgaacattcttaccattcttgcggaggtcttgtcggcagtcttctgcgaagtcatgaccggcaagccggagtacaccgaccacctgacacacaagctgaagcaccaccccggacagatagaggccgcggccatcatggagcacatcttggaaggaagctcgtacatgaagctggcgaagaagctcggcgaccttgacccgctgatgaagccgaagcaggaccggtacgcactccgcacatcgccacagtggctcggcccccagatcgaggtcatccgtgcctccaccaagtcaattgagcgtgagatcaactccgtcaacgacaacccgctcatcgatgtctcccgtggcaaggcgctccacggcggcaacttccagggcactcccatcggtgtgtccatggacaatacccgcctcgccatcgcggccattggcaagctcatgtttgcgcagttctcagagctcgttaacgacttctacaacaatggcctgccttccaacctgtccggtgggcgcaacccgagcttggactatggtttcaagggtgccgagatcgccatggcctcatactgctctgagctccagttcttggccaacccagtgaccaaccatgtccagagcgctgagcagcacaaccaggatgtcaactcgcttggccttatctcatccaggaagaccgccgaggcagtggacatactgaagctcatgtcgtcgacgttcttgatcgcattgtgccaggccattgacctgcgccaccttgaggagaacctcaagagcgcagtcaagaactgcgtggcgcaggttgccaagaaggcgctgaccttgaacacagtcggggacctccacaacgcacgcttcagcgagaaggacctactcacagcgatcgaccgtgaggcgttgttcgcgtacgcggacgacccgtgcaaccccaactacccgctgatgcagaaactgcgcgcggtactcgtcgagcatgccctcgccaacggcgaggctgagcacgtggccaccacctccgtgtttgccaagatcaccaagttcgaggaggaactgcgtgcgacactgcccaaggaggtcgaggcagcccgggtggccgtggagaacggcaccgcgcctacaccgaaccggatcaaggaatgccggtcataccctctgtaccggttcgtgcgcgaggagcttgggacagagtacttgaccggggagaaactgcggtctcctggtgaggagtgcaacaaggtgtttgtggccatcaaccagggcaagctcattgacccactgctggagtgcctcaaggagtggaacggtgagcccctgccaatctgctga

SEQ ID NO: 263 - DNA, 2163 bp, synthetic construct
  source 1..2163: mol_type = other DNA
atggagtgcgagaacggcaatgttgcagctgttgccgccgtcaacggcggcaatggcctgtgcttgcagaagccgcagcacgccgaccccctgaactgggggaaggcggcgggggagctgatggggagccatcttgaggaggtgaagaggatggtggcggagttccgggcgccggtggtgaagatcgagggcgccagcctgaggatcgcgcaggtggcggccgttgcggccggcgaggcggccgcggccaaggtggagctcgatgagtcctcccgcggccgcgtcaaggccagcagcgactgggtcatgagcagcatgatgaacggcaccgacagctacggtgtcaccactggctttggagccacctcccacaggaggaccaaggagggtggtgctctccaaagagagctaattagacacctcaatgccggagtcttcggcaccggctccgacggccacgtgctgccggccgcggcaacaagggcggcaatgctcgtccgcatcaacaccctcctccagggctactctggaatccgcttcgagatccttgaggcgatcactgcgctgctcaacgccggcgtcacgccgtgcctgcctctccgtggcacgatcaccgcgtcgggagacctggtcccgctgtcctacattgccggcctcattaccggccgcccgaactccgtggcagtcgcccccgacggcaggaaggtggacgccgctgaggcgttcaagatcgctggcatccagcacggcttcttcgagctgcagccaaaggaaggtctggccatggtgaacggcacggccgtgggttctggtcttgcatccacagtgctcttcgaggcgaacattcttaccattcttgcggaggtcttgtcggcagtcttctgcgaagtcatgaccggcaagccggagtacaccgaccacctgacacacaagctgaagcaccaccccggacagatagaggccgcggccatcatggagcacatcttggaaggaagctcgtacatgaagctggcgaagaagctcggcgaccttgacccgctgatgaagccgaagcaggaccggtacgcactccgcacatcgccacagtggctcggcccccagatcgaggtcatccgtgcctccaccaagtcaattgagcgtgagatcaactccgtcaacgacaacccgctcatcgatgtctcccgtggcaaggcgctccacggcggcaacttccagggcactcccatcggtgtgtccatggacaatacccgcctcgccatcgcggccattggcaagctcatgtttgcgcagttctcagagctcgttaacgacttctacaacaatggcctgccttccaacctgtccggtgggcgcaacccgagcttggactatggtttcaagggtgccgagatcgccatggcctcatactgctctgagctccagttcttggccaacccagtgaccaaccatgtccagagcgctgagcagcacaaccaggatgtcaactcgcttggccttatctcatccaggaagaccgccgaggcagtggacatactgaagctcatgtcgtcgacgttcttgatcgcattgtgccaggccattgacctgcgccaccttgaggagaacctcaagagcgcagtcaagaactgcgtggcgcaggttgccaagaaggcgctgaccttgaacacagtcggggacctccacaacgcacgcttcagcgagaaggacctactcacagcgatcgaccgtgaggcgttgttcgcgtacgcggacgacccgtgcaaccccaactacccgctgatgcagaaactgcgcgcggtactcgtcgagcatgccctcgccaacggcgaggctgagcacgtggccaccacctccgtgtttgccaagatcaccaagttcgaggaggaactgcgtgcgacactgcccaaggaggtcgaggcagcccgggtggccgtggagaacggcaccgcgcctacaccgaaccggatcaaggaatgccggtcataccctctgtaccggttcgtgcgcgaggagcttgggacagagtacttgaccggggagaaactgcggtctcctggtgaggagtgcaacaaggtgtttgtggccatcaaccagggcaagctcattgacccactgctggagtgcctcaaggagtggaacggtgagcccctgccaatctgctga

SEQ ID NO: 264 - DNA, 2178 bp, synthetic construct
  source 1..2178: mol_type = other DNA
atggagattaacggggcacacaagagcaacggaggaggagtggacgctatgttatgcggcggagacatcaagacaaagaacatggtgatcaacgcggaggatcctctcaactggggagctgcagcggagcaaatgaaaggtagccatttggatgaagtgaagagaatggttgctgagtttaggaagccagttgtgaatcttggtggtgagactctgaccattggacaagtggctgcgatctcaactattggtaacagtgtgaaggtggagctatcggagacagctagagccggtgtgaatgctagtagtgattgggttatggagagtatgaacaaaggcactgatatatatggtgttactactggttttggtgctacttctcatcggagaaccaaaaacggtgtcgcacttcagaaggaacttattagattccttaacgccggaatattcggaagcacgaaagaaacaagccacacattgccacactccgccacaagagccgccatgcttgtacgaatcaacactctcctccaaggattttccggtatccgatttgagattctcgaagcaattaccagtttcctcaacaacaacatcactccatctctccccctccgtggtacaatcaccgcctccggagatctcgttcctctctcctacatcgccggacttctcaccggtcgtcccaattccaaagctactggtcccaacggtgaagctttaacagcagaggaagctttcaaattagcaggaatcagctccggattctttgatctccagcctaaggaaggtctcgcgctagtcaatggcacggcggttggatctggaatggcgtcaatggtgttattcgaaacgaatgttctctctgttttggctgagattttgtcggcggttttcgcagaggtgatgagtggtaagcctgagttcaccgatcatctcactcacagacttaaacatcatcccggtcaaatcgaagcggcggcgataatggagcatatcctcgacggaagctcgtacatgaaattagctcagaagcttcacgagatggatccgttacagaaacctaaacaagatcgttacgctcttcgtacttctcctcaatggttaggtcctcaaatcgaagtgatccgttacgcaacgaaatcgatcgagcgtgagattaactccgtcaacgataatccgttgatcgatgtttcgaggaacaaggcgattcacggtggtaacttccaaggaacaccaatcggagtttcaatggataacacgagattggcgatagcagcgattggtaaactcatgtttgctcaattctcagagcttgtgaatgatttctacaacaatggtttaccctcgaatctaaccgcttcgaggaatccaagtttggattatggattcaagggagctgagattgcaatggcttcttattgttcagagcttcaatacttagctaatcctgtgactagccatgttcaatcagcagagcaacataaccaagatgtcaactctttgggactaatctcgtctcgcaaaacttctgaagctgttgatattctcaagcttatgtcaacaacgttcctcgttgcgatttgtcaagctgtggatttgagacatttggaggagaatttgagacagactgtgaagaacactgtctctcaagtggcgaagaaagttcttactactggagtcaatggtgagcttcatccttctcgcttctgcgaaaaggatttactcaaagttgtagaccgtgaacaagtctacacatacgcggatgatccttgtagcgcaacgtacccgttgattcagaagctgagacaagttattgttgaccatgctttgatcaatggtgagagtgagaagaatgcagtgacttcaatcttccataagattggagctttcgaggaggagcttaaggcagtgctaccgaaagaagtggaagcagcaagagcagcctacgataacggaacatcggctatcccgaacaggatcaaggaatgtaggtcgtatccattgtatagattcgtgagggaagagcttggaacagagcttttgaccggagagaaagtgacgtcgcctggagaagagttcgacaaggttttcacggcgatttgtgaaggtaaaatcattgatccgatgatggaatgtctcaacgagtggaacggagctcccattccaatatgttaa

SEQ ID NO: 265 - DNA, 2178 bp, synthetic construct
  source 1..2178: mol_type = other DNA
atggagattaacggggcacacaagagcaacggaggaggagtggacgctatgttatgcggcggagacatcaagacaaagaacatggtgatcaacgcggaggatcctctcaactggggagctgcagcggagcaaatgaaaggtagccatttggatgaagtgaagagaatggttgctgagtttaggaagccagttgtgaatcttggtggtgagactctgaccattggacaagtggctgcgatctcaactattggtaacagtgtgaaggtggagctatcggagacagctagagccggtgtgaatgctagtagtgattgggttatggagagtatgaacaaaggcactgatagttatggtgttactactggttttggtgctacttctcatcggagaaccaaaaacggtgtcgcacttcagaaggaacttattagacaccttaacgccggaatattcggaagcacgaaagaaacaagccacacattgccacactccgccacaagagccgccatgcttgtacgaatcaacactctcctccaaggattttccggtatccgatttgagattctcgaagcaattaccagtttcctcaacaacaacatcactccatctctccccctccgtggtacaatcaccgcctccggagatctcgttcctctctcctacatcgccggacttctcaccggtcgtcccaattccaaagctactggtcccaacggtgaagctttaacagcagaggaagctttcaaattagcaggaatcagctccggattctttgatctccagcctaaggaaggtctcgcgctagtcaatggcacggcggttggatctggaatggcgtcaatggtgttattcgaaacgaatgttctctctgttttggctgagattttgtcggcggttttcgcagaggtgatgagtggtaagcctgagttcaccgatcatctcactcacagacttaaacatcatcccggtcaaatcgaagcggcggcgataatggagcatatcctcgacggaagctcgtacatgaaattagctcagaagcttcacgagatggatccgttacagaaacctaaacaagatcgttacgctcttcgtacttctcctcaatggttaggtcctcaaatcgaagtgatccgttacgcaacgaaatcgatcgagcgtgagattaactccgtcaacgataatccgttgatcgatgtttcgaggaacaaggcgattcacggtggtaacttccaaggaacaccaatcggagtttcaatggataacacgagattggcgatagcagcgattggtaaactcatgtttgctcaattctcagagcttgtgaatgatttctacaacaatggtttaccctcgaatctaaccgcttcgaggaatccaagtttggattatggattcaagggagctgagattgcaatggcttcttattgttcagagcttcaatacttagctaatcctgtgactagccatgttcaatcagcagagcaacataaccaagatgtcaactctttgggactaatctcgtctcgcaaaacttctgaagctgttgatattctcaagcttatgtcaacaacgttcctcgttgcgatttgtcaagctgtggatttgagacatttggaggagaatttgagacagactgtgaagaacactgtctctcaagtggcgaagaaagttcttactactggagtcaatggtgagcttcatccttctcgcttctgcgaaaaggatttactcaaagttgtagaccgtgaacaagtctacacatacgcggatgatccttgtagcgcaacgtacccgttgattcagaagctgagacaagttattgttgaccatgctttgatcaatggtgagagtgagaagaatgcagtgacttcaatcttccataagattggagctttcgaggaggagcttaaggcagtgctaccgaaagaagtggaagcagcaagagcagcctacgataacggaacatcggctatcccgaacaggatcaaggaatgtaggtcgtatccattgtatagattcgtgagggaagagcttggaacagagcttttgaccggagagaaagtgacgtcgcctggagaagagttcgacaaggttttcacggcgatttgtgaaggtaaaatcattgatccgatgatggaatgtctcaacgagtggaacggagctcccattccaatatgttaa